(19) United States
(12) Patent Application Publication
(10) Pub. No.: US 2021/0139484 A1
(43) Pub. Date: May 13, 2021

(54) DIHYDROISOQUINOLINE-2(1H)-CARBOXAMIDE AND RELATED COMPOUNDS AND THEIR USE IN TREATING MEDICAL CONDITIONS

(71) Applicant: GOSSAMER BIO SERVICES, INC., San Diego, CA (US)

(72) Inventors: Thomas Daniel Aicher, Ann Arbor, MI (US); Donald J. Skalitzky, Saline, MI (US); Peter L. Toogood, Ann Arbor, MI (US); Chad A. Vanhuis, Hartland, MI (US)

(21) Appl. No.: 17/046,757

(22) PCT Filed: Apr. 11, 2019
(86) PCT No.: PCT/US2019/027017

§ 371 Date: Oct. 9, 2020

Related U.S. Application Data
(60) Provisional application No. 62/656,167, filed on Apr. 11, 2018.

(51) Int. Cl. C07D 487/04; C07D 401/12; C07D 471/04; C07D 401/14; C07D 519/00; C07F 7/08

(52) U.S. Cl. CPC C07D 487/04; C07D 401/12; C07F 7/0812; C07D 401/14; C07D 519/00; C07D 471/04

(57) ABSTRACT

The invention provides dihydroisoquinoline-2(1H)-carboxamide and related compounds, pharmaceutical compositions, and their use in the treatment of medical conditions, such as cancer, and in inhibiting HPK1 activity.

### CROSS-REFERENCE TO RELATED APPLICATIONS

This application claims the benefit of and priority to U.S. Provisional Patent Application Ser. No. 62/656,167, filed Apr. 11, 2018; the contents of which are hereby incorporated by reference.

### FIELD OF THE INVENTION

The invention provides dihydroisoquinoline-2(1H)-carboxamide and related compounds, pharmaceutical compositions, and their use in the treatment of medical conditions, such as cancer, and in inhibiting HPK1 activity.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. New therapies are needed to address this unmet need in cancer therapy.

Hematopoietic Progenitor Kinase 1 (HPK1) is a hematopoietic cell-restricted serine/threonine kinase, where inhibitory activity towards HPK1 has been reported as a therapeutic approach in cancer immunotherapy (see, for example, Sawasdikosol et al. in Immunol. Res. (2012) 54(1-3): 262-265; and international patent application publication WO 2016/205942). Signals generated by cell surface receptors on hematopoietic cells can induce HPK1 kinase activity. Such signals can be generated by ligand engagement, B-cell antigen receptor (see, e.g., Liou et al. in Immunity (2000) 12: 399), transforming growth factor β receptor (see, e.g., Wang et al. in J. Biol. Chem. (1997) 272: 22771); and Zhou et al. in J. Biol. Chem. (1999) 274: 13133), erythropoietin receptor (see, e.g., Nagata et al. in Blood (1999) 93: 3347), and Fas (see, e.g., Chen et al. in Oncogene (1999) 18: 7370). Compounds having inhibitory activity towards HPK1 are needed as therapeutic agents for the treatment of cancer.

The present invention addresses this need and provides other related advantages.

### SUMMARY

The invention provides dihydroisoquinoline-2(1H)-carboxamide and related compounds, pharmaceutical compositions, and their use in the treatment of medical conditions, such as cancer, and in inhibiting HPK1 activity. In particular, one aspect of the invention provides a collection of dihydroisoquinoline-2(1H)-carboxamide and related compounds, such as a compound represented by Formula I:

[chemical structure]

or a pharmaceutically acceptable salt thereof, where the variables are as defined in the detailed description. Another aspect of the invention provides a collection of 1,2,3,4-tetrahydroquinoline-2-carboxamide and related compounds represented by Formula II:

[chemical structure]

or a pharmaceutically acceptable salt thereof, where the variables are as defined in the detailed description. Further description of additional collections of dihydroisoquinoline-2(1H)-carboxamide, 1,2,3,4-tetrahydroquinoline-2-carboxamide, and related compounds are described in the detailed description. The compounds may be part of a pharmaceutical composition comprising a pharmaceutically acceptable carrier.

Another aspect of the invention provides a method of treating cancer in a subject. The method comprises administering a therapeutically effective amount of a compound described herein, such as a compound of Formula I, I-A, I-A1, II, II-A, or II-A1, to a subject in need thereof to treat the cancer. In certain embodiments, the cancer is a solid tumor, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. The compound may be used as monotherapy or as part of a combination therapy, to treat the cancer.

Another aspect of the invention provides a method of inhibiting the activity of HPK1. The method comprises exposing a HPK1 to an effective amount of a compound described herein, such as a compound of Formula I, I-A, I-A1, II, II-A, or II-A1, to inhibit the activity of said HPK1.

### DETAILED DESCRIPTION

The invention provides dihydroisoquinoline-2(1H)-carboxamide and related compounds, pharmaceutical compositions, and their use in the treatment of medical conditions, such as cancer, and in inhibiting HPK1 activity. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “halocycloalkyl” refers to a cycloalkyl group that is substituted with at least one halogen.

The term “cycloalkylene” refers to a diradical of a cycloalkyl group. Exemplary cycloalkylene groups include

[chemical structure]

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “aralkyl” refers to an alkyl group substituted with an aryl group. Exemplary aralkyl groups include

[chemical structure]

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containing at least one C—C double bond. In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “heteroaryl” is art-recognized and refers to aromatic groups that include at least one ring heteroatom. In certain instances, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocylic ring or a 9-10 membered bicyclic ring.

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

As used herein, the terms “heterocyclic” and “heterocyclyl” represent, for example, an aromatic or nonaromatic ring (e.g., a monocyclic or bicyclic ring) containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the heterocyclyl group is a 3-7 membered ring that, unless specified otherwise, is substituted or unsubstituted. In certain embodiments, the heterocyclyl group is a 3-7 membered ring that contains 1, 2, or 3 ring heteroatoms selected from oxygen, sulfur, and nitrogen. The term “aza-heterocyclyl” refers to a heterocyclyl group having at least one nitrogen atom in the heterocyclyl ring.

The term “heterocycloalkyl” refers to a saturated heterocyclyl group having, for example, 3-7 ring atoms selected from carbon and heteroatoms (e.g., O, N, or S). The term “aza-heterocycloalkyl” refers to a saturated heterocyclyl group having at least one nitrogen atom in the heterocycloalkyl ring.

The terms “amine” and “amino” are art-recognized and refer to both unsubstituted and substituted amines, e.g., a moiety that may be represented by the general formulas:

[chemical structure]

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “oxo” is art-recognized and refers to a “═O” substituent. For example, a cyclopentane substituted with an oxo group is cyclopentanone.

The symbol “ [symbol] ” indicates a point of attachment.

The term “substituted” means that one or more hydrogens on the atoms of the designated group are replaced with a selection from the indicated group, provided that the atoms' normal valencies under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

The abbreviation “THF” is art-recognized and refers to tetrahydrofuran. The abbreviation “DCM” is art-recognized and refers to dichloromethane. The abbreviation “DMF” is art-recognized and refers to dimethylformamide. The abbreviation “DMA” is art-recognized and refers to dimethylacetamide. The abbreviation “EDTA” is art-recognized and refers to ethylenediaminetetraacetic acid. The abbreviation “TFA” is art-recognized and refers to trifluoroacetic acid. The abbreviation “Ts” is art-recognized and refers to tosylate. The abbreviation “TBS” is art-recognized and refers to tert-butyldimethylsilyl. The abbreviation “DMSO” is art-recognized and refers to dimethylsulfoxide. The abbreviation “Tf” is art-recognized and refers to triflate, or trifluoromethylsulfonate. The abbreviation “Pin” is art-recognized and refers to pinacolato.

As a general matter, compositions specifying a percentage are by weight unless otherwise specified.

### I. Dihydroisoquinoline-2(1H)-carboxamide and Related Compounds

The invention provides dihydroisoquinoline-2(1H)-carboxamide and related compounds. The compounds may be used in the pharmaceutical compositions and therapeutic methods described herein. Exemplary compounds are described in the following sections, along with exemplary procedures for making the compounds. Additional exemplary compounds and synthetic procedures are described in the Examples.

One aspect of the invention provides a compound represented by Formula I:

[chemical structure]

or a pharmaceutically acceptable salt thereof; wherein:

R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);

R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, —(C₁₋₄ alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;

R³ represents independently for each occurrence hydrogen, C₁₋₄ alkyl, or C₃₋₅ cycloalkyl;

R⁴ represents independently for each occurrence halogen, C₁₋₄ alkyl, C₁₋₄ haloalkyl, cyano, C₁₋₄ alkoxyl, C₁₋₄ haloalkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring optionally substituted with 1 or 2 substituents independently selected from the group consisting of halogen, hydroxyl, C₁₋₄ alkyl, C₁₋₄ haloalkyl, cyano, and C₁₋₄ alkoxyl;

A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, cyano, C₁₋₄ alkoxyl, —(C₁₋₄ alkylene)-(C₁₋₄ alkoxyl), —(C₁₋₄ alkylene)-S(O)ₜ—(C₁₋₄ alkyl), —C(O)N(R³)(R⁵), —C(O)OR³, —C(O)R³, —N(R³)(R⁵), —N(R³)C(O)R⁵, —N(R³)CO₂(C₁₋₄ alkyl), —N(R³)C(O)-(5-6 membered heteroaryl), —(C₁₋₄ alkylene)-N(R³)(R⁵), —(C₁₋₄ alkylene)-N(R³)C(O)R⁵, —N(R³)-(5-6 membered heteroaryl), 3-7 membered heterocyclyl, 5-6 membered unsaturated oxo-heterocyclyl, and aryl; wherein said C₃₋₇ cycloalkyl, C₅₋₇ cycloalkenyl, 3-7 membered heterocyclyl, 5-6 membered heteroaryl, 5-6 membered unsaturated oxo-heterocyclyl, and aryl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), cyano, —N(R³)(R⁵), —C(O)R³ and —C(O)N(R³)R⁵;

A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;

X¹ is —O—, —N(R³)—, —S(O)ₜ—, or a bond;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, oxo, cyano, C₁₋₄ alkoxyl, —(C₁₋₄ alkylene)-(C₁₋₄ alkoxyl), C₃₋₇ cycloalkyl, 3-7 membered heterocycloalkyl, and 5-6 membered heteroaryl;

m and n are each independently 0, 1, 2, or 3; and

t represents independently for each occurrence 0, 1 or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

In certain embodiments, A¹ is a 5-6 membered heteroaromatic ring containing at least one ring nitrogen atom. In certain embodiments, A¹ is

[chemical structure]

In certain embodiments, A¹ is a 6-membered unsaturated oxo-heterocyclic ring containing at least one ring nitrogen atom.

In certain embodiments, A¹ is a 6-membered carbocyclic aromatic ring.

In certain embodiments, A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano. In certain embodiments, A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, —(C₁₋₄ alkylene)-(C₁₋₄ alkoxyl), C₃₋₇ cycloalkyl, 3-7 membered heterocycloalkyl, 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, cyano. In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is

[chemical structure]

optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is

[chemical structure]

optionally substituted with 1 substituent selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is a 5-10 membered heteroaryl optionally substituted with 1, 2, or 3 substituents independently selected from the group consisting of halogen, hydroxyl, C₁₋₄ alkyl, C₂₋₄ alkenyl, haloalkyl, hydroxyalkyl, C₃₋₄ hydroxyalkenyl, C₃₋₇ cycloalkyl, C₅₋₇ cycloalkenyl, cyano, C₁₋₄ alkoxyl, —(C₁₋₄ alkylene)-(C₁₋₄ alkoxyl), —(C₁₋₄ alkylene)-S(O)ₜ—(C₁₋₄ alkyl), —C(O)N(R³)(R⁵), —C(O)OR³, —C(O)R³, —N(R³)(R⁵), —N(R³)C(O)R⁵, —N(R³)CO₂(C₁₋₄ alkyl), —N(R³)C(O)-(5-6 membered heteroaryl), —(C₁₋₄ alkylene)-N(R³)(R⁵), —(C₁₋₄ alkylene)-N(R³)C(O)R⁵, —N(R³)-(5-6 membered heteroaryl), 3-7 membered heterocyclyl, 5-6 membered unsaturated oxo-heterocyclyl, and aryl; wherein said C₃₋₇ cycloalkyl, C₅₋₇ cycloalkenyl, 3-7 membered heterocyclyl, 5-6 membered heteroaryl, 5-6 membered unsaturated oxo-heterocyclyl, and aryl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), cyano, —N(R³)(R⁵), —C(O)R³ and —C(O)N(R³)R⁵.

In certain embodiments, A² is a 5-10 membered heteroaryl optionally substituted with 1, 2, or 3 substituents independently selected from the group consisting of halogen, hydroxyl, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, cyano, C₁₋₄ alkoxyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, hydroxyalkyl, —(C₁₋₄ alkylene)-N(R³)(R⁵), and —N(R³)(R⁵).

In certain embodiments, A² is a 5-10 membered heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is a 8-10 membered bicyclic heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is one of the following:

[chemical structure]

substituted with 1, 2, or 3 substituents independently selected from the group consisting of halogen, hydroxyl, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, cyano, C₁₋₄ alkoxyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), and —N(R³)(R⁵).

In certain embodiments, A² is one of the following:

[chemical structure]

substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is

[chemical structure]

substituted with 1, 2, or 3 substituents independently selected from the group consisting of halogen, hydroxyl, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, cyano, C₁₋₄ alkoxyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), and —N(R³)(R⁵). In certain embodiments, A² is

[chemical structure]

substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵). In certain embodiments, A² is

[chemical structure]

wherein R* is halogen or C₁₋₄ alkyl, and R** is C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, or 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), and —N(R³)(R⁵).

In certain embodiments, A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is 8-10 membered bicyclic oxo-heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴. In certain embodiments, A³ is phenyl substituted by (i) —(C₁₋₄ alkylene)-X² or —O—X², and (ii) 1 or 2 occurrences of R⁴. In certain embodiments, A³ is phenyl substituted by (i) —(C₁₋₄ alkylene)-X², and (ii) 1 or 2 occurrences of R⁴.

In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴.

In certain embodiments, X¹ is —O—. In certain embodiments, X¹ is —N(R³)— or —S—. In certain embodiments, X¹ is —S(O)—. In certain embodiments, X¹ is —S(O)₂—.

In certain embodiments, t is 0. In certain embodiments, t is 1. In certain embodiments, t is 2.

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R² is C₁₋₄ alkyl. In certain embodiments, R² is ethyl. In certain embodiments, R² is C₁₋₄ alkyl. In certain embodiments, R² is C₁₋₄ alkyl, and m is 1 or 2.

In certain embodiments, R³ is hydrogen.

In certain embodiments, R⁴ represents independently for each occurrence halogen or C₁₋₄ haloalkyl. In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, m is 1. In certain embodiments, m is 2.

In certain embodiments, n is 0. In certain embodiments, n is 1.

The description above describes multiple embodiments relating to compounds of Formula I. The patent application specifically contemplates all combinations of the embodiments.

Another aspect of the invention provides a compound represented by Formula I-i:

[chemical structure]

or a pharmaceutically acceptable salt thereof; wherein:

R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);

R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, —(C₁₋₄ alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;

R³ represents independently for each occurrence hydrogen or C₁₋₄ alkyl;

R⁴ represents independently for each occurrence halogen, C₁₋₄ alkyl, C₁₋₄ haloalkyl, cyano, C₁₋₄ alkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵;

A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;

X¹ is —O—, —N(R³)—, or —S(O)ₜ—;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, oxo, cyano, C₁₋₄ alkoxyl, —(C₁₋₄ alkylene)-(C₁₋₄ alkoxyl), C₃₋₇ cycloalkyl, 3-7 membered heterocycloalkyl, and 5-6 membered heteroaryl;

m and n are each independently 0, 1, 2, or 3; and

t is 0, 1 or 2.

The definitions of variables in Formula I-i above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-i.

In certain embodiments, A¹ is a 5-6 membered heteroaromatic ring containing at least one ring nitrogen atom. In certain embodiments, A¹ is

[chemical structure]

In certain embodiments, A¹ is a 6-membered unsaturated oxo-heterocyclic ring containing at least one ring nitrogen atom.

In certain embodiments, A¹ is a 6-membered carbocyclic aromatic ring.

In certain embodiments, A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano. In certain embodiments, A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, —(C₁₋₄ alkylene)-(C₁₋₄ alkoxyl), C₃₋₇ cycloalkyl, 3-7 membered heterocycloalkyl, 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, cyano. In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is 8-10 membered bicyclic oxo-heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴. In certain embodiments, A³ is phenyl substituted by (i) —(C₁₋₄ alkylene)-X² or —O—X², and (ii) 1 or 2 occurrences of R⁴.

In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴.

In certain embodiments, X¹ is —O—. In certain embodiments, X¹ is —N(R³)— or —S—. In certain embodiments, X¹ is —S(O)—. In certain embodiments, X¹ is —S(O)₂—.

In certain embodiments, t is 0. In certain embodiments, t is 1. In certain embodiments, t is 2.

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R² is C₁₋₄ alkyl. In certain embodiments, R² is ethyl. In certain embodiments, R² is C₁₋₄ alkyl. In certain embodiments, R² is C₁₋₄ alkyl, and m is 1 or 2.

In certain embodiments, R³ is hydrogen.

In certain embodiments, R⁴ represents independently for each occurrence halogen or C₁₋₄ haloalkyl. In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, m is 1. In certain embodiments, m is 2.

In certain embodiments, n is 0. In certain embodiments, n is 1.

The description above describes multiple embodiments relating to compounds of Formula I-i. The patent application specifically contemplates all combinations of the embodiments.

In a more specific embodiment, the invention provides collections of compounds defined by the following embodiments in connection with Formula I-i:

Embodiment No. 1. A compound represented by Formula I-i:

[chemical structure]

or a pharmaceutically acceptable salt thereof; wherein:

R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);

R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, —(C₁₋₄ alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;

R³ represents independently for each occurrence hydrogen or C₁₋₄ alkyl;

R⁴ represents independently for each occurrence halogen, C₁₋₄ alkyl, C₁₋₄ haloalkyl, cyano, C₁₋₄ alkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵;

A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;

X¹ is —O—, —N(R³)—, or —S(O)ₜ—;

X² is a 3-10 membered aza-heterocycloalkyl optionally substituted with 1, 2, or 3 substituents independently selected from the group consisting of halogen, C₁₋₄ alkyl, C₁₋₄ haloalkyl, hydroxyalkyl, hydroxyl, oxo, cyano, C₁₋₄ alkoxyl, alkylene)-(C₁₋₄ alkoxyl), C₃₋₇ cycloalkyl, 3-7 membered heterocycloalkyl, and 5-6 membered heteroaryl;

m and n are each independently 0, 1, 2, or 3; and

t is 0, 1 or 2.

Embodiment no. 2. The compound of embodiment 1, wherein A¹ is a 5-6 membered heteroaromatic ring containing at least one ring nitrogen atom.
Embodiment no. 3. The compound of embodiment 1, wherein A¹ is

[chemical structure]

Embodiment no. 4. The compound of embodiment 1, wherein A¹ is a 6-membered unsaturated oxo-heterocyclic ring containing at least one ring nitrogen atom.
Embodiment no. 5. The compound of embodiment 1, wherein A¹ is a 6-membered carbocyclic aromatic ring.
Embodiment no. 6. The compound of any one of embodiments 1-5, wherein A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, alkoxyl, alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)— (5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.
Embodiment no. 7. The compound of any one of embodiments 1-5, wherein A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, alkoxyl, and —(C₁₋₄ alkylene)-(C₁₋₄ alkoxyl).
Embodiment no. 8. The compound of any one of embodiments 1-5, wherein A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).
Embodiment no. 9. The compound of any one of embodiments 1-5, wherein A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.
Embodiment no. 10. The compound of any one of embodiments 1-5, wherein A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.
Embodiment no. 11. The compound of any one of embodiments 1-10, wherein A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.
Embodiment no. 12. The compound of any one of embodiments 1-10, wherein A³ is phenyl substituted by (i) —(C₁₋₄ alkylene)-X² or —O—X², and (ii) 1 or 2 occurrences of R⁴.
Embodiment no. 13. The compound of any one of embodiments 1-10, wherein A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.
Embodiment no. 14. The compound of any one of embodiments 1-10, wherein A³ is pyridinyl, pyrazinyl, or pyrimidinyl; each of which is substituted by (i) —(C₁₋₄ alkylene)-X² or —O—X², and (ii) 1 occurrence of R⁴.
Embodiment no. 15. The compound of any one of embodiments 1-14, wherein X¹ is —O—.
Embodiment no. 16. The compound of any one of embodiments 1-14, wherein X¹ is —N(R³)— or —S—.
Embodiment no. 17. The compound of any one of embodiments 1-16, wherein R² is C₁₋₄ alkyl, and m is 1 or 2.
Embodiment no. 18. The compound of any one of embodiments 1-17, wherein X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl.
Embodiment no. 19. The compound of any one of embodiments 1-17, wherein X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl.
Embodiment no. 20. The compound of any one of embodiments 1-17, wherein X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl.
Embodiment no. 21. The compound of any one of embodiments 1-17, wherein X² is piperidinyl or piperazinyl; each of which is optionally substituted with 1 or 2 substituents independently selected from the group consisting of C₁₋₄ alkyl and C₃₋₇ cycloalkyl.
Embodiment no. 22. The compound of any one of embodiments 1-21, wherein R³ is hydrogen.
Embodiment no. 23. The compound of any one of embodiments 1-22, wherein R⁴ is C₁₋₄ haloalkyl.
Embodiment no. 24. The compound of any one of embodiments 1-22, wherein R⁴ is trifluoromethyl.
Embodiment no. 25. The compound of any one of embodiments 1-24, wherein R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl).
Embodiment no. 26. The compound of any one of embodiments 1-25, wherein n is 0.
Embodiment no. 27. The compound of any one of embodiments 1-25, wherein n is 1.

Another aspect of the invention provides a compound represented by Formula I-x:

[chemical structure]

or a pharmaceutically acceptable salt thereof; wherein:

R₁ represents independently for each occurrence halogen or C₁₋₄ alkyl;

R^{2A}, R^{2B}, and R^{2C} each represent independently hydrogen, C₁₋₄ alkyl, C₁₋₄ haloalkyl, C₁₋₄ hydroxyalkyl, or —(C₁₋₄ alkylene)-(C₁₋₄ alkoxyl);

R³ represents independently for each occurrence hydrogen or C₁₋₄ alkyl;

R⁴ represents independently for each occurrence halogen, C₁₋₄ alkyl, C₁₋₄ haloalkyl, C₃₋₅ cycloalkyl, C₃₋₅ halocycloalkyl, -(cyclopropylene)-(C₁₋₄ haloalkyl), or -(cyclopropylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 6-membered heteroaromatic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is substituted with 1, 2, or 3 substituents independently selected from the group consisting of halogen, C₁₋₄ alkyl, C₂₋₄ alkenyl, C₁₋₄ haloalkyl, 4 hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, alkylene)-(C₁₋₄ alkoxyl), and C₃₋₇ cycloalkyl; and

n is 0, 1, or 2.

The definitions of variables in Formula I-x above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-x.

In certain embodiments, Y¹ is N. In certain embodiments, Y¹ is C(H).

In certain embodiments, A² is a 5-10 membered heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is a 8-10 membered bicyclic heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is

[chemical structure]

substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is

[chemical structure]

wherein R* is halogen or C₁₋₄ alkyl, and R** is C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, or 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), and —N(R³)(R⁵).

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{2C} is hydrogen. In certain embodiments, R^{2A} is ethyl; and R^{2C} is hydrogen. In certain embodiments, R^{2A} is methyl; and R^{2C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ represents independently for each occurrence halogen or C₁₋₄ haloalkyl. In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, n is 0. In certain embodiments, n is 1.

The description above describes multiple embodiments relating to compounds of Formula I-x. The patent application specifically contemplates all combinations of the embodiments.

Another aspect of the invention provides a compound represented by Formula I-A:

[chemical structure]

or a pharmaceutically acceptable salt thereof; wherein:

R¹ represents independently for each occurrence halogen or C₁₋₄ alkyl;

R^{2A}, R^{2B}, and R^{2C} each represent independently hydrogen, C₁₋₄ alkyl, C₁₋₄ haloalkyl, C₁₋₄ hydroxyalkyl, or —(C₁₋₄ alkylene)-(C₁₋₄ alkoxyl);

R³ represents independently for each occurrence hydrogen or C₁₋₄ alkyl;

R⁴ represents independently for each occurrence C₁₋₄ alkyl, C₁₋₄ haloalkyl, C₃₋₅ cycloalkyl, C₃₋₅ halocycloalkyl, -(cyclopropylene)-(C₁₋₄ haloalkyl), or -(cyclopropylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 6-membered heteroaromatic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl; and

n is 0, 1, or 2.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

In certain embodiments, A¹ is a 6-membered heteroaromatic ring containing at least one ring nitrogen atom. In certain embodiments, A¹ is

[chemical structure]

In certain embodiments, A¹ is a 6-membered carbocyclic aromatic ring.

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is phenyl substituted by (i) —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{5C} is hydrogen. In certain embodiments, R^{2A} is ethyl; and R^{5C} is hydrogen. In certain embodiments, R^{2A} is methyl; and R^{5C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, n is 0. In certain embodiments, n is 1.

The description above describes multiple embodiments relating to compounds of Formula I-A. The patent application specifically contemplates all combinations of the embodiments.

Another aspect of the invention provides a compound represented by Formula I-AA:

[chemical structure]

or a pharmaceutically acceptable salt thereof; wherein:

R¹ represents independently for each occurrence halogen or C₁₋₄ alkyl;

R^{2A}, R^{2B}, and R^{2C} each represent independently hydrogen, C₁₋₄ alkyl or C₁₋₄ haloalkyl;

R³ represents independently for each occurrence hydrogen or C₁₋₄ alkyl;

R⁴ represents independently for each occurrence C₁₋₄ alkyl or C₁₋₄ haloalkyl;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, alkylene)-(C₁₋₄ alkoxyl), and C₃₋₇ cycloalkyl;

Y¹ is N, C(H), or C(R¹); and

n is 0 or 1.

The definitions of variables in Formula I-AA above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-AA.

In certain embodiments, A¹ is a 6-membered heteroaromatic ring containing at least one ring nitrogen atom. In certain embodiments, A¹ is

[chemical structure]

In certain embodiments, A¹ is a 6-membered carbocyclic aromatic ring.

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, N(R³)(R⁵), C₁₋₄ alkoxyl, and —(C₁₋₄ alkylene)-(C₁₋₄ alkoxyl).

In certain embodiments, A² is 8-10 membered bicyclic heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is phenyl substituted by (i) —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{2C} is hydrogen. In certain embodiments, R^{2A} is ethyl; and R^{2C} is hydrogen. In certain embodiments, R^{2A} is methyl; and R^{2C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl. In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl).

In certain embodiments, n is 0. In certain embodiments, n is 1.

The description above describes multiple embodiments relating to compounds of Formula I-AA. The patent application specifically contemplates all combinations of the embodiments.

Another aspect of the invention provides a compound represented by Formula I-A*:

[chemical structure]

or a pharmaceutically acceptable salt thereof; wherein:

R¹ represents independently for each occurrence halogen or C₁₋₄ alkyl;

R^{2A}, R^{2B}, and R^{2C} each represent independently hydrogen, C₁₋₄ alkyl or C₁₋₄ haloalkyl; or R^{2A} and R^{2B} are taken together with the carbon atoms to which they are attached to form a 3-5 membered saturated carbocyclic ring;

R³ represents independently for each occurrence hydrogen or C₁₋₄ alkyl;

R⁴ represents independently for each occurrence C₁₋₄ alkyl or C₁₋₄ haloalkyl;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;

Y¹ is N, C(H), or C(R¹); and

n is 0 or 1.

The definitions of variables in Formula I-A* above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A*.

In certain embodiments, A¹ is a 6-membered heteroaromatic ring containing at least one ring nitrogen atom. In certain embodiments, A¹ is

[chemical structure]

In certain embodiments, A¹ is a 6-membered carbocyclic aromatic ring.

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is 8-10 membered bicyclic heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is phenyl substituted by (i) —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{2C} is hydrogen. In certain embodiments, R^{2A} is ethyl; and R^{2C} is hydrogen. In certain embodiments, R^{2A} is methyl; and R^{2C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl. In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl).

In certain embodiments, n is 0. In certain embodiments, n is 1.

The description above describes multiple embodiments relating to compounds of Formula I-A*. The patent application specifically contemplates all combinations of the embodiments.

Another aspect of the invention provides a compound represented by Formula I-A1:

[chemical structure]

or a pharmaceutically acceptable salt thereof; wherein:

R¹ represents independently for each occurrence halogen or C₁₋₄ alkyl;

R^{2A}, R^{2B}, and R^{2C} each represent independently hydrogen, C₁₋₄ alkyl or C₁₋₄ haloalkyl;

R³ represents independently for each occurrence hydrogen or C₁₋₄ alkyl;

R⁴ represents independently for each occurrence C₁₋₄ alkyl or C₁₋₄ haloalkyl;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;

Y¹ is N, C(H), or C(R¹); and

n is 0 or 1.

The definitions of variables in Formula I-A1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A1.

In certain embodiments, Y¹ is N. In certain embodiments, Y¹ is C(H).

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is phenyl substituted by (i) —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{2C} is hydrogen. In certain embodiments, R^{2A} is ethyl; and R^{2C} is hydrogen. In certain embodiments, R^{2A} is methyl; and R^{2C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, n is 0. In certain embodiments, n is 1.

The description above describes multiple embodiments relating to compounds of Formula I-A1. The patent application specifically contemplates all combinations of the embodiments.

Another aspect of the invention provides a compound represented by Formula I-A2:

[chemical structure]

or a pharmaceutically acceptable salt thereof; wherein:

R¹ represents independently for each occurrence halogen or C₁₋₄ alkyl;

R^{2A}, R^{2B}, and R^{2C} each represent independently hydrogen, C₁₋₄ alkyl or C₁₋₄ haloalkyl;

R³ represents independently for each occurrence hydrogen or C₁₋₄ alkyl;

R⁴ represents independently for each occurrence halogen, C₁₋₄ alkyl, or C₁₋₄ haloalkyl;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring optionally substituted with 1 or 2 substituents independently selected from the group consisting of halogen, hydroxyl, C₁₋₄ alkyl, C₁₋₄ haloalkyl, cyano, and C₁₋₄ alkoxyl;

A² is one of the following:
5-10 membered heteroaryl, or
8-10 membered partially unsaturated bicyclic heterocyclyl;
each of which is substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵);

A³ is one of the following:
phenyl substituted by (i) —(C₁₋₄ alkylene)-X² and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;

Y¹ is N, C(H), or C(R¹); and

n is 0 or 1.

The definitions of variables in Formula I-A2 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A2.

In certain embodiments, Y¹ is N. In certain embodiments, Y¹ is C(H).

In certain embodiments, A² is a 5-10 membered heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is a 8-10 membered bicyclic heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is

[chemical structure]

substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

In certain embodiments, A² is

[chemical structure]

wherein R* is halogen or C₁₋₄ alkyl, and R** is C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, or 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), and —N(R³)(R⁵).

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl; and R^{2C} is hydrogen. In certain embodiments, R^{2A} is ethyl; and R^{2C} is hydrogen. In certain embodiments, R^{2A} is methyl; and R^{2C} is hydrogen. In certain embodiments, R^{2B} is hydrogen. In certain embodiments, R^{2B} is C₁₋₄ alkyl.

In certain embodiments, R³ is hydrogen.

In certain embodiments, R⁴ represents independently for each occurrence halogen or C₁₋₄ haloalkyl. In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl). In certain embodiments, R⁵ is hydrogen, C₁₋₄ alkyl, C₁₋₄ haloalkyl, or C₁₋₄ hydroxyalkyl.

In certain embodiments, n is 0. In certain embodiments, n is 1.

The description above describes multiple embodiments relating to compounds of Formula I-A2. The patent application specifically contemplates all combinations of the embodiments.

Another aspect of the invention provides a compound represented by Formula I-1:

[chemical structure]

or a pharmaceutically acceptable salt thereof; wherein:

R¹ represents independently for each occurrence halogen or C₁₋₄ alkyl;

R² represents independently for each occurrence hydroxyl, C₁₋₄ alkyl, or C₁₋₄ haloalkyl;

R³ represents independently for each occurrence hydrogen or C₁₋₄ alkyl;

R⁴ represents independently for each occurrence C₁₋₄ alkyl or C₁₋₄ haloalkyl;

R⁵ is hydrogen, C₁₋₄ alkyl, C₁₋₄ haloalkyl, C₁₋₄ hydroxyalkyl, C₁₋₄ alkoxyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 6 membered heteroaromatic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-7 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl; and

m and p are independently 0 or 1;

n is 0, 1, or 2.

The definitions of variables in Formula I-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1.

In certain embodiments, A¹ is a 6-membered heteroaromatic ring containing at least one ring nitrogen atom. In certain embodiments, A¹ is

[chemical structure]

In certain embodiments, A¹ is a 6-membered carbocyclic aromatic ring.

In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X², and (ii) 1 or 2 occurrences of R⁴. In certain embodiments, A³ is phenyl substituted by (i) —(C₁₋₄ alkylene)-X², and (ii) 1 occurrence of R⁴. In certain embodiments, A³ is phenyl substituted by (i) —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R² is C₁₋₄ alkyl. In certain embodiments, R² is ethyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl.

In certain embodiments, m is 1.

In certain embodiments, n is 0. In certain embodiments, n is 1.

The description above describes multiple embodiments relating to compounds of Formula I-1. The patent application specifically contemplates all combinations of the embodiments.

Another aspect of the invention provides a compound represented by Formula II:

[chemical structure]

or a pharmaceutically acceptable salt thereof; wherein:

R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);

R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, —(C₁₋₄ alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;

R³ represents independently for each occurrence hydrogen or C₁₋₄ alkyl;

R⁴ represents independently for each occurrence halogen, C₁₋₄ alkyl, C₁₋₄ haloalkyl, cyano, C₁₋₄ alkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵;

A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;

X¹ is —O—, —N(R³)—, or —S(O)ₜ—;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, oxo, cyano, C₁₋₄ alkoxyl, —(C₁₋₄ alkylene)-(C₁₋₄ alkoxyl), C₃₋₇ cycloalkyl, 3-7 membered heterocycloalkyl, and 5-6 membered heteroaryl;

Y¹ is —C(R³)₂— or —N(R³)—; and

m and n are each independently 0, 1, 2, or 3; and

t is 0, 1 or 2;

provided that if m is 0 and Y¹ is —C(R³)₂—, then A² is not a 6-membered heteroaryl substituted with —C(O)N(R³)(R⁵), —N(R³)(R⁵), or —N(R³)C(O)R⁵.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

In certain embodiments, A¹ is a 5-6 membered heteroaromatic ring containing at least one ring nitrogen atom. In certain embodiments, A¹ is

[chemical structure]

In certain embodiments, A¹ is a 6-membered unsaturated oxo-heterocyclic ring containing at least one ring nitrogen atom.

In certain embodiments, A¹ is a 6-membered carbocyclic aromatic ring.

In certain embodiments, A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano. In certain embodiments, A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, —(C₁₋₄ alkylene)-(C₁₋₄ alkoxyl), C₃₋₇ cycloalkyl, 3-7 membered heterocycloalkyl, 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, cyano. In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵. In certain embodiments, A² is a 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)-(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R5. In certain embodiments, A² is 8-10 membered bicyclic oxo-heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —N(R³)-(5-6 membered heteroaryl), 3-7 membered heterocycloalkyl, and 5-6 membered heteroaryl; wherein said C₃₋₇ cycloalkyl, 3-7 membered heterocycloalkyl, and 5-6 membered heteroaryl are each optionally substituted with 1 or 2 substituents independently selected from the group consisting of halogen, hydroxyl, C₁₋₄ alkyl, C₁₋₄ haloalkyl, and cyano.

In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴.

In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴. In certain embodiments, A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1 or 2 occurrences of R⁴.

In certain embodiments, X¹ is —O—. In certain embodiments, X¹ is —N(R³)— or —S—. In certain embodiments, X¹ is —S(O)—. In certain embodiments, X¹ is —S(O)₂—.

In certain embodiments, t is 0. In certain embodiments, t is 1. In certain embodiments, t is 2.

In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, and hydroxyl.

In certain embodiments, Y¹ is —C(R³)₂—. In certain embodiments, Y¹ is —N(R³)—.

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl. In certain embodiments, R² is C₁₋₄ alkyl. In certain embodiments, R² is ethyl. In certain embodiments, R² is C₁₋₄ alkyl, and m is 1 or 2

In certain embodiments, R¹ is C₁₋₄ alkyl, and R² is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl.

In certain embodiments, R³ is hydrogen, and R⁵ is hydrogen, C₁₋₄ alkyl, C₁₋₄ haloalkyl, or C₁₋₄ hydroxyalkyl.

In certain embodiments, m is 1. In certain embodiments, m is 2.

In certain embodiments, n is 0. In certain embodiments, n is 1.

In certain embodiments, Y¹ is —C(R³)₂—. In certain embodiments, Y¹ is —N(R³)—.

The description above describes multiple embodiments relating to compounds of Formula II. The patent application specifically contemplates all combinations of the embodiments.

Another aspect of the invention provides a compound represented by Formula II-A:

[chemical structure]

or a pharmaceutically acceptable salt thereof; wherein:

R¹ represents independently for each occurrence halogen or C₁₋₄ alkyl;

R^{2A} and R^{2B} each represent independently hydrogen, C₁₋₄ alkyl, C₁₋₄ haloalkyl, C₁₋₄ hydroxyalkyl, or —(C₁₋₄ alkylene)-(C₁₋₄ alkoxyl);

R³ represents independently for each occurrence hydrogen or C₁₋₄ alkyl;

R⁴ represents independently for each occurrence C₁₋₄ alkyl, C₁₋₄ haloalkyl, C₃₋₅ cycloalkyl, C₃₋₅ halocycloalkyl, -(cyclopropylene)-(C₁₋₄ haloalkyl), or -(cyclopropylene)-CN;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A¹ is a 6-membered heteroaromatic ring containing at least one ring nitrogen atom, or a 6-membered carbocyclic aromatic ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;

Y¹ is —C(R³)₂— or —N(R³)—; and

provided that if R^{2A} and R^{2B} are both hydrogen and Y¹ is —C(R³)₂—, then A² is not a 6-membered heteroaryl substituted with —C(O)N(R³)(R⁵) or —N(R³)(R⁵).

The definitions of variables in Formula II-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II-A.

In certain embodiments, A¹ is a 6-membered heteroaromatic ring containing at least one ring nitrogen atom. In certain embodiments, A¹ is

[chemical structure]

In certain embodiments, A¹ is a 6-membered carbocyclic aromatic ring.

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, —N(R³)(R⁵), C₁₋₄ alkoxyl, and —(C₁₋₄ alkylene)-(C₁₋₄ alkoxyl).

In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X² or —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl. In certain embodiments, R^{2A} is ethyl. In certain embodiments, R^{2A} is methyl. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl.

In certain embodiments, R³ is hydrogen, and R⁵ is hydrogen, C₁₋₄ alkyl, C₁₋₄ haloalkyl, or C₁₋₄ hydroxyalkyl.

In certain embodiments, n is 0. In certain embodiments, n is 1.

In certain embodiments, Y¹ is —C(R³)₂—. In certain embodiments, Y¹ is —N(R³)—.

The description above describes multiple embodiments relating to compounds of Formula II-A. The patent application specifically contemplates all combinations of the embodiments.

Another aspect of the invention provides a compound represented by Formula II-A1:

[chemical structure]

or a pharmaceutically acceptable salt thereof; wherein:

R¹ represents independently for each occurrence halogen or C₁₋₄ alkyl;

R^{2A} and R^{2B} each represent independently hydrogen, C₁₋₄ alkyl or C₁₋₄ haloalkyl;

R³ represents independently for each occurrence hydrogen or C₁₋₄ alkyl;

R⁴ represents independently for each occurrence C₁₋₄ alkyl or C₁₋₄ haloalkyl;

R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;

A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);

A³ is one of the following:
phenyl substituted by (i) —(C₁₋₄ alkylene)-X², —N(R³)—X², or —O—X², and (ii) 1 or 2 occurrences of R⁴;
5-6 membered heteroaryl substituted by (i) —(C₁₋₄ alkylene)-X², —N(R³)—X², or —O—X², and (ii) 1 or 2 occurrences of R⁴;

X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;

Y¹ is —C(R³)₂— or —N(R³)—;

Y² is N, C(H), or C(R¹); and

n is 0, 1, or 2;

provided that if R^{2A} and R^{2B} are both hydrogen and Y¹ is —C(R³)₂—, then A² is not a 6-membered heteroaryl substituted with —C(O)N(R³)(R⁵) or —N(R³)(R⁵).

The definitions of variables in Formula II-A1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II-A1.

In certain embodiments, A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 5-10 membered heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A² is one of the following

[chemical structure]

each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl). In certain embodiments, A² is one of the following

[chemical structure]

each of which is optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is one of the following

[chemical structure]

each of which is optionally substituted with 1 or 2 substituents independently selected from the group consisting of C₁₋₄ alkyl, C₁₋₄ haloalkyl, C₃₋₇ cycloalkyl, and cyano.

In certain embodiments, A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano. In certain embodiments, A² is 8-10 membered partially unsaturated bicyclic heterocyclyl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

In certain embodiments, A³ is phenyl substituted by (i) —(C₁₋₄ alkylene)-X² or —O—X², and (ii) 1 occurrence of R⁴. In certain embodiments, A³ is

[chemical structure]

In certain embodiments, A³ is

[chemical structure]

In certain embodiments, A³ is 5-6 membered heteroaryl substituted by (i) —(C₁₋₄ alkylene)-X² or —O—X², and (ii) 1 occurrence of R⁴. In certain embodiments, A³ is 6-membered heteroaryl substituted by (i) —(C₁₋₄ alkylene)-X² or —O—X², and (ii) 1 occurrence of R⁴. In certain embodiments, A³ is pyridinyl, pyrazinyl, or pyrimidinyl; each of which is substituted by (i) —(C₁₋₄ alkylene)-X² or —O—X², and (ii) 1 occurrence of R⁴. In certain embodiments, A³ is pyridinyl substituted by (i) —(C₁₋₄ alkylene)-X² or —O—X², and (ii) 1 occurrence of R⁴. In certain embodiments, A³ is

[chemical structure]

In certain embodiments, X² is a 3-7 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl. In certain embodiments, X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl. In certain embodiments, X² is piperidinyl or piperazinyl; each of which is optionally substituted with 1 or 2 substituents independently selected from the group consisting of C₁₋₄ alkyl and C₃₋₇ cycloalkyl.

In certain embodiments, X² is

[chemical structure]

In certain embodiments, X² is

[chemical structure]

In certain embodiments, X² is

[chemical structure]

In certain embodiments, X² is

[chemical structure]

In certain embodiments, R¹ is C₁₋₄ alkyl.

In certain embodiments, R^{2A} is C₁₋₄ alkyl. In certain embodiments, R^{2A} is ethyl. In certain embodiments, R^{2A} is methyl. In certain embodiments, R^{2B} is hydrogen. In certain embodiments, R^{2B} is C₁₋₄ alkyl.

In certain embodiments, R³ is hydrogen.

In certain embodiments, R⁴ is C₁₋₄ haloalkyl. In certain embodiments, R⁴ is trifluoromethyl.

In certain embodiments, R⁵ is hydrogen, C₁₋₄ alkyl, C₁₋₄ haloalkyl, or C₁₋₄ hydroxyalkyl.

In certain embodiments, R³ is hydrogen, and R⁵ is hydrogen, C₁₋₄ alkyl, C₁₋₄ haloalkyl, or C₁₋₄ hydroxyalkyl.

In certain embodiments, n is 0. In certain embodiments, n is 1.

In certain embodiments, Y¹ is —C(R³)₂—. In certain embodiments, Y¹ is —N(R³)—.

In certain embodiments, Y² is N. In certain embodiments, Y² is C(H).

The description above describes multiple embodiments relating to compounds of Formula II-A1. The patent application specifically contemplates all combinations of the embodiments.

In certain other embodiments, the compound is one of the compounds listed in Table 1 below, or a pharmaceutically acceptable salt thereof. In certain embodiments, the compound is one of the compounds listed in Table 1 below. In certain other embodiments, the compound is one of the compounds listed in Tables 2-10 or 19 in the Examples, or a pharmaceutically acceptable salt thereof. In certain embodiments, the compound is one of the compounds listed in Tables 2-10 or 19 in the Examples. In yet other embodiments, the compound is a compound in any one of Tables 1-10 or 19 herein, or a pharmaceutically acceptable salt thereof. In certain other embodiments, the compound is one of the compounds listed in Tables 1A, 11-18 or 20 in the Examples, or a pharmaceutically acceptable salt thereof. In certain embodiments, the compound is one of the compounds listed in Tables 1A, 11-18 or 20 in the Examples.

TABLE 1

| Compound No. | Chemical Structure |
|---|---|
| I-1 | [chemical structure] |
| I-2 | [chemical structure] |
| I-3 | [chemical structure] |
| I-4 | [chemical structure] |
| I-5 | [chemical structure] |
| I-6 | [chemical structure] |
| I-7 | [chemical structure] |
| I-8 | [chemical structure] |
| I-9 | [chemical structure] |
| I-10 | [chemical structure] |
| I-11 | [chemical structure] |
| I-12 | [chemical structure] |
| I-13 | [chemical structure] |
| I-14 | [chemical structure] |
| I-15 | [chemical structure] |
| I-16 | [chemical structure] |
| I-17 | [chemical structure] |
| I-18 | [chemical structure] |
| I-19 | [chemical structure] |
| I-20 | [chemical structure] |
| I-21 | [chemical structure] |
| I-22 | [chemical structure] |
| I-23 | [chemical structure] |
| I-24 | [chemical structure] |
| I-25 | [chemical structure] |
| I-26 | [chemical structure] |
| I-27 | [chemical structure] |
| I-28 | [chemical structure] |
| I-29 | [chemical structure] |
| I-30 | [chemical structure] |
| I-31 | [chemical structure] |
| I-32 | [chemical structure] |
| I-33 | [chemical structure] |
| I-34 | [chemical structure] |
| I-35 | [chemical structure] |
| I-36 | [chemical structure] |
| I-37 | [chemical structure] |
| I-38 | [chemical structure] |
| I-39 | [chemical structure] |
| I-40 | [chemical structure] |
| I-41 | [chemical structure] |
| I-42 | [chemical structure] |
| I-43 | [chemical structure] |
| I-44 | [chemical structure] |
| I-45 | [chemical structure] |
| I-46 | [chemical structure] |
| I-47 | [chemical structure] |
| I-48 | [chemical structure] |
| I-49 | [chemical structure] |
| I-50 | [chemical structure] |
| I-51 | [chemical structure] |
| I-52 | [chemical structure] |
| I-53 | [chemical structure] |
| I-54 | [chemical structure] |
| I-55 | [chemical structure] |
| I-56 | [chemical structure] |
| I-57 | [chemical structure] |
| I-58 | [chemical structure] |
| I-59 | [chemical structure] |
| I-60 | [chemical structure] |
| I-61 | [chemical structure] |
| I-62 | [chemical structure] |
| I-63 | [chemical structure] |
| I-64 | [chemical structure] |

TABLE 1A

| Compound No. | Chemical Structure |
|---|---|
| I-1A | [chemical structure] |
| I-2A | [chemical structure] |
| I-3A | [chemical structure] |
| I-4A | [chemical structure] |
| I-5A | [chemical structure] |
| I-6A | [chemical structure] |
| I-7A | [chemical structure] |
| I-8A | [chemical structure] |
| I-9A | [chemical structure] |
| I-10A | [chemical structure] |

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or be prepared based on procedures described in the literature.

The synthetic route illustrated in Scheme 1 is a general method for preparing substituted dihydroisoquinoline carboxamides G. Selective protection of the substituted tetrahydroisoquinoline A with an amine protecting group, such as a tert-butyl carbamate is achieved either by direct mono-protection (for example, using di-tert-butylcarbonate and an organic base such as triethylamine in an aprotic solvent such as dichloromethane, tetrahydrofuran or acetonitrile), or alternatively by the installation of two or more protecting groups and selective cleavage (for example, base-mediated hydrolysis), to provide an intermediate such as B. The unprotected phenol then is available for coupling to an aryl or heteroaryl moiety, for example via a nucleophilic aromatic substitution (S_{N}Ar) reaction to form D. Such S_{N}Ar reactions may be conducted, for example, by combining an aryl or heteroaryl moiety (C) containing a leaving group X such as a halide, with the phenol in an organic solvent. The reaction may be facilitated by the use of an inorganic base and/or by applying heat. For example, such reactions may be conducted in DMSO at approximately 150° C., or in DMF at approximately 100° C. In some instances, the S_{N}Ar reaction may be facilitated by the use of a strong base such as potassium tert-butoxide or potassium hydroxide. In other cases, a weaker base such as cesium carbonate is sufficient, depending upon the electrophilicity of the aryl or heteroaryl moiety. Commonly, S_{N}Ar reactions are conducted under Ullman coupling conditions (see, for example, Rovira, M. et al., Journal of Organic Chemistry 2016, 81, 7315-7325) with the aid of a copper catalyst (e.g. copper oxide, copper acetate, or copper iodide) in the presence of an amine ligand (e.g. pyridine or 1,10-phenanthroline) in an organic solvent (e.g. dichloromethane). Some copper-mediated S_{N}Ar reactions may be conducted at temperatures less than 100° C. When using a copper catalyst, the aryl or heteroaryl moiety leaving group may be a boronic acid.

Alternatively, the phenol and the aryl or heteroaryl moiety may be coupled via a metal-mediated cross-coupling reaction to form D. For this approach, the leaving group on the aryl or heteroaryl moiety may be a halide or a sulfonate group such as a triflate. The most common catalysts are palladium-based catalysts, although other metals (e.g. zinc or nickel) also may be used. A variety of palladium catalysts and associated ligands have been explored for use in these ether-forming cross-coupling reactions. Reactions may be performed in organic or mixed aqueous organic solvents in the presence of an organic or inorganic base, with heating (see, for example, Frlan, R and Kikelj, D. Synthesis 2006, 14, 2271-2285).

Deprotection of D under appropriate deprotecting conditions (for example, strong acid, such as trifluoroacetic acid, in dichloromethane) provides amine E. Reaction of E with isocyanate F in the presence of an organic base (e.g. triethylamine or diisopropylethyl amine) in an apolar solvent such as dichloromethane yields the dihydroisoquinoline carboxamide G.

[chemical structure]

Scheme 2 illustrates general methods for preparing ureas via intermediate isocyanates or phenyl carbamates. The primary amine is acylated with a carbonyl source such as a chloroformate derivative in a polar organic solvent such as DMF containing an organic amine such as triethyl amine to form a carbamate (B). Alternatively, the primary amine is reacted with triphosgene, or a similar reagent, in a solvent such as dichloromethane to form an isocyanate (C). The carbamate (B) or isocyanate (C) in turn is reacted with an appropriate amine in a non-protic solvent, such as dichloromethane, containing an organic base, such as trimethylamine, to form a urea (D). See, for example, Bergeron, Phillippe et al. Med. Chem. Lett. 2016, 7, 595-600 or Shen, Weijun et al. J. Am. Chem. Soc. 2013, 135, 1669-1672.

[chemical structure]

Scheme 3 illustrates a general method of preparing optionally substituted tetrahydroisoquinoline F. Substituted phenylacetonitrile A can be alkylated with various electrophiles after treatment with an appropriate base (for example, lithium diisopropylamide) to form substituted phenylacetonitrile B. Reduction of phenylacetonitrile B to amine C may be performed by using, for example, a hydride reducing agent, such as lithium aluminum hydride. Reaction of amine C with an appropriate aldehyde, followed by treatment with, for example, strong acid or heat, promotes an intramolecular Friedel Crafts-like cyclization reaction to produce imine D, which can then be reduced to tetrahydroisoquinoline F using a reducing agent such as sodium cyanoborohydride. Alternatively, amine C can be reacted with a carbonyl source, (such as a chloroformate [e.g., ethyl chloroformate or phenyl chloroformate], carbonyl diimidazole, or triphosgene) followed by treatment with, for example, strong acid or heat, to produce lactam E. Reduction of lactam E, for example, using a hydride source such as lithium aluminum hydride, affords tetrahydroisoquinoline F (R³═H). Deprotection of the methyl ether of compound F (for example, using boron tribromide) affords a hydroxylated tetrahydroisoquinoline, which can be further elaborated via methods described in connection with Scheme 1.

[chemical structure]

Scheme 4 is a variation on Scheme 3 where the imine formation step is an intramolecular reaction. Formation of tetrahydroisoquinoline F may proceed with isolation of intermediate imine D or directly in one step via a reductive amination reaction.

[chemical structure]

Scheme 5 illustrates an approach to the preparation of 3-substituted tetrahydroisoquinolines. Reductive amination of ketone A to give the primary amine directly (e.g., NH₃, Ti(OⁱPr)₄) or via a protected amine (e.g., using benzyl amine with sodium triacetoxyborohydride, then cleavage of the protecting group), forms an amine B. Amine B may be converted to the tetrahydroisoquinoline via any of the methods described above in Schemes 3 and 4. Additionally, a number of methods have been described for enantioselective formation of amines B, for example using enzyme catalysis with for example amine dehydrogenase (see, for example, ACS Catalysis 2015, 5, 1119-1122) or amine transferase (see, for example, ACS Catalysis 2017, 7, 4768-4774).

[chemical structure]

Scheme 6 illustrates a general method for forming the enantioenriched 1-substituted tetrahydroisoisoquinoline E. Enantio-enriched amine A can be acylated with picolinic acid (for example, using a coupling reagent such as EDCI) to form amide B. A palladium-catalyzed alkylation of the aryl ring (see, for example, Zhao, Y., Chen, G. Org. Lett. 2011, Vol. 13, No. 18, pp. 4850-4853) affords substituted amide C. Deprotection of the benzyl alcohol (for example, with 10% palladium on carbon under a hydrogen atmosphere), followed by activation of the alcohol (for example, with tosyl chloride), followed by treatment with base (for example, sodium hydride) allows for the cyclization to form N-acylated tetrahydroisoquinoline D. Deprotection (for example, with sodium methoxide in methanol) affords enantio-enriched 1-substituted tetrahydroisoisoquinoline E.

[chemical structure]

Scheme 7 illustrates a general method of preparing substituted pyridyl phenylcarbamate G. Substituted 2-chloro-5-nitropyridine A is condensed with, for example, alcohol (Z═O) or amine (Z═NH) reactant B (for example, using cesium carbonate in dimethylformamide with heating) to form nitropyridine C. After reduction of C (for example, with iron and ammonium chloride) to form aminopyridine F, reaction of F with phenylchloroformate forms pyridyl phenylcarbamate G. Alternatively, to prepare nitropyridine C when Z is —CH₂—, a Suzuki coupling with 2-chloro-5-nitropyridine A and methyl borate (for example, with tetrakistriphenylphosphinepalladium(0) and potassium carbonate in 1,4-dioxane) affords 2-methylpyridine D. Oxidation of D (for example, with selenium dioxide) results in aldehyde E. Reductive amination of E with an amine affords C (where Z═—CH₂—), which then undergoes similar chemistry as described to afford G.

[chemical structure]

Scheme 8 illustrates a general method of preparing 8-substituted dihydro-1,6-naphthyridine carboxamides H. 2-Hydroxy-3,5-dinitropyridine A is first alkylated (for example, with methyl iodide and a base) and then reacted with substituted ketone B (see, for example, Guiadeen, Deodialsingh; et al, Tetrahedron Lett. 2008, 49, 6368-6370) affording Boc-protected tetrahydronaphthyridine C. The nitro group of C is then reduced (for example, with rhodium on carbon as catalyst) to form the amine D. Diazotization of D (for example, with sodium nitrite and hydrochloric acid) affords hydroxylated tetrahydronaphthyridine E. Reaction of D or E under palladium coupling or S_{N}Ar conditions (for example, as described in Scheme 1) forms Boc-protected intermediate F. After deprotection (for example, with trifluoroacetic acid), 8-substituted dihydro-1,6-naphthyridine carboxamide H can be formed by coupling with isocyanate G.

[chemical structure]

Scheme 9 illustrates a general method of preparing 5-substituted tetrahydro-1,6-naphthyridine carboxamide E. 2-Methylpyridine A is first brominated (for example, using benzoyl peroxide and bromine), followed by displacement of the bromide (for example, with sodium cyanide) to form pyridylacetonitrile B. Pyridylacetonitrile B can then be coupled to the appropriately substituted vinyl ether (for example, with tetrakistriphenylphosphinepalladium(0) and sodium carbonate in 1,4-dioxane/water) to form the ketone after acidic work up which is then protected as the ketal using ethyl glycol with catalytic tosic acid to form ketal C. Reduction of the nitrile group in compound C (using, for example, lithium aluminum hydride) affords amine D. Deprotection of the ketal of compound D (using, for example, an aqueous solution of hydrochloric acid), followed by reduction of the cyclized intermediate imine (using, for example, lithium aluminum hydride) yields 5-substituted tetrahydro-1,6-naphthyridine E.

[chemical structure]

Scheme 10 illustrates a general method of preparing chiral 4-substituted-1,2,3,4-tetrahydroisoquinolines E, G, and I. The optionally substituted (R,E)-3-(3-(4-methoxyphenyl)acryloyl)-4-phenyloxazolidin-2-one A is prepared from (R)-4-phenyl-2-oxazolidinone, a suitable base such as triethyl amine or n-butyl lithium, and the acid chloride of an optionally substituted 4-alkoxylcinnamic acid. Diastereoselective addition of an alkyl (R^{2x}) cuprate (such as that prepared from an alkyl magnesium halide in the presence of copper (I) bromide dimethyl sulfide complex) in the presence of a Lewis acid catalyst such as boron trifluoride etherate or trimethylsilyl chloride (see, for example, Q. Zhang, et al. Tetrahedron: Asymmetry, 2012, 23, 577-582) affords substituted (R)-3-((R)-3-(4-methoxyphenyl)alkanoyl)-4-phenyloxazolidin-2-one B (R^{2y}═H) An optional diastereoselective addition of R^{2y} can be achieved by treatment with a base such as sodium hexamethylsilazide followed by quenching of the anion with an alkyl halide to afford (R)-3-((2R,3R)-3-(4-methoxyphenyl)-2-substitutedalkanoyl)-4-phenyloxazolidin-2-one B. Cleavage of the imide bond in compound B with a suitable base (such as lithium hydroxide in the presence of hydrogen peroxide) affords a carboxylic acid, which can be converted to isocyanate C via a Curtius reaction (using, for example, diphenylphosphoryl azide). Treatment of isocyanate C with a Lewis acid (such as titanium tetrachloride, stannous chloride or boron trifluoride etherate) effects cyclization to substituted lactam D. Reduction of the lactam (for example, with a hydride such as lithium aluminum hydride) affords the chiral (S)-7-methoxy-4-substituted-1,2,3,4-tetrahydroisoquinoline E with optional 3-(R)-substitution of R^{2y}.

Chiral (S)-7-methoxy-4-substituted-1,2,3,4-tetrahydroisoquinoline G with optional 3-(S)-substitution of R^{2z} can be prepared by preparing the anion of B with a suitable base (such as sodium hexamethylsilazide or lithium dimethylamide) and diastereoselectively protonating by quenching with a hindered acid (such as a 2,6-disubstituted phenol or saturated aqueous sodium sulfate) to provide (R)-3-((2S,3R)-3-(4-methoxyphenyl)-2-substitutedalkanoyl)-4-phenyloxazolidin-2-one F. Utilizing the same sequence to convert compound B to compound E, compound F can be converted to compound G. Chiral (S)-7-methoxy-4-substituted-1,2,3,4-tetrahydroisoquinoline I with optional 3,3-disubstitution can be prepared in a diastereoselective manner by diastereoselectively alkylating the anion of B with an alkyl halide (R^{2y}—X). Utilizing the same sequence as above then affords I.

The enantiomers of E, G, and I can be prepared starting from (S)-4-phenyl-2-oxazolidinone. Depending on substitutents R^{2x}, R^{2y} and R^{2z}, the optimal chiral auxiliary may be modified to obtain higher diastereoselectivity. After deprotection of the methyl ether, tetrahydroisoquinolines E, G, and I can be converted to urea K via the methods described in Scheme 1.

[chemical structure]

[chemical structure]

[chemical structure]

[chemical structure]

Scheme 11 illustrates an alternative method of preparing chiral 4-substituted-1,2,3,4-tetrahydroisoquinoline G. Optionally substituted 2-halo-5-methoxybenzaldehyde A (X is halogen) can either be purchased or prepared via regioselective halogenation of substituted 3-methoxybenzaldehydes. Protection of the aldehyde with a diol (such as ethylene glycol) and acid catalysis under dehydrating conditions (such as refluxing with toluene with a Dean-Stark trap) yields ketal B. The optionally substituted ketal can be converted to chiral 4-substituted-1,2,3,4-tetrahydroisoquinoline G via reactions analogous to those in described in connection with Scheme 3 in international patent application publication WO 2009/148259. Palladium mediated Heck coupling of (R)-3-acryloyl-4-phenyloxazolidin-2-one to ketal B affords substituted (R,E)-3-(3-(2-(1,3-dioxolan-2-yl)-4-methoxyphenyl)acryloyl)-4-phenyloxazolidin-2-one C. Diastereoselective alkyl (R^{2x}) cuprate addition under Lewis acid catalysis and optional alkylation and protonation under conditions described in connection with Scheme 10 herein affords (R)-3-((R)-3-(2-(1,3-dioxolan-2-yl)-4-methoxyphenyl)alkanoyl)-4-phenyloxazolidin-2-one D with optional substitutions of R^{2y} and R^{2z}. Cleavage of the imide bond in compound D with a suitable base (such as lithium hydroxide in the presence of hydrogen peroxide) affords a carboxylic acid which can be converted to isocyanate E via a Curtius reaction (for example, by treatment with diphenylphosphoryl azide). Acid hydrolysis of both the isocyanate and ketal moieties affords imine F, which can be reduced with a suitable hydride (such as sodium borohydride or sodium triacetoxyborohydride) to afford the chiral 4-substituted-1,2,3,4-tetrahydroisoquinoline G. The enantiomers of G can be prepared from (S)-4-phenyl-2-oxazolidinone. Depending on substitutents R^{2x}, R^{2y} and R^{2z}, the optimal chiral auxiliary may be modified to obtain higher diastereoselectivity. Deprotection of the methyl ether of compound G affords hydroxylated tetrahydroisoquinoline H, which can be converted to urea I via methods described in connection with Scheme 1.

[chemical structure]

[chemical structure]

Scheme 12 illustrates a method of preparing optionally substituted chiral (R)-3-(benzyloxy)-8-substituted-5,6,7,8-tetrahydro-1,6-naphthyridine F. Alkylation of a substituted 5-bromo-6-chloropyridin-3-ol with a protecting group (such as methyl or benzyl) affords ether B. Metal insertion into the bromide and quenching with dimethyl formamide yields aldehyde C. Protection of the aldehyde with a diol (such as ethylene glycol) with acid catalysis under dehydrating conditions (such as refluxing with toluene with a Dean-Stark trap) yields ketal D. Palladium mediated Heck coupling of (R)-3-acryloyl-4-phenyloxazolidin-2-one to ketal D affords substituted (R,E)-3-(3-(5-(benzyloxy)-3-(1,3-dioxolan-2-yl)pyridin-2-yl)acryloyl)-4-phenyloxazolidin-2-one E. Diastereoselective cuprate addition under Lewis acid catalysis and optional alkylation and protonation followed by a Curtius rearrangement and reductive cyclizations under conditions described in connection with Schemes 10 and 11 affords substituted chiral (R)-3-(benzyloxy)-8-substituted-5,6,7,8-tetrahydro-1,6-naphthyridine F. The enantiomers of F can be prepared from (S)-4-phenyl-2-oxazolidinone. Depending on substitutents R^{2x}, R^{2y} and R^{2z}, the optimal chiral auxiliary may be modified to obtain higher diastereoselectivity. Deprotection of the benzyl ether of F affords a hydroxylated 8-substituted-5,6,7,8-tetrahydro-1,6-naphthyridine, which can be converted to urea G via the methods described in Scheme 1.

[chemical structure]

Scheme 13 illustrates a method of preparing optionally substituted tetrahydroisoquinoline D. Ketone A undergoes a reductive amination with aminoacetaldehyde dimethyl acetal to form acetal B. Acetal B is then reacted via a Friedel-Crafts cyclization (for example, with trifluoromethanesulfonic acid) to form substituted tetrahydroisoquinoline C. Intermediate C can then be deprotected (for example, with boron tribromide) to form optionally substituted tetrahydroisoquinoline D.

[chemical structure]

Scheme 14 illustrates a method of preparing optionally substituted methyl 7-methoxy-1,2,3,4-tetrahydronaphthalene-2-carboxylate G. Palladium-mediated Heck coupling of substituted 3-bromoanisole A to substituted dimethyl itaconate B yields cinnamate C. Hydrogenation of cinnamate C using a transition metal or alkyl (R³) cuprate addition to the carbon-carbon double bond affords substituted dimethyl succinate D. Treatment of compound D with a strong acid to effect a Friedel Crafts reaction gives ketone E. An R^{2x} substitution can be introduced via treatment with a Wittig reagent to afford alkene F. Hydrogenation of alkene F then affords optionally substituted methyl 7-methoxy-1,2,3,4-tetrahydronaphthalene-2-carboxylate G. For targets where R^{2x} is hydrogen, reduction of ketone E with a hydride, followed by hydrogenation affords G. Cleavage of the methyl ester and methyl ether in compound G (using, for example, a Lewis acid catalyst such as boron tribromide or trimethyl silyl iodide) affords carboxylic acid H. Amide coupling to form amide I may be performed before or after coupling to the desired heteroaryl moiety (X¹) (for example, as described in Scheme 1).

[chemical structure]

[chemical structure]

Scheme 15 illustrates a general method of preparing optionally substituted 5,6,7,8-tetrahydroquinoline-6-carboxamide F. 2-Hydroxy-3,5-dinitropyridine A is first alkylated (for example, with methyl iodide) and then reacted with substituted ketone B (see, for example, Guiadeen, Deodialsingh; et al, Tetrahedron Lett. 2008, 49, 6368-6370) to afford optionally substituted methyl 3-nitro-5,6,7,8-tetrahydroquinoline-6-carboxylate C. The nitro group in compound C is then reduced (for example, with rhodium on carbon as catalyst) to form amine D. Diazotization of compound D (for example, with sodium nitrite and hydrochloric acid) yields methyl 3-hydroxyl-5,6,7,8-tetrahydroquinoline-6-carboxylate E. After coupling to the appropriate heteroaryl moiety (X¹) (for example, as described in connection with Scheme 1), the methyl ester is converted to the carboxylic acid, and then amide formation affords optionally substituted 5,6,7,8-tetrahydroquinoline-6-carboxamide F.

[chemical structure]

Scheme 16 illustrates a general method of preparing 5,6,7,8-tetrahydroquinoline-6-carboxamide (where Y═N) and 1,2,3,4-tetrahydronaphthalene-2-carboxamide (where Y═CH or CR¹) I. Lactone B where R^{2z} is H can be prepared from L-glutamic acid (see, for example, M. Okabe et al, J. Org. Chem. 1988, 53, 4780-4786). Lactones where R^{2z} is alkyl can be readily prepared from α,β-butenolides (see, for example, W. Goldring et al. Synlett 2010, 4, 547-550). Diasteroselective alkylation of lactone B with benzyl halide A (for example, via analogy to the methods in Tetrahedron Asymmetry 1993, 4, 121-131 and J. Org Chem. 1987, 52, 1170-1172) affords 3-benzylated lactone C. Subjecting lactone C to methanol and acid affords methyl ester diol D, which may be further subjected to oxidative cleavage conditions (using, for example, an oxidant such as Jones' reagent, or sodium metaperiodate and ruthenium oxide followed by further oxidation with sodium hypochlorite) to yield carboxylic acid E. Subjecting compound E to acid-catalyzed ring closing conditions affords ketone F (for example, by the use of sulfuric acid or a Lewis acid such as titanium tetrachloride or boron trifluoride etherate). The oxygen atom of the ketone in compound F can be removed to form intermediate G (using, for example, hydride addition followed by a reduction (where R^{2x}═H) or via a Wittig olefination followed by hydrogenation (where R^{2x}═alkyl)). Cleavage of the methyl ester and methyl ether in compound G (for example, with acid catalysts such as boron tribromide or trimethyl silyl iodide) affords carboxylic acid H. Conversion of compound H to I may be performed following a similar sequence of reaction as described above (for example, as described in connection with Scheme 1).

[chemical structure]

[chemical structure]

Scheme 17 illustrates a general method of preparing chiral optionally substituted 5,6,7,8-tetrahydroquinoline-6-carboxamide (where Y═N) and 1,2,3,4-tetrahydronaphthalene-2-carboxamide (where Y═CH or CR′) H. The optionally substituted (R,E)-3-(3-(4-methoxyphenyl or 5-methoxypyridyl)acryloyl)-4-phenyloxazolidin-2-one A are prepared from (R)-4-phenyl-2-oxazolidinone, a suitable base (such as triethylamine or n-butyl lithium), and the acid chloride of an optionally substituted 4-alkoxylcinnamic acid. Alternatively, A can be prepared via a Heck reaction of the 5-methoxy-3-halopyridine or 3-methoxybromobenzene with (R)-3-acryloyl-4-phenyloxazolidin-2-one. Diastereoselective addition of an alkyl cuprate (see, for example, Q. Zhang, et al. Tetrahedron: Asymmetry, 2012, 23, 577-582) affords optionally substituted (R)-3-(3-(3-methoxyphenyl)propanoyl)-4-phenyloxazolidin-2-one or (R)-3-(3-(5-methoxypyridin-3-yl)propanoyl)-4-phenyloxazolidin-2-one B. Cleavage of the imide bond in compound B with a suitable base (such as lithium hydroxide in the presence of hydrogen peroxide) affords a carboxylic acid, which can be esterified with readily available chiral alcohols (available, for example, via the method of Sharpless, B. et al. J. Am. Chem. Soc. 1981, 103, 6237-6240) to afford allylic ester C. Diastereoselective Ireland-Claisen rearrangement of an intermediate (E)-trimethylsilylketene acetal of C (see Williams, et al. Org. Lett. 2016, 18, 420-423; and Ireland, et al. J. Org. Chem. 1991, 56, 650-657) affords alkene D. Alkene D can be oxidatively cleaved to carboxylic acid E using a suitable oxidant (such as the Jones' reagent; or sodium metaperiodate and ruthenium oxide to yield an aldehyde, which is then oxidized to the acid with sodium hypochlorite). Friedel Crafts cyclization of compound E affords ketone F (using, for example, an appropriate acid catalyst (such as sulfuric acid or a Lewis acid, such as titanium tetrachloride or boron trifluoride etherate)). The oxygen of the ketone in compound F can be removed, for example, by hydride addition followed by a reduction (where R^{2x}═H) or via a Wittig olefination followed by hydrogenation (where R^{2x}=alkyl). Cleavage of the methyl ester and methyl ether in compound F (for example, with acid catalysts such as boron tribromide or trimethyl silyl iodide) affords carboxylic acid G, which may be further elaborated as described previously.

[chemical structure]

[chemical structure]

Scheme 18 illustrates a general method of preparing optionally substituted chiral 1-alkyl-1,2,3,4-tetrahydroisoquinoline (where Y═CH or CR′) and chiral 5-alkyl-5,6,7,8-tetrahydro-1,6-naphthyridine (where Y═N) F. Reaction of substituted 1-(2-halo-5-methoxyphenyl)alkan-1-one and 1-(2-halo-5-methoxypyridin-3-yl)alkan-1-one A (prepared, for example, via metalation of 3-bromo-2-chloro-5-methoxypyridines and quenching with a Weinreb amide) with O-benzylhydroxylamine yields oxime B. Chiral reduction (for example, with a hydride in the presence of a spiroborate catalyst, see Ortiz-Marciales, et al. J. Org. Chem. 2008, 73, 6928-6931 and Org. Synthesis 2010, 87, 36-52) affords a chiral amine, which after protection with a carbamoylating reagent yields chiral carbamate C. Copper- or Pd-mediated addition of a dialkylmalonate (see, for example, F. Y. Kwong et al, Org. Lett. 2007, 9, 3469-3472; or J. F. Hartwig et al J. Org. Chem. 2002, 67, 541-555) to replace the halide of C affords malonate D. Hydrolysis of the carbamate, and concurrent lactam formation followed by decarboxylation affords lactam E. Alternatively, lactam E can be produced by reacting the chiral amine formed by the spiroborate catalyst with ethyl malonyl chloride, then utilizing copper or Pd-mediated intramolecular addition of the active methylene to form, after decarboxylation, lactam E. Optional alkylation of the enolate of lactam E introduces substituent R^{2x} and subsequent reduction (for example, with using a hydride reagent) affords amine F. Optional R^{2y} or R^{2z} can be introduced by carbamoylating E, followed by successive alkyl lithium and hydride additions to afford amine F. Alternatively, Lactam E can be reduced directly to amine F (for example, with a hydride, such as lithium aluminum hydride) with no optional substitution of R^{2x}, R^{2y} or R^{2z}. Deprotection of the methyl ether in compound F affords hydroxylated amine G, which can be converted to urea H via methods described in connection with Scheme 1.

[chemical structure]

[chemical structure]

Scheme 19 illustrates a general method of preparing optionally substituted chiral 7- and/or 8-alkyl-substituted 5,6,7,8-tetrahydro-1,6-naphthyridine G. In Part A of Scheme 19, reaction of substituted acetic acid A with dimethyl formamide and POCl₃ followed by sodium hexafluorophosphate (see, for example, Davies et al J. Org Chem. 2000, 65, 4571-4574 and references therein) affords vinylogous formamidinium salt B where X¹ can be, for example, —O— heteroaryl, —S-heteroaryl, or —N(H)-heteroaryl. Alternatively X¹ can be halogen or methoxy, which are later converted to —O-heteroaryl, —S-heteroaryl, or —N(H)-heteroaryl after the initial condensation and cyclization in Part B to intermediates analogous to the pyridone F or the amine G. In Part B, condensationn of the chiral β-aminoacid C with an alkyl malonic acid (for example, as its magnesium salt) yields ketoester D. Base-catalyzed addition of the ketoester D to the vinylogous formamidinium salt B (see, for example, Org. Proc. Res. Dev. 2010, 14, 849-858 and J. Org. Chem. 2001, 66, 4194-4199), followed by treatment with ammonium acetate gives substituted pyridyl ester E. Next, treatment of compound E with acid removes the carbamate, and treatment with a base (such as triethylamine) affords pyridone F. Subjecting compound F to reducing conditions (for example, with a hydride such as lithium aluminum hydride) affords amine G, which can be converted to urea H via methods described in connection with Scheme 1.

[chemical structure]

[chemical structure]

### II. Therapeutic Applications of Dihydroisoquinoline-2(1H)-Carboxamide and Related Compounds

It is contemplated that dihydroisoquinoline-2(1H)-carboxamides and related compounds described herein, such as a compound of Formula I, I-A, I-A1, II, II-A, II-A1, or other compounds in Section I, provide therapeutic benefits to subjects suffering from cancer. Accordingly, one aspect of the invention provides a method of treating cancer in a subject. The method comprises administering a therapeutically effective amount of a dihydroisoquinoline-2(1H)-carboxamide or related compound described herein, such as a compound of Formula I, I-A, I-A1, II, II-A, II-A1, or other compounds in Section I, to a subject in need thereof to treat the cancer. In certain embodiments, the particular compound of Formula I, I-A, I-A1, II, II-A, II-A1, is a compound defined by one of the embodiments described above.

In certain embodiments, the cancer is a solid tumor, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

Additional exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the subject is a human.

Another aspect of the invention provides for the use of a compound described herein (such as a compound of Formula I, I-A, I-A1, II, II-A, II-A1, or other compounds in Section I) in the manufacture of a medicament. In certain embodiments, the medicament is for treating a disorder described herein, such as cancer.

Another aspect of the invention provides for the use of a compound described herein (such as a compound of Formula I, I-A, I-A1, II, II-A, II-A1, or other compounds in Section I) for treating a medical disorder, such a medical disorder described herein (e.g., cancer).

Further, it is contemplated that dihydroisoquinoline-2(1H)-carboxamide and related compounds described herein, such as a compound of Formula I, I-A, I-A1, II, II-A, II-A1, or other compounds in Section I, can inhibit the activity of HPK1. Accordingly, another aspect of the invention provides a method of inhibiting the activity of HPK1. The method comprises exposing a HPK1 to an effective amount of an dihydroisoquinoline-2(1H)-carboxamide or related compound described herein, such as a compound of Formula I, I-A, I-A1, II, II-A, H-A1, or other compounds in Section I, to inhibit HPK1 activity. In certain embodiments, the particular compound of Formula I, I-A, I-A1, II, II-A, or II-A¹ is the compound defined by one of the embodiments described above.

### III. Combination Therapy

Another aspect of the invention provides for combination therapy. Dihydroisoquinoline-2(1H)-carboxamide and related compounds (e.g., a compound of Formula I, I-A, I-A1, II, II-A, II-A1, or other compounds in Section I) or their pharmaceutically acceptable salts may be used in combination with additional therapeutic agents to treat medical disorders, such as a cancer.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytoxic agents (e.g., tyrosine-kinase inhibitors).

Accordingly, another aspect of the invention provides a method of treating cancer in a patient, where the method comprises administering to the patient in need thereof (i) a therapeutically effective amount of an HPK1 inhibitor compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDCl7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDCl7 Inhibitor. In certain embodiments, the second anti-cancer agent is a CDCl7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 8106, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consistant of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

The doses and dosage regimen of the active ingredients used in the combination therapy may be determined by an attending clinician. In certain embodiments, the dihydroisoquinoline-2(1H)-carboxamide or related compound (e.g., a compound of any one of Formula I, I-A, I-A1, II, II-A, II-A1, or other compounds in Section I) and the additional therapeutic agent(s) are administered in doses commonly employed when such agents are used as monotherapy for treating the disorder. In other embodiments, the dihydroisoquinoline-2(1H)-carboxamide or related compound (e.g., a compound of any one of Formula I, I-A, I-A1, II, II-A, II-A1, or other compounds in Section I) and the additional therapeutic agent(s) are administered in doses lower than the doses commonly employed when such agents are used as monotherapy for treating the disorder. In certain embodiments, the dihydroisoquinoline-2(1H)-carboxamide or related compound (e.g., a compound of any one of Formula I, I-A, I-A1, II, II-A, II-A1, or other compounds in Section I) and the additional therapeutic agent(s) are present in the same composition, which is suitable for oral administration.

In certain embodiments, the dihydroisoquinoline-2(1H)-carboxamide or related compound (e.g., a compound of any one of Formula I, I-A, I-A1, II, II-A, II-A1, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the dihydroisoquinoline-2(1H)-carboxamide or related compound (e.g., a compound of any one of Formula I, I-A, I-A1, II, II-A, II-A1, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an dihydroisoquinoline-2(1H)-carboxamide or related compound described herein in a therapeutically effective amount for the treatment of a medical disorder described herein.

### Examples

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Synthesis of N-[4-[(4-Ethylpiperazin-1-yl)methyl]-3-(trifluoromethyl)phenyl]-7-[pyrrolo[2,1-f][1,2,4]triazin-4-yloxy]-1,2,3,4-tetrahydroisoquinoline-2-carboxamide (Compound No. 1)

[chemical structure]

### Part I—Synthesis of tert-Butyl 7-[pyrrolo[2,1-f][1,2,4]triazin-4-yloxy]-1,2,3,4-tetrahydroisoquinoline-2-carboxylate

[chemical structure]

Into a 25 mL round-bottom flask, was placed 4-chloropyrrolo[2,1-f][1,2,4]triazine (586 mg, 3.82 mmol), N,N-dimethylformamide (15 mL), cesium carbonate (959 mg, 2.94 mmol), and tert-butyl 7-hydroxy-1,2,3,4-tetrahydroisoquinoline-2-carboxylate (300 mg, 1.20 mmol). The resulting solution was stirred for 4 hours at room temperature, then diluted with water and extracted with ethyl acetate. The organic layers were combined and washed with sodium chloride (aq). The organic layer was concentrated under vacuum. The residue was applied onto a silica gel column with ethyl acetate/petroleum ether (1:2). The collected fractions were combined and concentrated under vacuum to yield tert-butyl 7-[pyrrolo[2,1-f][1,2,4]triazin-4-yloxy]-1,2,3,4-tetrahydroisoquinoline-2-carboxylate (430 mg, 98%).

### Part II—Synthesis of 7-[Pyrrolo[2,1-f][1,2,4]triazin-4-yloxy]-1,2,3,4-tetrahydroisoquinoline

[chemical structure]

Into a 25 mL round-bottom flask, was placed tert-butyl 7-[pyrrolo[2,1-f][1,2,4]triazin-4-yloxy]-1,2,3,4-tetrahydroisoquinoline-2-carboxylate (300 mg, 0.82 mmol), dichloromethane (10 mL), and trifluoroacetic acid (5 mL). The resulting solution was stirred for 2 hours at room temperature then concentrated under vacuum yielding 7-[pyrrolo[2,1-f][1,2,4]triazin-4-yloxy]-1,2,3,4-tetrahydroisoquinoline as the trifluoroacetic acid solvate (290 mg, 93%).

### Part III—Synthesis of N-[4-[(4-Ethylpiperazin-1-yl)methyl]-3-(trifluoromethyl)phenyl]-7-[pyrrolo[2,1-f][1,2,4]triazin-4-yloxy]-1,2,3,4-tetrahydroisoquinoline-2-carboxamide (Compound No. 1)

[chemical structure]

Into a 50 mL round-bottom flask, was placed 7-[pyrrolo[2,1-f][1,2,4]triazin-4-yloxy]-1,2,3,4-tetrahydroisoquinoline (290 mg, 1.09 mmol), dichloromethane (20 mL), diisopropylethylamine (1.7 g, 13.15 mmol), and 1-ethyl-4-([4-isocyanato-2-(trifluoromethyl)phenyl]methyl)piperazine (375 mg, 1.20 mmol). The resulting solution was stirred overnight at room temperature then concentrated under vacuum. The crude product was purified by preparatory HPLC to yield N-[4-[(4-ethylpiperazin-1-yl)methyl]-3-(trifluoromethyl)phenyl]-7-[pyrrolo[2,1-f][1,2,4]triazin-4-yloxy]-1,2,3,4-tetrahydroisoquinoline-2-carboxamide (81.9 mg, 13%). (ES, m/z): [M+H]⁺=580; ¹H NMR (300 MHz, DMSO-d₆, ppm): δ 8.92 (s, 1H), 8.11 (s, 1H), 8.07 (dd, J=2.6, 1.5 Hz, 1H), 7.91 (d, J=2.2 Hz, 1H), 7.76 (dd, J=8.4, 2.3 Hz, 1H), 7.58 (d, J=8.6 Hz, 1H), 7.31 (d, J=8.1 Hz, 1H), 7.20-7.13 (m, 2H), 7.07 (dd, J=4.5, 1.5 Hz, 1H), 6.96 (dd, J=4.5, 2.6 Hz, 1H), 4.68 (s, 2H), 3.76 (t, J=5.8 Hz, 2H), 3.52 (d, J=1.8 Hz, 2H), 2.91 (t, J=5.9 Hz, 2H), 2.49-2.13 (m, 10H), 0.98 (t, J=7.1 Hz, 3H).

### Example 2—Synthesis of Additional N-phenyl-3,4-dihydroisoquinoline-2(1H)-Carboxamides and Related Compounds

The compounds in Table 2 below were prepared based on experimental procedures described in Example 1 and in the detailed description.

TABLE 2

| | | Mass Spec. | |
|---|---|---|---|
| No. | Chemical Structure | (ES, m/z) | NMR Spectrum |
| II-1 | [chemical structure] | 570.2 [M + H]⁺ | ¹H NMR (300 MHz, DMSO-d6 ppm): δ 8.88 (s, 1H), 8.13 (s, 1H), 7.90 (d, J = 2.2 Hz, 1H), 7.75 (dd, J = 8.5, 2.2 Hz, 1H), 7.58 (d, J = 8.6 Hz, 1H), 7.26 (dd, J = 14.4, 6.5 Hz, 2H), 6.96 (d, J = 7.7 Hz, 2H), 5.78 (s, 1H), 4.65 (s, 2H), 3.73 (t, J = 5.9 Hz, 2H), 3.51 (s, 2H), 2.87 (t, J = 6.0 Hz, 2H), 2.77 (d, J = 4.7 Hz, 3H), 2.45-2.17 (m, 10H), 0.98 (t, J = 7.1 Hz, 3H). |
| II-2 | [chemical structure] | 660.3 [M + H]⁺ | ¹H NMR (400 MHz, DMSO-d6 ppm):δ 8.91 (s, 1H), 8.33 (d, J = 1.7 Hz, 1H), 8.14 (s, 1H), 8.07 (s, 1H), 7.91 (t, J = 1.4 Hz, 2H), 7.76 (dd, J = 8.4, 2.2 Hz, 1H), 7.58 (d, J = 8.6 Hz, 1H), 7.31 (d, J = 8.2 Hz, 1H), 7.26 (d, J = 1.7 Hz, 1H), 7.21-7.13 (m, 2H), 4.69 (s, 2H), 3.88 (s, 3H), 3.76 (t, J = 5.9 Hz, 2H), 3.52 (s, 2H), 2.91 (t, J = 5.9 Hz, 2H), 2.46-2.24 (m, 10H), 0.98 (t, J = 7.2 Hz, 3H). |
| II-3 | [chemical structure] | 580.4 [M + H]⁺ | ¹H NMR (400 MHz, DMSO-d6, ppm): δ 12.22 (s, 1H), 8.90 (s, 1H), 8.29 (s, 1H), 7.90 (d, J = 2.2 Hz, 1H), 7.75 (dd, J = 8.4, 2.2 Hz, 1H), 7.58 (d, J = 8.6 Hz, 1H), 7.47 (dd, J = 3.5, 2.3 Hz, 1H), 7.27 (d, J = 8.2 Hz, 1H), 7.13- 7.05 (m, 2H), 6.50 (dd, J = 3.5, 1.8 Hz, 1H), 4.67 (s, 2H), 3.75 (t, J = 5.9 Hz, 2H), 3.51 (s, 2H), 2.90 (t, J = 5.8 Hz, 2H), 2.48-2.13 (m, 10H), 0.98 (m, 3H). |
| II-4 | [chemical structure] | 580.2 [M + H]⁺ | ¹H NMR (400 MHz, DMSO-d6 , ppm) δ 13.70 (s, 1H), 8.90 (s, 1H), 8.35 (d, J = 5.4 Hz, 1H), 7.91 (d, J = 2.2 Hz, 1H), 7.81-7.72 (m, 2H), 7.58 (d, J = 8.6 Hz, 1H), 7.35 (d, J = 8.3 Hz, 1H), 7.20-7.10 (m, 2H), 6.46 (d, J = 5.3 Hz, 1H), 4.69 (s, 2H), 3.77 (t, J = 5.9 Hz, 2H), 3.51 (s, 2H), 2.93 (t, J = 5.9 Hz, 2H), 2.44-2.12 (m, 10H), 0.99 (t, J = 7.1 Hz, 3H). |
| II-5 | [chemical structure] | 581.15 [M + H]⁺ | ¹H NMR (400 MHz, DMSO-d6 , ppm) δ 8.91 (s, 1H), 8.49 (s, 1H), 8.13 (s, 1H), 7.91 (d, J = 2.2 Hz, 1H), 7.75 (dd, J = 8.5, 2.2 Hz, 1H), 7.58 (d, J = 8.6 Hz, 1H), 7.32 (d, J = 8.3 Hz, 1H), 7.20-7.11 (m, 2H), 4.68 (s, 2H), 3.77 (t, J = 5.9 Hz, 2H), 3.51 (s, 2H), 2.92 (t, J = 5.9 Hz, 2H), 2.42-2.17 (m, 10H), 0.99 (t, J = 7.2 Hz, 3H). |
| II-6 | [chemical structure] | 581.3 [M + H]⁺ | ¹H NMR (300 MHz, DMSO-d6) δ 8.91 (s, 1H), 8.44 (s, 1H), 8.38 (s, 1H), 7.91 (d, J = 2.2 Hz, 1H), 7.81-7.71 (m, 1H), 7.58 (d, J = 8.6 Hz, 1H), 7.29 (d, J = 8.1 Hz, 1H), 7.16-7.05 (m, 2H), 4.68 (s, 2H), 3.76 (t, J = 5.8 Hz, 2H), 3.51 (s, 2H), 2.90 (t, J = 5.9 Hz, 2H), 2.44-2.24 (m, 10H), 0.98 (t, J = 7.2 Hz, 3H) |
| II-7 | [chemical structure] | 594.25 [M + H]⁺ | ¹H NMR (300 MHz, Methanol-d4, ppm) δ 7.83 (s, 1H), 7.76-7.64 (m, 1H), 7.61-7.48 (m, 2H), 7.19 (d, J = 8.1 Hz, 1H), 7.06-6.89 (m, 2H), 6.85 (d, J = 4.4 Hz, 1H), 6.65-6.49 (m, 1H), 4.62 (s, 2H), 3.70 (t, J = 5.9 Hz, 2H), 3.53 (d, J = 1.4 Hz, 2H), 2.88 (t, J = 5.9 Hz, 2H), 2.74-1.98 (m, 13H), 1.04 (t, J = 7.3 Hz, 3H). |
| II-8 | [chemical structure] | 598.2 [M + H]⁺ | ¹H NMR (300 MHz, Methanol-d4) δ 8.02 (s, 1H), 7.88-7.75 (m, 2H), 7.66 (s, 2H), 7.32 (d, J = 8.0 Hz, 1H), 7.12 (d, J = 8.2 Hz, 2H), 6.78 (d, J = 1.9 Hz, 1H), 4.74 (s, 2H), 3.81 (t, J = 5.9 Hz, 2H), 3.62 (s, 2H), 2.99 (t, J = 5.9 Hz, 2H), 2.87-2.26 (m, 10H), 1.11 (t, J = 7.2 Hz, 3H). |
| II-9 | [chemical structure] | 598.15 [M + H]⁺ | N/A |
| II-10 | [chemical structure] | 624.15 [M + H]⁺ | N/A |
| II-11 | [chemical structure] | 609.4 [M + H]⁺ | ¹H NMR (300 MHz, Methanol-d4) δ 7.96 (d, J = 5.8 Hz, 1H), 7.82 (s, 1H), 7.66 (s, 2H), 7.31 (d, J = 8.3 Hz, 1H), 6.99 (s, 2H), 6.39 (dd, J = 5.9, 2.3 Hz, 1H), 4.72 (s, 2H), 3.80 (s, 2H), 3.63 (s, 2H), 3.19-2.91 (m, 4H), 2.79-2.19 (m, 12H), 1.18-1.00 (m, 3H). |
| II-12 | [chemical structure] | 607.4 [M + H]⁺ | ¹H NMR (400 MHz, Methanol-d4) δ 8.45-8.30 (m, 2H), 7.83 (d, J = 2.0 Hz, 1H), 7.77-7.61 (m, 2H), 7.38 (d, J = 8.0 Hz, 1H), 7.10 (d, J = 8.0 Hz, 2H), 6.70 (d, J = 9.6 Hz, 1H), 6.50 (d, J = 5.6 Hz, 1H), 4.76 (s, 2H), 3.84 (s, 2H), 3.64 (s, 2H), 3.02 (t, J = 5.9 Hz, 2H), 2.67-2.32 (m, 10H), 1.13 (t, J = 7.2 Hz, 3H). |
| II-13 | [chemical structure] | 569.3 [M + H]⁺ | N/A |
| II-14 | [chemical structure] | 597.25 [M + H]⁺ | N/A |
| II-15 | [chemical structure] | 623.3 [M + H]⁺ | N/A |
| II-16 | [chemical structure] | 567.25 [M + H]⁺ | ¹H NMR (400 MHz, Methanol-d4) δ 7.95 (s, 1H), 7.89 (dd, J = 2.6, 1.5 Hz, 1H), 7.68 (d, J = 2.7 Hz, 1H), 7.58 (dd, J = 8.9, 2.7 Hz, 1H), 7.34 (d, J = 8.0 Hz, 1H), 7.19-7.10 (m, 3H), 7.03 (dd, J = 4.5, 1.5 Hz, 1H), 6.93 (dd, J = 4.5, 2.6 Hz, 1H), 4.74 (s, 2H), 4.62 (s, 1H), 3.81 (t, J = 5.9 Hz, 2H), 3.01 (t, J = 5.8 Hz, 2H), 2.70 (s, 2H), 2.50 (s, 2H), 2.34 (s, 3H), 2.04 (m, 2H), 1.92 (s, 2H). |
| II-17 | [chemical structure] | 622.3 [M + H]⁺ | N/A |
| II-18 | [chemical structure] | 622.4 [M + H]⁺ | N/A |
| II-19 | [chemical structure] | 579.3 [M + H]⁺ | ¹H NMR (400 MHz, DMSO-d6) δ 9.26 (s, 1H), 8.97 (s, 1H), 8.01 (d, J = 5.3 Hz, 1H), 7.94 (d, J = 2.2 Hz, 1H), 7.90 (t, J = 2.2 Hz, 1H), 7.82 (dd, J = 8.4, 2.2 Hz, 1H), 7.60 (d, J = 8.6 Hz, 1H), 7.36 (d, J = 8.2 Hz, 1H), 7.20- 7.09 (m, 2H), 6.85 (dd, J = 4.3, 2.7 Hz, 1H), 6.69 (dd, J = 4.3, 1.6 Hz, 1H), 5.80 (d, J = 5.4 Hz, 1H), 4.70 (s, 2H), 3.77 (m, 2H), 3.65 (s, 2H), 3.47 (m, 2H), 3.15 (m, 2H), 3.01-2.89 (m, 6H), 2.38 (m, 2H), 1.21 (t, J = 7.3 Hz, 3H) |
| II-20 | [chemical structure] | 580.4 [M + H]⁺ | ¹H NMR (400 MHz, Methanol-d4) δ 8.35 (s, 1H), 8.23 (d, J = 6.0 Hz, 1H), 7.83 (s, 1H), 7.70-7.64 (m, 2H), 7.36 (d, J = 8.0 Hz, 1H), 7.11 (d, J = 8.1 Hz, 2H), 6.64 (d, J = 5.9 Hz, 1H), 3.84 (t, J = 6.3 Hz, 2H), 3.64 (s, 2H), 3.02 (d, J = 6.3 Hz, 2H), 2.55 (m, 10H), 1.31 (s, 2H), 1.13 (t, J = 7.2 Hz, 3H) |
| II-21 | [chemical structure] | 529.4 [M + H]⁺ | N/A |
| II-22 | [chemical structure] | 597.35 [M + H]⁺ | ¹H NMR (400 MHz, Methanol-d4) δ 7.94 (d, J = 5.5 Hz, 1H), 7.85 (d, J = 2.2 Hz, 1H), 7.77-7.60 (m, 3H), 7.36 (d, J = 9.0 Hz, 1H), 7.10 (d, J = 6.8 Hz, 2H), 6.48 (d, J = 1.9 Hz, 1H), 5.91 (d, J = 5.5 Hz, 1H), 4.75 (s, 2H), 3.82 (d, J = 5.9 Hz, 2H), 3.77 (s, 2H), 3.50 (s, 2H), 3.28-2.33 (m, 10H), 1.36 (t, J = 7.3 Hz, 3H). |
| II-23 | [chemical structure] | 613.4 [M + H]⁺ | N/A |
| II-24 | [chemical structure] | 659.05 [M + H]⁺ | ¹H NMR (400 MHz, Methanol-d4) δ 7.90 (d, J = 5.5 Hz, 1H), 7.81 (dd, J = 10.2, 1.9 Hz, 2H), 7.72-7.61 (m, 2H), 7.39-7.26 (m, 1H), 7.08 (d, J = 7.2 Hz, 2H), 6.74 (d, J = 1.8 Hz, 1H), 5.84 (d, J = 5.5 Hz, 1H), 4.74 (s, 2H), 3.82 (t, J = 5.9 Hz, 2H), 3.62 (d, J = 1.7 Hz, 2H), 2.99 (t, J = 5.9 Hz, 2H), 2.77-2.19 (m, 10H), 1.12 (t, J = 7.2 Hz, 3H). |
| II-25 | [chemical structure] | 645.3 [M + H]⁺ | ¹H NMR (400 MHz, Methanol-d4) δ 8.07 (d, J = 1.7 Hz, 1H), 7.93-7.79 (m, 2H), 7.77-7.60 (m, 3H), 7.47- 7.31 (m, 2H), 7.12 (d, J = 7.7 Hz, 2H), 6.98 (d, J = 1.8 Hz, 1H), 5.83 (d, J = 5.4 Hz, 1H), 4.76 (m, 3H), 3.84 (t, J = 5.9 Hz, 2H), 3.64 (s, 2H), 3.02 (t, J = 5.9 Hz, 2H), 2.76-2.33 (m, 9H), 1.12 (t, J = 7.2 Hz, 3H). |
| II-26 | [chemical structure] | 659.55 [M + H]⁺ | ¹H NMR (400 MHz, Methanol-d4) δ 8.02-7.89 (m, 2H), 7.89-7.75 (m, 3H), 7.66 (d, J = 3.0 Hz, 2H), 7.35 (d, J = 8.5 Hz, 1H), 7.10 (d, J = 6.9 Hz, 2H), 6.85 (d, J = 1.8 Hz, 1H), 5.80 (d, J = 5.4 Hz, 1H), 4.75 (s, 2H), 3.94 (s, 3H), 3.83 (t, J = 5.9 Hz, 2H), 3.63 (s, 2H), 3.00 (t, J = 5.8 Hz, 2H), 2.83- 2.21 (m, 10H), 1.12 (t, J = 7.2 Hz, 3H). |

### Example 3—Synthesis of 7-((1H-Pyrrolo[2,3-b]pyridin-4-yl)oxy)-N-(4-((4-ethylpiperazin-1-yl)methyl)-3-(trifluoromethyl)phenyl)-3,4-dihydroisoquinoline-2(1H)-carboxamide (Compound No. 2)

[chemical structure]

### Part I—Synthesis of 4-bromo-1-(triisopropylsilyl)-1H-pyrrolo[2,3-b]pyridine

[chemical structure]

Into a 50 mL round-bottom flask, was placed 4-bromo-1H-pyrrolo[2,3-b]pyridine (1.0 g, 5.08 mmol) in anhydrous tetrahydrofuran (20 mL) at 0° C. followed by the addition of sodium hydride (146 mg, 6.08 mmol). The resulting solution was stirred for 30 minutes at 0° C. To the mixture was added chlorotris(propan-2-yl)silane (1.17 g, 6.07 mmol). The resulting solution was stirred for an additional 2 h at room temperature. The reaction was then quenched by the addition of 50 mL of NH₄Cl (aq). The resulting mixture was extracted with ethyl acetate, and the organic layers were combined and concentrated under vacuum. The residue was purified by column chromatography eluting with ethyl acetate/petroleum ether. The collected fractions were combined and concentrated under vacuum to yield 4-bromo-1-(triisopropylsilyl)-1H-pyrrolo[2,3-b]pyridine (1.53 g, 85%).

### Part II—Synthesis of (1-(Triisopropylsilyl)-1H-pyrrolo[2,3-b]pyridin-4-yl)boronic Acid

[chemical structure]

Into a 50 mL, 3-necked round-bottom flask purged and maintained with an inert atmosphere of nitrogen, was placed 4-bromo-1-(triisopropylsilyl)-1H-pyrrolo[2,3-b]pyridine (400 mg, 1.13 mmol), tetrahydrofuran (20 mL) and trimethyl borate (233 mg, 2.24 mmol). The resulting solution was stirred for 5 min at −78° C., then a solution of n-butyllithium (1.4 mL, 3.5 mmol) in hexane was added into the flask. The resulting solution was allowed to react, with stirring, for an additional 1 hour while the temperature was maintained at −78° C. The reaction was then quenched by the addition of 50 mL of NH₄Cl (aq). The resulting mixture was extracted with ethyl acetate, and the organic layers were combined and concentrated under vacuum. The residue was purified by column chromatography eluting with ethyl acetate/petroleum ether (1:2). The collected fractions were combined and concentrated under vacuum to yield (1-(triisopropylsilyl)-1H-pyrrolo[2,3-b]pyridin-4-yl)boronic acid (200 mg, 56%).

### Part III—Synthesis of tert-Butyl 7-((1-(triisopropylsilyl)-1H-pyrrolo[2,3-b]pyridin-4-yl)oxy)-3,4-dihydroisoquinoline-2(1H)-carboxylate

[chemical structure]

Into a 25 mL round-bottom flask, was placed copper(II) acetate (60 mg, 0.33 mmol), dichloromethane (10 mL) and pyridine (120 mg, 1.52 mmol). The resulting solution was stirred for 20 minutes at room temperature. To the mixture was added (1-(triisopropylsilyl)-1H-pyrrolo[2,3-b]pyridin-4-yl)boronic acid (100 mg, 0.31 mmol), tert-butyl 7-hydroxy-1,2,3,4-tetrahydroisoquinoline-2-carboxylate (120 mg, 0.48 mmol, 1.40 equiv) and molecular sieves (4A) (600 mg). This mixture was stirred at room temperature. Then the solids were removed by filtration, and the filtrate was concentrated under vacuum. The residue was purified by column chromatography eluting with ethyl acetate/petroleum ether (1:2). The collected fractions were combined and concentrated under vacuum to yield tert-butyl 7-((1-(triisopropylsilyl)-1H-pyrrolo[2,3-b]pyridin-4-yl)oxy)-3,4-dihydroisoquinoline-2(1H)-carboxylate (90 mg, 55%).

### Part IV—Synthesis of 7-((1H-Pyrrolo[2,3-b]pyridin-4-yl)oxy)-1,2,3,4-tetrahydroisoquinoline

[chemical structure]

Into a 25 mL round-bottom flask was placed tert-butyl 7-((1-(triisopropylsilyl)-1H-pyrrolo[2,3-b]pyridin-4-yl)oxy)-3,4-dihydroisoquinoline-2(1H)-carboxylate (90 mg, 0.17 mmol), dichloromethane (4 mL) and trifluoroacetic acid (2 mL). The resulting solution was stirred for 2 hours at room temperature. The resulting mixture was concentrated under vacuum to yield 7-((1H-pyrrolo[2,3-b]pyridin-4-yl)oxy)-1,2,3,4-tetrahydroisoquinoline as the bis trifluoroacetic acid solvate (67 mg, 79%).

### Part V—Synthesis of 7-((1H-Pyrrolo[2,3-b]pyridin-4-yl)oxy)-N-(4-((4-ethylpiperazin-1-yl)methyl)-3-(trifluoromethyl)phenyl)-3,4-dihydroisoquinoline-2(1H)-carboxamide (Compound No. 2)

[chemical structure]

Into a 25 mL round-bottom flask, was placed 1-ethyl-4-[[4-isocyanato-2-(trifluoromethyl)phenyl]methyl]piperazine (67 mg, 0.21 mmol), dichloromethane (5 mL), triethylamine (80 mg, 0.79 mmol) and 7-((1H-pyrrolo[2,3-b]pyridin-4-yl)oxy)-1,2,3,4-tetrahydroisoquinoline bis(trifluoroacetic acid) (62 mg, 0.13 mmol). The resulting solution was stirred for 4 hours at room temperature. The resulting mixture was concentrated under vacuum. The crude product was purified by preparatory HPLC to yield 7-((1H-pyrrolo[2,3-b]pyridin-4-yl)oxy)-N-(4-((4-ethylpiperazin-1-yl)methyl)-3-(trifluoromethyl)phenyl)-3,4-dihydroisoquinoline-2(1H)-carboxamide (16.1 mg, 13%). (ES, m/z): [M+H]⁺=579; ¹H NMR (400 MHz, DMSO-d₆, ppm): δ 11.73 (s, 1H), 8.86 (s, 1H), 8.08 (d, J=5.4 Hz, 1H), 7.90 (d, J=2.2 Hz, 1H), 7.75 (dd, J=8.6, 2.2 Hz, 1H), 7.57 (d, J=8.6 Hz, 1H), 7.36 (dd, J=3.4, 2.4 Hz, 1H), 7.28 (d, J=8.2 Hz, 1H), 7.02 (d, J=7.4 Hz, 2H), 6.43 (d, J=5.4 Hz, 1H), 6.23 (dd, J=3.5, 1.9 Hz, 1H), 4.65 (s, 2H), 3.74 (t, J=5.9 Hz, 2H), 3.51 (s, 2H), 2.89 (t, J=5.8 Hz, 2H), 2.49-2.13 (m, 10H), 0.98 (t, J=7.1 Hz, 3H).

### Example 4—Synthesis of Additional N-phenyl-3,4-dihydroisoquinoline-2(1H)-carboxamides and Related Compounds

The compounds in Table 3 below were prepared based on experimental procedures described in Example 3 and in the detailed description.

TABLE 3

| | | Mass Spec. | |
|---|---|---|---|
| No. | Chemical Structure | (ES, m/z) | NMR Spectrum |
| III-1 | [chemical structure] | 593.25 [M + H]⁺ | ¹H NMR (400 MHz, DMSO- d6, ppm) δ 11.71 (s, 1H), 8.86 (s, 1H), 8.04 (d, J = 5.4 Hz, 1H), 7.90 (d, J = 2.3 Hz, 1H), 7.75 (dd, J = 8.5, 2.2 Hz, 1H), 7.58 (d, J = 8.6 Hz, 1H), 7.35 (dd, J = 3.5, 2.4 Hz, 1H), 7.21 (s, 1H), 6.96 (s, 1H), 6.29-6.22 (m, 2H), 4.61 (s, 2H), 3.74 (t, J = 5.9 Hz, 2H), 3.54-3.48 (m, 2H), 2.87 (t, J = 5.8 Hz, 2H), 2.49-2.18 (m, 10H), 2.10 (s, 3H), 0.98 (t, J = 7.2 Hz, 3H). |
| III-2 | [chemical structure] | 593.2 [M + H]⁺ | ¹H NMR (300 MHz, DMSO- d6) δ 11.71 (s, 1H), 8.95 (s, 1H), 8.02 (d, J = 5.4 Hz, 1H), 7.91 (d, J = 2.2 Hz, 1H), 7.80-7.70 (m, 1H), 7.59 (d, J = 8.6 Hz, 1H), 7.39-7.31 (m, 1H), 7.14 (d, J = 8.3 Hz, 1H), 7.00 (d, J = 8.3 Hz, 1H), 6.28 (dd, J = 3.5, 1.9 Hz, 1H), 6.18 (d, J = 5.4 Hz, 1H), 4.62 (s, 2H), 3.74 (t, J = 5.7 Hz, 2H), 3.52 (s, 2H), 3.32 (s, 2H), 2.90 (s, 2H), 2.45-2.19 (m, 8H), 2.07 (s, 3H), 0.98 (t, J = 7.2 Hz, 3H) |
| III-3 | [chemical structure] | 597.15 [M + H]⁺ | N/A |
| III-4 | [chemical structure] | 593.25 [M + H]⁺ | ¹H NMR (300 MHz, Methanol-d4) δ 7.90 (d, J = 5.6 Hz, 1H), 7.78 (d, J = 1.9 Hz, 1H), 7.69-7.45 (m, 2H), 7.24 (d, J = 9.1 Hz, 1H), 7.06-6.81 (m, 2H), 6.43 (d, J = 5.6 Hz, 1H), 5.93 (q, J = 1.0 Hz, 1H), 4.67 (s, 2H), 3.77 (t, J = 5.9 Hz, 2H), 3.59 (d, J = 1.6 Hz, 2H), 2.94 (t, J = 5.8 Hz, 2H), 2.74-2.38 (m, 10H), 2.37 (m, 3H), 1.08 (t, J = 7.2 Hz, 3H). |
| III-5 | [chemical structure] | 581.15 [M + H]⁺ | N/A |
| III-6 | [chemical structure] | 593.3 [M + H]⁺ | ¹H NMR (400 MHz, DMSO- d6, ppm) δ 11.63 (s, 1H), 8.80 (s, 1H), 8.14 (s, 1H), 7.89 (d, J = 2.2 Hz, 1H), 7.74 (dd, J = 8.6, 2.2 Hz, 1H), 7.57 (d, J = 8.6 Hz, 1H), 7.26 (dd, J = 3.5, 2.5 Hz, 1H), 7.18 (d, J = 8.3 Hz, 1H), 6.85-6.75 (m, 2H), 5.69 (dd, J = 3.5, 1.9 Hz, 1H), 4.58 (s, 2H), 3.71 (t, J = 5.9 Hz, 2H), 3.51 (s, 2H), 2.84 (t, J = 5.8 Hz, 2H), 2.48-2.26 (m, 10H), 2.25 (s, 3H), 0.98 (t, J = 7.2 Hz, 3H). |
| III-7 | [chemical structure] | 613.2 [M + H]⁺ | N/A |
| III-8 | [chemical structure] | 597.2 [M + H]⁺ | N/A |
| III-9 | [chemical structure] | 593.3 [M + H]⁺ | ¹H NMR (400 MHz, Methanol-d4) δ 8.04 (d, J = 5.6 Hz, 1H), 7.83 (d, J = 1.9 Hz, 1H), 7.75-7.56 (m, 2H), 7.27 (d, J = 3.5 Hz, 1H), 6.91 (dd, J = 26.1, 2.4 Hz, 2H), 6.48 (d, J = 5.6 Hz, 1H), 6.34 (d, J = 3.6 Hz, 1H), 4.72 (s, 2H), 3.86 (t, J = 6.0 Hz, 2H), 3.64 (s, 2H), 2.89 (t, J = 6.0 Hz, 2H), 2.86-2.37 (m, 10H), 2.33 (m, 3H), 1.13 (t, J = 7.3 Hz, 3H) |
| III-11 | [chemical structure] | 566.45 [M + H]⁺ | ¹H NMR (400 MHz, DMSO- d6) δ 11.75 (s, 1H), 8.69 (s, 1H), 8.09 (d, J = 5.4 Hz, 1H), 7.78 (d, J = 2.7 Hz, 1H), 7.68 (dd, J = 8.9, 2.7 Hz, 1H), 7.37 (d, J = 3.5 Hz, 1H), 7.29 (d, J = 9.0 Hz, 1H), 7.22 (d, J = 9.2 Hz, 1H), 7.03 (d, J = 6.4 Hz, 2H), 6.44 (d, J = 5.4 Hz, 1H), 6.24 (d, J = 3.5 Hz, 1H), 4.64 (s, 2H), 4.51 (s, 1H), 3.74 (t, J = 5.9 Hz, 2H), 3.34 (s, 2H), 2.89 (m, 2H), 2.23 (s, 2H), 2.16 (s, 3H), 1.88 (m, 2H), 1.67 (m, 2H). |
| III-12 | [chemical structure] | 593.25 [M + H]⁺ | N/A |
| III-13 | [chemical structure] | 607.5 [M + H]⁺ | N/A |

### Example 5—Synthesis of 7-((1H-Pyrrolo[2,3-b]pyridin-4-yl)oxy)-N-(4-((4-ethylpiperazin-1-yl)methyl)-3-(trifluoromethyl)phenyl)-6-fluoro-3,4-dihydroisoquinoline-2(1H)-carboxamide (Compound No. 3)

[chemical structure]

### Part I—Synthesis of 2-Fluoro-1-methoxy-4-(2-nitrovinyl)benzene

[chemical structure]

Into a 100 mL round-bottom flask purged and maintained with an inert atmosphere of nitrogen, was placed 3-fluoro-4-methoxybenzaldehyde (3.0 g, 19.46 mmol). This was followed by the addition of ammonium acetate (1.65 g, 21.41 mmol) at room temperature. To this mixture was added nitromethane (30 mL) at room temperature. The reaction was stirred for 3 hours at 100° C. in an oil bath then diluted with ethyl acetate and washed with water. The organics were concentrated under vacuum. The residue was purified by column chromatography eluting with ethyl acetate/petroleum ether (1:10). The collected fractions were combined and concentrated under vacuum to provide 2-fluoro-1-methoxy-4-(2-nitrovinyl)benzene (1.2 g, 31%).

### Part II—Synthesis of 2-(3-Fluoro-4-methoxyphenyl)ethan-1-amine

[chemical structure]

Into a 100 mL round-bottom flask was placed a solution of 2-fluoro-1-methoxy-4-(2-nitrovinyl)benzene (1 g, 5.08 mmol) in tetrahydrofuran (20 mL) followed by a 2.5 M solution of lithium aluminum hydride in tetrahydrofuran (10 mL, 25.4 mmol) dropwise with stirring at 0° C. The resulting solution was stirred for 2 hours at room temperature. The reaction was quenched by the addition of water (1 mL), 15% sodium hydroxide (aq) (1 ml), then water (3 mL) and the resulting mixture was stirred for 15 min. Magnesium sulfate was added and the mixture was stirred for an additional 15 min at room temperature. The solids were removed by filtration, and the filtrate was concentrated under vacuum. The residue was purified by column chromatography eluting with dichloromethane/methanol (10:1). The collected fractions were combined and concentrated under vacuum to yield 2-(3-fluoro-4-methoxyphenyl)ethan-1-amine (480 mg, 56%).

### Part III—Synthesis of 6-Fluoro-7-methoxy-1,2,3,4-tetrahydroisoquinoline

[chemical structure]

Into a 50 mL round-bottom flask, purged and maintained with an inert atmosphere of nitrogen, was placed a solution of 2-(3-fluoro-4-methoxyphenyl)ethan-1-amine (480 mg, 2.84 mmol) in toluene (4 mL) followed by trifluoroacetic acid (4 mL) at room temperature. To this mixture was added paraformaldehyde (480 mg) at room temperature. The resulting solution was stirred for 2 days at 60° C. then concentrated under reduced pressure. The resulting residue was purified by column chromatography eluting with dichloromethane/methanol (20:1). The collected fractions were combined and concentrated under vacuum to yield 6-fluoro-7-methoxy-1,2,3,4-tetrahydroisoquinoline (300 mg, 58%).

### Part IV—Synthesis of 6-Fluoro-1,2,3,4-tetrahydroisoquinolin-7-ol

[chemical structure]

Into a 100 mL round-bottom flask, was placed a solution of 6-fluoro-7-methoxy-1,2,3,4-tetrahydroisoquinoline (300 mg, 1.66 mmol) in dichloromethane (20 mL) followed by tribromoborane (2.0 g, 7.98 mmol) added dropwise with stirring at 0° C. The resulting solution was stirred for 2 hours at room temperature. The reaction then was quenched by the addition of methanol and concentrated under vacuum to yield 6-fluoro-1,2,3,4-tetrahydroisoquinolin-7-ol as a crude mixture (277 mg).

### Part V—Synthesis of tert-Butyl 7-((tert-butoxycarbonyl)oxy)-6-fluoro-3,4-dihydroisoquinoline-2(1H)-carboxylate

[chemical structure]

Into a 50 mL round-bottom flask, was placed a solution of 6-fluoro-1,2,3,4-tetrahydroisoquinolin-7-ol (270 mg, 1.62 mmol) in dichloromethane (10 mL) followed by triethylamine (490 mg, 4.84 mmol) at room temperature. To this mixture was added di-tert-butyl dicarbonate (349 mg, 1.60 mmol) and the resulting solution was stirred for 3 hours at room temperature before being concentrated under vacuum. The resulting residue was purified by column chromatography eluting with ethyl acetate/petroleum ether (1:13). The collected fractions were combined and concentrated under vacuum to yield tert-butyl 7-((tert-butoxycarbonyl)oxy)-6-fluoro-3,4-dihydroisoquinoline-2(1H)-carboxylate (220 mg, 37%).

### Part VI—Synthesis of tert-Butyl 6-fluoro-7-hydroxy-3,4-dihydroisoquinoline-2(1H)-carboxylate

[chemical structure]

Into a 25 mL round-bottom flask, was placed a solution of tert-butyl 7-((tert-butoxycarbonyl)oxy)-6-fluoro-3,4-dihydroisoquinoline-2(1H)-carboxylate (210 mg, 0.57 mmol) in methanol (4 mL) followed by sodium methoxide (1.5 mL, 1.5 mmol). The resulting solution was stirred for 3 hours at room temperature before being concentrated under vacuum. The residue was purified by column chromatography eluting with ethyl acetate/petroleum ether (1:2). The collected fractions were combined and concentrated under vacuum to yield tert-butyl 6-fluoro-7-hydroxy-3,4-dihydroisoquinoline-2(1H)-carboxylate (130 mg, 85%).

### Part VII—Synthesis of tert-Butyl 6-fluoro-7-((1-(triisopropylsilyl)-1H-pyrrolo[2,3-b]pyridin-4-yl)oxy)-3,4-dihydroisoquinoline-2(1H)-carboxylate

[chemical structure]

Into a 50 mL round-bottom flask purged and maintained with an inert atmosphere of nitrogen, was placed a solution of copper(II) acetate (81 mg, 0.45 mmol) in dichloromethane (10 mL) followed by pyridine (178 mg, 2.25 mmol). The resulting solution was stirred for 20 min at room temperature. To this mixture was added tert-butyl 6-fluoro-7-hydroxy-3,4-dihydroisoquinoline-2(1H)-carboxylate (120 mg, 0.45 mmol) at room temperature then (1-(triisopropylsilyl)-1H-pyrrolo[2,3-b]pyridin-4-yl)boronic acid (143 mg, 0.45 mmol) and molecular sieves (4A) (720 mg). The resulting mixture was stirred for an additional 2 days at room temperature. The mixture was concentrated to provide a residue, and then residue was purified by column chromatography eluting with ethyl acetate/petroleum ether (1:15). The collected fractions were combined and concentrated under vacuum to yield tert-butyl 6-fluoro-7-((1-(triisopropylsilyl)-1H-pyrrolo[2,3-b]pyridin-4-yl)oxy)-3,4-dihydroisoquinoline-2(1H)-carboxylate (33 mg, 14%).

### Part VIII—Synthesis of 7-((1H-Pyrrolo[2,3-b]pyridin-4-yl)oxy)-6-fluoro-1,2,3,4-tetrahydroisoquinoline

[chemical structure]

Into a 25 mL round-bottom flask was charged tert-butyl 6-fluoro-7-((1-(triisopropylsilyl)-1H-pyrrolo[2,3-b]pyridin-4-yl)oxy)-3,4-dihydroisoquinoline-2(1H)-carboxylate (33 mg, 0.06 mmol) in dichloromethane (1 mL) followed by trifluoroacetic acid (2 mL). The resulting solution was stirred overnight at room temperature then concentrated under vacuum to yield 7-((1H-pyrrolo[2,3-b]pyridin-4-yl)oxy)-6-fluoro-1,2,3,4-tetrahydroisoquinoline as a trifluoroacetate solvate (17 mg).

### Part IX—Synthesis of 7-((1H-Pyrrolo[2,3-b]pyridin-4-yl)oxy)-N-(4-((4-ethylpiperazin-1-yl)methyl)-3-(trifluoromethyl)phenyl)-6-fluoro-3,4-dihydroisoquinoline-2(1H)-carboxamide (Compound No. 3)

[chemical structure]

Into a 25 mL round-bottom flask, was placed a solution of 7-((1H-pyrrolo[2,3-b]pyridin-4-yl)oxy)-6-fluoro-1,2,3,4-tetrahydroisoquinoline (14 mg, 0.05 mmol) in dichloromethane (3 mL). This was followed by the addition of 1-ethyl-4-[[4-isocyanato-2-(trifluoromethyl)phenyl]methyl]piperazine (15.5 mg, 0.05 mmol) and triethylamine (15 mg, 0.15 mmol) at room temperature. The resulting solution was stirred for 2 hours at room temperature then concentrated under vacuum and purified by preparatory HPLC to yield 7-((1H-pyrrolo[2,3-b]pyridin-4-yl)oxy)-N-(4-((4-ethylpiperazin-1-yl)methyl)-3-(trifluoromethyl)phenyl)-6-fluoro-3,4-dihydroisoquinoline-2(1H)-carboxamide (4.2 mg, 14%). (ES, m/z): [M+H]⁺: 597; ¹H NMR (400 MHz, Chloroform-d, ppm) δ 9.51 (s, 1H), 8.18 (d, J=5.5 Hz, 1H), 7.65 (m, 3H), 7.26 (d, J=3.6 Hz, 1H), 7.08 (dd, J=14.2, 9.0 Hz, 2H), 6.66 (s, 1H), 6.47 (dd, J=14.5, 4.5 Hz, 2H), 4.66 (s, 2H), 3.80 (t, J=5.9 Hz, 2H), 3.64 (d, J=1.5 Hz, 2H), 3.00 (t, J=5.9 Hz, 2H), 2.62 (br s, 10H), 1.19 (t, J=7.3 Hz, 3H).

### Example 6—Synthesis of 7-((1H-pyrrolo[2,3-b]pyridin-4-yl)oxy)-N-(4-((4-ethylpiperazin-1-yl)methyl)-3-(trifluoromethyl)phenyl)-6-fluoro-4-methyl-3,4-dihydroisoquinoline-2(1H)-carboxamide (Compound No. 4)

[chemical structure]

### Part I—Synthesis of 2-(3-Fluoro-4-methoxyphenyl)propanenitrile

[chemical structure]

Into a 500-mL 3-necked round-bottom flask purged and maintained with an inert atmosphere of nitrogen was placed diisopropylamine (4.9 g, 48.44 mmol) in anhydrous tetrahydrofuran (60 mL). To this was added 2.5 M n-butyllithium in hexanes (18.2 mL, 46 mmol) dropwise with stirring at −78° C. The reaction was stirred for 30 min at −78° C., then 2-(3-fluoro-4-methoxyphenyl)acetonitrile (5.0 g, 30 mmol) was added dropwise with stirring at −78° C. The reaction was stirred for 30 min then iodomethane (4.7 g, 33 mmol) was added. The resulting solution was stirred for 3 hours at −78° C. The reaction was then quenched by the addition of saturated ammonium chloride, and the resulting mixture was extracted with ethyl acetate (3×200 mL). The organics were concentrated under vacuum. The residue was applied onto a silica gel column and eluted with ethyl acetate/petroleum ether (1:6.5) to yield 2-(3-fluoro-4-methoxyphenyl)propanenitrile (1.8 g, 33%).

### Part II—Synthesis of 2-(3-Fluoro-4-methoxyphenyl)propan-1-amine

[chemical structure]

Into a 100-mL round-bottom flask purged and maintained with an inert atmosphere of nitrogen, was placed 2-(3-fluoro-4-methoxyphenyl)propanenitrile (1.8 g, 10 mmol), tetrahydrofuran (50 mL), followed by 1 M borane-tetrahydrofuran (50 mL, 50 mmol) at 0° C. The resulting solution was stirred overnight at 65° C. The reaction was quenched with methanol (50 mL). The resulting mixture was concentrated under vacuum. The crude product was purified by Flash-Prep-HPLC with a gradient of acetonitrile in water with 0.5% trifluoroacetic acid to yield 2-(3-fluoro-4-methoxyphenyl)propan-1-amine as the trifluoroacetate salt (2.9 g, 97%).

### Part III—Synthesis of 6-Fluoro-7-hydroxy-4-methyl-3,4-dihydroisoquinolin-1(2H)-one

[chemical structure]

Into a 100 mL round-bottom flask was placed 2-(3-fluoro-4-methoxyphenyl)propan-1-amine trifluoroacetate (2.88 g, 9.69 mmol) in dichloromethane (40 mL). The solution was cooled to 0° C. and 1 M sodium hydroxide (10 mL, 10 mmol) was added followed by triphosgene (1.1 g, 3.9 mmol). The resulting solution was stirred for 1.5 hours at room temperature. The mixture was diluted with brine (100 mL) and extracted with dichloromethane (3×200 mL). The organics were concentrated under vacuum, then the crude product was dissolved in trifluoromethanesulfonic acid (20 mL). The solution was stirred at 100° C. for 2 hours. The resulting solution was concentrated under vacuum to yield crude 6-fluoro-7-hydroxy-4-methyl-3,4-dihydroisoquinolin-1(2H)-one (930 mg, 49%).

### Part IV—Synthesis of 6-Fluoro-4-methyl-1,2,3,4-tetrahydroisoquinolin-7-ol

[chemical structure]

Into a 100 mL round-bottom flask, purged and maintained with an inert atmosphere of nitrogen, was placed 6-fluoro-7-hydroxy-4-methyl-3,4-dihydroisoquinolin-1(2H)-one (916 mg, 4.69 mmol) in anhydrous tetrahydrofuran (20 mL). The solution was cooled to 0° C. and lithium aluminum hydride (1.8 g, 47 mmol) was added slowly. The mixture was stirred at 65° C. overnight. The reaction was then quenched by slow addition of water (3 mL), 15% sodium hydroxide (3 mL) and water (9 mL). The solids were filtered out. The filtrate was concentrated under vacuum, and the residue was purified on a silica gel column eluting with ethyl acetate/petroleum ether (0:100-20:80) to yield 6-fluoro-4-methyl-1,2,3,4-tetrahydroisoquinolin-7-ol (800 mg, 94%).

### Part V—Synthesis of tert-Butyl 6-fluoro-7-hydroxy-4-methyl-3,4-dihydroisoquinoline-2(1H)-carboxylate

[chemical structure]

Into a 100 mL round-bottom flask was placed 6-fluoro-4-methyl-1,2,3,4-tetrahydroisoquinolin-7-ol (800 mg, 4.41 mmol), dichloromethane (30 mL), 1 M sodium hydroxide (30 mL, 30 mmol), followed by di-tert-butyl dicarbonate (3.0 g, 13.8 mmol). The resulting mixture was stirred at room temperature overnight. The organic layer was concentrated under vacuum, and the residue was purified on a silica gel column eluting with ethyl acetate/petroleum ether (0:100-20:80) to yield tert-butyl 6-fluoro-7-hydroxy-4-methyl-3,4-dihydroisoquinoline-2(1H)-carboxylate (600 mg, 48%).

### Part VI—Synthesis of tert-Butyl 6-fluoro-4-methyl-7-((1-(triisopropylsilyl)-1H-pyrrolo[2,3-b]pyridin-4-yl)oxy)-3,4-dihydroisoquinoline-2(1H)-carboxylate

[chemical structure]

Into a 100 mL round-bottom flask, purged and maintained with an atmosphere of oxygen, was placed copper(II) acetate (129 mg, 0.71 mmol), dichloromethane (10 mL), and pyridine (0.29 mL, 3.55 mmol). The reaction was stirred for 20 min at room temperature. To the mixture was added 4 Å molecular sieves (2 g), tert-butyl 6-fluoro-7-hydroxy-4-methyl-3,4-dihydroisoquinoline-2(1H)-carboxylate (200 mg, 0.71 mmol), and (1-(triisopropylsilyl)-1H-pyrrolo[2,3-b]pyridin-4-yl)boronic acid (340 mg, 1.07 mmol). The resulting solution was stirred at room temperature overnight. The solids were filtered out, and the filtrate was concentrated under vacuum. The resulting residue was purified on a silica gel column eluting with ethyl acetate/petroleum ether (1:6) to yield tert-butyl 6-fluoro-4-methyl-7-((1-(triisopropylsilyl)-1H-pyrrolo[2,3-b]pyridin-4-yl)oxy)-3,4-dihydroisoquinoline-2(1H)-carboxylate (194 mg, 49%).

### Part VII—Synthesis of 7-((1H-Pyrrolo[2,3-b]pyridin-4-yl)oxy)-6-fluoro-4-methyl-1,2,3,4-tetrahydroisoquinoline

[chemical structure]

Into a 50 mL round-bottom flask was placed tert-butyl 6-fluoro-4-methyl-7-((1-(triisopropylsilyl)-1H-pyrrolo[2,3-b]pyridin-4-yl)oxy)-3,4-dihydroisoquinoline-2(1H)-carboxylate (194 mg, 0.35 mmol) and 1 M hydrogen chloride (6 mL). The resulting solution was stirred for 30 minutes at room temperature. The mixture was concentrated under vacuum to yield crude 7-((1H-pyrrolo[2,3-b]pyridin-4-yl)oxy)-6-fluoro-4-methyl-1,2,3,4-tetrahydroisoquinoline as the hydrochloride salt (114 mg).

### Part VIII—Synthesis of 7-((1H-Pyrrolo[2,3-b]pyridin-4-yl)oxy)-N-(4-((4-ethylpiperazin-1-yl)methyl)-3-(trifluoromethyl)phenyl)-6-fluoro-4-methyl-3,4-dihydroisoquinoline-2(1H)-carboxamide (Compound No. 4)

[chemical structure]

Into a 100 mL round-bottom flask, was placed 7-((1H-pyrrolo[2,3-b]pyridin-4-yl)oxy)-6-fluoro-4-methyl-1,2,3,4-tetrahydroisoquinoline (104 mg, 0.35 mmol) and 1-ethyl-4-(4-isocyanato-2-(trifluoromethyl)benzyl)piperazine (219 mg, 0.7 mmol), in dichloromethane (5 mL) and triethylamine (1 mL, 7.2 mmol). The resulting solution was stirred for 30 minutes at room temperature. The resulting mixture was concentrated and purified by preparatory HPLC to yield 7-((1H-pyrrolo[2,3-b]pyridin-4-yl)oxy)-N-(4-((4-ethylpiperazin-1-yl)methyl)-3-(trifluoromethyl)phenyl)-6-fluoro-4-methyl-3,4-dihydroisoquinoline-2(1H)-carboxamide (136 mg, 54%). (ES, m/z): [M+H]⁺=611. ¹H NMR (400 MHz, Methanol-d₄, ppm) δ 8.33 (dd, J=7.0, 2.0 Hz, 1H), 7.87 (d, J=2.2 Hz, 1H), 7.76-7.66 (m, 2H), 7.60 (d, J=3.6 Hz, 1H), 7.38 (dd, J=14.8, 9.5 Hz, 2H), 6.91-6.85 (m, 1H), 6.72 (d, J=3.6 Hz, 1H), 4.87 (d, J=16.5 Hz, 1H), 4.68 (d, J=16.4 Hz, 1H), 3.90-3.75 (m, 2H), 3.67 (m, 1H), 3.23 (m, 4H), 2.8 (m, 8H), 1.4 (d, 3H), 1.35 (t, 3H).

### Example 7—Synthesis of Additional N-phenyl-3,4-dihydroisoquinoline-2(1H)-carboxamides and Related Compounds

The compounds in Table 4 below were prepared based on experimental procedures described in Examples 5 and 6 and in the detailed description.

TABLE 4

| | | Mass Spec. | |
|---|---|---|---|
| No. | Chemical Structure | (ES, m/z) | NMR Spectrum |
| IV-1 | [chemical structure] | 593.4 [M + H]⁺ | N/A |
| IV-2 | [chemical structure] | 593.3 [M + H]⁺ | N/A |
| IV-3 | [chemical structure] | 593.3 [M + H]⁺ | N/A |
| IV-4 | [chemical structure] | 607.25 [M + H]⁺ | N/A |
| IV-5 | [chemical structure] | 607.25 [M + H]⁺ | N/A |
| IV-6 | [chemical structure] | 611.45 [M + H]⁺ | ¹H NMR (300 MHz, Methanol- d4) δ 8.06 (d, J = 5.6 Hz, 1H), 7.82 (s, 1H), 7.67 (s, 2H), 7.30 (s, 1H), 7.19 (m, 2H), 6.49-6.33 (m, 2H), 5.41-5.30 (m, 1H), 4.22 (m, 1H), 3.68 (s, 2H), 3.55-3.41 (m, 1H), 3.15-2.87 (m, 2H), 2.56 (m, 10H), 1.54 (m, 3H), 1.13 (t, J = 7.2 Hz, 3H). |
| IV-7 | [chemical structure] | 611.2 [M + H]⁺ | N/A |
| IV-8 | [chemical structure] | 581.4 [M + H]⁺ | ¹H NMR (300 MHz, Methanol- d4) δ 8.25 (d, J = 6.7 Hz, 1H), 8.06 (d, J = 9.2 Hz, 1H), 7.78 (t, J = 9.2 Hz, 1H), 7.50 (d, J = 3.6 Hz, 1H), 7.38 (d, J = 8.4 Hz, 1H), 7.20 (s, 1H), 7.14 (dd, J = 8.3, 2.5 Hz, 1H), 6.79 (d, J = 6.7 Hz, 1H), 6.57 (d, J = 3.6 Hz, 1H), 5.43 (q, J = 6.8 Hz, 1H), 4.8 (m, 1H), 4.21 (m, 1H), 3.68-3.42 (m, 3H), 3.18 (m, 2H), 3.09-2.96 (m, 2H), 2.92 (s, 3H), 2.45-2.23 (m, 2H), 2.04 (m, 2H), 1.55 (d, J = 6.8 Hz, 3H). |
| IV-9 | [chemical structure] | 581.15 [M + H]⁺ | ¹H NMR (300 MHz, Methanol- d4) δ 8.39-8.20 (m, 2H), 8.11 (d, J = 10.3 Hz, 1H), 7.50 (s, 1H), 7.38 (d, J = 8.3 Hz, 1H), 7.21 (s, 1H), 7.15 (d, J = 8.5 Hz, 1H), 6.80 (s, 1H), 6.57 (s, 1H), 5.43 (d, J = 6.7 Hz, 1H), 4.20 (d, J = 13.5 Hz, 1H), 3.66-3.46 (m, 3H), 3.29-3.10 (m, 3H), 3.00 (s, 2H), 2.92 (s, 3H), 2.34 (m, 2H), 2.2-1.9 (m, 2H), 1.56 (d, J = 6.7 Hz, 3H). |
| IV-10 | [chemical structure] | 608.45 [M + H]⁺ | ¹H NMR (400 MHz, Methanol- d4) δ 8.33 (d, J =6.5 Hz, 1H), 7.93-7.84 (m, 2H), 7.75-7.64 (m, 2H), 7.58 (d, J = 3.6 Hz, 1H), 6.85 (d, J = 6.5 Hz, 1H), 6.66 (d, J = 3.6 Hz, 1H), 5.53 (q, J = 6.7 Hz, 1H), 4.41 (m, 1H), 3.79 (d, J = 1.5 Hz, 2H), 3.59 (m, 1H), 3.28-3.08 (m, 5H), 2.86 (m, 3H), 2.53 (s, 3H), 1.58 (m, 3H), 1.36 (m, 4H). |
| IV-11 | [chemical structure] | 618.45 [M + H]⁺ | ¹H NMR (300 MHz, Methanol- d4) δ 8.30 (d, J = 6.6 Hz, 1H), 7.85 (s, 1H), 7.72-7.65 (m, 3H), 7.57 (dd, J = 10.2, 3.1 Hz, 2H), 6.86 (d, J = 6.6 Hz, 1H), 6.62 (d, J = 3.6 Hz, 1H), 5.51 (q, J = 6.8 Hz, 1H), 4.34 (d, J = 13.2 Hz, 1H), 3.76 (s, 2H), 3.66-3.40 (m, 3H), 3.28- 3.01 (m, 8H), 2.51 (s, 2H), 1.59 (m, 3H), 1.36 (t, J = 7.3 Hz, 3H) |
| IV-12 | [chemical structure] | 618.45 [M + H]⁺ | ¹H NMR (400 MHz, Methanol- d4) δ 8.34 (d, J = 6.6 Hz, 1H), 7.87-7.78 (m, 2H), 7.74-7.64 (m, 2H), 7.56 (d, J = 3.5 Hz, 1H), 7.43 (s, 1H), 6.93-6.84 (m, 1H), 6.58 (d, J = 3.5 Hz, 1H), 5.49 (d, J = 6.8 Hz, 1H), 4.24 (d, J = 13.5 Hz, 1H), 3.76 (s, 2H), 3.53 (m, 2H), 3.30- 2.91 (m, 8H), 2.55 (s, 2H), 1.56 (m, 3H), 1.36 (m, 4H). |
| IV-13 | [chemical structure] | 671 [M + H]⁺ | ¹H NMR (400 MHz, Methanol- d4) δ 8.25 (d, J = 6.6 Hz, 1H), 7.82 (d, J = 2.1 Hz, 1H), 7.67 (d, J = 7.9 Hz, 3H), 7.52 (s, 1H), 7.33 (s, 1H), 6.71 (d, J = 6.6 Hz, 1H), 6.55 (d, J = 3.6 Hz, 1H), 5.40 (d, J = 6.7 Hz, 1H), 4.19 (d, J = 14.1 Hz, 1H), 3.73 (s, 2H), 3.59-3.47 (m, 3H), 3.21 (m, 2H), 3.16- 2.93 (m, 6H), 2.48 (s, 2H), 1.54 (m, 3H), 1.34 (t, J = 7.3 Hz, 3H). |
| IV-14 | [chemical structure] | 611.2 [M + H]⁺ | N/A |
| IV-15 | [chemical structure] | 611.45 [M + H]⁺ | N/A |
| IV-16 | [chemical structure] | 611.5 [M + H]⁺ | N/A |
| IV-17 | [chemical structure] | 607.5 [M + H]⁺ | ¹H NMR (400 MHz, Methanol- d4) δ 8.16 (d, J = 6.9 Hz, 1H), 7.86 (d, J = 2.1 Hz, 1H), 7.77- 7.63 (m, 2H), 7.50 (d, J = 8.1 Hz, 1H), 7.19 (d, J = 8.0 Hz, 2H), 6.80 (d, J = 6.9 Hz, 1H), 6.35 (d, J = 1.2 Hz, 1H), 4.69 (m, 1H), 3.86-3.67 (m, 4H), 3.48 (m, 2H), 3.23 (m, 6H), 2.52 (m, 4H), 1.47-1.29 (m, 6H). |
| IV-18 | [chemical structure] | 607.5 [M + H]⁺ | N/A |
| IV-19 | [chemical structure] | 607.5 [M + H]⁺ | N/A |
| IV-20 | [chemical structure] | 607.25 [M + H]⁺ | ¹H NMR (400 MHz, Methanol- d4) δ 8.04 (d, J = 5.6 Hz, 1H), 7.81 (s, 1H), 7.65 (t, J = 1.8 Hz, 2H), 7.33 (d, J = 8.2 Hz, 1H), 7.26 (d, J = 3.5 Hz, 1H), 7.08-6.99 (m, 2H), 6.48 (d, J = 5.6 Hz, 1H), 6.31 (d, J = 3.5 Hz, 1H), 4.88 (d, J = 16.5 Hz, 1H), 4.55 (d, J = 16.5 Hz, 1H), 4.18 (m, 1H), 3.62 (m, 2H), 3.45 (m, 1H), 2.86 (m, 1H), 2.70-2.38 (m, 10H), 1.70 (m, 2H), 1.09 (m, 6H). |
| IV-21 | [chemical structure] | 607.2 [M + H]⁺ | N/A |
| IV-22 | [chemical structure] | 607.05 [M + H]⁺ | N/A |
| IV-23 | [chemical structure] | 605.05 [M + H]⁺ | ¹H NMR (300 MHz, Methanol- d4) δ 8.04 (d, J = 5.6 Hz, 1H), 7.80 (d, J = 1.8 Hz, 1H), 7.72- 7.58 (m, 2H), 7.26 (d, J = 3.5 Hz, 1H), 7.07-6.92 (m, 3H), 6.48 (d, J = 5.6 Hz, 1H), 6.32 (d, J = 3.6 Hz, 1H), 4.87-4.80 (m, 2H), 3.68-3.58 (m, 4H), 2.68-2.32 (m, 10H), 1.17-1.05 (m, 7H). |
| IV-24 | [chemical structure] | 607.45 [M + H]⁺ | ¹H NMR (400 MHz, Chloro- form-d) δ 9.15 (s, 1H), 8.18 (dd, J = 5.5, 3.2 Hz, 1H), 7.64 (d, J = 4.4 Hz, 3H), 7.08-6.97 (m, 2H), 6.59-6.47 (m, 2H), 6.42 (d, J = 3.5 Hz, 1H), 5.11 (m, 1H), 3.99 (m, 1H), 3.65 (s, 2H), 3.64-3.49 (m, 1H), 3.13-2.94 (m, 2H), 2.61 (s, 6H), 2.07-1.92 (m, 1H), 1.87 (m, 1H), 1.63 (s, 3H), 1.24 (m, 4H), 1.06 (m, 3H) |
| IV-25 | [chemical structure] | 625.5 [M + H]⁺ | ¹H NMR (300 MHz, Methanol- d4) δ 7.92 (d, J = 5.7 Hz, 1H), 7.80 (s, 1H), 7.65 (s, 2H), 7.19-7.08 (m, 2H), 6.39 (m, 1H), 5.99 (s, 1H), 5.31 (m, 1H), 4.19 (m, 1H), 3.62 (s, 2H), 3.54-3.38 (m, 1H), 3.06- 2.84 (m, 2H), 2.53 (m, 10H), 2.41 (s, 3H), 1.50 (d, J = 6.8 Hz, 3H), 1.11 (t, J = 7.3 Hz, 3H). |
| IV-26 | [chemical structure] | 625.45 [M + H]⁺ | N/A |
| IV-27 | [chemical structure] | 625.1 [M + H]⁺ | ¹H NMR (400 MHz, Methanol- d4) δ 8.19 (dd, J = 6.9, 1.2 Hz, 1H), 7.86 (d, J = 2.5 Hz, 1H), 7.77-7.64 (m, 2H), 7.35 (dd, J = 19.7, 9.5 Hz, 2H), 6.83 (d, J = 6.9 Hz, 1H), 6.40 (d, J = 1.2 Hz, 1H), 4.88 (m, 1H), 4.67 (m, 1H), 3.88-3.75 (m, 3H), 3.67 (m, 1H), 3.22 (m, 3H), 3.1-2.7 (m, 6H), 2.54 (d, J = 1.0 Hz, 3H), 1.45-1.30 (m, 6H). |
| IV-28 | [chemical structure] | 625.1 [M + H]⁺ | N/A |
| IV-29 | [chemical structure] | 625.15 [M + H]⁺ | N/A |
| IV-30 | [chemical structure] | 611.15 [M + H]⁺ | ¹H NMR (300 MHz, Methanol- d₄) δ 7.91-7.80 (m, 2H), 7.78- 7.61 (m, 3H), 7.21 (dd, J = 18.7, 9.3 Hz, 2H), 6.82 (dd, J = 4.4, 2.7 Hz, 1H), 6.71 (dd, J = 4.4, 1.6 Hz, 1H), 5.76-5.71 (m, 1H), 5.38 (q, J = 6.8 Hz, 1H), 4.26-4.13 (m, 1H), 3.74 (m, 2H), 3.65-3.38 (m, 2H), 3.27- 2.76 (m, 8H), 1.59-1.46 (m, 3H), 1.34 (t, J = 7.3 Hz, 3H). |
| IV-31 | [chemical structure] | 611.05 [M + H]⁺ | N/A |
| IV-32 | [chemical structure] | 611.5 [M + H]⁺ | N/A |
| IV-33 | [chemical structure] | 612.5 [M + H]⁺ | ¹H NMR (300 MHz, Methanol- d4) δ 7.94 (s, 1H), 7.89 (m, 1H), 7.80 (s, 1H), 7.65 (s, 2H), 7.24 (d, J = 7.6 Hz, 1H), 7.13 (d, J = 10.8 Hz, 1H), 7.05 (m, 1H), 6.93 (m, 1H), 5.38 (m, 1H), 4.38-4.02 (m, 1H), 3.63 (s, 2H), 3.55-3.36 (m, 1H), 3.17-2.80 (m, 2H), 2.84-2.23 (m, 10H), 1.55 (d, J = 6.8 Hz, 3H), 1.12 (t, J = 7.2 Hz, 3H). |
| IV-34 | [chemical structure] | 612.26 [M + H]⁺ | N/A |
| IV-35 | [chemical structure] | 612.26 [M + H]⁺ | N/A |
| IV-36 | [chemical structure] | 635.55 [M + H]⁺ | ¹H NMR (400 MHz, Chloro- form-d) δ 8.69 (s, 1H), 8.09 (d, J = 5.5 Hz, 1H), 7.67 (d, J = 5.8 Hz, 2H), 7.23 (d, J = 8.3 Hz, 1H), 7.07-7.00 (m, 1H), 6.97 (d, J = 2.5 Hz, 1H), 6.53 (d, J = 5.5 Hz, 1H), 6.47 (s, 1H), 6.03 (s, 1H), 4.72 (d, J = 15.4 Hz, 1H), 4.58 (d, J = 15.4 Hz, 1H), 4.35 (m, 1H), 3.65 (m, 2H), 3.34 (m, 1H), 2.72-2.40 (m, 10H), 2.02-1.90 (m, 1H), 1.55 (s, 2H), 1.16 (s, 2H) 1.08 (d, J = 6.7 Hz, 3H), 1.01 (d, J = 6.8 Hz, 3H) |
| IV-37 | [chemical structure] | 635.55 [M + H]⁺ | ¹H NMR (400 MHz, Chloro- form-d) δ 9.17 (s, 1H), 8.09 (d, J = 5.5 Hz, 1H), 7.65 (s, 3H), 7.24 (d, J = 8.3 Hz, 1H), 7.03 (dd, J = 8.3, 2.5 Hz, 1H), 6.95 (d, J = 2.4 Hz, 1H), 6.55-6.47 (m, 1H), 6.06 (s, 1H), 4.80 (d, J = 15.7 Hz, 1H), 4.55 (d, J = 15.6 Hz, 1H), 4.03 (m, 1H), 3.65 (m, 2H), 3.49 (m, 1H), 2.96 (m, 1H), 2.54 (m, 8H), 1.72-1.40 (m, 6H), 1.16 (s, 3H), 1.01 (t, J = 7.2 Hz, 3H). |
| IV-38 | [chemical structure] | 595.2 [M + H]⁺ | N/A |
| IV-39 | [chemical structure] | N/A | N/A |
| IV-40 | [chemical structure] | N/A | N/A |
| IV-41 | [chemical structure] | 611.25 [M + H]⁺ | N/A |

### Example 8—Synthesis of 7-((1H-pyrrolo[2,3-b]pyridin-4-yl)oxy)-N-(4-((4-ethylpiperazin-1-yl)methyl)-3-(trifluoromethyl)phenyl)-3-methyl-3,4-dihydroisoquinoline-2(1H)-carboxamide (Compound No. 5)

[chemical structure]

### Part I—Synthesis of 1-(4-Methoxyphenyl)propan-2-amine

[chemical structure]

Into a 100-mL round-bottom flask, was placed 1-(4-methoxyphenyl)propan-2-one (5 g, 30.45 mmol), ammonium formate (19.2 g, 304.49 mmol), in methanol/water (10:1) (70 mL). The mixture was degassed, and 10% palladium on carbon (2.0 g, 18.79 mmol) was added under a nitrogen atmosphere. The resulting mixture was stirred overnight at room temperature. The solids were removed by filtration through celite. The filtrate was concentrated. The residue was applied onto a silica gel column and eluted with dichloromethane/methanol (1:1) to yield 1-(4-methoxyphenyl)propan-2-amine (3.8 g, 76%).

### Part II—Synthesis of 7-Hydroxy-3-methyl-3,4-dihydroisoquinolin-1(2H)-one

[chemical structure]

Into a 100-mL round-bottom flask was placed 1-(4-methoxyphenyl)propan-2-amine (2 g, 12.1 mmol), dichloromethane (40 mL), and 2 M sodium hydroxide (4.2 mL, 8.4 mmol). This was followed by the addition of a solution of bis(trichloromethyl) carbonate (1.43 g, 4.8 mmol) in dichloromethane (5 mL) dropwise with stirring. The resulting mixture was stirred for 2 hours at room temperature. The mixture was extracted with 2×50 ml of dichloromethane and concentrated under reduced pressure. The residue was dissolved in trifluoromethanesulfonic acid (10 mL) and stirred for 2 hours at 100° C. The reaction was then quenched by the addition of water/ice. The pH value of the solution was adjusted to 3-4 with sodium carbonate. The mixture was extracted with 2×100 mL of ethyl acetate and concentrated. The residue was applied onto a silica gel column and eluted with dichloromethane/methanol (8:1) to yield 7-hydroxy-3-methyl-3,4-dihydroisoquinolin-1(2H)-one (700 mg, 33%).

### Part III—Synthesis of 3-Methyl-1,2,3,4-tetrahydroisoquinolin-7-ol

[chemical structure]

Into a 100-mL round-bottom flask, purged and maintained with an inert atmosphere of nitrogen, was placed 7-hydroxy-3-methyl-3,4-dihydroisoquinolin-1(2H)-one (700 mg, 3.95 mmol) and tetrahydrofuran (10 mL). This was followed by the addition of 2.5 M lithium aluminum hydride (2.5 mL, 6.3 mmol) in tetrahydrofuran dropwise with stirring. The resulting solution was heated at reflux overnight. The reaction was then quenched by the addition of water (0.7 mL), followed by 1 M sodium hydroxide (7 mL), and then water (2 mL). The solids were removed by filtration. The filtrate was concentrated, then the crude product was purified on a C18 silica gel column eluting with a gradient of acetonitrile in water (with 0.1% trifluoroacetic acid) to yield 3-methyl-1,2,3,4-tetrahydroisoquinolin-7-ol (300 mg, 47%).

### Part IV—Synthesis of tert-Butyl 7-hydroxy-3-methyl-3,4-dihydroisoquinoline-2(1H)-carboxylate

[chemical structure]

Into a 100-mL round-bottom flask charged with tetrahydrofuran (5 mL) was placed 3-methyl-1,2,3,4-tetrahydroisoquinolin-7-ol (300 mg, 1.84 mmol), di-tert-butyl dicarbonate (401 mg, 1.84 mmol) and 1 M sodium hydroxide (5 mL, 5 mmol). The resulting mixture was stirred for 3 hours at room temperature, then diluted with brine (20 mL), and extracted with ethyl acetate (2×50 ml). The organics were concentrated under reduced pressure. The residue was applied onto a silica gel column and eluted with ethyl acetate/petroleum ether (1:4) to yield tert-butyl 7-hydroxy-3-methyl-3,4-dihydroisoquinoline-2(1H)-carboxylate (260 mg, 54%).

### Part V—Synthesis of tert-Butyl 3-methyl-7-((1-(triisopropylsilyl)-1H-pyrrolo[2,3-b]pyridin-4-yl)oxy)-3,4-dihydroisoquinoline-2(1H)-carboxylate

[chemical structure]

Into a 100-mL round-bottom flask, purged and maintained with an atmosphere of oxygen, was placed copper(II) acetate (138 mg, 0.76 mmol), dichloromethane (2 mL), and pyridine (300 μL, 3.80 mmol). The mixture was stirred for 30 min at room temperature. Then 4 Å molecular sieves (2 g) were added, followed by tert-butyl 7-hydroxy-3-methyl-3,4-dihydroisoquinoline-2(1H)-carboxylate (200 mg, 0.76 mmol) and (1-(triisopropylsilyl)-1H-pyrrolo[2,3-b]pyridin-4-yl)boronic acid (290 mg, 0.91 mmol). The reaction was stirred for 18 hours at room temperature. The solids were filtered out, and the filtrate was concentrated under reduced pressure. The residue was applied onto a silica gel column and eluted with ethyl acetate in petroleum ether (1:5) to yield tert-butyl 3-methyl-7-((1-(triisopropylsilyl)-1H-pyrrolo[2,3-b]pyridin-4-yl)oxy)-3,4-dihydroisoquinoline-2(1H)-carboxylate (210 mg, 52%).

### Part VI—Synthesis of 7-((1H-Pyrrolo[2,3-b]pyridin-4-yl)oxy)-3-methyl-1,2,3,4-tetrahydroisoquinoline

[chemical structure]

Into a 50-mL round-bottom flask was placed tert-butyl 3-methyl-7-((1-(triisopropylsilyl)-1H-pyrrolo[2,3-b]pyridin-4-yl)oxy)-3,4-dihydroisoquinoline-2(1H)-carboxylate (210 mg, 0.75 mmol) followed by dichloromethane (3 mL) and trifluoroacetic acid (5 mL). The resulting solution was stirred for 3 hours at room temperature. The solution was concentrated to yield 7-((1H-pyrrolo[2,3-b]pyridin-4-yl)oxy)-3-methyl-1,2,3,4-tetrahydroisoquinoline (180 mg, quantitative crude yield).

### Part VII—Synthesis of 7-((1H-Pyrrolo[2,3-b]pyridin-4-yl)oxy)-N-(4-((4-ethylpiperazin-1-yl)methyl)-3-(trifluoromethyl)phenyl)-3-methyl-3,4-dihydroisoquinoline-2(1H)-carboxamide (Compound No. 5)

[chemical structure]

Into a 50-mL round-bottom flask, was placed 7-((1H-pyrrolo[2,3-b]pyridin-4-yl)oxy)-3-methyl-1,2,3,4-tetrahydroisoquinoline (120 mg, 0.43 mmol), 1-ethyl-4-(4-isocyanato-2-(trifluoromethyl)benzyl)piperazine (135 mg, 0.43 mmol), dichloromethane (10 mL), and trimethylamine (180 μL, 1.29 mmol). The resulting solution was stirred for 2 hours at room temperature. The resulting mixture was concentrated and purified by preparatory HPLC to yield 7-((1H-pyrrolo[2,3-b]pyridin-4-yl)oxy)-N-(4-((4-ethylpiperazin-1-yl)methyl)-3-(trifluoromethyl)phenyl)-3-methyl-3,4-dihydroisoquinoline-2(1H)-carboxamide (85 mg, 33%). (ES, m/z): [M+H]⁺=593.40. ¹H NMR (300 MHz, Methanol-d₄, ppm) δ 8.05 (d, J=5.6 Hz, 1H), 7.82 (s, 1H), 7.66 (d, J=2.6 Hz, 2H), 7.39-7.23 (m, 2H), 7.07 (d, J=5.9 Hz, 2H), 6.50 (d, J=5.6 Hz, 1H), 6.31 (d, J=3.6 Hz, 1H), 4.92 (s, 2H), 4.49 (d, J=16.5 Hz, 1H), 3.63 (s, 2H), 3.31 (s, 2H), 2.88-2.39 (m, 10H), 1.19 (d, J=6.6 Hz, 3H), 1.11 (t, J=7.3 Hz, 3H).

### Example 9—Synthesis of (S)-7-((1H-Pyrrolo[2,3-b]pyridin-4-yl)oxy)-N-(4-((4-ethylpiperazin-1-yl)methyl)-3-(trifluoromethyl)phenyl)-1-methyl-3,4-dihydroisoquinoline-2(1H)-carboxamide (Compound No. 6)

[chemical structure]

### Part I—Synthesis of (S)—N-(1-(3-Methoxyphenyl)ethyl)picolinamide

[chemical structure]

Into a 250-mL round-bottom flask was placed pyridine-2-carboxylic acid (9.8 g, 79 mmol), N,N-dimethylformamide (100 mL), N-(3-dimethylaminopropyl)-N′-ethylcarbodiimide hydrochloride (25.4 g, 132 mmol), 1-hydroxybenzotriazole hydrate (17.9 g, 132 mmol), and N,N-diisopropylethylamine (46 mL, 265 mmol). The resulting solution was stirred for 20 min at room temperature. To the mixture was added (S)-1-(3-methoxyphenyl)ethan-1-amine (10 g, 66 mmol), and the mixture was stirred overnight at room temperature. The reaction was diluted with ethyl acetate (400 mL). The resulting mixture was washed with brine (3×100 ml). The organics were concentrated, and the residue was purified on a silica gel column eluting with ethyl acetate/petroleum ether (1:2) to yield (S)—N-(1-(3-methoxyphenyl)ethyl)picolinamide (12 g, 71%).

### Part II—Synthesis of (S)—N-(1-(2-(2-(Benzyloxy)ethyl)-5-methoxyphenyl)ethyl)picolinamide

[chemical structure]

Into a 250-mL round-bottom flask was placed (S)—N-(1-(3-methoxyphenyl)ethyl)picolinamide (1.85 g, 7.22 mmol), 2-methylbutan-2-ol (145 mL), [(2-iodoethoxy)methyl]benzene (9.5 g, 36 mmol), palladium(II) acetate (0.2 g, 0.72 mmol), potassium carbonate (1.5 g, 10.8 mmol), and sodium trifluoromethanesulfonate (3.7 g, 21.5 mmol) in an oxygen atmosphere. The mixture was stirred at 125° C. overnight. The reaction was then diluted with ethyl acetate (50 mL), and the solids were removed by filtration. The filtrate was concentrated under reduced pressure, and the residue was applied onto a silica gel column and eluted with ethyl acetate/petroleum ether (1:2) to yield (S)—N-(1-(2-(2-(benzyloxy)ethyl)-5-methoxyphenyl)ethyl)picolinamide (1.7 g, 60%).

### Part III—Synthesis of (S)—N-(1-(2-(2-Hydroxyethyl)-5-methoxyphenyl)ethyl)picolinamide

[chemical structure]

Into a 500-mL round-bottom flask was placed (S)—N-(1-(2-(2-(benzyloxy)ethyl)-5-methoxyphenyl)ethyl)picolinamide (16.5 g, 42.3 mmol), methanol (150 mL), and concentrated hydrogen chloride (0.2 mL). The flask was degassed, then 10% palladium on carbon (6 g, 56 mmol) was added, and the flask was charged with hydrogen. The mixture was stirred under a balloon of hydrogen at room temperature overnight. The solids were filtered off, and the filtrate was concentrated under reduced pressure. The residue was applied onto a silica gel column and eluted with dichloromethane/methanol (5:1) to yield (S)—N-(1-(2-(2-hydroxyethyl)-5-methoxyphenyl)ethyl)picolinamide (8.7 g, 69%).

### Part IV—Synthesis of (S)-4-Methoxy-2-(1-(picolinamido)ethyl)phenethyl 4-methylbenzenesulfonate

[chemical structure]

Into a 500-mL round-bottom flask, was placed (S)—N-(1-(2-(2-hydroxyethyl)-5-methoxyphenyl)ethyl)picolinamide (8.7 g, 29 mmol), dichloromethane (150 mL), and triethylamine (16 mL, 116 mmol). This was followed by the addition of p-toluenesulfonyl chloride (11.0 g, 58 mmol) in portions over 5 minutes. The resulting solution was stirred overnight at room temperature. The mixture was concentrated then applied onto a silica gel column and eluted with ethyl acetate/petroleum ether (2:1) to yield (S)-4-methoxy-2-(1-(picolinamido)ethyl)phenethyl 4-methylbenzenesulfonate (9 g, 68%).

### Part V—Synthesis of (S)-(7-Methoxy-1-methyl-3,4-dihydroisoquinolin-2(1H)-yl)(pyridin-2-yl)methanone

[chemical structure]

Into a 2000-mL round-bottom flask was placed (S)-4-methoxy-2-(1-(picolinamido)ethyl)phenethyl 4-methylbenzenesulfonate (19 g, 42 mmol) and tetrahydrofuran (1000 mL). This was followed by the portionwise addition of sodium hydride (3.35 g, 83.4 mmol, 60% in mineral oil) over 20 min. The mixture was stirred overnight at room temperature. The reaction was then quenched by slow addition of ice water (400 mL). The resulting mixture was extracted with ethyl acetate (2×1000 mL). The combined extracts were washed with brine (3×300 mL), dried over anhydrous sodium sulfate, and concentrated to yield (S)-(7-methoxy-1-methyl-3,4-dihydroisoquinolin-2(1H)-yl)(pyridin-2-yl)methanone (12 g, 97% crude yield).

### Part VI—Synthesis of (S)-7-Methoxy-1-methyl-1,2,3,4-tetrahydroisoquinoline

[chemical structure]

Into a 250-mL round-bottom flask was placed (S)-(7-methoxy-1-methyl-3,4-dihydroisoquinolin-2(1H)-yl)(pyridin-2-yl)methanone (4.3 g, 15 mmol), methanol (50 mL), and a 25% sodium methoxide solution in methanol (50 mL). The resulting solution was stirred at 100° C. for 3 days. The reaction was cooled and quenched by the addition of ice water (150 mL). The mixture was extracted with ethyl acetate (3×100 mL). The organics were concentrated under reduced pressure, and the residue was applied onto a silica gel column and eluted with dichloromethane/methanol (10:1) to yield (S)-7-methoxy-1-methyl-1,2,3,4-tetrahydroisoquinoline (2.6 g, 95%).

### Part VII—Synthesis of (S)-1-methyl-1,2,3,4-tetrahydroisoquinolin-7-ol

[chemical structure]

Into a 500-mL round-bottom flask, was placed (S)-7-methoxy-1-methyl-1,2,3,4-tetrahydroisoquinoline (2.7 g, 15 mmol) and dichloromethane (200 mL). This was followed by the dropwise addition of 1 M boron tribromide (60 mL, 60 mmol) in dichloromethane. The resulting solution was stirred for 2 hours at room temperature. The reaction was then quenched by the addition of methanol (150 mL). The mixture was concentrated and used without purification in the next step.

### Part VIII—Synthesis of tert-Butyl (S)-7-hydroxy-1-methyl-3,4-dihydroisoquinoline-2(1H)-carboxylate

[chemical structure]

Into a 100-mL round-bottom flask was dissolved (S)-1-methyl-1,2,3,4-tetrahydroisoquinolin-7-ol (2.5 g, 15 mmol) in tetrahydrofuran (30 mL). A 1 M solution of sodium hydroxide (30 mL, 30 mmol) was added, followed by di-tert-butyl dicarbonate (3.3 g, 15 mmol). The resulting mixture was stirred overnight at room temperature. The mixture was extracted with ethyl acetate (3×40 mL). The organics were concentrated under reduced pressure, and the residue was purified on a silica gel column eluting with ethyl acetate/petroleum ether (1:10) to yield tert-butyl (S)-7-hydroxy-1-methyl-3,4-dihydroisoquinoline-2(1H)-carboxylate (2.4 g, 60%).

### Part IX—Synthesis of tert-Butyl (S)-1-methyl-7-((1-(triisopropylsilyl)-1H-pyrrolo[2,3-b]pyridin-4-yl)oxy)-3,4-dihydroisoquinoline-2(1H)-carboxylate

[chemical structure]

Into a 100-mL round-bottom flask, purged and maintained with an atmosphere of oxygen, was placed copper(II) acetate (0.8 g, 4.56 mmol), pyridine (1.8 mL, 22.8 mmol), and dichloromethane (80 mL). The mixture was stirred for 30 min at room temperature, then 4 Å molecular sieves (5 g) were added, followed by tert-butyl (S)-7-hydroxy-1-methyl-3,4-dihydroisoquinoline-2(1H)-carboxylate (1.2 g, 4.56 mmol) and (1-(triisopropylsilyl)-1H-pyrrolo[2,3-b]pyridin-4-yl)boronic acid (2.0 g, 6.38 mmol). The reaction was stirred for 18 hours at room temperature. The solids were removed by filtration, and the filtrate was concentrated. The residue was applied onto a silica gel column and eluted with ethyl acetate in petroleum ether (1:5) to yield tert-butyl (S)-1-methyl-7-((1-(triisopropylsilyl)-1H-pyrrolo[2,3-b]pyridin-4-yl)oxy)-3,4-dihydroisoquinoline-2(1H)-carboxylate (1.4 g, 57%).

### Part X—Synthesis of (S)-7-((1H-Pyrrolo[2,3-b]pyridin-4-yl)oxy)-1-methyl-1,2,3,4-tetrahydroisoquinoline

[chemical structure]

Into a 25-mL round-bottom flask, was placed tert-butyl (S)-1-methyl-7-((1-(triisopropylsilyl)-1H-pyrrolo[2,3-b]pyridin-4-yl)oxy)-3,4-dihydroisoquinoline-2(1H)-carboxylate (2.8 g, 5.23 mmol) and concentrated hydrogen chloride (aq., 10 mL). The solution was stirred for 15 min at room temperature and concentrated to yield crude (S)-7-((1H-pyrrolo[2,3-b]pyridin-4-yl)oxy)-1-methyl-1,2,3,4-tetrahydroisoquinoline as the hydrochloride salt (2.0 g, quantitative crude yield).

### Part XI—Synthesis of (S)-7-((1H-Pyrrolo[2,3-b]pyridin-4-yl)oxy)-N-(4-((4-ethylpiperazin-1-yl)methyl)-3-(trifluoromethyl)phenyl)-1-methyl-3,4-dihydroisoquinoline-2(1H)-carboxamide (Compound No. 6)

[chemical structure]

Into a 250-mL round-bottom flask, was placed (S)-7-((1H-pyrrolo[2,3-b]pyridin-4-yl)oxy)-1-methyl-1,2,3,4-tetrahydroisoquinoline (2.5 g, 7.9 mmol), dichloromethane (80 mL), and triethylamine (2.2 mL, 15.8 mmol), followed by 1-ethyl-4-(4-isocyanato-2-(trifluoromethyl)benzyl)piperazine (3.5 g, 14.2 mmol). The reaction was stirred for 2 hours at room temperature. The mixture was concentrated, and the residue was purified on a silica gel column eluting with dichloromethane/methanol (92:8) to yield (S)-7-((1H-pyrrolo[2,3-b]pyridin-4-yl)oxy)-N-(4-((4-ethylpiperazin-1-yl)methyl)-3-(trifluoromethyl)phenyl)-1-methyl-3,4-dihydroisoquinoline-2(1H)-carboxamide (2.8 g, 60%). (ES, m/z): [M+H]⁺=593.35. ¹H NMR (300 MHz, Methanol-d₄, ppm) δ 8.32 (d, J=6.9 Hz, 1H), 8.12 (d, J=2.2 Hz, 1H), 8.02 (d, J=8.6 Hz, 1H), 7.90 (dd, J=8.5, 2.3 Hz, 1H), 7.57 (d, J=3.6 Hz, 1H), 7.43 (d, J=8.4 Hz, 1H), 7.26 (d, J=2.5 Hz, 1H), 7.19 (dd, J=8.3, 2.5 Hz, 1H), 6.87 (d, J=6.9 Hz, 1H), 6.64 (d, J=3.6 Hz, 1H), 5.48 (q, J=6.7 Hz, 1H), 4.61 (m, 2H), 4.25 (s, 1H), 3.8-3.5 (m, 9H), 3.44-3.32 (m, 2H), 3.20-2.94 (m, 2H), 1.58 (d, J=6.8 Hz, 3H), 1.43 (t, J=7.3 Hz, 3H).

### Example 10—Synthesis of Additional N-phenyl-3,4-dihydroisoquinoline-2(1H)-carboxamides and Related Compounds

The compounds in Table 5 below were prepared based on experimental procedures described in Example 9 and in the detailed description. All compounds in Table 5 were prepared as single enantiomers with the absolute stereochemistry as shown.

TABLE 5

| | | Mass Spec. | |
|---|---|---|---|
| No. | Chemical Structure | (ES, m/z) | NMR Spectrum |
| V-1 | [chemical structure] | 565.25 [M + H]⁺ | N/A |
| V-2 | [chemical structure] | 579.5 [M + H]⁺ | N/A |
| V-3 | [chemical structure] | 559.4 [M + H]⁺ | ¹H NMR (400 MHz, Methanol- d4) δ 8.28 (d, J = 6.8 Hz, 1H), 7.65-7.62 (m, 1H), 7.54 (d, J = 3.5 Hz, 1H), 7.44-7.37 (m, 3H), 7.23 (d, J = 2.4 Hz, 1H), 7.17 (dd, J = 8.3, 2.5 Hz, 1H), 6.83 (d, J = 6.8 Hz, 1H), 6.61 (d, J = 3.6 Hz, 1H), 5.43 (q, J = 6.8 Hz, 1H), 4.20 (m, 1H), 3.81 (m, 2H), 3.57-3.52 (m, 1H), 3.31-2.86 (m, 12H), 1.57 (d, J = 6.8 Hz, 3H), 1.35 (t, J = 7.3 Hz, 3H). |
| V-4 | [chemical structure] | 575.3 [M + H]⁺ | N/A |
| V-5 | [chemical structure] | 594.1 [M + H]⁺ | N/A |
| V-6 | [chemical structure] | 593.3 [M + H]⁺ | ¹H NMR (300 MHz, Methanol- d4) δ 7.87 (d, J = 5.3 Hz, 1H), 7.82 (d, J = 2.2 Hz, 1H), 7.75- 7.62 (m, 3H), 7.32 (d, J = 8.3 Hz, 1H), 7.13-7.03 (m, 2H), 6.80 (dd, J = 4.4, 2.7 Hz, 1H), 6.68 (dd, J = 4.4, 1.6 Hz, 1H), 5.76 (d, J = 5.4 Hz, 1H), 5.38 (d, J = 7.1 Hz, 1H), 4.19 (m, 1H), 3.77-3.68 (m, 2H), 3.49 (t, J = 9.8 Hz, 3H), 3.21 (m, 3H), 3.09-2.86 (m, 5H), 2.47 (m, 2H), 1.55 (d, J = 6.7 Hz, 3H), 1.34 (t, J = 7.3 Hz, 3H). |
| V-7 | [chemical structure] | 673.35 [M + H]⁺ | ¹H NMR (400 MHz, Methanol- d4) δ 7.99-7.90 (m, 2H), 7.90- 7.83 (m, 2H), 7.80 (s, 1H), 7.76-7.62 (m, 2H), 7.34 (d, J = 8.2 Hz, 1H), 7.19-7.07 (m, 2H), 6.85 (d, J = 1.7 Hz, 1H), 5.80 (d, J = 5.4 Hz, 1H), 5.49-5.33 (m, 1H), 4.21 (m, 1H), 3.95 (s, 3H), 3.76 (s, 2H), 3.52 (t, J = 11.8 Hz, 2H), 3.28-2.93 (m, 9H), 2.50 (m, 2H), 1.58 (d, J = 6.7 Hz, 3H), 1.37 (t, J = 7.3 Hz, 3H) |
| V-8 | [chemical structure] | 618.45 [M + H]⁺ | ¹H NMR (300 MHz, Methanol- d4) δ 8.32 (d, J = 1.8 Hz, 1H), 8.09 (d, J = 5.4 Hz, 1H), 7.84 (s, 1H), 7.68 (d, J = 1.7 Hz, 2H), 7.37 (d, J = 8.3 Hz, 1H), 7.22-7.02 (m, 3H), 5.93 (d, J = 5.5 Hz, 1H), 5.43 (q, J = 6.8 Hz, 1H), 4.22 (d, J = 13.7 Hz, 1H), 3.66 (s, 2H), 3.58- 3.44 (m, 1H), 3.19-2.38 (m, 12H), 2.5 (m, 2H), 1.58 (d, J = 6.8 Hz, 3H), 1.17 (t, J = 7.2 Hz, 3H) |
| V-9 | [chemical structure] | 619.4 [M + H]⁺ | ¹H NMR (300 MHz, Methanol- d4) δ 8.44 (d, J = 1.6 Hz, 1H), 8.13 (s, 1H), 7.83 (s, 1H), 7.68 (s, 2H), 7.44 (d, J = 1.6 Hz, 1H), 7.32 (d, J = 8.2 Hz, 1H), 7.26-7.08 (m, 2H), 5.42 (d, J = 6.7 Hz, 1H), 4.20 (d, J = 13.6 Hz, 1H), 3.64 (m, 2H), 3.52 (t, J = 10.6 Hz, 1H), 3.12-2.89 (m, 2H), 2.79-2.27 (m, 10H), 1.58 (d, J = 6.8 Hz, 3H), 1.13 (t, J = 7.2 Hz, 3H) |
| V10 | [chemical structure] | 618.2 [M + H]⁺ | ¹H NMR (300 MHz, Methanol- d4) δ 8.24 (d, J = 5.6 Hz, 1H), 7.80 (d, J = 1.7 Hz, 1H), 7.65 (s, 2H), 7.31 (d, J = 8.3 Hz, 1H), 7.17-6.99 (m, 3H), 6.49 (d, J = 5.6 Hz, 1H), 5.38 (q, J = 6.8 Hz, 1H), 4.25-4.13 (m, 1H), 3.61 (d, J = 1.6 Hz, 2H), 3.48 (m, 1H), 3.12-2.86 (m, 2H), 2.52 (m, 10H), 1.53 (d, J = 6.7 Hz, 3H), 1.10 (t, J = 7.2 Hz, 3H) |
| V-11 | [chemical structure] | 579.5 [M + H]⁺ | N/A |
| V-12 | [chemical structure] | 597.2 [M + H]⁺ | N/A |
| V-13 | [chemical structure] | 611.25 [M + H]⁺ | ¹H NMR (400 MHz, DMSO- d6) δ 8.93 (d, J = 4.9 Hz, 1H), 8.80 (q, J = 4.8 Hz, 1H), 8.52 (d, J = 5.6 Hz, 1H), 7.93 (d, J = 2.2 Hz, 1H), 7.82 (dd, J = 8.5, 2.2 Hz, 1H), 7.59 (d, J = 8.6 Hz, 1H), 7.42-7.29 (m, 2H), 7.22-7.14 (m, 2H), 7.06 (dd, J = 8.3, 2.5 Hz, 1H), 5.39 (q, J = 6.6 Hz, 1H), 4.20 (d, J = 13.3 Hz, 1H), 3.66 (s, 2H), 3.47 (d, J = 11.9 Hz, 2H), 3.36 (t, J = 10.3 Hz, 1H), 3.14 (m, 2H), 3.07-2.82 (m, 6H), 2.79 (d, J = 4.8 Hz, 3H), 2.38 (m, 2H), 1.45 (d, J = 6.7 Hz, |
| | | | 3H), 1.21 (t, J = 7.3 Hz, 3H) |
| V-14 | [chemical structure] | 548.25 [M + H]⁺ | N/A |
| V-15 | [chemical structure] | 562.25 [M + H]⁺ | N/A |
| V-16 | [chemical structure] | 576.5 [M + H]⁺ | N/A |
| V-17 | [chemical structure] | 610.15 [M + H]⁺ | ¹H NMR (300 MHz, Chloro- form-d) δ 13.87 (s, 1H), 8.00 (d, J = 6.8 Hz, 1H), 7.68-7.52 (m, 2H), 7.40-7.27 (m, 2H), 7.12-7.01 (m, 2H), 6.93 (d, J = 8.9 Hz, 1H), 6.65 (d, J = 6.8 Hz, 1H), 6.50-6.37 (m, 1H), 5.35 (q, J = 6.9 Hz, 1H), 4.84 (s, 1H), 4.07-3.95 (m, 1H), 4.00 (s, 2H), 3.62-3.46 (m, 3H), 3.23-2.93 (m, 6H), 2.44 (t, J = 13.9 Hz, 2H), 2.20 (d, J = 15.1 Hz, 2H), 1.57 (d, J = 6.8 Hz, 3H). |
| V-18 | [chemical structure] | 591.25 [M + H]⁺ | ¹H NMR (400 MHz, Methanol- d4) δ 8.30 (d, J = 6.8 Hz, 1H), 7.78 (d, J = 2.2 Hz, 1H), 7.62- 7.52 (m, 2H), 7.39-7.14 (m, 5H), 6.85 (d, J = 6.8 Hz, 1H), 6.62 (d, J = 3.6 Hz, 1H), 5.45 (q, J = 6.7 Hz, 1H), 4.22 (d, J = 13.3 Hz, 1H), 3.93-3.85 (m, 2H), 3.81 (m, 2H), 3.54 (m, 1H), 3.40 (m, 4H), 3.18- 2.96 (m, 2H), 2.95-2.68 (m, 5H), 1.57 (d, J = 6.8 Hz, 3H). |
| V-19 | [chemical structure] | 609.15 [M + H]⁺ | N/A |
| V-20 | [chemical structure] | 607.5 [M + H]⁺ | N/A |
| V-21 | [chemical structure] | 621.45 [M + H]⁺ | N/A |
| V-22 | [chemical structure] | 621.45 [M + H]⁺ | ¹H NMR (300 MHz, Methanol- d₄) δ 8.14 (d, J = 6.9 Hz, 1H), 7.84 (d, J = 2.0 Hz, 1H), 7.72- 7.63 (m, 2H), 7.38 (d, J = 8.3 Hz, 1H), 7.21-7.10 (m, 2H), 6.78 (d, J = 6.9 Hz, 1H), 6.29 (t, J = 1.0 Hz, 1H), 5.42 (q, J = 6.7 Hz, 1H), 4.25-4.13 (m, 1H), 3.74 (m, 2H), 3.59- 3.44 (m, 2H), 3.29 (m, 1H), 3.21 (m, 3H), 3.14-2.91 (m, 4H), 2.90-2.76 (m, 3H), 2.73- 2.34 (m, 2H), 1.55 (d, J = 6.8 Hz, 3H), 1.34 (m, 6H). |
| V-23 | [chemical structure] | 665.05 [M + H]⁺ | ¹H NMR (300 MHz, DMSO- d₆) δ 11.80 (d, J = 2.2 Hz, 1H), 8.81 (s, 1H), 8.04 (d, J = 5.5 Hz, 1H), 7.88 (d, J = 2.3 Hz, 1H), 7.75 (d, J = 8.9 Hz, 1H), 7.56 (d, J = 8.5 Hz, 1H), 7.25 (d, J = 8.4 Hz, 1H), 7.06 (d, J = 2.5 Hz, 1H), 6.98 (dd, J = 8.3, 2.5 Hz, 1H), 6.44 (s, 1H), 6.41-6.32 (m, 2H), 5.35 (d, J = 6.8 Hz, 1H), 4.81 (d, J = 6.6 Hz, 2H), 4.68 (d, J = 6.5 Hz, 2H), 4.12 (m, 1H), 3.50 (s, 2H), 3.15 (m, 1H), |
| | | | 2.84 (m, 2H), 2.37 (m, 10H), |
| | | | 1.41 (d, J = 6.7 Hz, 3H), 0.97 |
| | | | (t, J = 7.1 Hz, 3H). |
| V-24 | [chemical structure] | 637.2 [M + H]⁺ | ¹H NMR (300 MHz, Methanol- d4) δ 8.16 (d, J = 6.8 Hz, 1H), 7.84 (d, J = 2.0 Hz, 1H), 7.76- 7.62 (m, 2H), 7.39 (d, J = 8.3 Hz, 1H), 7.23-7.08 (m, 2H), 6.79 (d, J = 6.8 Hz, 1H), 6.39 (d, J = 0.9 Hz, 1H), 5.43 (q, J = 6.7 Hz, 1H), 4.20 (m, 1H), 3.90 (t, J = 6.3 Hz, 2H), 3.78- 3.71 (m, 2H), 3.62-3.43 (m, 3H), 3.22 (m, 3H), 3.14-2.85 (m, 7H), 2.53-2.46 (m, 2H), 1.56 (d, J = 6.8 Hz, 3H), 1.35 (t, J = 7.3 Hz, 3H). |
| V-25 | [chemical structure] | 651.35 [M + H]⁺ | ¹H NMR (400 MHz, Methanol- d₄) δ 7.97 (d, J = 5.6 Hz, 1H), 7.82 (d, J = 1.8 Hz, 1H), 7.66 (d, J = 2.5 Hz, 2H), 7.27 (d, J = 8.8 Hz, 1H), 7.00 (d, J = 6.8 Hz, 2H), 6.48 (d, J = 5.6 Hz, 1H), 6.03 (s, 1H), 5.35 (q, J = 6.7 Hz, 1H), 4.20 (d, J = 13.7 Hz, 1H), 3.70 (t, J = 6.5 Hz, 2H), 3.66-3.61 (m, 2H), 3.56-3.42 (m, 1H), 3.37 (s, 3H), 3.09-2.85 (m, 2H), 2.88-2.04 (m, 9H), 1.53 (d, J = 6.8 Hz, 3H), 1.12 (t, J = 7.2 Hz, 3H). |
| V-26 | [chemical structure] | 633.1 [M + H]⁺ | ¹H NMR (300 MHz, Methanol- d₄) δ 7.95 (d, J = 5.6 Hz, 1H), 7.83 (d, J = 1.8 Hz, 1H), 7.67 (d, J = 2.3 Hz, 2H), 7.28 (d, J = 9.0 Hz, 1H), 7.04-6.95 (m, 2H), 6.47 (d, J = 5.7 Hz, 1H), 5.88 (s, 1H), 5.37 (t, J = 6.6 Hz, 1H), 4.27-4.16 (m, 1H), 3.64 (d, J = 1.6 Hz, 2H), 3.50 (m, 1H), 3.11-2.87 (m, 2H), 2.63-2.45 (m, 10H), 2.03 (m, 1H), 1.54 (d, J = 6.7 Hz, 3H), 1.13 (t, J = 7.2 Hz, 3H), 1.08- 0.90 (m, 2H), 0.86-0.74 (m, 2H). |
| V-27 | [chemical structure] | 673.55 [M + H]⁺ | ¹H NMR (300 MHz, Methanol- d₄) δ 8.14 (d, J = 4.5 Hz, 2H), 7.96 (s, 1H), 7.85 (s, 1H), 7.69 (t, J = 7.5 Hz, 2H), 7.40 (d, J = 8.3 Hz, 1H), 7.23-7.11 (m, 2H), 6.79-6.67 (m, 2H), 5.49-5.38 (m, 1H), 4.22 (d, J = 12.9 Hz, 1H), 4.00 (s, 3H), 3.76 (m, 2H), 3.56 (m, 2H), 3.24 (m, 2H), 3.14-2.93 (m, 6H), 2.49 (m, 2H), 1.58 (m, 3H), 1.37 (t, J = 7.3 Hz, 3H). |
| V-28 | [chemical structure] | 608.3 [M + H]⁺ | N/A |
| V-29 | [chemical structure] | N/A | N/A |

### Example 11—Synthesis of (S)-7-((1H-pyrrolo[2,3-b]pyridin-4-yl)oxy)-1-methyl-N-(6-((1-methylpiperidin-4-yl)oxy)-5-(trifluoromethyl)pyridin-3-yl)-3,4-dihydroisoquinoline-2(1H)-carboxamide (Compound No. 7)

[chemical structure]

### Part I—Synthesis of 2-((1-Methylpiperidin-4-yl)oxy)-5-nitro-3-(trifluoromethyl)pyridine

[chemical structure]

Into a 100-mL round-bottom flask purged and maintained with an inert atmosphere of nitrogen was dissolved 1-methylpiperidin-4-ol (760 mg, 6.6 mmol) in N,N-dimethylformamide (30 mL), and the solution was cooled to 0° C. Sodium hydride (210 mg, 8.8 mmol) was added at 0° C. in portions. This was followed by the addition of 2-chloro-5-nitro-3-(trifluoromethyl)pyridine (1 g, 4.4 mmol). The reaction was stirred at 80° C. overnight. The mixture was diluted with water (100 mL), and extracted with ethyl acetate (3×100 mL). The combined extracts were dried over anhydrous sodium sulfate and concentrated. The residue was purified on a silica gel column eluting with ethyl acetate/petroleum ether (0:100-17:83) to yield 2-((1-methylpiperidin-4-yl)oxy)-5-nitro-3-(trifluoromethyl)pyridine (286 mg, 21%).

### Part II—Synthesis of 5-Amino-2-((1-methylpiperidin-4-yl)oxy)-3-(trifluoromethyl)pyridine

[chemical structure]

Into a 100-mL round-bottom flask was placed 2-((1-methylpiperidin-4-yl)oxy)-5-nitro-3-(trifluoromethyl)pyridine (165 mg, 0.54 mmol) in methanol (10 mL). The flask was purged with nitrogen, 10% rhodium on carbon (165 mg, 1.60 mmol) was added, and the flask was charged with hydrogen. The reaction was stirred under a hydrogen balloon at room temperature overnight. The flask was evacuated, and the solids were removed by filtration. The filtrate was concentrated to yield 5-amino-2-((1-methylpiperidin-4-yl)oxy)-3-(trifluoromethyl)pyridine (153 mg, quantitative crude yield).

### Part III—Synthesis of Phenyl (6-((1-methylpiperidin-4-yl)oxy)-5-(trifluoromethyl)pyridin-3-yl)carbamate

[chemical structure]

Into a 100-mL round-bottom flask was dissolved 5-amino-2-((1-methylpiperidin-4-yl)oxy)-3-(trifluoromethyl)pyridine (220 mg, 0.80 mmol) in dichloromethane (20 mL). Then N,N-diisopropylethylamine (280 μL, 1.6 mmol) was added, followed by phenyl chloroformate (250 mg, 1.60 mmol). The resulting solution was stirred at room temperature for 1 hour. The solvents were removed under reduced pressure. The residue was applied onto a silica gel column and eluted with dichloromethane/methanol (3:1) to yield phenyl (6-((1-methylpiperidin-4-yl)oxy)-5-(trifluoromethyl)pyridin-3-yl)carbamate (310 mg, 98%).

### Part IV—Synthesis of (S)-7-((1H-Pyrrolo[2,3-b]pyridin-4-yl)oxy)-1-methyl-N-(6-((1-methylpiperidin-4-yl)oxy)-5-(trifluoromethyl)pyridin-3-yl)-3,4-dihydroisoquinoline-2(1H)-carboxamide (Compound No. 7)

[chemical structure]

Into a 25-mL round-bottom flask was dissolved (S)-7-((1H-pyrrolo[2,3-b]pyridin-4-yl)oxy)-1-methyl-1,2,3,4-tetrahydroisoquinoline (50 mg, 0.18 mmol) in dichloromethane (5 mL). Triethylamine (83 μL, 0.59 mmol) was added, followed by phenyl (6-((1-methylpiperidin-4-yl)oxy)-5-(trifluoromethyl)pyridin-3-yl)carbamate (90 mg, 0.23 mmol). The solution was stirred at room temperature for 1 hour and then concentrated. The crude product was purified by preparatory HPLC to yield (S)-7-((1H-pyrrolo[2,3-b]pyridin-4-yl)oxy)-1-methyl-N-(6-((1-methylpiperidin-4-yl)oxy)-5-(trifluoromethyl)pyridin-3-yl)-3,4-dihydroisoquinoline-2(1H)-carboxamide (64.7 mg, 49%). (ES, m/z): [M+H]⁺=581.4. ¹H NMR (400 MHz, Methanol-d₄, ppm) δ 8.43 (d, J=2.6 Hz, 1H), 8.29 (d, J=6.8 Hz, 1H), 8.18 (dd, J=15.5, 2.6 Hz, 1H), 7.53 (d, J=3.6 Hz, 1H), 7.41 (d, J=8.3 Hz, 1H), 7.28-7.13 (m, 2H), 6.83 (d, J=6.8 Hz, 1H), 6.60 (d, J=3.6 Hz, 1H), 5.65-5.31 (m, 2H), 4.20 (d, J=12.9 Hz, 1H), 3.70-3.39 (m, 3H), 3.22 (d, J=15.9 Hz, 2H), 3.09 (ddd, J=16.1, 10.4, 5.5 Hz, 1H), 3.02 (s, 1H), 2.95 (d, J=5.0 Hz, 3H), 2.32 (s, 2H), 2.18 (s, 2H), 1.61-1.18 (m, 3H).

### Example 12—Synthesis of Additional N-phenyl-3,4-dihydroisoquinoline-2(1H)-carboxamides and Related Compounds

The compounds in Table 6 below were prepared based on experimental procedures described in Examples 9 and 11 and in the detailed description. Unless otherwise noted, the compounds in Table 6 were prepared as single enantiomers with the absolute stereochemistry as shown.

TABLE 6

| | | Mass Spec. | |
|---|---|---|---|
| No. | Chemical Structure | (ES, m/z) | NMR Spectrum |
| VI-1 | [chemical structure] | 581.4 [M + H]⁺ | ¹H NMR (400 MHz, Methanol- d4) δ 8.42 (d, J = 2.6 Hz, 1H), 8.20-8.12 (m, 2H), 7.39 (d, J = 8.3 Hz, 1H), 7.24-7.08 (m, 2H), 6.79 (d, J = 6.8 Hz, 1H), 6.29 (d, J = 1.2 Hz, 1H), 5.53 (dd, J = 6.1, 3.1 Hz, 1H), 5.41 (q, J = 6.7 Hz, 1H), 4.18 (d, J = 13.5 Hz, 1H), 3.62-3.47 (m, 1H), 3.35 (d, J = 4.2 Hz, 1H), 3.31 (m, 1H), 3.14-2.94 (m, 2H), 2.51 (m, 3H), 2.18 (m, 4H), 1.56 (d, J = 6.8 Hz, 3H). |
| VI-2 | [chemical structure] | 595.45 [M + H]⁺ | ¹H NMR (300 MHz, Methanol- d4) δ 8.47-8.40 (m, 1H), 8.23- 8.11 (m, 2H), 7.40 (d, J = 8.3 Hz, 1H), 7.22-7.09 (m, 2H), 6.79 (d, J = 6.9 Hz, 1H), 6.29 (d, J = 1.2 Hz, 1H), 5.62-5.29 (m, 2H), 4.19 (d, J = 13.2 Hz, 1H), 3.70-3.44 (m, 3H), 3.22 (m, 2H), 3.04 (m, 2H), 2.94 (m, 3H), 2.51 (m, 3H), 2.34 (m, 2H), 2.17 (m, 1H), 1.96 (d, J = 12.5 Hz, 1H), 1.57 (d, J = 6.8 Hz, 3H). |
| VI-3 | [chemical structure] | 609.45 [M + H]⁺ | ¹H NMR (300 MHz, Chloro-form-d) δ 13.60 (s, 1H), 11.97 (s, 1H), 8.31 (d, J = 11.9 Hz, 2H), 7.88 (d, J = 6.7 Hz, 1H), 7.31-7.13 (s, 1H), 7.03 (d, J = 6.9 Hz, 2H), 6.60 (d, J = 6.7 Hz, 1H), 6.15 (s, 1H), 5.57 (m, 1H), 5.42 (d, J = 6.8 Hz, 1H), 4.13 (d, J = 12.8 Hz, 1H), 3.55 (m, 1H), 3.53-3.41 (m, 2H), 3.10 (m, 5H), 3.08-2.92 (m, 2H), 2.52 (m, 3H), 2.42 (m, 2H), 2.25 (m, 1H), 1.57 (d, J = 6.5 Hz, 3H), 1.41 (t, J = 7.2 Hz, 3H) |
| VI-4 | [chemical structure] | 609.45 [M + H]⁺ | ¹H NMR (300 MHz, Methanol- d4) δ 8.42 (d, J = 2.6 Hz, 1H), 8.23-8.09 (m, 2H), 7.40 (d, J = 8.4 Hz, 1H), 7.24-7.10 (m, 2H), 6.79 (d, J = 6.8 Hz, 1H), 6.29 (d, J = 1.3 Hz, 1H), 5.50 (m, 1H), 5.41 (m, 1H), 4.19 (m, 1H), 3.64-3.49 (m, 1H), 3.45 (m, 2H), 3.13-2.96 (m, 4H), 2.94 (s, 3H), 2.51 (m, 3H), 2.38 (m, 2H), 2.13 (m, 1H), 1.57 (m, 3H), 1.08 (m, 3H). |
| VI-5 | [chemical structure] | 609.45 [M + H]⁺ | ¹H NMR (300 MHz, Methanol- d4) δ 8.36 (d, J = 2.7 Hz, 1H), 8.11 (d, J = 2.7 Hz, 1H), 7.96 (d, J = 5.6 Hz, 1H), 7.28 (d, J = 8.9 Hz, 1H), 7.01 (d, J = 6.8 Hz, 2H), 6.48 (d, J = 5.7 Hz, 1H), 5.96 (m, 1H), 5.33 (m, 1H), 4.80 (m, 1H), 4.19 (m, 1H), 3.58-3.44 (m, 1H), 3.13-2.85 (m, 4H), 2.42 (m, 3H), 2.29 (m, 5H), 2.13-1.88 (m, 2H), 1.77-1.49 (m, 4H), 1.00 (d, J = 6.2 Hz, 3H) |
| VI-6 | [chemical structure] | 595.45 [M + H]⁺ | ¹H NMR (300 MHz, Chloro- form-d) δ 9.13 (s, 1H), 8.19- 8.01 (m, 3H), 7.18 (d, J = 8.2 Hz, 1H), 7.03- 6.90 (m, 2H), 6.51-6.41 (m, 1H), 6.03 (s, 1H), 5.24 (m, 2H), 4.05 (m, 1H), 3.58-3.42 (m, 1H), 3.17 (m, 1H), 3.10-2.92 (m, 2H), 2.85 (m, 1H), 2.45 (s, 3H), 2.39 (m, 3H), 2.13 (m, 3H), 1.81 (m, 3H), 1.54 (d, J = 6.8 Hz, 3H). |
| VI-7 | [chemical structure] | 594.15 [M + H]⁺ | ¹H NMR (400 MHz, Methanol- d4) δ 8.29 (d, J = 2.5 Hz, 1H), 8.13 (d, J = 6.9 Hz, 1H), 7.87 (d, J = 2.5 Hz, 1H), 7.37 (d, J = 8.3 Hz, 1H), 7.21-7.04 (m, 2H), 6.77 (d, J = 6.9 Hz, 1H), 6.28 (d, J = 1.2 Hz, 1H), 5.37 (m, 1H), 4.40-4.24 (m, 1H), 4.14 (m, 1H), 3.65-3.39 (m, 3H), 3.17 (m, 2H), 3.11-2.86 (m, 5H), 2.49 (s, 3H), 2.31 (m, 2H), 1.84 (m, 2H), 1.54 (d, J = 6.8 Hz, 3H). |
| VI-8 | [chemical structure] | 611.45 [M + H]⁺ | ¹H NMR (400 MHz, Methanol- d4) δ 8.36 (d, J = 2.6 Hz, 1H), 8.13 (d, J = 2.7 Hz, 1H), 7.95 (d, J = 5.6 Hz, 1H), 7.27 (d, J = 8.9 Hz, 1H), 7.00 (d, J = 6.7 Hz, 2H), 6.48 (d, J = 5.6 Hz, 1H), 5.95 (d, J = 1.2 Hz, 1H), 5.51 (m, 1H), 5.32 (m, 1H), 4.22-4.14 (m, 1H), 3.99 (m, 1H), 3.56-3.44 (m, 1H), 3.10-2.88 (m, 2H), 2.73 (m, 1H), 2.63-2.50 (m, 2H), 2.41 (m, 4H), 2.36 (m, 3H), 2.16- 2.06 (m, 1H), 1.92-1.80 (m, 1H), 1.54 (d, J = 6.8 Hz, 3H). |
| VI-9 | [chemical structure] | 611.05 [M + H]⁺ | ¹H NMR (300 MHz, Methanol- d₄) δ 8.38 (d, J = 2.6 Hz, 1H), 8.14 (d, J = 2.7 Hz, 1H), 7.95 (d, J = 5.6 Hz, 1H), 7.27 (d, J = 8.6 Hz, 1H), 7.00 (d, J = 6.8 Hz, 2H), 6.47 (d, J = 5.7 Hz, 1H), 5.96 (d, J = 1.3 Hz, 1H), 5.33 (m, 1H), 5.23-5.10 (m, 1H), 4.24-4.13 (m, 1H), 4.02- 3.90 (m, 1H), 3.67-3.42 (m, 1H), 3.10-2.91 (m, 3H), 2.92- 2.78 (m, 1H), 2.58 (m, 2H), 2.48 (s, 3H), 2.43-2.38 (m, 3H), 2.37-2.20 (m, 1H), 1.89-1.79 (m, 1H), 1.54 (m, 3H). |
| VI-10 | [chemical structure] | 613.15 [M + H]⁺ | ¹H NMR (400 MHz, Methanol- d₄) δ 8.45 (d, J = 2.6 Hz, 1H), 8.22-8.11 (m, 2H), 7.40 (d, J = 8.4 Hz, 1H), 7.21-7.10 (m, 2H), 6.79 (d, J = 6.8 Hz, 1H), 6.29 (d, J = 1.2 Hz, 1H), 5.42 (m, 3H), 4.18 (m, 1H), 3.93 (m, 1H), 3.70-3.48 (m, 3H), 3.19-3.01 (m, 3H), 2.98 (s, 3H), 2.51 (m, 3H), 2.37 (m, 2H), 1.57 (d, J = 6.8 Hz, 3H). |
| VI-11 | [chemical structure] | 613.45 [M + H]⁺ | ¹H NMR (400 MHz, Methanol- d₄) δ 8.44 (d, J = 2.6 Hz, 1H), 8.21 (d, J = 2.6 Hz, 1H), 8.14 (d, J = 6.9 Hz, 1H), 7.38 (d, J = 8.4 Hz, 1H), 7.23-7.05 (m, 2H), 6.78 (d, J = 6.9 Hz, 1H), 6.27 (d, J = 1.3 Hz, 1H), 5.60 (s, 1H), 5.40 (m, 1H), 5.31-5.09 (m, 1H), 4.17 (m, 1H), 3.86 (m, 1H), 3.62-3.35 (m, 3H), 3.27-3.14 (m, 1H), 3.11-2.88 (m, 5H), 2.55-2.23 (m, 5H), 1.55 (d, J = 6.8 Hz, 3H). |
| VI-12 | [chemical structure] | N/A | N/A |
| VI-13 | [chemical structure] | N/A | N/A |
| VI-14 | [chemical structure] | N/A | N/A |
| VI-15 | [chemical structure] | 569.15 [M + H]⁺ | N/A |
| VI-16 | [chemical structure] | 583.1 [M + H]⁺ | N/A |
| VI-17 | [chemical structure] | 592.05 [M + H]⁺ | N/A |
| VI-18 | [chemical structure] | N/A | N/A |

### Example 13—Synthesis of N-[4-[(4-Ethylpiperazin-1-yl)methyl]-3-(trifluoromethyl)phenyl]-3-[pyrrolo[2,1-f][1,2,4]triazin-4-yloxy]-5,6,7,8-tetrahydro-1,6-naphthyridine-6-carboxamide (Compound No. 8)

[chemical structure]

### Part I—Synthesis of 1-Methyl-3,5-dinitro-1,2-dihydropyridin-2-one

[chemical structure]

Into a 50 mL round-bottom flask was placed 3,5-dinitropyridin-2-ol (2 g, 10.81 mmol), N,N-dimethylformamide (10 mL), potassium carbonate (3 g, 21.71 mmol), and iodomethane (2.3 g, 16.2 mmol). The resulting solution was stirred overnight at 55° C. The crude material was purified by preparatory HPLC to yield 1-methyl-3,5-dinitro-1,2-dihydropyridin-2-one (1.2 g, 56%).

### Part II—Synthesis of tert-Butyl 3-nitro-5,6,7,8-tetrahydro-1,6-naphthyridine-6-carboxylate

[chemical structure]

Into a 20 mL vial was placed 1-methyl-3,5-dinitro-1,2-dihydropyridin-2-one (600 mg, 3.01 mmol), methanolic ammonia (10 mL), and tert-butyl 4-oxopiperidine-1-carboxylate (600 mg, 3.01 mmol). The reaction mixture was irradiated with microwave radiation for 30 min at 80° C. then was concentrated under vacuum. The resulting residue was purified by column chromatography eluting with ethyl acetate/petroleum ether (1:1). The collected fractions were combined and concentrated under vacuum to yield tert-butyl 3-nitro-5,6,7,8-tetrahydro-1,6-naphthyridine-6-carboxylate (560 mg, 67%).

### Part III—Synthesis of tert-Butyl 3-amino-5,6,7,8-tetrahydro-1,6-naphthyridine-6-carboxylate

[chemical structure]

Into a 50 mL round-bottom flask was placed tert-butyl 3-nitro-5,6,7,8-tetrahydro-1,6-naphthyridine-6-carboxylate (560 mg, 2.0 mmol), tetrahydrofuran (10 mL), and 10% rhodium on carbon (560 mg, 0.54 mmol). The flask was purged, then charged with hydrogen gas. The resulting mixture was stirred overnight at room temperature. The solids were removed by filtration, and the filtrate was concentrated under vacuum to yield tert-butyl 3-amino-5,6,7,8-tetrahydro-1,6-naphthyridine-6-carboxylate (500 mg, 100% crude yield).

### Part IV—Synthesis of 5,6,7,8-Tetrahydro-1,6-naphthyridin-3-ol

[chemical structure]

Into a 50 mL, 3-necked round-bottom flask, purged and maintained with an inert atmosphere of nitrogen, was placed tert-butyl 3-amino-5,6,7,8-tetrahydro-1,6-naphthyridine-6-carboxylate (510 mg, 2.05 mmol), water (10 mL), 12 M hydrogen chloride solution (1.2 mL, 14.4 mmol) and sodium nitrite (211 mg, 3.06 mmol). The resulting solution was stirred for 2 hours at room temperature then for an additional 1 hour at 80° C. The resulting mixture was concentrated under vacuum. The crude product was purified by preparatory HPLC to yield 5,6,7,8-tetrahydro-1,6-naphthyridin-3-ol (300 mg, 98%).

### Part V—Synthesis of tert-Butyl 3-hydroxy-5,6,7,8-tetrahydro-1,6-naphthyridine-6-carboxylate

[chemical structure]

Into a 25 mL round-bottom flask was placed 5,6,7,8-tetrahydro-1,6-naphthyridin-3-ol (60 mg, 0.40 mmol), tetrahydrofuran (4 mL), 1 M sodium hydroxide (4 mL) and di-tert-butyl dicarbonate (87 mg, 0.40 mmol). The resulting solution was stirred overnight at room temperature then concentrated under vacuum. The residue was purified by column chromatography eluting with ethyl acetate/petroleum ether (1:1). The collected fractions were combined and concentrated under vacuum to yield tert-butyl 3-hydroxy-5,6,7,8-tetrahydro-1,6-naphthyridine-6-carboxylate (30 mg, 30%).

### Part VI—Synthesis of tert-Butyl 3-[pyrrolo[2,1-f][1,2,4]triazin-4-yloxy]-5,6,7,8-tetrahydro-1,6-naphthyridine-6-carboxylate

[chemical structure]

Into a 25 mL round-bottom flask was placed tert-butyl 3-hydroxy-5,6,7,8-tetrahydro-1,6-naphthyridine-6-carboxylate (75 mg, 0.30 mmol), N,N-dimethylformamide (5 mL), cesium carbonate (196 mg, 0.60 mmol) and 4-chloropyrrolo[2,1-f][1,2,4]triazine (91 mg, 0.59 mmol). The resulting mixture was stirred overnight at room temperature then diluted with ethyl acetate and washed with saturated sodium chloride (aq). The organic layer was dried over anhydrous sodium sulfate and concentrated under vacuum. The residue was purified by column chromatography eluting with ethyl acetate/petroleum ether (1:2). The collected fractions were combined and concentrated under vacuum to yield tert-butyl 3-[pyrrolo[2,1-f][1,2,4]triazin-4-yloxy]-5,6,7,8-tetrahydro-1,6-naphthyridine-6-carboxylate (60 mg, 55%).

### Part VII—Synthesis of 3-[Pyrrolo[2,1-f][1,2,4]triazin-4-yloxy]-5,6,7,8-tetrahydro-1,6-naphthyridine

[chemical structure]

Into a 25 mL round-bottom flask, was placed tert-butyl 3-[pyrrolo[2,1-f][1,2,4]triazin-4-yloxy]-5,6,7,8-tetrahydro-1,6-naphthyridine-6-carboxylate (60 mg, 0.16 mmol) and dichloromethane (6 mL) followed by trifluoroacetic acid (2 mL). The resulting solution was stirred overnight at room temperature then concentrated under vacuum to yield 3-[pyrrolo[2,1-f][1,2,4]triazin-4-yloxy]-5,6,7,8-tetrahydro-1,6-naphthyridine as a trifluoroacetate solvate (40 mg, 92%).

### Part VIII—Synthesis of N-[4-[(4-Ethylpiperazin-1-yl)methyl]-3-(trifluoromethyl)phenyl]-3-[pyrrolo[2,1-f][1,2,4]triazin-4-yloxy]-5,6,7,8-tetrahydro-1,6-naphthyridine-6-carboxamide (Compound No. 8)

[chemical structure]

Into a 25 mL round-bottom flask was placed 3-[pyrrolo[2,1-f][1,2,4]triazin-4-yloxy]-5,6,7,8-tetrahydro-1,6-naphthyridine (40 mg, 0.15 mmol), dichloromethane (5 mL), triethylamine (30 mg, 0.30 mmol) and 1-ethyl-4-([4-isocyanato-2-(trifluoromethyl)phenyl]methyl)piperazine (94 mg, 0.30 mmol). The resulting solution was stirred for 2 hours at room temperature then concentrated under vacuum. The resulting residue was purified by preparatory HPLC to yield N-[4-[(4-ethylpiperazin-1-yl)methyl]-3-(trifluoromethyl)phenyl]-3-[pyrrolo[2,1-f][1,2,4]triazin-4-yloxy]-5,6,7,8-tetrahydro-1,6-naphthyridine-6-carboxamide (14.7 mg, 17%). (ES, m/z): [M+H]⁺=581.3. ¹H NMR (300 MHz, DMSO-d₆, ppm): δ 9.02 (s, 1H), 8.45 (d, J=2.6 Hz, 1H), 8.14 (s, 1H), 8.11 (dd, J=2.6, 1.5 Hz, 1H), 7.90 (d, J=2.2 Hz, 1H), 7.79-7.71 (m, 2H), 7.59 (d, J=8.6 Hz, 1H), 7.12 (dd, J=4.5, 1.5 Hz, 1H), 6.99 (dd, J=4.5, 2.6 Hz, 1H), 4.73 (s, 2H), 3.88 (t, J=5.9 Hz, 2H), 3.51 (s, 2H), 3.01 (t, J=5.7 Hz, 2H), 2.46-2.15 (m, 10H), 0.97 (t, J=7.1 Hz, 3H).

### Example 14—Synthesis of Additional N-phenyl-5,6,7,8-tetrahydro-1,6-naphthyridine-6-carboxamides and Related Compounds

The compounds in Table 7 below were prepared based on experimental procedures described in Example 13 and in the detailed description.

TABLE 7

| | | Mass Spec. |
|---|---|---|
| No. | Chemical Structure | (ES, m/z) |
| VII-1 | [chemical structure] | 580.2 [M + H]⁺ |
| VII-2 | [chemical structure] | 581.15 [M + H]⁺ |
| VII-3 | [chemical structure] | 608.1 [M + H]⁺ |
| VII-4 | [chemical structure] | 608.3 [M + H]⁺ |
| | Enantiomer A | |
| VII-5 | [chemical structure] | 608.25 [M + H]⁺ |
| | Enantiomer B | |
| VII-6 | [chemical structure] | 596.05 [M + H]⁺ |
| VII-7 | [chemical structure] | 596.05 [M + H]⁺ |
| | Enantiomer A | |
| VII-8 | [chemical structure] | 596.25 [M + H]⁺ |
| | Enantiomer B | |
| VII-9 | [chemical structure] | 583.25 [M + H]⁺ |
| VII-10 | [chemical structure] | 583.25 [M + H]⁺ |
| | Enantiomer A | |
| VII-11 | [chemical structure] | 583.1 [M + H]⁺ |
| | Enantiomer B | |
| VII-12 | [chemical structure] | 597 [M + H]⁺ |
| VII-13 | [chemical structure] | 597.05 [M + H]⁺ |
| | Enantiomer A | |
| VII-14 | [chemical structure] | 597.1 [M + H]⁺ |
| | Enantiomer B | |
| VII-15 | [chemical structure] | 595.25 [M + H]⁺ |
| VII-16 | [chemical structure] | 595.05 [M + H]⁺ |
| | Enantiomer A | |
| VII-17 | [chemical structure] | 595.05 [M + H]⁺ |
| | Enantiomer B | |
| VII-18 | [chemical structure] | N/A |
| VII-19 | [chemical structure] | 612.25 [M + H]⁺ |
| | 3,4-cis Relative Stereochemistry on | |
| | Piperidinyl Ring, and | |
| | Mixture of Stereoisomers at | |
| | Methyl-bearing Stereocenter | |
| VII-20 | [chemical structure] | N/A |
| | Enantiomer A | |
| VII-21 | [chemical structure] | N/A |
| | Enantiomer B | |
| VII-22 | [chemical structure] | N/A |
| | Enantiomer A | |
| VII-23 | [chemical structure] | N/A |
| | Enantiomer B | |

### Example 15—Synthesis of 3-((1H-pyrrolo[2,3-b]pyridin-4-yl)oxy)-N-(4-((4-ethylpiperazin-1-yl)methyl)-3-(trifluoromethyl)phenyl)-5-methyl-7,8-dihydro-1,6-naphthyridine-6(5H)-carboxamide (Compound No. 9)

[chemical structure]

### Part I—Synthesis of 3-Bromo-5-methoxy-2-methylpyridine

[chemical structure]

Into a 100-mL round-bottom flask, was placed a solution of 5-bromo-6-methylpyridin-3-ol (3 g, 16 mmol) in N,N-dimethylformamide (30 mL). Potassium carbonate (3.3 g, 24 mmol) was added, followed by iodomethane (3.4 g, 24 mmol). The mixture was stirred at room temperature overnight then diluted with ethyl acetate (100 mL), washed with water (3×100 mL), dried over anhydrous sodium sulfate, and concentrated. The residue was purified on a silica gel column eluting with petroleum ether/ethyl acetate (4:1) to yield 3-bromo-5-methoxy-2-methylpyridine (1.5 g, 47%).

### Part II—Synthesis of 3-Bromo-2-(bromomethyl)-5-methoxypyridine

[chemical structure]

Into a 100-mL round-bottom flask was placed a solution of 3-bromo-5-methoxy-2-methylpyridine (1.5 g, 7.4 mmol) in carbon tetrachloride (20 mL), followed by addition of N-bromosuccinimide (1.5 g, 8.1 mmol) and benzoyl peroxide (181 mg, 0.07 mmol). The mixture was stirred at 80° C. for 1 hour. The solvents were removed, and the residue was purified on a silica gel column eluting with petroleum ether/ethyl acetate (4:1) to yield 3-bromo-2-(bromomethyl)-5-methoxypyridine (1.4 g, 67%).

### Part III—Synthesis of 2-(3-Bromo-5-methoxypyridin-2-yl)acetonitrile

[chemical structure]

Into a 100-mL round-bottom flask was dissolved 3-bromo-2-(bromomethyl)-5-methoxypyridine (1.4 g, 5 mmol) in tetrahydrofuran (20 mL). To this solution was added 1 M tetrabutylammonium fluoride in tetrahydrofuran (7.5 mL, 7.5 mmol) and trimethylsilylcyanide (0.8 g, 7.5 mmol). The solution was stirred at 80° C. for 1 hour then quenched by the addition of excess ammonium hydroxide. The solvents were removed under reduced pressure, and the residue was purified on a silica gel column eluting with petroleum ether/ethyl acetate (4:1) to yield 2-(3-bromo-5-methoxypyridin-2-yl)acetonitrile (800 mg, 70%).

### Part IV—Synthesis of 2-(5-Methoxy-3-(2-methyl-1,3-dioxolan-2-yl)pyridin-2-yl)acetonitrile

[chemical structure]

Into a 25-mL round-bottom flask purged and maintained with an inert atmosphere of nitrogen, was placed 2-(3-bromo-5-methoxypyridin-2-yl)acetonitrile (1 g, 4.4 mmol), 2-(vinyloxy)ethan-1-ol (1.9 g, 21.6 mmol), 1,1′-bis(diphenylphosphino)ferrocene (146 mg, 0.26 mmol), and palladium(II) acetate (30 mg, 0.13 mmol). This was followed by the room temperature addition of ethylene glycol (10 mL) and triethylamine (1.2 mL, 8.9 mmol). The mixture was stirred at 140° C. for 30 minutes, then diluted with water (50 mL) and extracted with ethyl acetate (2×50 mL). The combined organics were dried over anhydrous sodium sulfate, concentrated, and then purified on a silica gel column eluting with petroleum ether/ethyl acetate (2:1) to yield 2-(5-methoxy-3-(2-methyl-1,3-dioxolan-2-yl)pyridin-2-yl)acetonitrile (500 mg, 48%).

### Part V—Synthesis of 2-(5-Methoxy-3-(2-methyl-1,3-dioxolan-2-yl)pyridin-2-yl)ethan-1-amine

[chemical structure]

Into a 50-mL round-bottom flask, was dissolved 2-(5-methoxy-3-(2-methyl-1,3-dioxolan-2-yl)pyridin-2-yl)acetonitrile (500 mg, 2.1 mmol) in ethanol (10 mL). The flask was evacuated and filled with nitrogen. Raney-nickel (500 mg, 5.8 mmol) was added, then the flask was evacuated and refilled with hydrogen gas. The resulting solution was stirred at room temperature for 2 hours under a hydrogen ballon. The flask was evacuated and refilled with nitrogen gas. Solids were removed by filtration, and the filtrate was concentrated under reduced pressure to yield 2-(5-methoxy-3-(2-methyl-1,3-dioxolan-2-yl)pyridin-2-yl)ethan-1-amine (450 mg, 88%).

### Part VI—Synthesis of 3-Methoxy-5-methyl-7,8-dihydro-1,6-naphthyridine

[chemical structure]

Into a 25-mL round-bottom flask was placed 2-(5-methoxy-3-(2-methyl-1,3-dioxolan-2-yl)pyridin-2-yl)ethan-1-amine (450 mg, 1.89 mmol) in 6 M hydrogen chloride (4 mL). The solution was stirred at 100° C. for 2 hours. The pH was adjusted to 9 with sodium bicarbonate. The solution was extracted with ethyl acetate (3×50 mL). The combined organics were dried over anhydrous sodium sulfate, decanted, and concentrated. The residue was applied onto a silica gel column and eluted with ethyl acetate (100%) to yield 3-methoxy-5-methyl-7,8-dihydro-1,6-naphthyridine (290 mg, 87%).

### Part VII—Synthesis of 3-Methoxy-5-methyl-5,6,7,8-tetrahydro-1,6-naphthyridine

[chemical structure]

Into a 50-mL round-bottom flask was dissolved 3-methoxy-5-methyl-7,8-dihydro-1,6-naphthyridine (292 mg, 1.7 mmol) in methanol (5 mL). Sodium borohydride (313 mg, 8.27 mmol) was added, and the mixture was stirred at room temperature for 2 hours. The mixture was concentrated and then applied onto a silica gel column and eluted with dichloromethane/methanol (10:1) to yield 3-methoxy-5-methyl-5,6,7,8-tetrahydro-1,6-naphthyridine (210 mg, 71%).

### Part VIII—Synthesis of 5-Methyl-5,6,7,8-tetrahydro-1,6-naphthyridin-3-ol

[chemical structure]

Into a 50-mL round-bottom flask was placed 3-methoxy-5-methyl-5,6,7,8-tetrahydro-1,6-naphthyridine (210 mg, 1.2 mmol) in dichloromethane (10 ml). Boron tribromide 1 M in dichloromethane (6 mL, 6 mmol) was added. The solution was stirred at room temperature overnight. The reaction was quenched by the addition of excess methanol. The mixture was concentrated under reduced pressure to yield 5-methyl-5,6,7,8-tetrahydro-1,6-naphthyridin-3-ol (200 mg, quantitative crude yield) that was carried onto the next step without further purification.

### Part IX—Synthesis of tert-Butyl 3-((tert-butoxycarbonyl)oxy)-5-methyl-7,8-dihydro-1,6-naphthyridine-6(5H)-carboxylate

[chemical structure]

Into a 50-mL round-bottom flask was dissolved 5-methyl-5,6,7,8-tetrahydro-1,6-naphthyridin-3-ol (200 mg, 1.2 mmol) in dichloromethane (10 mL). Triethylamine (0.8 mL, 5.7 mmol) and di-tert-butyl dicarbonate (0.5 g, 2.3 mmol) were added. The solution was stirred at room temperature for 2 hours. The mixture was diluted with water (30 mL), extracted with dichloromethane (3×30 mL), and the combined organics were dried over anhydrous sodium sulfate and concentrated to yield crude tert-butyl 3-((tert-butoxycarbonyl)oxy)-5-methyl-7,8-dihydro-1,6-naphthyridine-6(5H)-carboxylate (380 mg).

### Part X—Synthesis of tert-Butyl 3-hydroxy-5-methyl-7,8-dihydro-1,6-naphthyridine-6(5H)-carboxylate

[chemical structure]

Into a 25-mL round-bottom flask was placed crude tert-butyl 3-((tert-butoxycarbonyl)oxy)-5-methyl-7,8-dihydro-1,6-naphthyridine-6(5H)-carboxylate (380 mg) and a 30% solution of sodium methoxide in methanol (4 mL) was added. The solution was stirred at room temperature for 2 hours, then quenched by the addition of ice water. The mixture was extracted with ethyl acetate (3×50 mL). The combined organics were dried over anhydrous sodium sulfate then concentrated. The residue was applied onto a silica gel column and eluted with dichloromethane/methanol (10:1) to yield tert-butyl 3-hydroxy-5-methyl-7,8-dihydro-1,6-naphthyridine-6(5H)-carboxylate (170 mg, 62%)

### Part XI—Synthesis of tert-Butyl 5-methyl-3-((1-(triisopropylsilyl)-1H-pyrrolo[2,3-b]pyridin-4-yl)oxy)-7,8-dihydro-1,6-naphthyridine-6(5H)-carboxylate

[chemical structure]

Into a 50-mL round-bottom flask, purged and maintained with an atmosphere of oxygen, was placed copper(II) acetate (110 mg, 0.61 mmol), pyridine (0.24 mL, 3.1 mmol), and dichloromethane (5 mL). The mixture was stirred for 30 min at room temperature, then 4 Å molecular sieves (0.32 g) were added, followed by tert-butyl 3-hydroxy-5-methyl-7,8-dihydro-1,6-naphthyridine-6(5H)-carboxylate (160 mg, 0.61 mmol) and (1-(triisopropylsilyl)-1H-pyrrolo[2,3-b]pyridin-4-yl)boronic acid (300 mg, 0.94 mmol). The reaction was stirred for 18 hours at room temperature. The solids were removed by filtration, and the filtrate was concentrated. The residue was applied onto a silica gel column and eluted with ethyl acetate in petroleum ether (10:1) to yield tert-butyl 5-methyl-3-((1-(triisopropylsilyl)-1H-pyrrolo[2,3-b]pyridin-4-yl)oxy)-7,8-dihydro-1,6-naphthyridine-6(5H)-carboxylate (150 mg, 46%).

### Part XII—Synthesis of 3-((1H-Pyrrolo[2,3-b]pyridin-4-yl)oxy)-5-methyl-5,6,7,8-tetrahydro-1,6-naphthyridine

[chemical structure]

Into a 25-mL round-bottom flask, was placed tert-butyl 5-methyl-3-((1-(triisopropylsilyl)-1H-pyrrolo[2,3-b]pyridin-4-yl)oxy)-7,8-dihydro-1,6-naphthyridine-6(5H)-carboxylate (150 mg, 0.28 mmol), and 6 M hydrogen chloride (aq., 4 mL). The solution was stirred for 2 hours at room temperature then concentrated to yield crude 3-((1H-pyrrolo[2,3-b]pyridin-4-yl)oxy)-5-methyl-5,6,7,8-tetrahydro-1,6-naphthyridine as the hydrochloride salt (70 mg, 89%).

### Part XIII—Synthesis of 3-((1H-Pyrrolo[2,3-b]pyridin-4-yl)oxy)-N-(4-((4-ethylpiperazin-1-yl)methyl)-3-(trifluoromethyl)phenyl)-5-methyl-7,8-dihydro-1,6-naphthyridine-6(5H)-carboxamide (Compound No. 9)

[chemical structure]

Into a 250-mL round-bottom flask, was placed 3-((1H-pyrrolo[2,3-b]pyridin-4-yl)oxy)-5-methyl-5,6,7,8-tetrahydro-1,6-naphthyridine (50 mg, 0.18 mmol), dichloromethane (5 mL), and triethylamine (0.12 mL, 0.89 mmol) followed by 1-ethyl-4-(4-isocyanato-2-(trifluoromethyl)benzyl)piperazine (56 mg, 0.18 mmol). The reaction was stirred for 2 hours at room temperature. The mixture was concentrated and purified by preparatory HPLC to yield 3-((1H-pyrrolo[2,3-b]pyridin-4-yl)oxy)-N-(4-((4-ethylpiperazin-1-yl)methyl)-3-(trifluoromethyl)phenyl)-5-methyl-7,8-dihydro-1,6-naphthyridine-6(5H)-carboxamide (38 mg, 36%). (ES, m/z): [M+H]⁺=594. ¹H NMR (400 MHz, Methanol-d₄, ppm) δ 8.32 (m, 1H), 8.14 (m, 1H), 7.82 (m, 1H), 7.68 (m, 2H), 7.60 (m, 1H), 7.34 (m, 1H), 6.60 (m, 1H), 6.34 (m, 1H), 5.48 (m, 1H), 4.37 (m, 1H), 3.65-3.50 (m, 3H), 3.2-3.0 (m, 2H), 2.65-2.45 (m, 10H), 1.58 (d, 3H), 1.14 (t, 3H).

### Example 16—Synthesis of Additional N-phenyl-5-methyl-7,8-dihydro-1,6-naphthyridine-6(5H)-carboxamides and Related Compounds

The compounds in Table 8 below were prepared based on experimental procedures described in Example 15 and in the detailed description.

TABLE 8

| | | Mass Spec. |
|---|---|---|
| No. | Chemical Structure | (ES, m/z) |
| VIII-1 | [chemical structure] | 608.5 [M + H]⁺ |
| VIII-2 | [chemical structure] | N/A |
| | Enantiomer A | |
| VIII-3 | [chemical structure] | 595.1 [M + H]⁺ |
| VIII-4 | [chemical structure] | 595.15 [M + H]⁺ |
| | Enantiomer A | |
| VIII-5 | [chemical structure] | 595.15 [M + H]⁺ |
| | Enantiomer B | |
| VIII-6 | [chemical structure] | N/A |

### Example 17—Synthesis of 3-((1H-Pyrrolo[2,3-b]pyridin-4-yl)oxy)-N-(4-((4-ethylpiperazin-1-yl)methyl)-3-(trifluoromethyl)phenyl)-8-methyl-7,8-dihydro-1,6-naphthyridine-6(5H)-carboxamide (Compound No. 10)

[chemical structure]

### Part I—Synthesis of 2-(3-Bromo-5-methoxypyridin-2-yl)propanenitrile

[chemical structure]

Into a 100-mL round-bottom flask was dissolved 2-(3-bromo-5-methoxypyridin-2-yl)acetonitrile (1 g, 4.40 mmol) in anhydrous N,N-dimethylformamide (10 mL). The solution was cooled to 0° C. and sodium hydride (0.2 g, 4.84 mmol) was added in portions. After sodium hydride addition was complete, iodomethane (0.7 g, 4.84 mmol) was added. The mixture was stirred at 0° C. for 2 hours then quenched by the addition of water. The mixture was extracted with ethyl acetate (2×10 mL), then the combined organics were dried over anhydrous sodium sulfate and concentrated. The residue was applied onto a silica gel column and eluted with petroleum ether/ethyl acetate (4:1) to yield 2-(3-bromo-5-methoxypyridin-2-yl)propanenitrile (800 mg, 75%).

### Part II—Synthesis of 2-(5-Methoxy-3-vinylpyridin-2-yl)propanenitrile

[chemical structure]

Into a 50-mL round-bottom flask purged and maintained with an inert atmosphere of nitrogen was placed 2-(3-bromo-5-methoxypyridin-2-yl)propanenitrile (1.2 g, 5.0 mmol), 2-ethenyl-4,4,5,5-tetramethyl-1,3,2-dioxaborolane (1.1 g, 7.5 mmol), sodium carbonate (1.6 g, 15 mmol), tetrakis(triphenylphosphine)palladium(0) (0.6 g, 0.50 mmol) in 1,4-dioxane (20 mL) and water (5 mL). The reaction was stirred at 100° C. overnight. The reaction was diluted with water (50 mL), extracted with ethyl acetate (3×50 mL). The combined organics were dried over anhydrous sodium sulfate then concentrated. The residue was applied onto a silica gel column and eluted with petroleum ether/ethyl acetate (4:1) to yield 2-(5-methoxy-3-vinylpyridin-2-yl)propanenitrile (750 mg, 80%).

### Part III—Synthesis of 2-(3-Formyl-5-methoxypyridin-2-yl)propanenitrile

[chemical structure]

Into a 100-mL round-bottom flask was dissolved 2-(5-methoxy-3-vinylpyridin-2-yl)propanenitrile (800 mg, 4.3 mmol) in 1,4-dioxane (15 mL) and water (5 mL). This was followed by the addition of osmium tetroxide (54 mg, 0.21 mmol) and sodium periodate (1.8 g, 8.50 mmol). The mixture was stirred at room temperature overnight. The reaction was then quenched by the addition of excess sodium bisulfite. The mixture was extracted with ethyl acetate (3×100 mL), and the combined organics were dried over anhydrous sodium sulfate and concentrated. The residue was applied onto a silica gel column and eluted with petroleum ether/ethyl acetate (4:1) to yield 2-(3-formyl-5-methoxypyridin-2-yl)propanenitrile (560 mg, 69%).

### Part IV—Synthesis of 2-(3-(1,3-Dioxolan-2-yl)-5-methoxypyridin-2-yl)propanenitrile

[chemical structure]

Into a 50-mL round-bottom flask was placed a solution of 2-(3-formyl-5-methoxypyridin-2-yl)propanenitrile (740 mg, 3.9 mmol) in toluene (15 mL). Ethylene glycol (724 mg, 12 mmol) and p-toluenesulfonic acid (134 mg, 0.78 mmol) were added. The solution was stirred at 110° C. for 3 hours. The solvents were removed, and the residue was purified on a silica gel column eluting with petroleum ether/ethyl acetate (5:1) to yield 2-(3-(1,3-dioxolan-2-yl)-5-methoxypyridin-2-yl)propanenitrile (680 mg, 75%).

### Part V—Synthesis of 2-(3-(1,3-Dioxolan-2-yl)-5-methoxypyridin-2-yl)propan-1-amine

[chemical structure]

Into a 50-mL round-bottom flask was dissolved 2-(3-(1,3-dioxolan-2-yl)-5-methoxypyridin-2-yl)propanenitrile (680 mg, 2.90 mmol) in ethanol (10 mL). The flask was purged with nitrogen, and Raney nickel (680 mg, 7.94 mmol) was added. The flask was then evacuated, and refilled with hydrogen gas. The reaction was stirred under a hydrogen balloon at room temperature for 2 hours. The flask was evacuated and refilled with nitrogen. The solids were removed by filtration, the filtrate was concentrated to yield 2-(3-(1,3-dioxolan-2-yl)-5-methoxypyridin-2-yl)propan-1-amine (650 mg, 94%).

### Part VI—Synthesis of 3-Methoxy-8-methyl-7,8-dihydro-1,6-naphthyridine

[chemical structure]

Into a 25-mL round-bottom flask was placed 2-(3-(1,3-dioxolan-2-yl)-5-methoxypyridin-2-yl)propan-1-amine (650 mg, 3.7 mmol) in 6 M hydrogen chloride (4 mL). The solution was stirred at 100° C. for 2 hours. The pH was adjusted to 9 with sodium bicarbonate. The solution was extracted with ethyl acetate (2×50 mL). The combined organics were dried over anhydrous sodium sulfate, decanted, and concentrated. The residue was applied onto a silica gel column and eluted with ethyl acetate (100%) to yield 3-methoxy-8-methyl-7,8-dihydro-1,6-naphthyridine (312 mg, 47%).

### Part VII—Synthesis of 3-Methoxy-8-methyl-5,6,7,8-tetrahydro-1,6-naphthyridine

[chemical structure]

Into a 25-mL round-bottom flask was dissolved 3-methoxy-8-methyl-7,8-dihydro-1,6-naphthyridine (350 mg, 2 mmol) in methanol (5 mL). Sodium borohydride (378 mg, 10 mmol) was added, and the mixture was stirred at room temperature for 2 hours. The mixture was concentrated. The residue was applied onto a silica gel column and eluted with dichloromethane/methanol (10:1) to yield 3-methoxy-8-methyl-5,6,7,8-tetrahydro-1,6-naphthyridine (280 mg, 79%).

### Part VIII—Synthesis of 8-Methyl-5,6,7,8-tetrahydro-1,6-naphthyridin-3-ol

[chemical structure]

Into a 50-mL round-bottom flask was placed 3-methoxy-8-methyl-5,6,7,8-tetrahydro-1,6-naphthyridine (280 mg, 1.6 mmol) in dichloromethane (10 mL). Boron tribromide 1 M in dichloromethane (7.9 mL, 7.9 mmol) was added. The solution was stirred at room temperature overnight. The reaction was quenched by the addition of excess methanol. The mixture was concentrated under reduced pressure to yield 8-methyl-5,6,7,8-tetrahydro-1,6-naphthyridin-3-ol (190 mg, 74%) that was carried onto the next step crude.

### Part IX—Synthesis of tert-Butyl 3-((tert-butoxycarbonyl)oxy)-8-methyl-7,8-dihydro-1,6-naphthyridine-6(5H)-carboxylate

[chemical structure]

Into a 50-mL round-bottom flask was dissolved 8-methyl-5,6,7,8-tetrahydro-1,6-naphthyridin-3-ol (200 mg, 1.22 mmol) in dichloromethane (10 mL). Triethylamine (0.79 mL, 5.70 mmol) and di-tert-butyl dicarbonate (0.5 g, 2.3 mmol) were added. The solution was stirred at room temperature for 2 hours. The mixture was diluted with water (30 mL) and extracted with dichloromethane (2×50 mL). The combined organics were dried over anhydrous sodium sulfate and concentrated to yield crude tert-butyl 3-((tert-butoxycarbonyl)oxy)-8-methyl-7,8-dihydro-1,6-naphthyridine-6(5H)-carboxylate (380 mg, 86%).

### Part X—Synthesis of tert-Butyl 3-hydroxy-8-methyl-7,8-dihydro-1,6-naphthyridine-6(5H)-carboxylate

[chemical structure]

Into a 25-mL round-bottom flask was placed crude tert-butyl 3-((tert-butoxycarbonyl)oxy)-8-methyl-7,8-dihydro-1,6-naphthyridine-6(5H)-carboxylate (380 mg, 1.04 mmol) and a 30% solution of sodium methoxide in methanol (4 mL) was added. The solution was stirred at room temperature for 2 hours, then quenched by the addition of ice water. The mixture was extracted with ethyl acetate (3×50 mL). The combined organics were dried over anhydrous sodium sulfate then concentrated. The residue was applied onto a silica gel column and eluted with dichloromethane/methanol (10:1) to yield tert-butyl 3-hydroxy-8-methyl-7,8-dihydro-1,6-naphthyridine-6(5H)-carboxylate (170 mg, 62%)

### Part XI—Synthesis of tert-Butyl 8-methyl-3-((1-(triisopropylsilyl)-1H-pyrrolo[2,3-b]pyridin-4-yl)oxy)-7,8-dihydro-1,6-naphthyridine-6(5H)-carboxylate

[chemical structure]

Into a 50-mL round-bottom flask, purged and maintained with an atmosphere of oxygen, was placed copper(II) acetate (110 mg, 0.61 mmol), pyridine (238 mg, 3.5 mmol), and dichloromethane (5 mL). The mixture was stirred for 30 min at room temperature, then 4 Å molecular sieves (0.4 g) were added, followed by tert-butyl 3-hydroxy-8-methyl-7,8-dihydro-1,6-naphthyridine-6(5H)-carboxylate (160 mg, 0.61 mmol) and (1-(triisopropylsilyl)-1H-pyrrolo[2,3-b]pyridin-4-yl)boronic acid (300 mg, 0.94 mmol). The reaction was stirred for 18 hours at room temperature. The solids were filtered out, and the filtrate was concentrated. The residue was applied onto a silica gel column and eluted with ethyl acetate in petroleum ether (10:1) to yield tert-butyl 8-methyl-3-((1-(triisopropylsilyl)-1H-pyrrolo[2,3-b]pyridin-4-yl)oxy)-7,8-dihydro-1,6-naphthyridine-6(5H)-carboxylate (160 mg, 49%).

### Part XII—Synthesis of 3-((1H-Pyrrolo[2,3-b]pyridin-4-yl)oxy)-8-methyl-5,6,7,8-tetrahydro-1,6-naphthyridine

[chemical structure]

Into a 25-mL round-bottom flask, was placed tert-butyl 8-methyl-3-((1-(triisopropylsilyl)-1H-pyrrolo[2,3-b]pyridin-4-yl)oxy)-7,8-dihydro-1,6-naphthyridine-6(5H)-carboxylate (150 mg, 0.28 mmol) and 6 M hydrogen chloride (aq., 4 mL). The solution was stirred for 2 hours at room temperature then concentrated to yield crude 3-((1H-pyrrolo[2,3-b]pyridin-4-yl)oxy)-8-methyl-5,6,7,8-tetrahydro-1,6-naphthyridine as the hydrochloride salt (65 mg, 83%).

### Part XIII—Synthesis of 3-((1H-pyrrolo[2,3-b]pyridin-4-yl)oxy)-N-(4-((4-ethylpiperazin-1-yl)methyl)-3-(trifluoromethyl)phenyl)-8-methyl-7,8-dihydro-1,6-naphthyridine-6(5H)-carboxamide (Compound No. 10)

[chemical structure]

Into a 250-mL round-bottom flask, was placed 3-((1H-pyrrolo[2,3-b]pyridin-4-yl)oxy)-8-methyl-5,6,7,8-tetrahydro-1,6-naphthyridine (50 mg, 0.18 mmol), dichloromethane (5 mL), and triethylamine (0.12 mL, 0.89 mmol) followed by 1-ethyl-4-(4-isocyanato-2-(trifluoromethyl)benzyl)piperazine (56 mg, 0.18 mmol). The reaction was stirred for 2 hours at room temperature. The mixture was concentrated and purified by preparatory HPLC to yield 3-((1H-pyrrolo[2,3-b]pyridin-4-yl)oxy)-N-(4-((4-ethylpiperazin-1-yl)methyl)-3-(trifluoromethyl)phenyl)-8-methyl-7,8-dihydro-1,6-naphthyridine-6(5H)-carboxamide (17.2 mg, 17%). (ES, m/z): [M+H]⁺=594. ¹H NMR (300 MHz, Methanol-d₄, ppm) δ 8.32 (d, J=2.7 Hz, 1H), 8.10 (d, J=5.5 Hz, 1H), 7.79 (d, J=1.9 Hz, 1H), 7.71-7.58 (m, 2H), 7.47 (d, J=2.7 Hz, 1H), 7.31 (d, J=3.5 Hz, 1H), 6.57 (d, J=5.6 Hz, 1H), 6.32 (d, J=3.6 Hz, 1H), 4.89 (s, 1H), 4.68 (d, J=17.0 Hz, 1H), 3.83 (dd, J=5.3, 1.5 Hz, 2H), 3.61 (d, J=1.5 Hz, 2H), 3.19 (q, J=5.8 Hz, 1H), 2.63-2.29 (m, 10H), 1.40 (d, J=7.0 Hz, 3H), 1.10 (t, J=7.3 Hz, 3H).

### Example 18—Synthesis of N-(4-((4-Ethylpiperazin-1-yl)methyl)-3-(trifluoromethyl)phenyl)-7-(pyrrolo[2,1-f][1,2,4]triazin-4-ylamino)-3,4-dihydroisoquinoline-2(1H)-carboxamide (Compound No. 11)

[chemical structure]

### Part I—Synthesis of tert-Butyl 7-nitro-3,4-dihydroisoquinoline-2(1H)-carboxylate

[chemical structure]

Into a 25 mL round-bottom flask was placed a solution of 7-nitro-1,2,3,4-tetrahydroisoquinoline (200 mg, 1.12 mmol) in dichloromethane (10 mL) followed by di-tert-butyl dicarbonate (489 mg, 2.24 mmol) and triethylamine (339 mg, 3.35 mmol). The reaction mixture was stirred overnight at room temperature then concentrated under vacuum. The residue was purified by column chromatography eluting with ethyl acetate/petroleum ether (1:10). The collected fractions were combined and concentrated under vacuum to yield tert-butyl 7-nitro-3,4-dihydroisoquinoline-2(1H)-carboxylate (290 mg, 93%).

### Part II—Synthesis of tert-Butyl 7-amino-1,2,3,4-tetrahydroisoquinoline-2-carboxylate

[chemical structure]

Into a 50 mL round-bottom flask purged and maintained with an inert atmosphere of hydrogen was placed a solution of tert-butyl 7-nitro-3,4-dihydroisoquinoline-2(1H)-carboxylate (290 mg, 1.04 mmol, 1.00 equiv) in ethyl acetate (10 mL) followed by rhodium on carbon (250 mg) at room temperature. The resulting mixture was stirred overnight at room temperature. The solids were removed by filtration, and the filtrate was concentrated under vacuum to yield tert-butyl 7-amino-1,2,3,4-tetrahydroisoquinoline-2-carboxylate (200 mg, 77%).

### Part III—Synthesis of tert-Butyl 7-(pyrrolo[2,1-f][1,2,4]triazin-4-ylamino)-3,4-dihydroisoquinoline-2(1H)-carboxylate

[chemical structure]

Into a 50 mL round-bottom flask was placed a solution of tert-butyl 7-amino-1,2,3,4-tetrahydroisoquinoline-2-carboxylate (190 mg, 0.77 mmol) in N,N-dimethylformamide (4 mL) followed by 4-chloropyrrolo[2,1-f][1,2,4]triazine (116 mg, 0.76 mmol) at room temperature. To this mixture was added cesium carbonate (753 mg, 2.31 mmol), and the whole mixture was stirred overnight at room temperature. The mixture was diluted with ethyl acetate and washed with water. The organic layer was concentrated under vacuum and purified by column chromatography eluting with ethyl acetate/petroleum ether (1:4). The collected fractions were combined and concentrated under vacuum to yield tert-butyl 7-(pyrrolo[2,1-f][1,2,4]triazin-4-ylamino)-3,4-dihydroisoquinoline-2(1H)-carboxylate (220 mg, 79%).

### Part IV—Synthesis of N-[Pyrrolo[2,1-f][1,2,4]triazin-4-yl]-1,2,3,4-tetrahydroisoquinolin-7-amine

[chemical structure]

Into a 25 mL round-bottom flask was placed a solution of tert-butyl 7-(pyrrolo[2,1-f][1,2,4]triazin-4-ylamino)-3,4-dihydroisoquinoline-2(1H)-carboxylate (210 mg, 0.57 mmol) in dichloromethane (6 mL) followed by trifluoroacetic acid (1 mL). The resulting solution was stirred for 1 hour at room temperature then concentrated under vacuum to yield N-[pyrrolo[2,1-f][1,2,4]triazin-4-yl]-1,2,3,4-tetrahydroisoquinolin-7-amine as a trifluoroacetate solvate (152 mg, 100%).

### Part V—Synthesis of N-(4-((4-Ethylpiperazin-1-yl)methyl)-3-(trifluoromethyl)phenyl)-7-(pyrrolo[2,1-f][1,2,4]triazin-4-ylamino)-3,4-dihydroisoquinoline-2(1H)-carboxamide (Compound No. 11)

[chemical structure]

Into a 25 mL round-bottom flask was placed a solution of N-[pyrrolo[2,1-f][1,2,4]triazin-4-yl]-1,2,3,4-tetrahydroisoquinolin-7-amine (50 mg, 0.19 mmol) in dichloromethane (5 mL) followed by 1-ethyl-4-[[4-isocyanato-2-(trifluoromethyl)phenyl]methyl]piperazine (59 mg, 0.19 mmol) and triethylamine (57 mg, 0.56 mmol) at room temperature. The resulting solution was stirred for 2 hours at room temperature then concentrated under vacuum. The residue was purified by preparatory HPLC to yield N-(4-((4-ethylpiperazin-1-yl)methyl)-3-(trifluoromethyl)phenyl)-7-(pyrrolo[2,1-f][1,2,4]triazin-4-ylamino)-3,4-dihydroisoquinoline-2(1H)-carboxamide (23.7 mg, 22%). (ES, m/z): [M+H]⁺: 579. ¹H NMR (300 MHz, DMSO-d₆, ppm) δ 9.81 (s, 1H), 8.90 (s, 1H), 8.01 (s, 1H), 7.93 (d, J=2.2 Hz, 1H), 7.84-7.71 (m, 3H), 7.67-7.54 (m, 2H), 7.26-7.14 (m, 2H), 6.74 (dd, J=4.4, 2.6 Hz, 1H), 4.69 (s, 2H), 3.74 (t, J=5.9 Hz, 2H), 3.52 (s, 2H), 2.85 (t, J=5.9 Hz, 2H), 2.48-2.24 (m, 9H), 2.08 (s, 1H), 0.98 (t, J=7.1 Hz, 3H).

### Example 19—Synthesis of Additional N-phenyl-3,4-dihydroisoquinoline-2(1H)-carboxamides and Related Compounds

The compounds in Table 9 below were prepared based on experimental procedures described in Example 18 and in the detailed description.

TABLE 9

| | | Mass Spec. | |
|---|---|---|---|
| No. | Chemical Structure | (ES, m/z) | NMR Spectrum |
| IX-1 | [chemical structure] | 578.3 [M + H]⁺ | ¹H NMR (300 MHz, DMSO- d6, ppm) δ 12.45 (d, J = 2.5 Hz, 1H), 10.23 (s, 1H), 9.00 (s, 1H), 8.08-7.92 (m, 2H), 7.82 (dd, J = 8.6, 2.2 Hz, 1H), 7.59 (d, J = 8.6 Hz, 1H), 7.45- 7.29 (m, 2H), 7.23 (d, J = 5.8 Hz, 2H), 6.85 (s, 1H), 6.69 (d, J = 7.0 Hz, 1H), 4.70 (m, 2H), 3.77 (m, 2H), 3.65 (m, 2H), 3.46 (m, 2H), 3.14 (m, 2H), 3.06-2.88 (m, 6H), 2.39 (t, J = 12.3 Hz, 2H), 1.21 (t, J = 7.3 Hz, 3H). |
| IX-2 | [chemical structure] | N/A | N/A |
| | Enantiomer A | | |
| IX-3 | [chemical structure] | N/A | N/A |
| | Enantiomer B | | |

[chemical structure]

### Example 20—Synthesis of N-(4-((4-ethylpiperazin-1-yl)methyl)-3-(trifluoromethyl)phenyl)-7-((6-(methylamino)pyrimidin-4-yl)oxy)-1,2,3,4-tetrahydronaphthalene-2-carboxamide (Compound No. 12)

[chemical structure]

### Part I—Synthesis of N-(4-((4-ethylpiperazin-1-yl)methyl)-3-(trifluoromethyl)phenyl)-7-hydroxy-1,2,3,4-tetrahydronaphthalene-2-carboxamide

[chemical structure]

Into a 25-mL round-bottom flask was placed a solution of 7-hydroxy-1,2,3,4-tetrahydronaphthalene-2-carboxylic acid (150 mg, 0.78 mmol) in dimethylsulfoxide (5 mL), then 1-hydroxybenotriazole hydrate (211 mg, 1.56 mmol) and N-(3-dimethylaminopropyl)-N′-ethylcarbodiimide hydrochloride (448 mg, 2.34 mmol) were added at room temperature. To the mixture was added 4-[(4-ethylpiperazin-1-yl)methyl]-3-(trifluoromethyl)aniline (224 mg, 0.78 mmol). The solution was stirred for 3 hours at room temperature. The mixture was diluted with dichloromethane (40 mL), washed with water (2×30 mL), and the organic layer was concentrated under vacuum. The residue was applied onto a silica gel column and eluted with dichloromethane/methanol (6:1) to yield N-(4-((4-ethylpiperazin-1-yl)methyl)-3-(trifluoromethyl)phenyl)-7-hydroxy-1,2,3,4-tetrahydronaphthalene-2-carboxamide (240 mg, 67%).

### Part II—Synthesis of 7-((6-chloropyrimidin-4-yl)oxy)-N-(4-((4-ethylpiperazin-1-yl)methyl)-3-(trifluoromethyl)phenyl)-1,2,3,4-tetrahydronaphthalene-2-carboxamide

[chemical structure]

Into a 25-mL round-bottom flask was placed a solution of N-(4-((4-ethylpiperazin-1-yl)methyl)-3-(trifluoromethyl)phenyl)-7-hydroxy-1,2,3,4-tetrahydronaphthalene-2-carboxamide (60 mg, 0.13 mmol) in N,N-dimethylformamide (4 mL) followed by the addition of 4,6-dichloropyrimidine (19 mg, 0.13 mmol) and cesium carbonate (127 mg, 0.39 mmol) at room temperature. The mixture was stirred for 1 hour at 45° C. then diluted with dichloromethane (35 mL) and washed with water (2×30 mL). The organic layer was concentrated under vacuum, and the residue was applied onto a silica gel column and eluted with dichloromethane/methanol (10:1) to yield 7-((6-chloropyrimidin-4-yl)oxy)-N-(4-((4-ethylpiperazin-1-yl)methyl)-3-(trifluoromethyl)phenyl)-1,2,3,4-tetrahydronaphthalene-2-carboxamide (50 mg, 67%).

### Part III—Synthesis of N-(4-((4-ethylpiperazin-1-yl)methyl)-3-(trifluoromethyl)phenyl)-7-((6-(methylamino)pyrimidin-4-yl)oxy)-1,2,3,4-tetrahydronaphthalene-2-carboxamide (Compound No. 12)

[chemical structure]

Into a 50-mL high pressure tube was placed 7-((6-chloropyrimidin-4-yl)oxy)-N-(4-((4-ethylpiperazin-1-yl)methyl)-3-(trifluoromethyl)phenyl)-1,2,3,4-tetrahydronaphthalene-2-carboxamide (40 mg, 0.07 mmol) followed by the addition of a 30% solution of methylamine in ethanol (4 mL) at room temperature. The tube was sealed, and the solution was stirred for 3 hours at 50° C. Upon cooling, the solvent was removed under vacuum. The residue was purified by prep-HPLC to yield N-(4-((4-ethylpiperazin-1-yl)methyl)-3-(trifluoromethyl)phenyl)-7-((6-(methylamino)pyrimidin-4-yl)oxy)-1,2,3,4-tetrahydronaphthalene-2-carboxamide (7.7 mg, 19%). (ES, m/z): [M+H]⁺: 569.55; ¹H NMR (400 MHz, dimethylsulfoxide-d₆, ppm) δ 10.31 (s, 1H), 8.10 (d, J=2.2 Hz, 2H), 7.81 (dd, J=8.4, 2.1 Hz, 1H), 7.67 (d, J=8.5 Hz, 1H), 7.29 (q, J=4.8 Hz, 1H), 7.15 (d, J=8.3 Hz, 1H), 6.94-6.84 (m, 2H), 5.75 (s, 1H), 3.57-3.51 (m, 2H), 2.93 (d, J=7.9 Hz, 2H), 2.88-2.74 (m, 6H), 2.48-2.23 (m, 10H), 2.10 (d, J=12.8 Hz, 1H), 1.78 (dq, J=11.4, 5.8 Hz, 1H), 0.98 (t, J=7.2 Hz, 3H).

### Example 21—Synthesis of N-(4-((4-ethylpiperazin-1-yl)methyl)-3-(trifluoromethyl)phenyl)-7-(pyrrolo[2,1-f][1,2,4]triazin-4-yloxy)-1,2,3,4-tetrahydronaphthalene-2-carboxamide (Compound No. 13)

[chemical structure]

Into a 25-mL round-bottom flask was placed a solution of N-(4-((4-ethylpiperazin-1-yl)methyl)-3-(trifluoromethyl)phenyl)-7-hydroxy-1,2,3,4-tetrahydronaphthalene-2-carboxamide (50 mg, 0.11 mmol) in N,N-dimethylformamide (4 mL) followed by the addition of 4-chloropyrrolo[2,1-f][1,2,4]triazine (17 mg, 0.11 mmol) and cesium carbonate (106 mg, 0.32 mmol) at room temperature. The mixture was stirred at 45° C. overnight. The resulting solution was diluted with dichloromethane (35 mL) and washed with water (2×30 mL). The organic layer was concentrated under vacuum, and the residue was purified by prep-HPLC to yield N-(4-((4-ethylpiperazin-1-yl)methyl)-3-(trifluoromethyl)phenyl)-7-(pyrrolo[2,1-f][1,2,4]triazin-4-yloxy)-1,2,3,4-tetrahydronaphthalene-2-carboxamide (19.3 mg, 31%). (ES, m/z): [M+H]⁺: 579.25; ¹H NMR (400 MHz, dimethylsulfoxide-d₆, ppm) δ 10.31 (s, 1H), 8.08 (m, 3H), 7.80 (d, J=8.6 Hz, 1H), 7.67 (d, J=8.5 Hz, 1H), 7.21 (d, J=8.2 Hz, 1H), 7.14-7.01 (m, 3H), 6.95 (dt, J=4.2, 1.9 Hz, 1H), 3.32 (d, J=1.4 Hz, 2H), 3.02-2.77 (m, 5H), 2.47-2.26 (m, 10H), 2.18-2.09 (m, 1H), 1.83 (dq, J=12.2, 6.3 Hz, 1H), 0.99 (td, J=7.2, 1.4 Hz, 3H).

### Example 22—Synthesis of 7-((1H-pyrrolo[2,3-b]pyridin-4-yl)oxy)-N-(4-((4-ethylpiperazin-1-yl)methyl)-3-(trifluoromethyl)phenyl)-1,2,3,4-tetrahydro-1,4-(epiminomethano)naphthalene-10-carboxamide (Compound No. 14)

[chemical structure]

### Part I—Synthesis of Dimethyl 2-(4-methoxyphenyl)pentanedioate

[chemical structure]

Into a 250-mL round-bottom flask, purged and maintained with an inert atmosphere of nitrogen, was placed methyl 2-(4-methoxyphenyl)acetate (10 g, 55.5 mmol), toluene (100 mL), methyl prop-2-enoate (9.6 g, 111 mmol) and 25% sodium methoxide in methanol solution (21 mL, 111 mmol). The reaction was stirred at 55° C. overnight. The reaction was quenched with water (250 mL), extracted with ethyl acetate (4×500 mL). The organics were dried over anhydrous sodium sulfate and concentrated. The residue was applied onto a silica gel column and eluted with ethyl acetate/hexane (0:100-10:90) to yield dimethyl 2-(4-methoxyphenyl)pentanedioate (9 g, 61%).

### Part II—Synthesis of 2-(4-Methoxyphenyl)pentanedioic acid

[chemical structure]

Into a 250-mL round-bottom flask was placed dimethyl 2-(4-methoxyphenyl)pentanedioate (8 g, 30 mmol), water (100 mL), and potassium hydroxide (16.9 g, 300 mmol). The solution was stirred at 100° C. overnight. The pH of the solution was adjusted to 6 with 1M hydrogen chloride, and the volume of water was reduced under vacuum. Solids formed which were collected by filtration to yield 2-(4-methoxyphenyl)pentanedioic acid (5.5 g, 68%).

### Part III—Synthesis of 3-(4-Methoxyphenyl)dihydro-2H-pyran-2,6(3H)-dione

[chemical structure]

Into a 50-mL round-bottom flask, purged and maintained with an inert atmosphere of nitrogen, was placed 2-(4-methoxyphenyl)pentanedioic acid (2 g, 8.4 mmol) and acetic anhydride (15 mL). The solution was stirred at 130° C. for 3 hours, and then the solvent was removed under vacuum. The residue was triturated with diethyl ether (80 mL), and then the solids were collected by filtration to yield 3-(4-methoxyphenyl)dihydro-2H-pyran-2,6(3H)-dione (2.5 g, quant. crude yield).

### Part IV—Synthesis of 6-Methoxy-4-oxo-1,2,3,4-tetrahydronaphthalene-1-carboxylic acid

[chemical structure]

Into a 250-mL round-bottom flask was placed aluminum chloride (2.4 g, 18 mmol) in 1,2-dichloroethane (30 mL). This was followed by the dropwise addition of a solution of 3-(4-methoxyphenyl)dihydro-2H-pyran-2,6(3H)-dione (1.5 g, 6.8 mmol) in 1,2-dichloroethane (30 mL) at 0° C. The mixture was stirred at room temperature for 1 hour. The mixture was diluted with water (200 mL) and extracted with ethyl acetate (3×200 mL). The organics were dried over anhydrous sodium sulfate then concentrated to yield crude 6-methoxy-4-oxo-1,2,3,4-tetrahydronaphthalene-1-carboxylic acid (1.0 g, 66%).

### Part V—Synthesis of Methyl 6-methoxy-4-oxo-1,2,3,4-tetrahydronaphthalene-1-carboxylate

[chemical structure]

Into a 250-mL round-bottom flask, purged and maintained with an inert atmosphere of nitrogen, was placed 6-methoxy-4-oxo-1,2,3,4-tetrahydronaphthalene-1-carboxylic acid (2 g, 9.1 mmol), methanol (60 mL), and sulfuric acid (1 mL). The solution was stirred at 70° C. overnight and then concentrated under vacuum. The residue was diluted with water (100 mL) and extracted with ethyl acetate (3×300 mL). The organics were dried over anhydrous sodium sulfate then concentrated. The residue was applied onto a silica gel column and eluted with ethyl acetate/petroleum ether (0:100-8:92) to yield methyl 6-methoxy-4-oxo-1,2,3,4-tetrahydronaphthalene-1-carboxylate (1 g, 47%).

### Part VI—Synthesis of Methyl 4-(hydroxyimino)-6-methoxy-1,2,3,4-tetrahydronaphthalene-1-carboxylate

[chemical structure]

Into a 50-mL round-bottom flask was placed hydroxylamine hydrochloride (445 mg, 6.4 mmol) and sodium ethoxide (872 mg, 12.8 mmol) in ethanol (10 mL). A solution of methyl 6-methoxy-4-oxo-1,2,3,4-tetrahydronaphthalene-1-carboxylate (1 g, 4.27 mmol) in ethanol (20 mL) was then added. The reaction was stirred for 2 hours at 90° C. The mixture was concentrated, and the residue was applied onto a silica gel column and eluted with ethyl acetate/petroleum ether (2:1) to yield methyl 4-(hydroxyimino)-6-methoxy-1,2,3,4-tetrahydronaphthalene-1-carboxylate (850 mg, 80%).

### Part VII—Synthesis of Methyl 4-amino-6-methoxy-1,2,3,4-tetrahydronaphthalene-1-carboxylate

[chemical structure]

Into a 30-mL high pressure reactor was placed methyl 4-(hydroxyimino)-6-methoxy-1,2,3,4-tetrahydronaphthalene-1-carboxylate (850 mg, 3.4 mmol), methanol (15 mL), concentrated hydrochloric acid (1.0 mL), and 10% palladium on carbon (43 mg, 0.04 mmol). The reactor was charged with hydrogen gas and reacted for 6 hours at room temperature. After purging hydrogen from the reactor, the solids were filtered off, and the solvents were removed under vacuum to yield methyl 4-amino-6-methoxy-1,2,3,4-tetrahydronaphthalene-1-carboxylate as the hydrochloride salt (790 mg, 80%).

### Part VIII—Synthesis of 4-Amino-6-methoxy-1,2,3,4-tetrahydronaphthalene-1-carboxylic Acid

[chemical structure]

Into a 50-mL round-bottom flask was placed methyl 4-amino-6-methoxy-1,2,3,4-tetrahydronaphthalene-1-carboxylate (790 mg, 2.9 mmol) and 2M hydrochloric acid (10 mL). The solution was stirred at 100° C. overnight. The mixture was concentrated to yield crude 4-amino-6-methoxy-1,2,3,4-tetrahydronaphthalene-1-carboxylic acid as the hydrochloride salt (730 mg, 97%).

### Part IX—Synthesis of 7-Methoxy-1,2,3,4-tetrahydro-1,4-(epiminomethano)naphthalen-9-one

[chemical structure]

Into a 250-mL round-bottom flask, purged and maintained with an inert atmosphere of nitrogen, was placed 4-amino-6-methoxy-1,2,3,4-tetrahydronaphthalene-1-carboxylic acid hydrochloride (730 mg, 2.8 mmol), acetonitrile (100 mL), pyridine (1.1 mL, 14 mmol) and dicyclohexylcarbodiimide (700 mg, 3.4 mmol). The solution was stirred at 80° C. for 1 hour, cooled to room temperature, and the solids were filtered off. The filtrates were concentrated to yield crude 7-methoxy-1,2,3,4-tetrahydro-1,4-(epiminomethano)naphthalen-9-one (560 mg, 99%).

### Part X—Synthesis of 7-Methoxy-1,2,3,4-tetrahydro-1,4-(epiminomethano)naphthalene

[chemical structure]

Into a 50-mL round-bottom flask was placed 7-methoxy-1,2,3,4-tetrahydro-1,4-(epiminomethano)naphthalen-9-one (560 mg, 2.8 mmol) and tetrahydrofuran (20 mL). A 2.5M solution of lithium aluminum hydride in tetrahydrofuran (5.6 mL, 14 mmol) was added dropwise with stirring at 0° C. After complete addition, the solution was heated at 70° C. overnight. After cooling to room temperature, the reaction was then quenched by the sequential addition of water (0.5 mL), 15% aqueous sodium hydroxide (0.5 mL), and water (1.5 mL). The suspension was stirred for 30 minutes at 0° C., then the solids were filtered out. The filtrate was concentrated to yield 7-methoxy-1,2,3,4-tetrahydro-1,4-(epiminomethano)naphthalene (380 mg, 72%).

### Part XI—Synthesis of tert-Butyl 7-methoxy-1,2,3,4-tetrahydro-1,4-(epiminomethano)naphthalene-10-carboxylate

[chemical structure]

Into a 100-mL round-bottom flask was placed 7-methoxy-1,2,3,4-tetrahydro-1,4-(epiminomethano)naphthalene (560 mg, 3.0 mmol), dichloromethane (20 mL), triethylamine (1.5 g, 14.8 mmol), and di-tert-butyl dicarbonate (2.0 g, 8.9 mmol). The mixture was stirred overnight at room temperature, diluted with ethyl acetate (200 mL), and washed with brine (3×50 mL). The separated organics were dried over anhydrous sodium sulfate then concentrated. The residue was purified on a silica gel column eluted with ethyl acetate/petroleum ether (0:100-30:70) to yield tert-butyl 7-methoxy-1,2,3,4-tetrahydro-1,4-(epiminomethano)naphthalene-10-carboxylate (350 mg, 41%).

### Part XII—Synthesis of 1,2,3,4-Tetrahydro-1,4-(epiminomethano)naphthalen-7-ol

[chemical structure]

Into a 100-mL round-bottom flask was placed tert-butyl 7-methoxy-1,2,3,4-tetrahydro-1,4-(epiminomethano)naphthalene-10-carboxylate (350 mg, 1.2 mmol) and dichloromethane (20 mL), followed by the dropwise addition of a 1M solution of boron tribromide in dichloromethane (12.1 mL, 12.10 mmol) at 0° C. The mixture was stirred for 3 hours at room temperature. The reaction was quenched with methanol (5 mL), and the mixture was concentrated under vacuum to yield crude 1,2,3,4-tetrahydro-1,4-(epiminomethano)naphthalen-7-ol (200 mg, 94%).

### Part XIII—Synthesis of tert-Butyl 7-hydroxy-1,2,3,4-tetrahydro-1,4-(epiminomethano)naphthalene-10-carboxylate

[chemical structure]

Into a 100-mL round-bottom flask was placed 1,2,3,4-tetrahydro-1,4-(epiminomethano)naphthalen-7-ol (200 mg, 1.14 mmol), dichloromethane (10 mL), and 1M aqueous sodium hydroxide (10 mL, 10 mmol), followed by di-tert-butyl dicarbonate (250 mg, 1.14 mmol). The solution was stirred at room temperature overnight, then extracted with ethyl acetate (3×150 mL). The organics were dried over anhydrous sodium sulfate and concentrated. The residue was applied onto a silica gel column and eluted with ethyl acetate/petroleum ether (1:3) to yield tert-butyl 7-hydroxy-1,2,3,4-tetrahydro-1,4-(epiminomethano)naphthalene-10-carboxylate (250 mg, 80%).

### Part XIV—Synthesis of tert-Butyl 7-((1-(triisopropylsilyl)-1H-pyrrolo[2,3-b]pyridin-4-yl)oxy)-1,2,3,4-tetrahydro-1,4-(epiminomethano)naphthalene-10-carboxylate

[chemical structure]

Into a 100-mL round-bottom flask was placed dichloromethane (30 mL), copper(II) acetate (146 mg, 0.81 mmol), and pyridine (0.46 mL, 5.6 mmol). The flask was charged with oxygen. The mixture was stirred for 20 min at room temperature, followed by addition of 4 Å molecular sieves (2 g), (1-(triisopropylsilyl)-1H-pyrrolo[2,3-b]pyridin-4-yl)boronic acid (436 mg, 1.37 mmol) and tert-butyl 7-hydroxy-1,2,3,4-tetrahydro-1,4-(epiminomethano)naphthalene-10-carboxylate (222 mg, 0.81 mmol). The mixture was stirred at room temperature overnight. The solids were filtered out, and the filtrate was concentrated. The residue was applied onto a silica gel column and eluted with ethyl acetate/petroleum ether (1:8) to yield tert-butyl 7-((1-(triisopropylsilyl)-1H-pyrrolo[2,3-b]pyridin-4-yl)oxy)-1,2,3,4-tetrahydro-1,4-(epiminomethano)naphthalene-10-carboxylate (265 mg, 60%).

### Part XV—Synthesis of 7-((1H-pyrrolo[2,3-b]pyridin-4-yl)oxy)-1,2,3,4-tetrahydro-1,4-(epiminomethano)naphthalene

[chemical structure]

Into a 50-mL round-bottom flask was placed tert-butyl 7-((1-(triisopropylsilyl)-1H-pyrrolo[2,3-b]pyridin-4-yl)oxy)-1,2,3,4-tetrahydro-1,4-(epiminomethano)naphthalene-10-carboxylate (160 mg, 0.29 mmol) and 35% hydrochloric acid (5 mL). The solution was stirred for 30 min at room temperature and concentrated to yield crude 7-((1H-pyrrolo[2,3-b]pyridin-4-yl)oxy)-1,2,3,4-tetrahydro-1,4-(epiminomethano)naphthalene as the hydrochloride salt (120 mg, quant. crude yield).

### Part XVI—Synthesis of 7-((1H-pyrrolo[2,3-b]pyridin-4-yl)oxy)-N-(4-((4-ethylpiperazin-1-yl)methyl)-3-(trifluoromethyl)phenyl)-1,2,3,4-tetrahydro-1,4-(epiminomethano)naphthalene-10-carboxamide (Compound No. 14)

[chemical structure]

Into a 50-mL round-bottom flask was placed 7-((1H-pyrrolo[2,3-b]pyridin-4-yl)oxy)-1,2,3,4-tetrahydro-1,4-(epiminomethano)naphthalene hydrochloride (120 mg, 0.37 mmol), dichloromethane (15 mL), triethylamine (0.29 mL, 2.06 mmol), and 1-ethyl-4-[[4-isocyanato-2-(trifluoromethyl)phenyl]methyl]piperazine (258 mg, 0.82 mmol). The solution was stirred for 2 hours at room temperature then concentrated under vacuum. The residue was purified by prep-HPLC to yield 7-((1H-pyrrolo[2,3-b]pyridin-4-yl)oxy)-N-(4-((4-ethylpiperazin-1-yl)methyl)-3-(trifluoromethyl)phenyl)-1,2,3,4-tetrahydro-1,4-(epiminomethano)naphthalene-10-carboxamide (6.4 mg, 3%). (ES, m/z): [M+H]⁺=605.2; (300 MHz, methanol-d₄, ppm) δ 8.04 (d, J=5.7 Hz, 1H), 7.81 (s, 1H), 7.65 (s, 2H), 7.43 (d, J=7.8 Hz, 1H), 7.26 (d, J=3.5 Hz, 1H), 7.15 (d, J=9.2 Hz, 2H), 6.48 (d, J=5.6 Hz, 1H), 6.33 (d, J=3.6 Hz, 1H), 5.39 (m, 1H), 3.75 (d, J=9.1 Hz, 1H), 3.63 (s, 2H), 3.47 (s, 1H), 3.28 (m, 2H), 2.76-2.37 (m, 9H), 2.29 (m, 1H), 2.06 (m, 1H), 1.63 (d, J=8.9 Hz, 2H), 1.12 (t, J=7.2 Hz, 3H).

### Example 23—Synthesis of N-(4-((4-ethylpiperazin-1-yl)methyl)-3-(trifluoromethyl)phenyl)-7-((2-methyl-1H-pyrrolo[2,3-b]pyridin-4-yl)oxy)-1,2,3,4-tetrahydroquinoline-2-carboxamide (Compound No. 15)

[chemical structure]

### Part I—Synthesis of 7-Hydroxyquinoline 1-oxide

[chemical structure]

Into a 100-mL round-bottom flask was placed quinolin-7-ol (4 g, 28 mmol), ethyl acetate (60 mL) and m-chloroperoxybenzoic acid (7.1 g, 41 mmol). The solution was stirred at room temperature for 3 hours, then saturated sodium bicarbonate (60 mL) was added. A solid remained suspended which was filtered off and dried to yield 7-hydroxyquinoline 1-oxide (3.6 g, 81%).

### Part II—Synthesis of 7-Hydroxyquinoline-2-carbonitrile

[chemical structure]

Into a 500-mL round-bottom flask was placed 7-hydroxyquinoline 1-oxide (3.9 g, 24.2 mmol), acetonitrile (150 mL), triethylamine (15.6 mL, 0.11 mmol), and trimethylsilyl cyanide (10.3 g, 0.10 mmol). The solution was stirred at room temperature for 10 hours. The mixture was concentrated under vacuum to yield crude 7-hydroxyquinoline-2-carbonitrile (3.5 g, 85%).

### Part III—Synthesis of 7-Hydroxyquinoline-2-carboxylic Acid

[chemical structure]

Into a 100-mL round-bottom flask was placed 7-hydroxyquinoline-2-carbonitrile (3.5 g, 20 mmol) and concentrated hydrogen chloride (50 mL). The resulting solution was stirred at 100° C. overnight. The mixture was concentrated to yield crude 7-hydroxyquinoline-2-carboxylic acid (4 g, 99%).

### Part IV—Synthesis of Methyl 7-hydroxyquinoline-2-carboxylate

[chemical structure]

Into a 250-mL round-bottom flask was placed 7-hydroxyquinoline-2-carboxylic acid (3 g, 16 mmol), methanol (60 mL), and concentrated hydrogen chloride (10 mL). The solution was stirred at 80° C. overnight. The solvent was removed under vacuum to yield methyl 7-hydroxyquinoline-2-carboxylate (3 g, 93%).

### Part V—Synthesis of Methyl 7-hydroxy-1,2,3,4-tetrahydroquinoline-2-carboxylate

[chemical structure]

Into a 250-mL round-bottom flask was placed methyl 7-hydroxyquinoline-2-carboxylate (3 g, 15 mmol), methanol (150 mL), and 10% platinum on carbon (3 g, 1.5 mmol). The flask was purged and maintained with an atmosphere of hydrogen. The mixture was stirred at 45° C. overnight, the solids were filtered off, and the filtrate was concentrated to yield methyl 7-hydroxy-1,2,3,4-tetrahydroquinoline-2-carboxylate (3 g, 98%).

### Part VI—Synthesis of Methyl 7-((tert-butoxycarbonyl)oxy)-1,2,3,4-tetrahydroquinoline-2-carboxylate

[chemical structure]

Into a 250-mL round-bottom flask was placed methyl 7-hydroxy-1,2,3,4-tetrahydroquinoline-2-carboxylate (3 g, 14.5 mmol), dichloromethane (60 mL), triethylamine (6 mL, 43 mmol), and di-tert-butyl dicarbonate (6.3 g, 29 mmol). The solution was stirred at room temperature overnight then concentrated. The residue was purified on a silica gel column eluting with ethyl acetate/petroleum ether (1:10) to yield methyl 7-((tert-butoxycarbonyl)oxy)-1,2,3,4-tetrahydroquinoline-2-carboxylate (1.9 g, 43%).

### Part VII—Synthesis of 1-(tert-Butyl) 2-methyl 7-((tert-butoxycarbonyl)oxy)-3,4-dihydroquinoline-1,2(2H)-dicarboxylate

[chemical structure]

Into a 50-mL round-bottom flask was placed methyl 7-((tert-butoxycarbonyl)oxy)-1,2,3,4-tetrahydroquinoline-2-carboxylate (100 mg, 0.33 mmol), dichloromethane (20 mL), 4-(dimethylamino)pyridine (20 mg, 0.16 mmol), triethylamine (0.09 mL, 0.65 mmol), and di-tert-butyl dicarbonate (142 mg, 0.65 mmol). The solution was stirred at room temperature overnight then concentrated. The residue was purified on a silica gel column eluting with ethyl acetate/petroleum ether (1:2) to yield 1-(tert-butyl) 2-methyl 7-((tert-butoxycarbonyl)oxy)-3,4-dihydroquinoline-1,2(2H)-dicarboxylate (125 mg, 94%).

### Part VIII—Synthesis of 1-(tert-Butyl) 2-methyl 7-hydroxy-3,4-dihydroquinoline-1,2(2H)-dicarboxylate

[chemical structure]

Into a 25-mL round-bottom flask was placed 1-(tert-butyl) 2-methyl 7-((tert-butoxycarbonyl)oxy)-3,4-dihydroquinoline-1,2(2H)-dicarboxylate (1.8 g, 4.4 mmol), 25% sodium methoxide in methanol (1.5 mL, 6.6 mmol), and methanol (15 mL). The solution was stirred at room temperature for 1 hour, diluted with brine (30 mL), and extracted with ethyl acetate (3×50 mL). The organics were concentrated to yield 1-(tert-butyl) 2-methyl 7-hydroxy-3,4-dihydroquinoline-1,2(2H)-dicarboxylate (1.5 g, 99%).

### Part IX—Synthesis of 1-(tert-Butyl) 2-methyl 7-((2-methyl-1-(phenylsulfonyl)-1H-pyrrolo[2,3-b]pyridin-4-yl)oxy)-3,4-dihydroquinoline-1,2(2H)-dicarboxylate

[chemical structure]

Into a 50-mL round-bottom flask, purged and maintained with an inert atmosphere of nitrogen, was placed 1-(tert-butyl) 2-methyl 7-hydroxy-3,4-dihydroquinoline-1,2(2H)-dicarboxylate (600 mg, 1.95 mmol), toluene (20 mL), 1-(benzenesulfonyl)-4-bromo-2-methyl-1H-pyrrolo[2,3-b]pyridine (686 mg, 1.95 mmol), potassium carbonate (810 mg, 5.86 mmol), 2-dicyclohexylphosphino-2′,4′,6′-triisopropylbiphenyl (93 mg, 0.20 mmol) and tris(dibenzylideneacetone)dipalladium(0) (89 mg, 0.10 mmol). The mixture was stirred at 90° C. overnight then concentrated. The residue was applied onto a silica gel column and eluted with dichloromethane/methanol (10:1) to yield 1-(tert-butyl) 2-methyl 7-((2-methyl-1-(phenylsulfonyl)-1H-pyrrolo[2,3-b]pyridin-4-yl)oxy)-3,4-dihydroquinoline-1,2(2H)-dicarboxylate (500 mg, 44%).

### Part X—Synthesis of 1-(tert-Butoxycarbonyl)-7-((2-methyl-1-(phenylsulfonyl)-1H-pyrrolo[2,3-b]pyridin-4-yl)oxy)-1,2,3,4-tetrahydroquinoline-2-carboxylic acid

[chemical structure]

Into a 100-mL round-bottom flask was placed 1-(tert-butyl) 2-methyl 7-((2-methyl-1-(phenylsulfonyl)-1H-pyrrolo[2,3-b]pyridin-4-yl)oxy)-3,4-dihydroquinoline-1,2(2H)-dicarboxylate (500 mg, 0.87 mmol), water (15 mL), tetrahydrofuran (15 mL), and lithium hydroxide (124 mg, 5.2 mmol). The solution was stirred at room temperature overnight. The pH of the solution was adjusted to <8 with 1M aqueous hydrogen chloride. This mixture was extracted with ethyl acetate (3×50 mL), and the organics were concentrated to yield 1-(tert-butoxycarbonyl)-7-((2-methyl-1-(phenylsulfonyl)-1H-pyrrolo[2,3-b]pyridin-4-yl)oxy)-1,2,3,4-tetrahydroquinoline-2-carboxylic acid (350 mg, 72%).

### Part XI—Synthesis of tert-Butyl 2-((4-((4-ethylpiperazin-1-yl)methyl)-3-(trifluoromethyl)phenyl)carbamoyl)-7-((2-methyl-1-(phenylsulfonyl)-1H-pyrrolo[2,3-b]pyridin-4-yl)oxy)-3,4-dihydroquinoline-1(2H)-carboxylate

[chemical structure]

Into a 50-mL round-bottom flask was placed 1-(tert-butoxycarbonyl)-7-((2-methyl-1-(phenylsulfonyl)-1H-pyrrolo[2,3-b]pyridin-4-yl)oxy)-1,2,3,4-tetrahydroquinoline-2-carboxylic acid (300 mg, 0.53 mmol), dichloromethane (20 mL), 4-[(4-ethylpiperazin-1-yl)methyl]-3-(trifluoromethyl)aniline (153 mg, 0.53 mmol), N-[(dimethylamino)-1H-1,2,3-triazolo-[4,5-b]pyridin-1-ylmethylene]-N-methylmethanaminium hexafluorophosphate N-oxide (405 mg, 1.06 mmol) and N,N-diisopropylethylamine (0.37 mL, 2.1 mmol). The solution was stirred at 45° C. overnight then concentrated. The residue was purified on a silica gel column eluting with ethyl acetate/petroleum ether (1:1) to yield tert-butyl 2-((4-((4-ethylpiperazin-1-yl)methyl)-3-(trifluoromethyl)phenyl)carbamoyl)-7-((2-methyl-1-(phenylsulfonyl)-1H-pyrrolo[2,3-b]pyridin-4-yl)oxy)-3,4-dihydroquinoline-1(2H)-carboxylate (180 mg, 41%).

### Part XII—Synthesis of tert-Butyl 2-((4-((4-ethylpiperazin-1-yl)methyl)-3-(trifluoromethyl)phenyl)carbamoyl)-7-((2-methyl-1H-pyrrolo[2,3-b]pyridin-4-yl)oxy)-3,4-dihydroquinoline-1(2H)-carboxylate

[chemical structure]

Into a 50-mL round-bottom flask was placed tert-butyl 2-((4-((4-ethylpiperazin-1-yl)methyl)-3-(trifluoromethyl)phenyl)carbamoyl)-7-((2-methyl-1-(phenylsulfonyl)-1H-pyrrolo[2,3-b]pyridin-4-yl)oxy)-3,4-dihydroquinoline-1(2H)-carboxylate (400 mg, 0.48 mmol), ethanol (15 mL), magnesium (58 mg, 2.4 mmol), and ammonium chloride (257 mg, 4.80 mmol). The mixture was stirred at room temperature overnight, then the solids were filtered off, and the filtrate was concentrated to yield tert-butyl 2-((4-((4-ethylpiperazin-1-yl)methyl)-3-(trifluoromethyl)phenyl)carbamoyl)-7-((2-methyl-1H-pyrrolo[2,3-b]pyridin-4-yl)oxy)-3,4-dihydroquinoline-1(2H)-carboxylate (180 mg, 54%).

### Part XIII—Synthesis of N-(4-((4-ethylpiperazin-1-yl)methyl)-3-(trifluoromethyl)phenyl)-7-((2-methyl-1H-pyrrolo[2,3-b]pyridin-4-yl)oxy)-1,2,3,4-tetrahydroquinoline-2-carboxamide (Compound No. 15)

[chemical structure]

Into a 50-mL round-bottom flask was placed tert-butyl 2-((4-((4-ethylpiperazin-1-yl)methyl)-3-(trifluoromethyl)phenyl)carbamoyl)-7-((2-methyl-1H-pyrrolo[2,3-b]pyridin-4-yl)oxy)-3,4-dihydroquinoline-1(2H)-carboxylate (150 mg, 0.22 mmol), dichloromethane (10 mL), and trifluoroacetic acid (3 mL). The solution was stirred at room temperature for 2 hours then concentrated. The residue was purified by prep-HPLC to yield N-(4-((4-ethylpiperazin-1-yl)methyl)-3-(trifluoromethyl)phenyl)-7-((2-methyl-1H-pyrrolo[2,3-b]pyridin-4-yl)oxy)-1,2,3,4-tetrahydroquinoline-2-carboxamide (35.2 mg, 27%). (ES, m/z): [M+H]⁺: 593.25; ¹H NMR (400 MHz, methanol-d₄, ppm) δ 8.05 (d, J=2.2 Hz, 1H), 7.92 (d, J=5.7 Hz, 1H), 7.85-7.77 (m, 1H), 7.74 (d, J=8.6 Hz, 1H), 7.01 (d, J=8.2 Hz, 1H), 6.53-6.46 (m, 2H), 6.41 (dd, J=8.1, 2.4 Hz, 1H), 6.04 (d, J=1.1 Hz, 1H), 4.12 (dd, J=6.1, 4.7 Hz, 1H), 3.68-3.63 (m, 2H), 2.93-2.44 (m, 12H), 2.42 (d, J=1.0 Hz, 3H), 2.30 (dd, J=11.9, 6.0 Hz, 1H), 2.15-2.03 (m, 1H), 1.13 (t, J=7.2 Hz, 3H).

### Example 24—Synthesis of N-(4-((4-ethylpiperazin-1-yl)methyl)-3-(trifluoromethyl)phenyl)-1-methyl-7-((2-methyl-1H-pyrrolo[2,3-b]pyridin-4-yl)oxy)-1,2,3,4-tetrahydroquinoline-2-carboxamide (Compound No. 16)

[chemical structure]

### Part I—Synthesis of 7-Hydroxy-1,2,3,4-tetrahydroquinoline-2-carboxylic Acid

[chemical structure]

Into a 100-mL round-bottom flask was placed 7-hydroxyquinoline-2-carboxylic acid (300 mg, 1.586 mmol) and methanol (20 mL, 0.62 mmol), followed by the addition of 10% platinum on carbon (300 mg, 0.15 mmol). The flask was purged and charged with hydrogen gas, then the mixture was stirred at room temperature overnight. After evacuating the hydrogen gas, the solids were filtered out, and the filtrate was concentrated to yield 7-hydroxy-1,2,3,4-tetrahydroquinoline-2-carboxylic acid (220 mg, 72%).

### Part II—Synthesis of Methyl 7-methoxy-1-methyl-1,2,3,4-tetrahydroquinoline-2-carboxylate

[chemical structure]

Into a 40-mL high pressure tube was placed 7-hydroxy-1,2,3,4-tetrahydroquinoline-2-carboxylic acid (300 mg, 1.6 mmol), acetonitrile (18 mL), potassium carbonate (0.44 g, 3.2 mmol), and iodomethane (0.5 mL, 8 mmol). The tube was sealed, and the mixture was stirred at 60° C. for 12 hours. After cooling, the solids were filtered out, and the filtrate was concentrated under vacuum. The residue was applied onto a silica gel column and eluted with ethyl acetate/petroleum ether (1:10-1:5) to yield methyl 7-methoxy-1-methyl-1,2,3,4-tetrahydroquinoline-2-carboxylate (265 mg, 73%).

### Part III—Synthesis of 7-Hydroxy-1-methyl-1,2,3,4-tetrahydroquinoline-2-carboxylic Acid

[chemical structure]

Into a 100-mL round-bottom flask was placed methyl 7-methoxy-1-methyl-1,2,3,4-tetrahydroquinoline-2-carboxylate (750 mg, 3.6 mmol) in dichloromethane (20 mL). Boron tribromide in dichloromethane (1M, 20 mL, 20 mmol) was added. The solution was stirred for 2 hours at room temperature, then the reaction was quenched with methanol (50 mL). The mixture was diluted with dichloromethane (100 mL). The pH value of the solution was adjusted to 7 with trimethylamine, then the solution was concentrated. The residue was purified on a silica gel column eluting with ethyl acetate/petroleum ether (1:10 to 1:1) to yield 7-hydroxy-1-methyl-1,2,3,4-tetrahydroquinoline-2-carboxylic acid (306 mg, 46%).

### Part IV—Synthesis of Methyl 7-hydroxy-1-methyl-1,2,3,4-tetrahydroquinoline-2-carboxylate

[chemical structure]

Into a 100-mL round-bottom flask was placed 7-hydroxy-1-methyl-1,2,3,4-tetrahydroquinoline-2-carboxylic acid (100 mg, 0.48 mmol), methanol (5 mL), and tetrahydrofuran (5 mL), then 2M (trimethylsilyl)diazomethane solution in hexanes (0.5 mL, 1 mmol) was added. The solution was stirred at room temperature for 2 hours, then concentrated to yield methyl 7-hydroxy-1-methyl-1,2,3,4-tetrahydroquinoline-2-carboxylate (100 mg, 94%).

### Part V—Synthesis of Methyl 1-methyl-7-((2-methyl-1-(phenylsulfonyl)-1H-pyrrolo[2,3-b]pyridin-4-yl)oxy)-1,2,3,4-tetrahydroquinoline-2-carboxylate

[chemical structure]

Into a 100-mL round-bottom flask, purged and maintained with an inert atmosphere of nitrogen, was placed methyl 7-hydroxy-1-methyl-1,2,3,4-tetrahydroquinoline-2-carboxylate (70 mg, 0.32 mmol), toluene (10 mL), 1-(benzenesulfonyl)-4-bromo-2-methyl-1H-pyrrolo[2,3-b]pyridine (122 mg, 0.35 mmol), potassium carbonate (110 mg, 0.80 mmol), 2-dicyclohexylphosphino-2′,4′,6′-triisopropylbiphenyl (15 mg, 0.03 mmol), and tris(dibenzylideneacetone)dipalladium(0) (17 mg, 0.02 mmol). The mixture was stirred at 110° C. overnight, then the solids were filtered out, and the filtrate was concentrated. The residue was applied onto a silica gel column and eluted with ethyl acetate/petroleum ether (1:10 to 1:3) to yield methyl 1-methyl-7-((2-methyl-1-(phenylsulfonyl)-1H-pyrrolo[2,3-b]pyridin-4-yl)oxy)-1,2,3,4-tetrahydroquinoline-2-carboxylate (80 mg, 51%).

### Part VI—Synthesis of 1-Methyl-7-((2-methyl-1H-pyrrolo[2,3-b]pyridin-4-yl)oxy)-1,2,3,4-tetrahydroquinoline-2-carboxylic Acid

[chemical structure]

Into a 50-mL round-bottom flask was placed methyl 1-methyl-7-((2-methyl-1-(phenylsulfonyl)-1H-pyrrolo[2,3-b]pyridin-4-yl)oxy)-1,2,3,4-tetrahydroquinoline-2-carboxylate (80 mg, 0.16 mmol), water (5 mL), and sodium hydroxide (65 mg, 1.6 mmol). The resulting solution was stirred at 80° C. for 2 hours. The mixture was cooled to 0° C., and the pH value of the solution was adjusted to 7 with 1M aqueous hydrogen chloride. The mixture was concentrated, and the residue was purified on a silica gel column eluting with ethyl acetate/petroleum ether (1:10 to 1:1) to yield 1-methyl-7-((2-methyl-1H-pyrrolo[2,3-b]pyridin-4-yl)oxy)-1,2,3,4-tetrahydroquinoline-2-carboxylic acid (50 mg, 91%).

### Part VII—Synthesis of N-(4-((4-ethylpiperazin-1-yl)methyl)-3-(trifluoromethyl)phenyl)-1-methyl-7-((2-methyl-1H-pyrrolo[2,3-b]pyridin-4-yl)oxy)-1,2,3,4-tetrahydroquinoline-2-carboxamide (Compound No. 16)

[chemical structure]

Into a 100-mL round-bottom flask was placed 1-methyl-7-((2-methyl-1H-pyrrolo[2,3-b]pyridin-4-yl)oxy)-1,2,3,4-tetrahydroquinoline-2-carboxylic acid (50 mg, 0.15 mmol), N,N-dimethylformamide (5 mL), 4-[(4-ethylpiperazin-1-yl)methyl]-3-(trifluoromethyl)aniline (128 mg, 0.45 mmol), 4-(dimethylamino)pyridine (54 mg, 0.45 mmol), and N-(3-dimethylaminopropyl)-N′-ethylcarbodiimide hydrochloride (85 mg, 0.44 mmol). The mixture was stirred at room temperature overnight then concentrated. The residue was applied onto a silica gel column and eluted with ethyl acetate/petroleum ether (1:5 to 1:0). The resulting product was further purified by prep-HPLC to yield N-(4-((4-ethylpiperazin-1-yl)methyl)-3-(trifluoromethyl)phenyl)-1-methyl-7-((2-methyl-1H-pyrrolo[2,3-b]pyridin-4-yl)oxy)-1,2,3,4-tetrahydroquinoline-2-carboxamide (7.5 mg, 8%). (ES, m/z): [M+H]⁺=607.30; ¹H NMR (400 MHz, methanol-d₄, ppm) δ 8.02 (d, J=2.1 Hz, 1H), 7.92 (d, J=5.7 Hz, 1H), 7.82 (d, J=8.5 Hz, 1H), 7.75 (d, J=8.6 Hz, 1H), 7.01 (d, J=8.1 Hz, 1H), 6.57 (d, J=2.3 Hz, 1H), 6.50 (d, J=5.6 Hz, 1H), 6.41 (dd, J=8.0, 2.3 Hz, 1H), 6.07 (d, J=1.1 Hz, 1H), 4.11 (t, J=4.9 Hz, 1H), 3.66 (s, 2H), 2.95 (s, 3H), 2.77 (m, 2H), 2.55 (m, 9H), 2.54-2.41 (m, 2H), 2.39-2.16 (m, 2H), 1.33 (m, 2H), 1.13 (t, J=7.2 Hz, 3H).

### Example 25—Synthesis of N-(4-((4-ethylpiperazin-1-yl)methyl)-3-(trifluoromethyl)phenyl)-7-((2-methyl-1H-pyrrolo[2,3-b]pyridin-4-yl)oxy)-4-oxo-3,4-dihydroisoquinoline-2(1H)-carboxamide (Compound No. 17)

[chemical structure]

### Part I—Synthesis of 4-Bromo-2-methyl-1-(triisopropylsilyl)-1H-pyrrolo[2,3-b]pyridine

[chemical structure]

Into a 100-mL round-bottom flask, purged and maintained with an inert atmosphere of nitrogen, was placed 4-bromo-2-methyl-1H-pyrrolo[2,3-b]pyridine (256 mg, 1.2 mmol) and N,N-dimethylformamide (5 mL). The solution was cooled to 0° C., then sodium hydride (58 mg, 1.5 mmol, 60% dispersion in mineral oil) was added portionwise. The mixture was stirred at 0° C. for 30 minutes, then triisopropylsilyl chloride (0.52 mL, 2.4 mmol) was added dropwise with stirring at 0° C. The solution was stirred at room temperature overnight then quenched with water (30 mL). The mixture was extracted with ethyl acetate (3×30 mL), and the organics were concentrated under vacuum. The residue was purified on a silica gel column eluting with 0-5% ethyl acetate in petroleum ether to yield 4-bromo-2-methyl-1-(triisopropylsilyl)-1H-pyrrolo[2,3-b]pyridine (400 mg, 90%).

### Part II—Synthesis of (2-Methyl-1-(triisopropylsilyl)-1H-pyrrolo[2,3-b]pyridin-4-yl)boronic Acid

[chemical structure]

Into a 250-mL, 3-necked round-bottom flask, purged and maintained with an inert atmosphere of nitrogen, was placed 4-bromo-2-methyl-1-(triisopropylsilyl)-1H-pyrrolo[2,3-b]pyridine (2.73 g, 7.43 mmol) in anhydrous tetrahydrofuran (60 mL). The solution was cooled to −78° C., then 2.5M n-butyllithium in hexanes (9 mL, 22.3 mmol) was added dropwise. The mixture was stirred at −78° C. for 30 minutes, then trimethyl borate (1.6 mL, 14.8 mmol) was added dropwise. After complete addition, the solution was allowed to warm to room temperature and was stirred at room temperature for 2 hours. The reaction was then quenched by the addition of saturated aqueous ammonium chloride, and the mixture was extracted with ethyl acetate (3×100 mL). The organics were dried over anhydrous sodium sulfate then concentrated under vacuum. The residue was applied onto a silica gel column and eluted with 10-20% ethyl acetate in petroleum ether to yield (2-methyl-1-(triisopropylsilyl)-1H-pyrrolo[2,3-b]pyridin-4-yl)boronic acid (900 mg, 36%).

### Part III—Synthesis of N-(3-(Benzyloxy)benzyl)-2,2-dimethoxyethan-1-amine

[chemical structure]

Into a 250-mL round-bottom flask was placed 3-(benzyloxy)benzaldehyde (5 g, 24 mmol), dichloromethane (100 mL), 2,2-dimethoxyethan-1-amine (5 g, 48 mmol), acetic acid (0.13 mL, 2 mmol), and sodium triacetoxyborohydride (15 g, 71 mmol). The solution was stirred at room temperature overnight then concentrated under vacuum. The residue was purified on a silica gel column eluting with 20-30% ethyl acetate in petroleum ether to yield N-(3-(benzyloxy)benzyl)-2,2-dimethoxyethan-1-amine (7 g, 99%).

### Part IV—Synthesis of 7-(Benzyloxy)-1,2,3,4-tetrahydroisoquinolin-4-ol

[chemical structure]

Into a 250-mL round-bottom flask was placed N-(3-(benzyloxy)benzyl)-2,2-dimethoxyethan-1-amine (5 g, 20 mmol) in 6M aqueous hydrogen chloride (100 mL) and stirred at 40° C. overnight. The mixture was concentrated under vacuum, and the residue was treated with 5% dichloromethane in hexanes (20 mL). The resulting solids were collected by filtration to yield 7-(benzyloxy)-1,2,3,4-tetrahydroisoquinolin-4-ol as the hydrochloride salt (2.3 g, 54%).

### Part V—Synthesis of 1,2,3,4-Tetrahydroisoquinoline-4,7-diol

[chemical structure]

Into a 100-mL round-bottom flask was placed 7-(benzyloxy)-1,2,3,4-tetrahydroisoquinolin-4-ol (2 g, 7.8 mmol) in methanol (40 mg), then 10% palladium on carbon (1 g) was added. The flask was purged then charged with hydrogen gas. The mixture was stirred at room temperature overnight. After evacuating hydrogen from the flask, the solids were filtered out, and the filtrate was concentrated under vacuum to yield crude 1,2,3,4-tetrahydroisoquinoline-4,7-diol (1.5 g, quant. yield).

### Part VI—Synthesis of tert-Butyl 7-((tert-butoxycarbonyl)oxy)-4-hydroxy-3,4-dihydroisoquinoline-2(1H)-carboxylate

[chemical structure]

Into a 100-mL round-bottom flask was placed 1,2,3,4-tetrahydroisoquinoline-4,7-diol (1.5 g, 9.1 mmol), tetrahydrofuran (20 mL), water (5 mL), and triethylamine (6.3 mL, 45 mmol), followed by the addition of di-tert-butyl dicarbonate (7.9 g, 36 mmol). The resulting solution was stirred at room temperature for 5 hours then concentrated. The residue was applied onto a silica gel column and eluted with 10-20% ethyl acetate in petroleum ether to yield tert-butyl 7-((tert-butoxycarbonyl)oxy)-4-hydroxy-3,4-dihydroisoquinoline-2(1H)-carboxylate (1.5 g, 45%).

### Part VII—Synthesis of tert-Butyl 7-((tert-butoxycarbonyl)oxy)-4-oxo-3,4-dihydroisoquinoline-2(1H)-carboxylate

[chemical structure]

Into an 8-mL vial was placed tert-butyl 7-((tert-butoxycarbonyl)oxy)-4-hydroxy-3,4-dihydroisoquinoline-2(1H)-carboxylate (100 mg, 0.27 mmol) in dichloromethane (5 mL) followed by 1,1,1-tris(acetyloxy)-1,1-dihydro-1,2-benziodoxol-3-(1H)-one (174 mg, 0.41 mmol). The solution was stirred at room temperature for 2 days. The mixture was concentrated, and the residue was purified on a silica gel column eluting with ethyl acetate/petroleum ether (1:2) to yield tert-butyl 7-((tert-butoxycarbonyl)oxy)-4-oxo-3,4-dihydroisoquinoline-2(1H)-carboxylate (30 mg, 30%).

### Part VIII—Synthesis of tert-Butyl 7-hydroxy-4-oxo-3,4-dihydroisoquinoline-2(1H)-carboxylate

[chemical structure]

Into a 100-mL round-bottom flask was placed tert-butyl 7-((tert-butoxycarbonyl)oxy)-4-oxo-3,4-dihydroisoquinoline-2(1H)-carboxylate (1 g, 3.0 mmol) and methanol (10 mL), followed by the dropwise addition of a 25% solution of sodium methoxide in methanol (0.15 mL, 0.66 mmol) at room temperature. The solution was stirred for 2 hours, then concentrated. The residue was purified on a silica gel column eluting with a gradient of 2-5% methanol in dichloromethane to yield tert-butyl 7-hydroxy-4-oxo-3,4-dihydroisoquinoline-2(1H)-carboxylate (300 mg, 38%).

### Part IX—Synthesis of tert-Butyl 7-((2-methyl-1-(triisopropylsilyl)-1H-pyrrolo[2,3-b]pyridin-4-yl)oxy)-4-oxo-3,4-dihydroisoquinoline-2(1H)-carboxylate

[chemical structure]

Into a 50-mL round-bottom flask was placed tert-butyl 7-hydroxy-4-oxo-3,4-dihydroisoquinoline-2(1H)-carboxylate (200 mg, 0.76 mmol), dichloromethane (10 mL), (2-methyl-1-(triisopropylsilyl)-1H-pyrrolo[2,3-b]pyridin-4-yl)boronic acid (278 mg, 0.84 mmol), pyridine (0.31 mL, 3.8 mmol), copper(II) acetate (138 mg, 0.76 mmol), and 4 Å molecular sieves (200 mg). The flask was purged, then charged with oxygen gas. The mixture was stirred at room temperature overnight then concentrated under vacuum. The residue was purified on a silica gel column eluting with ethyl acetate/petroleum ether (1:2) to yield tert-butyl 7-((2-methyl-1-(triisopropylsilyl)-1H-pyrrolo[2,3-b]pyridin-4-yl)oxy)-4-oxo-3,4-dihydroisoquinoline-2(1H)-carboxylate (200 mg, 48%).

### Part X—Synthesis of 7-((2-Methyl-1H-pyrrolo[2,3-b]pyridin-4-yl)oxy)-2,3-dihydroisoquinolin-4(1H)-one

[chemical structure]

Into a 50-mL round-bottom flask was placed tert-butyl 7-((2-methyl-1-(triisopropylsilyl)-1H-pyrrolo[2,3-b]pyridin-4-yl)oxy)-4-oxo-3,4-dihydroisoquinoline-2(1H)-carboxylate (120 mg, 0.2 mmol) in concentrated hydrochloric acid (2 mL). The solution was stirred at room temperature for 2 hours, then concentrated to yield crude 7-((2-methyl-1H-pyrrolo[2,3-b]pyridin-4-yl)oxy)-2,3-dihydroisoquinolin-4(1H)-one as the hydrochloride salt (60 mg, quant. yield).

### Part XI—Synthesis of N-(4-((4-ethylpiperazin-1-yl)methyl)-3-(trifluoromethyl)phenyl)-7-((2-methyl-1H-pyrrolo[2,3-b]pyridin-4-yl)oxy)-4-oxo-3,4-dihydroisoquinoline-2(1H)-carboxamide (Compound No. 17)

[chemical structure]

Into a 50-mL round-bottom flask was placed 7-((2-methyl-1H-pyrrolo[2,3-b]pyridin-4-yl)oxy)-2,3-dihydroisoquinolin-4(1H)-one (58 mg, 0.2 mmol) in dichloromethane (5 mL), followed by addition of triethylamine (0.08 mL, 0.6 mmol) and phenyl N-[4-[(4-ethylpiperazin-1-yl)methyl]-3-(trifluoromethyl)phenyl]carbamate (120 mg, 0.3 mmol). The solution was stirred at room temperature overnight. The mixture was concentrated then purified by prep-HPLC to yield N-(4-((4-ethylpiperazin-1-yl)methyl)-3-(trifluoromethyl)phenyl)-7-((2-methyl-1H-pyrrolo[2,3-b]pyridin-4-yl)oxy)-4-oxo-3,4-dihydroisoquinoline-2(1H)-carboxamide (10 mg, 8%). (ES, m/z): [M+H]⁺ 607.1; ¹H NMR (300 MHz, methanol-d₄, ppm) δ 8.10-8.02 (m, 2H), 7.75 (d, J=2.0 Hz, 1H), 7.62 (m, 2H), 7.17-7.05 (m, 2H), 6.77-6.69 (m, 1H), 5.89 (s, 1H), 4.90 (s, 2H), 4.45 (s, 2H), 3.60 (s, 2H), 2.54 (m, 10H), 2.37 (s, 3H), 1.10 (t, J=7.2 Hz, 3H).

### Example 26—Synthesis of N-(4-((4-ethylpiperazin-1-yl)methyl)-3-(trifluoromethyl)phenyl)-4-hydroxy-7-((2-methyl-1H-pyrrolo[2,3-b]pyridin-4-yl)oxy)-3,4-dihydroisoquinoline-2(1H)-carboxamide (Compound No. 18)

[chemical structure]

Into a 25-mL round-bottom flask, purged and maintained with an inert atmosphere of nitrogen, was placed N-(4-((4-ethylpiperazin-1-yl)methyl)-3-(trifluoromethyl)phenyl)-7-((2-methyl-1H-pyrrolo[2,3-b]pyridin-4-yl)oxy)-4-oxo-3,4-dihydroisoquinoline-2(1H)-carboxamide (80 mg, 0.13 mmol) in methanol (3 mL), followed by the portionwise addition of sodium borohydride (20 mg, 0.53 mmol) at 0° C. The reaction was then stirred at room temperature for 2 hours. The mixture was concentrated, and the residue was purified by prep-HPLC to yield N-(4-((4-ethylpiperazin-1-yl)methyl)-3-(trifluoromethyl)phenyl)-4-hydroxy-7-((2-methyl-1H-pyrrolo[2,3-b]pyridin-4-yl)oxy)-3,4-dihydroisoquinoline-2(1H)-carboxamide (10 mg, 10%). (ES, m/z): [M+H]⁺ 609.3; ¹H NMR (300 MHz, methanol-d₄, ppm) δ 8.14 (d, J=6.9 Hz, 1H), 7.83 (s, 1H), 7.73-7.61 (m, 3H), 7.21 (m, 2H), 6.80 (d, J=6.8 Hz, 1H), 6.31 (s, 1H), 4.96-4.86 (m, 1H), 4.66 (d, J=16.9 Hz, 1H), 3.88 (d, J=4.7 Hz, 2H), 3.74 (s, 2H), 3.6-2.7 (m, 11H), 2.50 (s, 3H), 1.34 (t, J=7.3 Hz, 3H).

### Example 27—Synthesis of Additional N-phenyl-3,4-dihydroisoquinoline-2(1H)-carboxamides and Related Compounds

The compounds in Table 10 below were prepared based on experimental procedures described in Examples 1-20 and in the detailed description.

TABLE 10

| | | Mass Spec. | |
|---|---|---|---|
| No. | Chemical Structure | (ES, m/z) | NMR Spectrum |
| X-1 | [chemical structure] | 579.4 [M + H]⁺ | N/A |
| X-2 | [chemical structure] | 593.3 [M + H]⁺ | N/A |
| X-3 | [chemical structure] | 596.4 [M + H]⁺ | ¹H NMR (300 MHz, Methanol-d4) δ 8.02 (s, 1H), 7.83 (s, 2H), 7.67 (s, 2H), 7.49 (d, J = 10.9 Hz, 2H), 7.36 (d, J = 7.8 Hz, 1H), 6.91 (dd, J = 4.5, 2.6 Hz, 1H), 6.89-6.81 (m, 1H), 4.77 (m, 2H), 3.84 (t, J = 5.9 Hz, 2H), 3.64 (m, 2H), 3.04 (m, 2H), 2.66-2.39 (m, 9H), 1.12 (t, J = 7.2 Hz, 3H) |
| X-4 | [chemical structure] | 607.5 [M + H]⁺ | N/A |
| | Mixture of Stereoisomers at 4-Position | | |
| X-5 | [chemical structure] | 594.45 [M + H]⁺ | ¹H NMR (300 MHz, Methanol-d4) δ 8.08 (d, J = 5.6 Hz, 1H), 7.82 (s, 1H), 7.67 (d, J = 3.2 Hz, 2H), 7.42 (s, 1H), 7.33 (d, J = 3.5 Hz, 1H), 6.43 (d, J = 5.6 Hz, 1H), 6.37 (d, J = 3.6 Hz, 1H), 4.73 (m, 2H), 3.94 (m, 2H), 3.63 (m, 2H), 3.10 (m, 2H), 2.76-2.33 (m, 13H), 1.12 (t, J = 7.2 Hz, 3H). |
| X-6 | [chemical structure] | 561.45 [M + H]⁺ | ¹H NMR (400 MHz, Methanol-d4) δ 8.14 (dd, J = 7.9, 5.0 Hz, 2H), 7.90 (d, J = 2.1 Hz, 1H), 7.67 (dd, J = 9.1, 2.2 Hz, 1H), 7.57 (d, J = 8.2 Hz, 1H), 7.45-7.37 (m, 2H), 7.20 (d, J = 2.5 Hz, 1H), 7.13 (dd, J = 7.7, 3.7 Hz, 2H), 6.78 (d, J = 6.7 Hz, 1H), 6.28 (s, 1H), 5.48 (m, 1H), 4.26 (m, 1H), 3.69 (m, 2H), 3.63-3.46 (m, 5H), 3.24-3.11 (m, 3H), 3.06 (s, 3H), 3.04-2.96 (m, 1H), 2.51 (m, 3H), 1.60 (d, J = 6.8 Hz, 3H). |
| X-7 | [chemical structure] | N/A | N/A |
| X-8 | [chemical structure] | N/A | N/A |

### Example 28—Synthesis of Additional N-phenyl-3,4-dihydroisoquinoline-2(1H)-carboxamides and Related Compounds

The compounds in Table 11 below were prepared based on experimental procedures described in the Examples above and in the detailed description.

TABLE 11

| No. | Chemical Structure |
|---|---|
| XI-1 | [chemical structure] |
| XI-2 | [chemical structure] |
| XI-3 | [chemical structure] |
| XI-4 | [chemical structure] |
| XI-5 | [chemical structure] |
| XI-6 | [chemical structure] |
| XI-7 | [chemical structure] |
| XI-8 | [chemical structure] |
| XI-9 | [chemical structure] |
| | Enantiomer B |
| X-10 | [chemical structure] |
| XI-11 | [chemical structure] |
| XI-12 | [chemical structure] |

### Example 29—4-Bromo-1-tosyl-1H-pyrrolo[2,3-b]pyridine

[chemical structure]

To a solution of 4-bromo-1H-pyrrolo[2,3-b]pyridine (6.4 g, 32.5 mmol) in dichloromethane (100 mL) was added N,N-dimethylpyridin-4-amine (0.4 g, 3.2 mmol), triethylamine (5.4 mL, 39 mmol), followed by 4-methylbenzenesulfonyl chloride (6.8 g, 35.7 mmol). The mixture was stirred at ambient temperature for 18 hours, then concentrated onto silica. The mixture was purified by column chromatography eluting with a gradient of 0-40% ethyl acetate in hexanes. Pure fractions were combined and concentrated to yield the title compound (8.55 g, 75%).

### Example 30—4-Bromo-2-iodo-1-tosyl-1H-pyrrolo[2,3-b]pyridine

[chemical structure]

To a solution of N,N-diisopropylamine (1.8 g, 18.2 mmol) in anhydrous tetrahydrofuran (40 mL) under nitrogen at −78° C. was added a solution of 2.5M n-butyllithium (7.3 mL, 18.2 mmol) in hexanes slowly. Stirred at −78° C. for 20 minutes, then added 4-bromo-1-(p-tolylsulfonyl)pyrrolo[2,3-b]pyridine (4 g, 11.4 mmol) as a solid. Stirred at −78° C. for 30 minutes, then added iodine (5.8 g, 22.8 mmol) as a solid. Stirred at −78° C. for 2 hours, then quenched the reaction with sat. ammonium chloride, extracted with ethyl acetate. Dried extracts with sodium sulfate, decanted and concentrated onto silica. The mixture was purified by column chromatography eluting with a gradient of 0-40% ethyl acetate in hexanes. Pure fractions were combined and concentrated to yield the title compound (2.05 g, 38%).

### Example 31—4-Bromo-1-tosyl-1H-pyrrolo[2,3-b]pyridine-2-carbaldehyde

[chemical structure]

To a solution of N,N-diisopropylamine (2.3 mL, 16.3 mmol) in anhydrous tetrahydrofuran (40 mL) under at nitrogen atmosphere at −78° C. was added 2.5M n-butyllithium (6.5 mL, 16.3 mmol) in hexanes dropwise. Stirred for 5 minutes, then added 4-bromo-1-(p-tolylsulfonyl)pyrrolo[2,3-b]pyridine (3.44 g, 10.2 mmol) in portions as a solid. Stirred at −78° C. for 30 minutes, then added dimethylformamide (3.9 mL g, 51.0 mmol) and continued to stir at −78° C. for one hour. Allowed the solution to warm to ambient temperature and stirred there for 2 hours. Quenched with saturated ammonium chloride (50 mL), extracted into ethyl acetate (150 mL), washed with water, brine, dried with anhydrous sodium sulfate, filtered and concentrated to a volume of about 20 mL. Added hexanes until slightly cloudy. A solid forms which was filtered off, washing with hexanes to yield title compound (2.2 g 59%).

### Example 32—3-(4-Bromo-1-tosyl-1H-pyrrolo[2,3-b]pyridin-2-yl)oxetan-3-ol

[chemical structure]

To a solution of N,N-diisopropylamine (1.8 g, 18.222 mmol) in anhydrous tetrahydrofuran (40 mL) under at nitrogen atmosphere at −78° C. was added 2.5M n-butyllithium (7.3 mL, 18.2 mmol) in hexanes dropwise. Stirred for 5 minutes, then added 4-bromo-1-(p-tolylsulfonyl)pyrrolo[2,3-b]pyridine (4 g, 11.4 mmol) dissolved in anhydrous tetrahydrofuran (20 mL). Stirred at −78° C. for 30 minutes. To the solution was then added 3-oxetanone (1.6 g, 22.8 mmol) and continued to stir at −78° C. for one hour. Allowed the solution to warm to ambient temperature and stirred there for 2 hours. Quenched with saturated ammonium chloride (50 mL). Attempted to extract with ethyl acetate, however a solid remained suspended in both layers. Filtered mixture through filter paper, separated organic layer, dried with Na2SO4, filtered and concentrated onto silica. The mixture was purified by column chromatography eluting with a gradient of 0-40% ethyl acetate in hexanes. Pure fractions were combined and concentrated to yield the title compound (2.06 g, 43%).

### Example 33—Methyl 4-bromo-1-((2-(trimethylsilyl)ethoxy)methyl)-1H-pyrrolo[2,3-b]pyridine-2-carboxylate

[chemical structure]

### Part I—Synthesis of 1-(Benzenesulfonyl)-4-bromo-1H-pyrrolo[2,3-b]pyridine

[chemical structure]

Into a 100 mL round-bottom flask, was placed 4-bromo-1H-pyrrolo[2,3-b]pyridine (1.5 g, 7.61 mmol), dichloromethane (30 mL), triethylamine (1.59 mL, 11.4 mmol), N,N-dimethylpyridin-4-amine (93.0 mg, 0.76 mmol), benzenesulfonyl chloride (1.6 g, 9.14 mmol). The resulting solution was stirred overnight at room temperature. The mixture was concentrated, and applied onto a silica gel column, then eluted with ethyl acetate/petroleum ether (0%-10%) to yield the title compound (2.2 g, 86%).

### Part II—Synthesis of Ethyl 1-(benzenesulfonyl)-4-bromo-1H-pyrrolo[2,3-b]pyridine-2-carboxylate

[chemical structure]

Into a 250 mL 3-necked round-bottom flask purged and maintained with an inert atmosphere of nitrogen was placed 1-(benzenesulfonyl)-4-bromo-1H-pyrrolo[2,3-b]pyridine (15.0 g, 44.4 mmol) and tetrahydrofuran (150 mL). This was followed by the addition of 2M lithium N,N-diisopropylamine (33.5 mL, 67 mmol) dropwise with stirring at −78 degrees C. in 20 minutes. The resulting solution was stirred for 1 hour at −30° C. in a liquid nitrogen bath. To this was added ethyl carbonochloridate (7.24 g, 66.7 mmol) dropwise with stirring at −78° C. in 5 min. The resulting solution was stirred for an additional 1 hour while the temperature was maintained at −78° C. in a liquid nitrogen bath. The reaction was then quenched by the addition of 200 mL of ammonium chloride (aq). The resulting solution was extracted with 150 mL of ethyl acetate, washed with 3×100 ml of NaCl(aq), and concentrated under vacuum to yield the title compound (12 g, 66%).

### Part III—Synthesis of Ethyl 4-bromo-1H-pyrrolo[2,3-b]pyridine-2-carboxylate

[chemical structure]

Into a 100 mL round-bottom flask was placed ethyl 1-(benzenesulfonyl)-4-bromo-1H-pyrrolo[2,3-b]pyridine-2-carboxylate (6.7 g, 16.4 mmol), tetrahydrofuran (100 mL), and 1M tetrabutylammonium fluoride (21.3 mL, 21.3 mmol) in tetrahydrofuran. The resulting solution was stirred for 2 hours at 65° C. The mixture was concentrated under vacuum, washed solids with methanol (65 mL) to yield the title compound (4 g, 91%) as a solid.

### Part IV—Synthesis of Ethyl 4-bromo-1-[[2-(trimethylsilyl)ethoxy]methyl]-1H-pyrrolo[2,3-b]pyridine-2-carboxylate

[chemical structure]

Into a 250 mL round-bottom flask was dissolved ethyl 4-bromo-1H-pyrrolo[2,3-b]pyridine-2-carboxylate (5.20 g, 19.3 mmol) in N,N-dimethylformamide (118 mL, 1600 mmol) at 0° C. Added sodium hydride (0.93 g, 23.2 mmol, 60% in oil) in portions and stirred for 30 min at 0° C. To the mixture was added (2-(chloromethoxy)ethyl)trimethylsilane (4.8 g, 29 mmol) and allowed to react for an additional 2 hours while the temperature was maintained at 0° C. The reaction was then quenched by the addition of 50 mL of water. The crude product was purified by column chromatography to yield the title compound (6 g, (78%) as an oil.

### Example 34—4-Bromo-5-fluoro-2-iodo-1-(phenylsulfonyl)-1H-pyrrolo[2,3-b]pyridine

[chemical structure]

### Part I—Synthesis of 1-(Benzenesulfonyl)-4-bromo-5-fluoro-1H-pyrrolo[2,3-b]pyridine

[chemical structure]

Into a 250 mL round-bottom flask was placed 4-bromo-5-fluoro-1H-pyrrolo[2,3-b]pyridine (2 g, 9.3 mmol), dichloromethane (50 mL), triethylamine (3.2 mL, 23.3 mmol), benzenesulfonyl chloride (2.46 g, 14 mmol), and N,N-dimethylpyridin-4-amine (114 mg, 0.93 mmol). The resulting solution was stirred for 3 hours at room temperature, then concentrated. The residue was purified on a silica gel column eluting with ethyl acetate/petroleum ether (0:100-20:80) to yield the title compound (3.3 g, 99%) as a solid.

### Part II—Synthesis of 1-(Benzenesulfonyl)-4-bromo-5-fluoro-2-iodo-1H-pyrrolo[2,3-b]pyridine

[chemical structure]

Into a 100 mL 3-necked round-bottom flask purged and maintained with an inert atmosphere of nitrogen, was placed N,N-diisopropylamine (210 mg, 2.1 mmol) in tetrahydrofuran (15 mL) and cooled to −78° C. This was followed by the addition of 2.5M n-butyllithium (0.73 mL, 1.8 mmol) dropwise with stirring at −78° C. The reaction was stirred for 30 minutes, then added a solution of 1-(benzenesulfonyl)-4-bromo-5-fluoro-1H-pyrrolo[2,3-b]pyridine (500 mg, 1.4 mmol) in tetrahydrofuran (3 mL) dropwise with stirring at −78° C. The reaction was stirred for 30 minutes then added a solution of iodine (715 mg, 2.8 mmol) in tetrahydrofuran (5 mL) dropwise. The resulting solution was stirred for 2 hours at −78° C. The reaction was then quenched by the addition of 100 mL of aqueous ammonium chloride, extracted with ethyl acetate, dried over anhydrous sodium sulfate and concentrated. The residue was purified on a silica gel column eluting with ethyl acetate/hexane (5:1) to yield the title compound (300 mg, 44%) as a solid.

### Part III—Synthesis of 4-[1-(Benzenesulfonyl)-4-bromo-5-fluoro-1H-pyrrolo[2,3-b]pyridin-2-yl]-1-methyl-1H-pyrazole

[chemical structure]

Into a 25 mL vial purged and maintained with an inert atmosphere of nitrogen was placed 1-(benzenesulfonyl)-4-bromo-5-fluoro-2-iodo-1H-pyrrolo[2,3-b]pyridine (500 mg, 1.04 mmol), 1-methyl-4-(4,4,5,5-tetramethyl-1,3,2-dioxaborolan-2-yl)-1H-pyrazole (216 mg, 1.04 mmol), [1,1′-bis(diphenylphosphino)ferrocene]dichloropalladium(II) (46 mg, 0.06 mmol), sodium carbonate (275 mg, 2.6 mmol), toluene (7.8 mL), ethanol (2.2 mL), and water (1.1 mL). The resulting solution was stirred for 5 hours at 80° C. The mixture was concentrated, and purified on a silica gel column eluting with ethyl acetate/petroleum ether (1:1). Desired fractions were combined and concentrated to yield the title compound (200 mg 44%) as a solid.

### Example 35—6-Bromo-4-chloropyrrolo[2,1-f][1,2,4]triazine

[chemical structure]

Into a 50-mL round-bottom flask purged and maintained with an inert atmosphere of nitrogen, was placed 6-bromo-3H,4H-pyrrolo[2,1-f][1,2,4]triazin-4-one (1.0 g, 4.67 mmol, 1.00 equiv), phosphoroyl trichloride (5.0 mL). The resulting solution was stirred for 3.0 h at 130° C. The resulting mixture was concentrated under vacuum. The resulting mixture was washed with sodium bicarbonate (aq). The resulting solution was extracted with 3×100 mL of ethyl acetate and the organic layers combined and dried over anhydrous sodium sulfate and concentrated under vacuum. This resulted in 0.8 g (74%) of 6-bromo-4-chloropyrrolo[2,1-f][1,2,4]triazine as a yellow solid.

### Example 36—6-Bromo-4-chloropyrrolo[1,2-b]pyridazine

[chemical structure]

### Part I—Synthesis of Methyl 1-amino-4-bromo-1H-pyrrole-2-carboxylate

[chemical structure]

A mixture of ammonium chloride (9 g, 112 mmol) in diethyl ether (100 mL) was cooled to −5° C., concentrated aq. Ammonium hydroxide (14 mL) was added dropwise with vigorous stirring. This was followed by the addition of aq. sodium hypochlorite solution (217 mL) via addition funnel at a rate such that the internal temperature was maintained below 0° C. The mixture was then stirred for 15 minutes, then the layers were separated, and the organic layer was washed with brine and dried over anhydrous calcium chloride. The resulting solution of chloramine was used directly as follows: In a separate flask containing a solution of methyl 4-bromo-1H-pyrrole-2-carboxylate (10 g, 49 mmol) in N,N-dimethylformamide (100 mL) at 0° C. was added sodium hydride (2.9 g, 49 mmol, 60% in oil) and the resulting mixture was allowed to warm to ambient temperature and stir for 45 minutes. At this time, a portion of the previously prepared chloramine solution (100 mL) was added via syringe over 1 minute. After stirring for an additional 1.5 hours at ambient temperature, the reaction mixture was quenched by adding saturated aqueous sodium thiosulfate followed by diluting with water and extracting with diethyl ether. The mixture was further extracted with ethyl acetate The residue was applied onto a silica gel column and eluted with ethyl acetate/petroleum ether (1:10). Desired fractions were combined and concentrated to yield the title compound (8.5 g, 79%) as a solid.

### Part II—Synthesis of Ethyl 4-bromo-1-[[(1E)-3-methoxy-3-oxoprop-1-en-1-yl]amino]-1H-pyrrole-2-carboxylate

[chemical structure]

Into a 250 mL round-bottom flask purged and maintained with an inert atmosphere of nitrogen was dissolved methyl 1-amino-4-bromo-1H-pyrrole-2-carboxylate (3 g, 13.7 mmol) in methanol (150 mL) and added ethyl prop-2-ynoate (13.4 g, 137 mmol). The solution was heated to reflux overnight. The mixture was concentrated, and purified on a silica gel column eluting with ethyl acetate/petroleum ether (1:10). Desired fractions were combined and concentrated to yield the title compound (2 g, 46%) as an oil.

### Part III—Synthesis of Ethyl 6-bromo-4-oxo-1H,4H-pyrrolo[1,2-b]pyridazine-3-carboxylate

[chemical structure]

Into a 250 mL round-bottom flask was dissolved ethyl 4-bromo-1-[[(1E)-3-methoxy-3-oxoprop-1-en-1-yl]amino]-1H-pyrrole-2-carboxylate (2 g, 6.31 mmol) in ethanol (25 mL), added potassium tert-butoxide (1.4 g, 12.6 mmol). The resulting solution was heated to reflux for 2 hours. The reaction was then quenched by the addition of water/ice, and the solids were collected by filtration to yield the title compound (1.8 g, 99%).

### Part IV—Synthesis of 6-Bromo-4-oxo-1H,4H-pyrrolo[1,2-b]pyridazine-3-carboxylic acid

[chemical structure]

In a 250 mL round-bottom flask was dissolved ethyl 6-bromo-4-oxo-1H,4H-pyrrolo[1,2-b]pyridazine-3-carboxylate (1.8 g, 6.3 mmol) in ethanol (50 mL). Added 2M aqeous sodium hydroxide (50 mL, 100 mmol), and heated to reflux overnight. The reaction mixture was cooled, concentrated, adjusted the pH value of the solution to 5-6 with 3M hydrogen chloride. The solids were collected by filtration to yield the title compound (1.3 g, 80%).

### Part V—Synthesis of 6-Bromo-1H,4H-pyrrolo[1,2-b]pyridazin-4-one

[chemical structure]

Into a 100 mL round-bottom flask purged and maintained with an inert atmosphere of nitrogen was added 6-bromo-4-oxo-1H,4H-pyrrolo[1,2-b]pyridazine-3-carboxylic acid (1.3 g, 5.1 mmol) and diphenyler ether (50 mL). The resulting solution was heated to reflux for 1 hour then concentrated. The residue was purified on a silica gel column eluting with ethyl acetate/petroleum ether (1:5). Desired fractions were combined and concentrated to yield the title compound (650 mg, 60%) as a solid.

### Part VI—Synthesis of 6-Bromo-4-chloropyrrolo[1,2-b]pyridazine

[chemical structure]

Into a 50 mL round-bottom flask was placed 6-bromo-1H,4H-pyrrolo[1,2-b]pyridazin-4-one (213 mg, 1.0 mmol) in acetonitrile (5 mL). Added benzyltriethylammonium chloride (456 mg, 2.0 mmol), N,N-diethylaniline (224 mg, 1.50 mmol), and phosphoroyl trichloride (770 mg, 5.0 mmol), The reaction was heated to reflux for 4 hours, quenched by the addition of water/ice. The resulting mixture was extracted with ethyl acetate (3×50 ml), then concentrated. The residue was purified on a silica gel column eluting with ethyl acetate/petroleum ether (1:5). Desired fractions were combined and concentrated to yield the title compound (160 mg, 69%) as a solid.

### Example 37—Ethyl 8-bromo-6-chloroimidazo[1,2-b]pyridazine-2-carboxylate

[chemical structure]

In a 250 mL round-bottom flask was dissolved 4-bromo-6-chloropyridazin-3-amine (2.0 g, 9.595 mmol) in dimethylformamide (20 mL). Added ethyl 3-bromo-2-oxopropanoate (2.81 g, 14.4 mmol) and stirred overnight at 70° C. The solution was diluted with of water (40 mL), extracted with ethyl acetate (3×200 mL), dried over anhydrous sodium sulfate and concentrated under vacuum. The residue was purified on a silica gel column eluting with ethyl acetate/petroleum ether (0:100-20:80). Desired fractions were combined and concentrated to yield the title compound (890 mg, 30%) as a solid.

### Example 38—tert-Butyl 7-hydroxy-1,2,3,4-tetrahydroisoquinoline-2-carboxylate

[chemical structure]

### Part I—Synthesis of tert-Butyl 7-(4,4,5,5-tetramethyl-1,3,2-dioxaborolan-2-yl)-3,4-dihydro-1H-isoquinoline-2-carboxylate

[chemical structure]

Into a 100 mL round-bottom flask purged and maintained with an inert atmosphere of nitrogen was placed tert-butyl 7-bromo-3,4-dihydro-1H-isoquinoline-2-carboxylate (2.7 g, 8.5 mmol), dioxane (50 mL), bis(pinacolato)diboron (3.23 g, 12.7 mmol), [1,1′-bis(diphenylphosphino)ferrocene]dichloropalladium(II) (0.62 g, 0.85 mmol), and potassium acetate (1.66 g, 16.9 mmol). The reaction was stirred for 5 hours at 90° C. After cooling to room temperature, the resulting mixture was concentrated and purified on a silica gel column eluting with ethyl acetate/petroleum ether (1/10). Pure fractions were combined and concentrated to yield the title compound (3.0 g, 88%) as an oil.

### Part II—Synthesis of tert-Butyl 7-hydroxy-3,4-dihydro-1H-isoquinoline-2-carboxylate

[chemical structure]

Into a 100 mL round-bottom flask was placed tert-butyl 7-(4,4,5,5-tetramethyl-1,3,2-dioxaborolan-2-yl)-3,4-dihydro-1H-isoquinoline-2-carboxylate (3.0 g, 7.5 mmol), dichloromethane (30 mL), followed by 30% hydrogen peroxide (2.56 g, 75.2 mmol). The reaction was stirred for 16 hours at ambient temperature. The mixture was diluted with water (30 mL), extracted with ethyl acetate (3×20 mL) and concentrated. The residue was purified on a silica gel column eluting with ethyl acetate/petroleum ether (1/2). The collected fractions were combined and concentrated to yield the title compound (2.0 g, 96%) as a solid.

### Example 39—tert-Butyl 8-ethyl-3-hydroxy-5,6,7,8-tetrahydro-1,6-naphthyridine-6-carboxylate

[chemical structure]

### Part I—Synthesis of Methyl 1-benzyl-3-ethyl-4-oxopiperidine-3-carboxylate

[chemical structure]

Into a 250 mL round-bottom flask purged and maintained with an inert atmosphere of nitrogen was placed methyl 1-benzyl-4-oxopiperidine-3-carboxylate hydrochloride (50 g, 176 mmol), acetonitrile (700 mL), potassium tert-butoxide (41.5 g, 370 mmol), followed by iodoethane (41.2 g, 264 mmol). The solution was stirred overnight at room temperature. The reaction was quenched by the addition of saturated aqueous ammonium chloride (500 mL). The mixture was extracted with ethyl acetate (3×800 mL), dried over anhydrous sodium sulfate and concentrated to yield the crude title compound (50 g, quant. crude yield) as an oil.

### Part II—Synthesis of 1-Benzyl-3-ethylpiperidin-4-one

[chemical structure]

Into a 250 mL round-bottom flask was placed methyl 1-benzyl-3-ethyl-4-oxopiperidine-3-carboxylate (50 g, 182 mmol, 1 equiv) and 6M aqueous hydrogen chloride (500 mL). The solution was stirred overnight at 100°. After cooling to room temperature, the pH value of the solution was adjusted to 8 with 5M sodium hydroxide. The solution was extracted with ethyl acetate (3×1500 mL), dried over anhydrous sodium sulfate and concentrated. The residue was purified on a silica gel column eluting with ethyl acetate/petroleum ether (0:100-70:30). Pure fractions were combined and concentrated to yield the title compound (28 g, 71%) as an oil.

### Part III—Synthesis of 6-Benzyl-8-ethyl-3-nitro-5,6,7,8-tetrahydro-1,6-naphthyridine

[chemical structure]

Into a 1000 mL pressure tank reactor purged and maintained with an inert atmosphere of nitrogen was placed 1-benzyl-3-ethylpiperidin-4-one (28 g, 129 mmol), 1-methyl-3,5-dinitro-1,2-dihydropyridin-2-one (25.7 g, 129 mmol) and 7M ammonia in methanol (500 mL). The resulting solution was stirred overnight at 100° C. in an oil bath. The cooled reaction was concentrated to yield crude title compound (45 g, quant. crude yield) as a solid.

### Part IV—Synthesis of 6-Benzyl-8-ethyl-5,6,7,8-tetrahydro-1,6-naphthyridin-3-amine

[chemical structure]

Into a 250 mL round-bottom flask was combined 6-benzyl-8-ethyl-3-nitro-5,6,7,8-tetrahydro-1,6-naphthyridine (45 g, 151 mmol), methanol (800 mL), water (200 mL), ammonium chloride (648 g, 12 mol), and iron (68 g, 1.2 mol). The mixture was stirred overnight at 65° C. The solids were filtered out, and the filtrates were concentrated. The residue was purified onto a silica gel column eluting with ethyl acetate/petroleum either (0:100-80:20). Desired fractions were combined and concentrated to yield the title compound (25 g, 62%) as a solid.

### Part V—Synthesis of 6-Benzyl-8-ethyl-5,6,7,8-tetrahydro-1,6-naphthyridin-3-ol

[chemical structure]

Into a 100 mL round-bottom flask was combined 6-benzyl-8-ethyl-5,6,7,8-tetrahydro-1,6-naphthyridin-3-amine (25 g, 93.5 mmol), water (200 mL), and concentrated aqueous hydrogen chloride (20 mL). This was followed by the addition of sodium nitrite (7.1 g, 103 mmol) at 0° C. The reaction was stirred for 2 hours at room temperature, then heated overnight at 80° C. The pH value of the solution was adjusted to 8 with sodium bicarbonate(s). The mixture was extracted with ethyl acetate (3×1500 mL), dried over anhydrous sodium sulfate and concentrated. The residue was purified by column chromatography eluting with ethyl acetate in petroleum ether. Desired fractions were combined and concentrated to yield the title compound (5.5 g, 22%) as a solid.

### Part VI—Synthesis of 8-Ethyl-5,6,7,8-tetrahydro-1,6-naphthyridin-3-ol

[chemical structure]

Into a 50 mL round-bottom flask was placed 6-benzyl-8-ethyl-5,6,7,8-tetrahydro-1,6-naphthyridin-3-ol (5.5 g, 20.5 mmol), acetic acid (50 mL), and 10% palladium on carbon (5.5 g, 52 mmol). Hydrogen gas was introduced in at room temperature and the reaction was stirred overnight at room temperature. The solids were filtered out, and the filtrates were concentrated to yield the title compound (3.2 g, 88%) as an oil.

### Part VII—Synthesis of tert-Butyl 3-[[(tert-butoxy)carbonyl]oxy]-8-ethyl-5,6,7,8-tetrahydro-1,6-naphthyridine-6-carboxylate

[chemical structure]

Into a 100 mL round-bottom flask was placed 8-ethyl-5,6,7,8-tetrahydro-1,6-naphthyridin-3-ol (3.5 g, 20 mmol), dichloromethane (150 mL), triethylamine (22 mL, 158 mmol), followed by di-tert-butyl dicarbonate (17.1 g, 78.5 mmol) and N,N-dimethylpyridin-4-amine (240 mg, 2 mmol). The resulting solution was stirred for 4 hours at room temperature, then concentrated. The residue was purified on a silica gel column eluting with ethyl acetate/petroleum ether (0:100-30:70). Pure fractions were combined and concentrated to yield the title compound (5 g, 67%) as a solid.

### Part VIII—Synthesis of tert-Butyl 8-ethyl-3-hydroxy-5,6,7,8-tetrahydro-1,6-naphthyridine-6-carboxylate

[chemical structure]

Into a 25 mL round-bottom flask was placed tert-butyl 3-[[(tert-butoxy)carbonyl]oxy]-8-ethyl-5,6,7,8-tetrahydro-1,6-naphthyridine-6-carboxylate (8 g, 21 mmol), methanol (16 mL) and 30% sodium methoxide in methanol (16 mL). The resulting solution was stirred for 1 hour at room temperature. The mixture was concentrated and purified on a silica gel column eluting with dichloromethane/methanol (100:0-90:10). Desired fractions were combined and concentrated to yield the title compound (5.02 g, 85%) as a solid.

### Example 40—tert-Butyl 4-ethyl-7-hydroxy-1,2,3,4-tetrahydroisoquinoline-2-carboxylate

[chemical structure]

### Part I—Synthesis of 2-(4-Methoxyphenyl)butanenitrile

[chemical structure]

Into a 500 mL round-bottom flask purged and maintained with an inert atmosphere of nitrogen was placed tetrahydrofuran (200 mL) and 2-(4-methoxyphenyl)acetonitrile (5 g, 34 mmol). This was followed by the addition of 1M lithium bis(trimethylsilyl)amide in tetrahydrofuran (40 mL, 40 mmol) dropwise with stirring at −78° C. The reaction was stirred for 1 h at −78° C., then added iodoethane (5.8 g, 37 mmol). The reaction was stirred for 1 hour at −78° C., then quenched by the addition of saturated aqueous ammonium chloride (100 mL). Extracted with ethyl acetate (800 mL), washed with brine (3×150 mL), dried over anhydrous sodium sulfate and concentrated. The residue was purified on a silica gel column with eluting with ethyl acetate/petroleum ether (0:100-10:90). Pure fractions were combined and concentrated to yield the title compound (3.2 g, 54%) as a solid.

### Part II—Synthesis of 2-(4-Methoxyphenyl)butan-1-amine

[chemical structure]

Into a 100 mL round-bottom flask purged and maintained with an inert atmosphere of nitrogen was placed 2-(4-methoxyphenyl)butanenitrile (3.2 g, 18.3 mmol), methanol (20 mL), Raney Nickel (6.4 g, 75 mmol), ammonium hydroxide (0.3 mL). The atmosphere was purged and replaced with hydrogen gas. The mixture was stirred for 3 hours at room temperature, degassed and the solids were filtered out. The crude product was reverse phase chromatography to yield the title compound (5 g, 67%) as a solid.

### Part III—Synthesis of 1-(1-Isocyanatobutan-2-yl)-4-methoxybenzene

[chemical structure]

Into a 100 mL round-bottom flask was placed 2-(4-methoxyphenyl)butan-1-amine; bis(trifluoroacetic acid) (2.5 g, 6.1 mmol), dichloromethane (40 mL) and 2M sodium hydroxide (12 mL, 24 mmol). This was followed by the addition of triphosgene (0.7 g, 2.46 mmol) portionwise at room temperature. The reaction was stirred for 2 hours at room temperature, extracted with dichloromethane (3×150 mL), dried over anhydrous sodium sulfate and concentrated to yield crude title compound (800 mg, 64%) as a solid.

### Part IV—Synthesis of 4-Ethyl-7-hydroxy-1,2,3,4-tetrahydroisoquinolin-1-one

[chemical structure]

Into a 100 mL round-bottom flask purged and maintained with an inert atmosphere of nitrogen was dissolved 1-(1-isocyanatobutan-2-yl)-4-methoxybenzene (800 mg, 3.90 mmol) in trifluoromethanesulfonic acid (5 mL). The reaction was heated for 1 hour at 100° C. The reaction was then quenched by the addition of ice water (100 mL), adjusted the pH value to 8 with sodium bicarbonate (aq), extracted with ethyl acetate (3×150 mL), dried over anhydrous sodium sulfate and concentrated. The residue was purified on a silica gel column eluting with dichloromethane/methanol (100:0-90:10). Pure fractions were combined and concentrated to yield the title compound (500 mg, 67%) as a solid.

### Part V—Synthesis of 4-Ethyl-1,2,3,4-tetrahydroisoquinolin-7-ol

[chemical structure]

Into a 100 mL round-bottom flask was dissolved 4-ethyl-7-hydroxy-1,2,3,4-tetrahydroisoquinolin-1-one (500 mg, 2.61 mmol) in tetrahydrofuran (25 mL) and cooled to 0° C. This was followed by the addition of 1M lithium aluminum hydride in tetrahydrofuran (26 mL, 26 mmol) dropwise. The reaction was stirred overnight at 70° C. in an oil bath, cooled and quenched carefully by the addition of 1/1/3 ratio of water/15% NaOH/water at 0° C. The solids were filtered out and the filtrate was concentrated to yield the title compound (580 mg, quant. crude yield) as a solid.

### Part VI—Synthesis of tert-Butyl 7-[[(tert-butoxy)carbonyl]oxy]-4-ethyl-1,2,3,4-tetrahydroisoquinoline-2-carboxylate

[chemical structure]

Into a 100 mL round-bottom flask purged and maintained with an inert atmosphere of nitrogen, was dissolved 4-ethyl-1,2,3,4-tetrahydroisoquinolin-7-ol (580 mg, 3.27 mmol) in dichloromethane (15 mL), added triethylamine (3.6 mL, 26 mmol) and di-tert-butyl dicarbonate (2.1 g, 9.8 mmol). The reaction was stirred at overnight at room temperature, diluted with dichloromethane (200 mL), washed with brine (3×50 mL), dried over anhydrous sodium sulfate and concentrated. The residue was purified on a silica gel column eluting with ethyl acetate/petroleum ether (1:6). Pure fractions were combined and concentrated to yield the title compound (500 mg, 40%) as a solid.

### Part VII—Synthesis of tert-Butyl 4-ethyl-7-hydroxy-1,2,3,4-tetrahydroisoquinoline-2-carboxylate

[chemical structure]

Into a 50 mL round-bottom flask was dissolve tert-butyl 7-[[(tert-butoxy)carbonyl]oxy]-4-ethyl-1,2,3,4-tetrahydroisoquinoline-2-carboxylate (500 mg, 1.32 mmol, 1 equiv) in methanol (3 mL). Added 30% sodium methoxide in methanol (3 mL). The solution was stirred for 1 hour at room temperature, diluted with of ethyl acetate (500 mL), washed with brine (4×50 mL). The mixture was dried over anhydrous sodium sulfate and concentrated. The residue was purified on a silica gel column eluting with ethyl acetate/petroleum ether (1:3). Desired fractions were combined and concentrated to yield the title compound (360 mg, 98%) as a solid.

### Example 41—tert-Butyl (7R)-3-hydroxy-7-methyl-5,6,7,8-tetrahydro-1,6-naphthyridine-6-carboxylate

[chemical structure]

### Part I—Synthesis of tert-Butyl (2R)-2-methyl-4,6-dioxopiperidine-1-carboxylate

[chemical structure]

Into a 250 mL round-bottom flask was dissolved (3R)-3-[[(tert-butoxy)carbonyl]amino]butanoic acid (5 g, 24.6 mmol), 2,2-dimethyl-1,3-dioxane-4,6-dione (3.9 g, 27 mmol) in dichloromethane (50 mL), added N-(3-dimethylaminopropyl)-N′-ethylcarbodiimide hydrochloride (5.6 g, 29.5 mmol), N,N-dimethylpyridin-4-amine (4.5 g, 37 mmol). The reaction was stirred overnight at room temperature, diluted with dichloromethane (50 mL), washed with of 1M sodium hydrogen sulfate (2×200 mL). The resulting solution was diluted with ethyl acetate (50 mL), and heated for 3 hours at 80° C. The solution was concentrated to yield the title compound (5 g, 89%) as a solid.

### Part II—Synthesis of [(2E)-3-(Dimethylamino)-2-methoxyprop-2-en-1-ylidene]dimethylazanium perchlorate

[chemical structure]

Into a 250 mL round-bottom flask was placed N,N-dimethylformamide (36 g, 0.493 mmol), dichloroethane (0.01 mL, 0.124 mmol, 1.03 equiv). This was followed by the addition of POCl₃ (0.01 g, 0.084 mmol, 0.56 equiv) dropwise with stirring at 0° C. in 30 min. To this was added (2,2-dimethoxyethyl)dimethylamine (20 g, 0.150 mmol, 1 equiv) at 0° C. The resulting solution was stirred for overnight at 70° C. in an oil bath. The reaction was then quenched by the addition of 150 g of water/ice. The resulting mixture was washed with 2×300 ml of Hexane and this mixture was agitated and the organic phase separated and wased with 100 ml cold water. The combined aqueous phases were treated with 30 g sodium perchlorate monohydrate and dissolved in 30 ml dissolved water. The solids were collected by filtration. This resulted in 20 g (51.89%) of [(2E)-3-(dimethylamino)-2-methoxyprop-2-en-1-ylidene]dimethylazanium perchlorate as a yellow solid.

### Part III—Synthesis of (7R)-3-Ethoxy-7-methyl-5,6,7,8-tetrahydro-1,6-naphthyridin-5-one

[chemical structure]

Into a 100 mL round-bottom flask was dissolved tert-butyl (2R)-2-methyl-4,6-dioxopiperidine-1-carboxylate (2.5 g, 11 mmol) in tetrahydrofuran (50 mL), added 1M potassium tert-butoxide in tetrahydrofuran (11 mL, 11 mmol) at 0° C. To this was added [(2E)-3-(dimethylamino)-2-ethoxyprop-2-en-1-ylidene]dimethylazanium perchlorate (4.5 g, 17.6 mmol) at ambient temperature and stirred there for 3 hours. The resulting mixture was concentrated, added N,N-dimethylformamide (50 mL) and ammonium acetate (2.5 g, 32 mmol) and stirred overnight at 120° C. Solvents were removed under vacuum and the residue was purified on a silica gel column eluting with ethyl acetate:petroleum ether (3:7). Pure fractions were combined and concentrated to yield the title compound (1.4 g, 62%) as a solid.

### Part IV—Synthesis of (7R)-3-Hydroxy-7-methyl-5,6,7,8-tetrahydro-1,6-naphthyridin-5-one

[chemical structure]

Into a 25 mL round-bottom flask was dissolved (7R)-3-ethoxy-7-methyl-5,6,7,8-tetrahydro-1,6-naphthyridin-5-one (400 mg, 1.9 mmol) in trifluoromethanesulfonic acid (4 mL) and heated to 100° C. for 2 hours. The cooled reaction was quenched with ice water and the pH was adjusted to 9 with sodium bicarbonate. The mixture was extracted with ethyl acetate (2×50 mL), dried over anhydrous sodium sulfate and concentrated. The residue was purified on a silica gel column eluting with dichloromethane/methanol (20:1). Desired fractions were combined and concentrated to yield the title compound (300 mg, 87%) as a solid.

### Part V—Synthesis of (7R)-7-Methyl-5,6,7,8-tetrahydro-1,6-naphthyridin-3-ol

[chemical structure]

Into a 50 mL round-bottom flask was dissolved (7R)-3-hydroxy-7-methyl-5,6,7,8-tetrahydro-1,6-naphthyridin-5-one (300 mg, 1.7 mmol) in 1,4-dioxane (10 mL), added of 1M borane-tetrahydrofuran complex (17 mL, 17 mmol) at 0° C. The reaction was stirred for 3 hours at 65° C. After cooling to 0° C., 2M aqueous hydrogen chloride (2 mL) was added carefully. Stirred for an additional 2 hours at 65° C., then concentrated to yield the title compound (250 mg, 90%) as a solid.

### Part VI—Synthesis of tert-Butyl (7R)-3-[[(tert-butoxy)carbonyl]oxy]-7-methyl-5,6,7,8-tetrahydro-1,6-naphthyridine-6-carboxylate

[chemical structure]

Into a 50 mL round-bottom flask was placed (7R)-7-methyl-5,6,7,8-tetrahydro-1,6-naphthyridin-3-ol (250 mg, 1.5 mmol), 1,4-dioxane (8 mL), 2M sodium hydroxide (2 mL), and di-tert-butyl dicarbonate (665 mg, 3 mmol). The solution was stirred for 2 hours at room temperature, diluted with dichloromethane (50 mL), washed with water (2×50 mL), dried over anhydrous sodium sulfate and concentrated to yield the title compound (400 mg, 72%) as a solid.

### Part VII—Synthesis of tert-Butyl (7R)-3-hydroxy-7-methyl-5,6,7,8-tetrahydro-1,6-naphthyridine-6-carboxylate

[chemical structure]

Into a 50 mL round-bottom flask was dissolved tert-butyl (7R)-3-[[(tert-butoxy)carbonyl]oxy]-7-methyl-5,6,7,8-tetrahydro-1,6-naphthyridine-6-carboxylate (400 mg, 1.1 mmol) in methanol (5 mL), and added 30% sodium methoxide in methanol (3 mL). The solution was stirred for 1 hour at room temperature and concentrated. The residue was purified on a silica gel column eluting with dichloromethane/methanol (10:1). Pure fractions were combined and concentrated to yield the title compound (200 mg, 69%) a solid.

### Example 42—Phenyl (4-((1-methylpiperidin-4-yl)oxy)-3-(trifluoromethyl)phenyl)carbamate

[chemical structure]

### Part I—Synthesis of 1-Methyl-4-[4-nitro-2-(trifluoromethyl)phenoxy]piperidine

[chemical structure]

Into a 250 mL round-bottom flask was dissolved 1-methylpiperidin-4-ol (1.5 g, 13.3 mmol) in tetrahydrofuran (50 mL) and cooled to 0° C. Added sodium hydride (0.5 g, 0.02 mmol, 60% dispersion in oil). The mixture was stirred for 30 min at 0° C., then added 1-chloro-4-nitro-2-(trifluoromethyl)benzene (3 g, 13.3 mmol). The solution was stirred overnight at room temperature. The reaction was then quenched by the addition of cold brine (40 mL), extracted with ethyl acetate (3×40 mL), then concentrated. The residue was purified on a silica gel column eluting with dichloromethane/methanol (100:6). Desired fractions were combined and concentrated to yield the title compound (850 mg, 21%) as an oil.

### Part II—Synthesis of 4-[(1-Methylpiperidin-4-yl)oxy]-3-(trifluoromethyl)aniline

[chemical structure]

Into a 100 mL round-bottom flask was dissolved 1-methyl-4-[4-nitro-2-(trifluoromethyl)phenoxyl]piperidine (850 mg, 2.79 mmol) in tetrahydrofuran (40 mL), added rodium on carbon (800 mg, 7.77 mmol), purged atmosphere and replaced with hydrogen gas. The mixture was stirred overnight at room temperature, filtered solids, then concentrated the filtrates to yield the title compound (600 mg, 78%) as a solid.

### Part III—Synthesis of Phenyl (4-((1-methylpiperidin-4-yl)oxy)-3-(trifluoromethyl)phenyl)carbamate

[chemical structure]

Into a 50 mL round-bottom flask was placed 4-[(1-methylpiperidin-4-yl)oxy]-3-(trifluoromethyl)aniline (150 mg, 0.55 mmol), pyridine (20 mL) followed by phenyl chloroformate (171.2 mg, 1.09 mmol). The solution was stirred overnight at room temperature, and concentrated. The residue was purified on a silica gel column with dichloromethane/methanol (10:1) and the desired fractions were combined and concentrated to yield the title compound.

### Example 43—1-[2-[(tert-Butyldiphenylsilyl)oxy]ethyl]-4-[[2-(difluoromethyl)-4-isocyanatophenyl]methyl]piperazine

[chemical structure]

### Part I—Synthesis of 2-(Difluoromethyl)-1-methyl-4-nitrobenzene

[chemical structure]

Into a 100 mL round-bottom flask purged and maintained with an inert atmosphere of nitrogen was dissolved 2-methyl-5-nitrobenzaldehyde (1.1 g, 6.66 mmol) in dichloromethane (20 mL) and cooled to 0° C., added diethylaminosulfur trifluoride (0.98 mL, 8 mmol) dropwise. The resulting solution was stirred overnight at room temperature, then concentrated under vacuum. The residue was purified on a silica gel column eluting with ethyl acetate/petroleum ether (1/100). Pure fractions were combined and concentrated to yield the title compound (1.16 g, 93%) as an oil.

### Part II—Synthesis of 1-(Bromomethyl)-2-(difluoromethyl)-4-nitrobenzene

[chemical structure]

Into a 100 mL round-bottom flask purged and maintained with an inert atmosphere of nitrogen was dissolved 2-(difluoromethyl)-1-methyl-4-nitrobenzene (1.16 g, 6.20 mmol) in dichloroethane (30 mL), added 2,2′-azobis(2-methylpropionitrile) (410 mg, 2.50 mmol) followed by N-bromosuccinimide (1.66 g, 9.33 mmol). The mixture was heated to reflux overnight, cooled, diluted with ethyl acetate, and washed with water (3×20 mL) The organic layers were dried over anhydrous sodium sulfate and concentrated under vacuum. The residue was purified on a silica gel column eluting with ethyl acetate/petroleum ether (1/100). Desired fractions were combined and concentrated to yield the title compound (1.27 g, 77%) as an oil.

### Part III—Synthesis of 2-(4-[[2-(Difluoromethyl)-4-nitrophenyl]methyl]piperazin-1-yl)ethan-1-ol

[chemical structure]

Into a 100 mL round-bottom flask was dissolved 1-(bromomethyl)-2-(difluoromethyl)-4-nitrobenzene (1.27 g, 4.77 mmol) in dichloromethane (30 mL), added 2-(piperazin-1-yl)ethan-1-ol (680 mg, 5.22 mmol) and N,N-diisopropylethylamine (1.18 mL, 6.1 mmol). The resulting solution was stirred overnight at room temperature, concentrated under vacuum, and purified on a silica gel column with dichloromethane/methanol (1/20). Desired fractions were combined and concentrated to yield the title compound (800 mg, 53%) as a solid.

### Part IV—Synthesis of 1-[2-[(tert-Butyldiphenylsilyl)oxy]ethyl]-4-[[2-(difluoromethyl)-4-nitrophenyl]methyl]piperazine

[chemical structure]

Into a 100 mL round-bottom flask was dissolved 2-(4-[[2-(difluoromethyl)-4-nitrophenyl]methyl]piperazin-1-yl)ethan-1-ol (900 mg, 2.85 mmol) in dichloromethane (20 mL), added triethylamine (481.4 mg, 4.76 mmol) followed by tert-butyldiphenylsilyl chloride (650 mg, 4.3 mmol). The reaction was stirred overnight at room temperature, concentrated under vacuum, and purified on a silica gel column eluting with ethyl acetate/petroleum ether (1/100). Pure fractions were combined and concentrated to yield the title compound (1.3 g, 82%) as an oil.

### Part V—Synthesis of 4-[(4-[2-[(tert-Butyldiphenylsilyl)oxy]ethyl]piperazin-1-yl)methyl]-3-(difluoromethyl)aniline

[chemical structure]

Into a 100 mL round-bottom flask purged and maintained with an inert atmosphere of nitrogen was dissolved 1-[2-[(tert-butyldiphenylsilyl)oxy]ethyl]-4-[[2-(difluoromethyl)-4-nitrophenyl]methyl]piperazine (400 mg, 0.72 mmol) in methanol (30 mL) and water (15 mL). Added powdered iron (202 mg, xx mmol) and ammonium chloride (966 mg, 18 mmol) and heated to 70° C. overnight. The solids were filtered out and the filtrates were concentrated under vacuum. The residue was purified on a silica gel column eluting with ethyl acetate/petroleum ether (1/20). Desired fractions were combined and concentrated to yield the title compound (240 mg, 63%) as an oil.

### Part VI—Synthesis of 1-[2-[(tert-Butyldiphenylsilyl)oxy]ethyl]-4-[[2-(difluoromethyl)-4-isocyanatophenyl]methyl]piperazine

[chemical structure]

Into a 50 mL round-bottom flask purged and maintained with an inert atmosphere of nitrogen was dissolved 4-[(4-[2-[(tert-butyldiphenylsilyl)oxy]ethyl]piperazin-1-yl)methyl]-3-(difluoromethyl)aniline (220 mg, 0.42 mmol) in toluene (10 mL), added triphosgene (624 mg, 2.1 mmol). The reaction was stirred for 2 hours at room temperature, then concentrated under vacuum to yield crude title compound (300 mg, quant. crude yield) as a solid.

### Example 44—Phenyl N-[2-fluoro-4-[(4-methylpiperazin-1-yl)methyl]-5-(trifluoromethyl)phenyl]carbamate

[chemical structure]

### Part I—Synthesis of 1-[[5-Fluoro-2-(trifluoromethyl)phenyl]methyl]-4-methylpiperazine

[chemical structure]

Into a 100 mL round-bottom flask was placed 2-(bromomethyl)-4-fluoro-1-(trifluoromethyl)benzene (1.00 g, 3.891 mmol), dichloromethane (20 mL), N,N-diisopropylethylamine (1.4 mL, 7.8 mmol), and 1-methylpiperazine (0.43 g, 4.3 mmol). The solution was stirred overnight at room temperature, concentrated, and purified on a silica gel column eluting with ethyl acetate/petroleum ether (0:100-30:70). Desired fractions were combined and concentrated to yield the title compound (0.9 g, 84%) as a solid.

### Part II—Synthesis of 1-[[5-Fluoro-4-nitro-2-(trifluoromethyl)phenyl]methyl]-4-methylpiperazine

[chemical structure]

Into a 100 mL round-bottom flask purged and maintained with an inert atmosphere of nitrogen was dissolved 1-[[5-fluoro-2-(trifluoromethyl)phenyl]methyl]-4-methylpiperazine (0.90 g, 3.258 mmol) in concentrated sulfuric acid (15 mL). This was followed by the addition of nitric acid (0.63 g, 6.5 mmol) dropwise with stirring at 0° C. The reaction was stirred overnight at 60° C., then quenched by the addition of ice water (100 mL). The pH of the solution was adjusted to 8 with sodium carbonate, extracted with ethyl acetate (3×100 mL), dried over anhydrous sodium sulfate and concentrated. The residue was purified on a silica gel column eluting with ethyl acetate/hexane (0:100-70:30). Desired fractions were combined and concentrated to yield the title compound (600 mg, 57%) as a solid.

### Part III—Synthesis of 2-Fluoro-4-[(4-methylpiperazin-1-yl)methyl]-5-(trifluoromethyl)aniline

[chemical structure]

Into a 100 mL round-bottom flask was placed 1-[[5-fluoro-4-nitro-2-(trifluoromethyl)phenyl]methyl]-4-methylpiperazine (600.00 mg, 1.868 mmol, 1.00 equiv), methanol (15 mL), water (5 mL), ammonium chloride (4 g, 75 mmol), powdered iron (520 mg, 9.3 mmol). The suspension was stirred overnight at 60° C., filtered, and concentrated. The residue was purified on a silica gel column eluting with dichloromethane/methanol (100:0-80:20). Desired fractions were combined and concentrated to yield the title compound (400 mg, 74%) as a solid.

### Part IV—Synthesis of Phenyl N-[2-fluoro-4-[(4-methylpiperazin-1-yl)methyl]-5-(trifluoromethyl)phenyl]carbamate

[chemical structure]

Into a 25 mL round-bottom flask was dissolved 2-fluoro-4-[(4-methylpiperazin-1-yl)methyl]-5-(trifluoromethyl)aniline (400.00 mg, 1.373 mmol, 1.00 equiv), in dichloromethane (10 mL), added pyridine (0.33 mL, 4.1 mmol) followed by phenyl chloroformate (237 mg, 1.5 mmol). The solution was stirred overnight at room temperature, then concentrated. The residue was purified on a silica gel column eluting with dichloromethane/methanol (100:0-80:20). Desired fractions were combined and concentrated to yield the title compound (400 mg, 71%) as a solid.

### Example 44A—Phenyl (6-((4-methylpiperazin-1-yl)methyl)-5-(trifluoromethyl)pyridin-3-yl)carbamate

[chemical structure]

### Part I—Synthesis of 6-((4-Methylpiperazin-1-yl)methyl)-5-(trifluoromethyl)pyridin-3-amine

[chemical structure]

Into a 250 mL round-bottom flask was dissolved 5-amino-3-(trifluoromethyl)pyridine-2-carbaldehyde hydrochloride (8 g, 35 mmol) in dichloroethane (100 mL) and acetic acid (2 mL), added 1-methylpiperazine (7.07 g, 70.615 mmol, 2 equiv) stirred for 30 min at room temperature, then added sodium triacetoxyborohydride (15.0 g, 71 mmol). The reaction was stirred for 2 hours at room temperature, then concentrated under vacuum. The crude product was purified by reverse phase chromatography to yield the title compound (6.2 g, 45%) as the trifluoroacetate salt.

### Part II—Synthesis of Phenyl N-[6-((4-methylpiperazin-1-yl)methyl]-5-(trifluoromethyl)pyridin-3-yl]carbamate

[chemical structure]

Into a 250 mL round-bottom flask was dissolved 6-((4-methylpiperazin-1-yl)methyl)-5-(trifluoromethyl)pyridin-3-amine; trifluoroacetic acid (6.2 g, 16.0 mmol) in dichloromethane (8 mL) and pyridine (6.5 mL, 80 mmol). Added phenyl carbonochloridate (2.51 g, 15.979 mmol, 1 equiv) and stirred for 2 hours at room temperature. The mixture was concentrated under vacuum and purified on a silica gel column eluting with dichloromethane/methanol (100:0-80:20). Desired fractions were combined and concentrated to yield the title compound (1.23 g, 19%) as a solid.

### Example 45—tert-Butyl (1S)-7-[[1-(benzenesulfonyl)-1H-pyrrolo[2,3-b]pyridin-4-yl]oxy]-1-methyl-1,2,3,4-tetrahydroisoquinoline-2-carboxylate

[chemical structure]

Into a 50 mL round-bottom flask purged and maintained with an inert atmosphere of nitrogen was placed 1-(benzenesulfonyl)-4-bromo-1H-pyrrolo[2,3-b]pyridine (600 mg, 1.78 mmol), tert-butyl (1S)-7-hydroxy-1-methyl-1,2,3,4-tetrahydroisoquinoline-2-carboxylate (468.6 mg, 1.78 mmol), toluene (20 mL), tris(dibenzylideneacetone)dipalladium(0) (92 mg, 0.10 mmol), 2-dicyclohexylphosphino-2′,4′,6′-triisopropylbiphenyl (85 mg, 0.18 mmol) and potassium carbonate (0.62 g, 4.5 mmol). The mixture was stirred for 2 hour at 110° C., then concentrated under vacuum. The residue was purified on a silica gel column eluting with ethyl acetate/petroleum ether (20%-30%). Desired fractions were combined and concentrated to yield the title compound (500 mg, 54%) as a light yellow solid.

### Example 46—tert-Butyl (1S)-7-[[1-(benzenesulfonyl)-2-iodo-1H-pyrrolo[2,3-b]pyridin-4-yl]oxy]-1-methyl-1,2,3,4-tetrahydroisoquinoline-2-carboxylate

[chemical structure]

Into a 100 mL 3-necked round-bottom flask purged and maintained with an inert atmosphere of nitrogen was dissolved tert-butyl (1S)-7-[[1-(benzenesulfonyl)-1H-pyrrolo[2,3-b]pyridin-4-yl]oxy]-1-methyl-1,2,3,4-tetrahydroisoquinoline-2-carboxylate (820 mg, 1.58 mmol) in tetrahydrofuran (20 mL) and cooled to −78° C. Added lithium diisopropylethylamine solution (338 mg, 3.16 mmol) and stirred for 30 minutes at −78° C. To this was added iodine (1.2 g, 4.7 mmol). The resulting solution was stirred for 5 hours at room temperature, then quenched by the addition of saturated ammonium chloride. Extracted with ethyl acetate (3×20 mL), dried over anhydrous sodium sulfate and concentrated. The residue was purified on a silica gel column eluting with ethyl acetate/petroleum ether (0-10%). Desired fractions were combined and concentrated to yield the title compound (500 mg, 49%) as a solid.

### Example 47—tert-Butyl 7-[(2-amino-3-bromo-5-fluoropyridin-4-yl)oxy]-3,4-dihydro-1H-isoquinoline-2-carboxylate

[chemical structure]

Into a 100 mL round-bottom flask purged and maintained with an inert atmosphere of nitrogen was dissolved tert-butyl 7-hydroxy-3,4-dihydro-1H-isoquinoline-2-carboxylate (700 mg, 2.8 mmol) N,N-dimethylacetamide (30.00 mL), added cesium carbonate (2.3 g, 7 mmol), 3-bromo-4-chloro-5-fluoropyridin-2-amine (760 mg, 3.4 mmol) and stirred at 120° C. for 3 hours. The cooled reaction mixture was diluted with water (40 mL) and extracted with ethyl acetate (3×100 mL). The combined extracts were dried over anhydrous sodium sulfate and concentrated under vacuum. The residue was purified on a silica gel column eluting with ethyl acetate/petroleum ether (0:100-10:90). Desired fractions were combined and concentrated to yield the title compound (1 g, 81%) as an oil.

### Example 48—tert-Butyl 8-ethyl-3-((1-(phenylsulfonyl)-1H-pyrrolo[2,3-b]pyridin-4-yl)oxy)-7,8-dihydro-1,6-naphthyridine-6(5H)-carboxylate

[chemical structure]

Into a 50-mL round-bottom flask purged and maintained with an inert atmosphere of nitrogen, was placed tert-butyl 8-ethyl-3-hydroxy-5,6,7,8-tetrahydro-1,6-naphthyridine-6-carboxylate (750 mg, 2.7 mmol), toluene (20 mL), 1-(benzenesulfonyl)-4-bromo-1H-pyrrolo[2,3-b]pyridine (1.3 g, 3.8 mmol), tris(dibenzylideneacetone)dipalladium(0) (250 mg, 0.27 mmol), potassium carbonate (1.1 g, 8.1 mmol), and 2-dicyclohexylphosphino-2′,4′,6′-triisopropylbiphenyl (257 mg, 0.54 mmol). The reaction was stirred for 2 days at 90° C., then concentrated. The residue was purified on a silica gel column eluting with dichloromethane/methanol (10:1). Desired fractions were combined and concentrated to yield the title compound (124 mg, 43%).

### Example 49—tert-Butyl (1S)-7-[[1-(benzenesulfonyl)-2-formyl-1H-pyrrolo[2,3-b]pyridin-4-yl]oxy]-1-methyl-1,2,3,4-tetrahydroisoquinoline-2-carboxylate

[chemical structure]

Into a 50 mL 3-necked round-bottom flask purged and maintained with an inert atmosphere of nitrogen was added tetrahydrofuran (15 mL) and diisopropylamine (90 mg, 0.89 mmol), cooled to −78° C., and added 2.5M n-butyllithium (3 mL, 1.8 mmol) dropwise. The mixture was stirred for 30 minutes at −78° C., then added tert-butyl (1S)-7-[[1-(benzenesulfonyl)-1H-pyrrolo[2,3-b]pyridin-4-yl]oxy]-1-methyl-1,2,3,4-tetrahydroisoquinoline-2-carboxylate (200 mg, 0.38 mmol) The mixture was stirred for 30 minutes at −78° C., then added N,N-dimethylformamide (90 μL, 1.15 mmol). Stirred for 2 hours at −78° C. then quenched by the addition of saturated ammonium chloride (10 mL). Extracted with ethyl acetate (3×50 mL), concentrated, and purified on a silica gel column with ethyl acetate/petroleum ether (1:3). Desired fractions were combined and concentrated to yield the title compound (150 mg, 71%) as a solid.

### Example 50—tert-Butyl (1S)-7-[[2-(2-methoxyethyl)-1H-pyrrolo[2,3-b]pyridin-4-yl]oxy]-1-methyl-1,2,3,4-tetrahydroisoquinoline-2-carboxylate

[chemical structure]

### Part I—Synthesis of tert-Butyl (1S)-7-[[1-(benzenesulfonyl)-2-(2-methoxyethenyl)-1H-pyrrolo[2,3-b]pyridin-4-yl]oxy]-1-methyl-1,2,3,4-tetrahydroisoquinoline-2-carboxylate

[chemical structure]

In a 50 mL 3-necked round-bottom flask purged and maintained with an inert atmosphere of nitrogen was dissolved (methoxymethyl)triphenylphosphanium chloride (281.7 mg, 0.82 mmol) in tetrahydrofuran (15 mL), cooled to 0° C., and added potassium tert-butoxide (74 mg, 0.66 mmol). The reaction was stirred for 20 minutes, then added tert-butyl (1S)-7-[[1-(benzenesulfonyl)-2-formyl-1H-pyrrolo[2,3-b]pyridin-4-yl]oxy]-1-methyl-1,2,3,4-tetrahydroisoquinoline-2-carboxylate (300 mg, 0.55 mmol). The reaction was stirred overnight at room temperature, quenched with saturated ammonium chloride (50 mL), extracted with ethyl acetate (3×50 mL), dried over anhydrous sodium sulfate and concentrated. The residue was purified on a silica gel column eluting with ethyl acetate/petroleum ether (1:2). Desired fractions were combined and concentrated to yield the title compound (220 mg, 70%) as a solid, as a mixture of cis and trans olefin isomers.

### Part II—Synthesis of tert-butyl (1S)-7-[[2-(2-methoxyethenyl)-1H-pyrrolo[2,3-b]pyridin-4-yl]oxy]-1-methyl-1,2,3,4-tetrahydroiso-quinoline-2-carboxylate

[chemical structure]

In a 25 mL microwave tube, purged and maintained with an inert atmosphere of nitrogen, was placed tert-butyl (1S)-7-[[1-(benzenesulfonyl)-2-(2-methoxyethenyl)-1H-pyrrolo[2,3-b]pyridin-4-yl]oxy]-1-methyl-1,2,3,4-tetrahydroisoquinoline-2-carboxylate (220 mg, 0.38 mmol), tetrahydrofuran (6 mL), trifluoroethanol (3 mL), and cesium carbonate (310 mg, 0.96 mmol). The mixture was heated in a microwave for 2 hours at 100° C. The mixture was concentrated and purified on a silica gel column eluting with dichloromethane/methanol (5:1). Desired fractions were combined and concentrated to yield the title compound (80 mg, 48%) as a solid, as a mixture of cis and trans olefin isomers.

### Part III—Synthesis of tert-Butyl (1S)-7-[[2-(2-methoxyethyl)-1H-pyrrolo[2,3-b]pyridin-4-yl]oxy]-1-methyl-1,2,3,4-tetrahydroisoquinoline-2-carboxylate

[chemical structure]

Into a 50 mL round-bottom flask was dissolved tert-butyl (1S)-7-[[2-(2-methoxyethenyl)-1H-pyrrolo[2,3-b]pyridin-4-yl]oxy]-1-methyl-1,2,3,4-tetrahydroisoquinoline-2-carboxylate (80 mg, 0.18 mmol), in methanol (15 mL) under nitrogen. Added 10% palladium on carbon (80 mg), then purged atmosphere replacing with hydrogen gas. The suspension was stirred overnight at room temperature, filtered through celite, then concentrated to yield the title compound (50 mg, 62%) as an oil.

### Example 51—tert-Butyl (1S)-7-[[1-(benzenesulfonyl)-2-cyclopropyl-1H-pyrrolo[2,3-b]pyridin-4-yl]oxy]-1-methyl-1,2,3,4-tetrahydroisoquinoline-2-carboxylate

[chemical structure]

Into a 25 mL round-bottom flask purged and maintained with an inert atmosphere of nitrogen was placed tert-butyl (1S)-7-[[1-(benzenesulfonyl)-2-iodo-1H-pyrrolo[2,3-b]pyridin-4-yl]oxy]-1-methyl-1,2,3,4-tetrahydroisoquinoline-2-carboxylate (60 mg, 0.09 mmol), toluene (5 mL), cyclopropylboronic acid (10.4 mg, 0.12 mmol), tricyclohexylphosphane (5.2 mg, 0.02 mmol), potassium triphosphate (69 mg, 0.33 mmol), and palladium(II) acetate (2 mg, 0.01 mmol). The reaction was stirred overnight at 100° C. then concentrated. The residue was purified on a silica gel column eluting with ethyl acetate/petroleum ether (1:2). Desired fractions were combined and concentrated to yield the title compound (70 mg, 94%) as a solid.

### Example 52—tert-Butyl (1S)-1-methyl-7-[[2-(1-methyl-1H-pyrazol-4-yl)-1H-pyrrolo[2,3-b]pyridin-4-yl]oxy]-1,2,3,4-tetrahydroisoquinoline-2-carboxylate

[chemical structure]

Into a 25 mL round-bottom flask purged and maintained with an inert atmosphere of nitrogen was placed tert-butyl (1S)-7-[[1-(benzenesulfonyl)-2-iodo-1H-pyrrolo[2,3-b]pyridin-4-yl]oxy]-1-methyl-1,2,3,4-tetrahydroisoquinoline-2-carboxylate (60 mg, 0.09 mmol), water (5 mL), 1,4-dioxane (5 mL), (1-methyl-1H-pyrazol-4-yl)boronic acid (18.7 mg, 0.15 mmol), [1,1′-bis(diphenylphosphino)ferrocene]dichloropalladium(II) (10.2 mg, 0.01 mmol), and potassium carbonate (25.7 mg, 0.19 mmol). The reaction was stirred overnight at 90° C., then concentrated. The residue was purified on a silica gel column eluting with dichloromethane/methanol (10:1). Desired fractions were combined and concentrated to yield the title compound (60 mg, 90%) as a solid.

### Example 53—(8S)-8-Ethyl-3-[[2-(1H-pyrazol-4-yl)-1H-pyrrolo[2,3-b]pyridin-4-yl]oxy]-5,6,7,8-tetrahydro-1,6-naphthyridine

[chemical structure]

### Part I—Synthesis of 1-(Oxan-2-yl)-4-[2-(trimethylsilyl)ethynyl]pyrazole

[chemical structure]

Into a 100 mL round-bottom flask, was placed 4-bromo-1-(oxan-2-yl)pyrazole (1.50 g, 6.491 mmol), trimethylsilylacetylene (2.55 g, 25.964 mmol), copper(I) iodide (0.12 g, 0.649 mmol), tetrakis(triphenylphosphine)palladium(0) (0.75 g, 0.65 mmol), bis(triphenylphosphine)palladium(II) dichloride (0.91 g, 1.3 mmol), trimethylamine (15 mL), and N,N-dimethylformamide (15 mL). The reaction was stirred overnight at 90° C. then concentrated under vacuum. The residue was purified on a silica gel column eluting with ethyl acetate/petroleum ether (1:2). Desired fractions were combined and concentrated to yield the title compound (1 g, 62%) as an oil.

### Part II Synthesis of 4-Ethynyl-1-(oxan-2-yl)

[chemical structure]

Into a 100 mL round-bottom flask, was placed 1-(oxan-2-yl)-4-[2-(trimethylsilyl)ethynyl]pyrazole (1.70 g, 6.844 mmol), potassium carbonate (0.09 g, 0.684 mmol) in methanol (20 mL). The resulting solution was stirred overnight at room temperature, then concentrated under vacuum. The residue was purified on a silica gel column eluting with ethyl acetate/petroleum ether (1:2). Desired fractions were combined and concentrated to yield the title compound (1 g, 83%) as an oil.

### Part III—Synthesis of tert-Butyl (8S)-3-[(2-amino-3-[2-[1-(oxan-2-yl)pyrazol-4-yl]ethynyl]pyridin-4-yl)oxy]-8-ethyl-7,8-dihydro-5H-1,6-naphthyridine-6-carboxylate

[chemical structure]

Into a 50 mL round-bottom flask was added 4-ethynyl-1-(oxan-2-yl)pyrazole (153 mg, 0.87 mmol), tert-butyl (8S)-3-[(2-amino-3-iodopyridin-4-yl)oxy]-8-ethyl-7,8-dihydro-5H-1,6-naphthyridine-6-carboxylate (218 mg, 0.87 mmol), tetrakis(triphenylphosphine)palladium(0) (44 mg, 0.09 mmol), bis(triphenylphosphine)palladium(II) dichloride (31 mg, 0.09 mmol), copper(I) iodide (22 mg, 0.18 mmol), triethylamine (5 mL), and N,N-dimethylformamide (5 mL). The reaction was stirred for 1.5 hours at 90° C., then concentrated under vacuum. The residue was purified on a silica gel column eluting with ethyl acetate/petroleum ether (1:1). Desired fractions were combined and concentrated to yield the title compound (130 mg, 54%) as a solid.

### Part IV—Synthesis of tert-Butyl (8S)-8-ethyl-3-([2-[1-(oxan-2-yl)pyrazol-4-yl]-1H-pyrrolo[2,3-b]pyridin-4-yl]oxy)-7,8-dihydro-5H-1,6-naphthyridine-6-carboxylate

[chemical structure]

Into a 20 mL vial was dissolved tert-butyl (8S)-3-[(2-amino-3-[2-[1-(oxan-2-yl)pyrazol-4-yl]ethynyl]pyridin-4-yl)oxy]-8-ethyl-7,8-dihydro-5H-1,6-naphthyridine-6-carboxylate (50 mg, 0.09 mmol) in N,N-dimethylformamide (4 mL). Added potassium tert-butoxide solution (21 mg, 0.18 mmol) and stirred for 1 hour at 100° C. The mixture was extracted with ethyl acetate (3×50 mL), washed with brine (3×50 mL), dried over anhydrous sodium sulfate, and concentrated. The residue was purified on a silica gel column eluting with dichloromethane/methanol (20:1). Desired fractions were combined and concentrated to yield the title compound (25 mg, 50%) as a solid.

### Part V—Synthesis of (8S)-8-ethyl-3-[[2-(1H-pyrazol-4-yl)-1H-pyrrolo[2,3-b]pyridin-4-yl]oxy]-5,6,7,8-tetrahydro-1,6-naphthyridine

[chemical structure]

Into a 50 mL round-bottom flask was dissolved tert-butyl (8S)-8-ethyl-3-([2-[1-(oxan-2-yl)pyrazol-4-yl]-1H-pyrrolo[2,3-b]pyridin-4-yl]oxy)-7,8-dihydro-5H-1,6-naphthyridine-6-carboxylate (20 mg, 0.04 mmol), in dichloromethane (4 mL). Added trifluoroacetic acid (1 mL) and stirred at room temperature for 1 hour. The resulting mixture was concentrated under vacuum to yield the title compound (12 mg, 91%).

### Example 54—tert-Butyl 7-([5-fluoro-2-[1-(oxan-2-yloxy)cyclopropyl]-1H-pyrrolo[2,3-b]pyridin-4-yl]oxy)-3,4-dihydro-1H-isoquinoline-2-carboxylate

[chemical structure]

### Part I—Synthesis of tert-Butyl 7-[(2-amino-5-fluoro-3-[2-[1-(oxan-2-yloxy)cyclopropyl]ethynyl]pyridin-4-yl)oxy]-3,4-dihydro-1H-isoquinoline-2-carboxylate

[chemical structure]

Into a 20 mL vial purged and maintained with an inert atmosphere of nitrogen was placed tert-butyl 7-[(2-amino-3-bromo-5-fluoropyridin-4-yl)oxy]-3,4-dihydro-1H-isoquinoline-2-carboxylate (250 mg, 0.57 mmol), N,N-dimethylformamide (4 mL), 2-(1-ethynylcyclopropoxy)oxane (380 mg, 2.3 mmol), tetrakis(triphenylphosphine)palladium(0) (66 mg, 0.057 mmol), bis(triphenylphosphine)palladium(II) dichloride (40 mg, 0.057 mmol), copper(I) iodide (22 mg, 0.114 mmol), and triethylamine (3 mL). The reaction was stirred for 3 hours at 90° C., then concentrated under vacuum. The residue was purified on a silica gel column eluting with ethyl acetate/petroleum ether (1:4). Desired fractions were combined and concentrated to yield the title compound (160 mg, 54%) as a solid.

### Part II—Synthesis of tert-Butyl 7-([5-fluoro-2-[1-(oxan-2-yloxy)cyclopropyl]-1H-pyrrolo[2,3-b]pyridin-4-yl]oxy)-3,4-dihydro-1H-isoquinoline-2-carboxylate

[chemical structure]

Into a 50 mL round-bottom flask was placed tert-butyl 7-[(2-amino-5-fluoro-3-[2-[1-(oxan-2-yloxy)cyclopropyl]ethynyl]pyridin-4-yl)oxy]-3,4-dihydro-1H-isoquinoline-2-carboxylate (160 mg, 0.31 mmol), N,N-dimethylformamide (10 mL), potassium tert-butoxide (69 mg, 0.61 mmol). The reaction was stirred for 1 hour at 100° C., cooled, and diluted with ethyl acetate (300 mL). Washed with water (3×50 mL), dried over anhydrous sodium sulfate, and concentrated under vacuum. The residue was purified on a silica gel column eluting with ethyl acetate/petroleum ether (1:3). Desired fractions were combined and concentrated to yield the title compound (60 mg, 38%) as a solid.

### Example 55—tert-Butyl 7-[[2-(ethoxycarbonyl)-5-fluoro-1H-pyrrolo[2,3-b]pyridin-4-yl]oxy]-3,4-dihydro-1H-isoquinoline-2-carboxylate

[chemical structure]

### Part I—Synthesis of tert-Butyl 7-[[2-amino-3-(3-ethoxy-3-oxoprop-1-yn-1-yl)-5-fluoropyridin-4-yl]oxy]-3,4-dihydro-1H-isoquinoline-2-carboxylate

[chemical structure]

Into a 20 mL vial purged and maintained with an inert atmosphere of nitrogen was added tert-butyl 7-[(2-amino-3-bromo-5-fluoropyridin-4-yl)oxy]-3,4-dihydro-1H-isoquinoline-2-carboxylate (300 mg, 0.7 mmol), 1,4-dioxane (8 mL), 3,3,3-triethoxyprop-1-yne (350 mg, 2.1 mmol), cesium carbonate (580 mg, 1.78 mmol), copper(I) iodide (13 mg, 0.07 mmol), 2-dicyclohexylphosphino-2′,4′,6′-triisopropylbiphenyl (65 mg, 0.14 mmol), and bis(acetonitrile)dichloropalladium(II) (18 mg, 0.07 mmol). The reaction was stirred for 3 hous at 90° C., cooled, and concentrated under vacuum. The residue was purified on a silica gel column eluting with ethyl acetate/petroleum ether (0:100-40:60). Desired fractions were combined and concentrated to yield the title compound (230 mg, 74%) as a solid.

### Part II—Synthesis of tert-Butyl 7-[[2-(ethoxycarbonyl)-5-fluoro-1H-pyrrolo[2,3-b]pyridin-4-yl]oxy]-3,4-dihydro-1H-isoquinoline-2-carboxylate

[chemical structure]

Into a 100 mL round-bottom flask was dissolved tert-butyl 7-[[2-amino-3-(3-ethoxy-3-oxoprop-1-yn-1-yl)-5-fluoropyridin-4-yl]oxy]-3,4-dihydro-1H-isoquinoline-2-carboxylate (230 mg, 0.51 mmol) in N,N-dimethylformamide (10 mL). Added potassium tert-butoxide (110 mg, 1.01 mmol) and stirred for 2 hous at 100° C. The mixture was diluted with water (20 mL), extracted with ethyl acetate (3×100 mL), dried over anhydrous sodium sulfate and concentrated under vacuum. The residue was purified on a silica gel column eluting with ethyl acetate/petroleum ether (1:1). Desired fractions were combined and concentrated to yield the title compound (130 mg, 57%) as a solid.

### Example 56—tert-Butyl (8S)-3-[[2-(dimethylcarbamoyl)-1-[[2-(trimethylsilyl)ethoxy]methyl]pyrrolo[2,3-b]pyridin-4-yl]oxy]-8-ethyl-7,8-dihydro-5H-1,6-naphthyridine-6-carboxylate

[chemical structure]

### Part I—Synthesis of 4-[[(8S)-6-[(tert-Butoxy)carbonyl]-8-ethyl-5,6,7,8-tetrahydro-1,6-naphthyridin-3-yl]oxy]-1-[[2-(trimethylsilylethoxy]methyl]-1H-pyrrolo[2,3-b]pyridine-2-carboxylic acid

[chemical structure]

Into a 50 mL round-bottom flask was dissolved tert-butyl (8S)-3-[[2-(ethoxycarbonyl)-1-[[2-(trimethylsilyl)ethoxy]methyl]-1H-pyrrolo[2,3-b]pyridin-4-yl]oxy]-8-ethyl-5,6,7,8-tetrahydro-1,6-naphthyridine-6-carboxylate (100 mg, 0.17 mmol) in methanol (2 mL) and water (2 mL). Added lithium hydroxide (40 mg, 1.7 mmol) and stirred for 3 hours at room temperature. The mixture was diluted with water (20 mL), extracted with ethyl acetate (4×20 mL), dried over anhydrous sodium sulfate and concentrated under vacuum. Desired fractions were combined and concentrated to yield the title compound (80 mg, 84%) as a solid.

### Part II—Synthesis of tert-Butyl (8S)-3-[[2-(dimethylcarbamoyl)-1-[[2-(trimethylsilyl)ethoxy]methyl]pyrrolo[2,3-b]pyridin-4-yl]oxy]-8-ethyl-7,8-dihydro-5H-1,6-naphthyridine-6-carboxylate

[chemical structure]

Into a 25 mL round-bottom flask was dissolved 4-[[(8S)-6-(tert-butoxycarbonyl)-8-ethyl-7,8-dihydro-5H-1,6-naphthyridin-3-yl]oxy]-1-[[2-(trimethylsilyl)ethoxy]methyl]pyrrolo[2,3-b]pyridine-2-carboxylic acid (1.5 g, 2.6 mmol) in N,N-dimethylformamide (30 mL). Added, 1-[bis(dimethylamino)methylene]-1H-1,2,3-triazolo[4,5-b]pyridinium 3-oxid hexafluorophosphate (1.5 g, 4 mmol) and diisopropylethylamine (2 mL, 12 mmol), stirred at ambient temperature for 5 minutes, then added dimethylamine (450 mg, 10 mmol). Stirred for 2 hous at room temperature, diluted with ethyl acetate (100 mL), washed with water (3×100 mL), dried over anhydrous sodium sulfate and concentrated. The residue was purified on a silica gel column eluting with petroleum ether:ethyl acetate (1:3). Desired fractions were combined and concentrated to yield the title compound (800 mg, 51%) as a solid.

### Example 57—tert-Butyl (8S)-3-[(2-cyano-1-[[2-(trimethylsilyl)ethoxy]methyl]-1H-pyrrolo[2,3-b]pyridin-4-yl)oxy]-8-ethyl-5,6,7,8-tetrahydro-1,6-naphthyridine-6-carboxylate

[chemical structure]

### Part I—Synthesis of tert-Butyl (8S)-3-[(2-carbamoyl-1-[[2-(trimethylsilyl)ethoxy]methyl]pyrrolo[2,3-b]pyridin-4-yl)oxy]-8-ethyl-7,8-dihydro-5H-1,6-naphthyridine-6-carboxylate

[chemical structure]

Into a 250 mL round-bottom flask was dissolved 4-[[(8S)-6-(tert-butoxycarbonyl)-8-ethyl-7,8-dihydro-5H-1,6-naphthyridin-3-yl]oxy]-1-[[2-(trimethylsilyl)ethoxy]methyl]pyrrolo[2,3-b]pyridine-2-carboxylic acid (12 g, 21 mmol) in N,N-dimethylformamide (200 mL). Added N,N-diisopropylethylamine (14.7 mL, 84 mmol), 1-[bis(dimethylamino)methylene]-1H-1,2,3-triazolo[4,5-b]pyridinium 3-oxid hexafluorophosphate (16 g, 42 mmol), ammonium chloride (2.3 g, 42 mmol), then stirred for 2 hours at room temperature. The reaction was diluted with ethyl acetate (400 mL), washed with brine (3×150 mL), separated layers and concentrated organics under vacuum. The residue was purified on a silica gel column eluting with ethyl acetate in petroleum ether. Desired fractions were combined and concentrated to yield the title compound (7.5 g, 63%) as an oil.

### Part II—4. Synthesis of tert-Butyl (8S)-3-[(2-cyano-1-[[2-(trimethylsilyl)ethoxy]methyl]-1H-pyrrolo[2,3-b]pyridin-4-yl)oxy]-8-ethyl-5,6,7,8-tetrahydro-1,6-naphthyridine-6-carboxylate

[chemical structure]

Into a 250 mL round-bottom flask was dissolved tert-butyl (8S)-3-[(2-carbamoyl-1-[[2-(trimethylsilyl)ethoxy]methyl]-1H-pyrrolo[2,3-b]pyridin-4-yl)oxy]-8-ethyl-5,6,7,8-tetrahydro-1,6-naphthyridine-6-carboxylate (7 g, 12 mmol) in 1,4-dioxane (110 mL) Added triethylamine (5.2 mL, 37 mmol) and trifluoroacetic anhydride (5.7 g, 27 mmol). The reaction was stirred overnight at room temperature, diluted with ethyl acetate (300 mL), washed with brine (3×150 mL), then purified on a silica gel column eluting with ethyl acetate in petroleum ether. Desired fractions were combined and concentrated to yield the title compound (6.5 g, 96%) as an oil.

### Example 58—7-[[5-Fluoro-2-(1-methylpyrazol-4-yl)-1H-pyrrolo[2,3-b]pyridin-4-yl]oxy]-N-[4-[(4-methylpiperazin-1-yl)methyl]-3-(trifluoromethyl)phenyl]-3,4-dihydro-1H-isoquinoline-2-carboxamide (Compound No. 19)

[chemical structure]

### Part I—Synthesis of tert-Butyl 7-([2-amino-5-fluoro-3-[2-(1-methylpyrazol-4-yl)ethynyl]pyridin-4-yl]oxy)-3,4-dihydro-1H-isoquinoline-2-carboxylate

[chemical structure]

In a vial purged and maintained with an inert atmosphere of nitrogen was added tert-butyl 7-[(2-amino-3-bromo-5-fluoropyridin-4-yl)oxy]-3,4-dihydro-1H-isoquinoline-2-carboxylate (300 mg, 0.68 mmol), 4-ethynyl-1-methylpyrazole (220 mg, 2.1 mmol), 2-dicyclohexylphosphino-2′,4′,6′-triisopropylbiphenyl (65 mg, 0.14 mmol), bis(acetonitrile)dichloropalladium(II) (18 mg, 0.07 mmol), cesium carbonate (580 mg, 1.8 mmol), 1,4-dioxane (15 mL), and copper(I) iodide (13 mg, 0.07 mmol). The mixture was stirred overnight at 90° C., then concentrated. The residue was purified on a silica gel column eluting with ethyl acetate/petroleum ether. Desired fractions were combined and concentrated to yield the title compound (250 mg, 79%) as a solid.

### Part II—Synthesis of tert-Butyl 7-[[5-fluoro-2-(1-methylpyrazol-4-yl)-1H-pyrrolo[2,3-b]pyridin-4-yl]oxy]-3,4-dihydro-1H-isoquinoline-2-carboxylate

[chemical structure]

Into a 25 mL vial purged and maintained with an inert atmosphere of nitrogen was dissolved tert-butyl 7-([2-amino-5-fluoro-3-[2-(1-methylpyrazol-4-yl)ethynyl]pyridin-4-yl]oxy)-3,4-dihydro-1H-isoquinoline-2-carboxylate (200 mg, 0.43 mmol) in N,N-dimethylformamide (10 mL), added potassium tert-butoxide (97 mg, 0.86 mmol) and stirred for 2 hours at 100° C. The cooled mixture was diluted with ethyl acetate (100 mL), washed with brine (3×30 ml), dried over anhydrous sodium sulfate and concentrated. The residue was purified on a silica gel column eluting with ethyl acetate/hexane (100%). Desired fractions were combined and concentrated to yield the title compound (130 mg, 65%) as a solid.

### Part III—Synthesis of 7-[[5-Fluoro-2-(1-methylpyrazol-4-yl)-1H-pyrrolo[2,3-b]pyridin-4-yl]oxy]-1,2,3,4-tetrahydroisoquinoline

[chemical structure]

In round-bottom flask was dissolved tert-butyl 7-[[5-fluoro-2-(1-methylpyrazol-4-yl)-1H-pyrrolo[2,3-b]pyridin-4-yl]oxy]-3,4-dihydro-1H-isoquinoline-2-carboxylate (100 mg, 0.22 mmol) in dichloromethane (5 mL), added trifluoroacetic acid (1 mL), and stirred at room temperature for 1 hour. The resulting mixture was concentrated to yield the crude title compound (70 mg, 89%) as a solid.

### Part IV—Synthesis of 7-[[5-Fluoro-2-(1-methylpyrazol-4-yl)-1H-pyrrolo[2,3-b]pyridin-4-yl]oxy]-N-[4-[(4-methylpiperazin-1-yl)methyl]-3-(trifluoromethyl)phenyl]-3,4-dihydro-1H-isoquinoline-2-carboxamide (Compound No. 19)

[chemical structure]

In a 25 mL round-bottom flask was dissolved 7-[[5-fluoro-2-(1-methylpyrazol-4-yl)-1H-pyrrolo[2,3-b]pyridin-4-yl]oxy]-1,2,3,4-tetrahydroisoquinoline (35 mg, 0.1 mmol) in dichloromethane (5 mL), added triethylamine (0.11 mL, 0.8 mmol) and phenyl N-[4-[(4-methylpiperazin-1-yl)methyl]-3-(trifluoromethyl)phenyl]carbamate (49 mg, 0.13 mmol). The solution was stirred overnight at room temperature, concentrated, and purified by preparatory-HPLC. Desired fractions were combined and concentrated to yield the title compound (26 mg, 34%) as the trifluoroacetate salt. H-NMR— (400 MHz, Methanol-d₄) δ 8.13 (d, J=3.8 Hz, 1H), 7.97 (s, 1H), 7.86-7.78 (m, 2H), 7.72-7.61 (m, 2H), 7.24 (d, J=8.3 Hz, 1H), 6.97 (dd, J=8.3, 2.7 Hz, 1H), 6.93 (d, J=2.6 Hz, 1H), 6.08 (d, J=0.8 Hz, 1H), 4.68 (s, 2H), 3.92 (s, 3H), 3.81 (t, J=5.9 Hz, 2H), 3.74 (d, J=1.7 Hz, 2H), 3.56-3.40 (m, 2H), 3.30-3.04 (m, 4H), 2.97 (t, J=5.9 Hz, 2H), 2.92 (s, 3H), 2.61-2.32 (m, 2H).

### Example 59—Synthesis of Additional N-phenyl-3,4-dihydroisoquinoline-2(1H)-carboxamides and Related Compounds

The compounds in Table 12 below were prepared based on experimental procedures described in Example 58 and in the detailed description above.

TABLE 12

| | | Mass | |
|---|---|---|---|
| | | Spec. | |
| No. | Chemical Structure | (ES, m/z) | ¹H NMR Spectrum |
| XII-1 | [chemical structure] | 664.40 [M + H]⁺ | (400 MHz, Methanol-d4) δ 8.91 (d, J = 2.4 Hz, 1H), 8.34 (d, J = 2.4 Hz, 1H), 8.13 (d, J = 3.9 Hz, 1H), 7.97 (s, 1H), 7.82 (d, J = 0.8 Hz, 1H), 7.25 (d, J = 8.3 Hz, 1H), 7.03-6.89 (m, 2H), 6.09 (s, 1H), 4.70 (s, 2H), 3.92 (s, 5H), 3.83 (t, J = 5.9 Hz, 2H), 3.32-3.20 (m, 5H), 2.98 (t, J = 5.9 Hz, 2H), 2.89 (s, 6H). |
| XII-2 | [chemical structure] | 691.40 [M + H]⁺ | (400 MHz, Methanol-d4, ppm) δ 8.14 (d, J = 3.9 Hz, 1H), 7.97 (s, 1H), 7.82 (dd, J = 5.0, 1.5 Hz, 2H), 7.76-7.63 (m, 2H), 7.24 (d, J = 9.1 Hz, 1H), 6.96 (d, J = 6.5 Hz, 2H), 6.03 (d, J = 1.1 Hz, 1H), 5.32 (q, J = 6.8 Hz, 1H), 4.24-4.13 (m, 1H), 3.92 (s, 3H), 3.75 (s, 2H), 3.64- 3.44 (m, 3H), 3.23 (q, J = 7.3 Hz, 2H), 3.18-2.84 (m, 6H), 2.56-2.39 (m, 2H), 1.51 (d, J = 6.8 Hz, 3H), 1.36 (t, J = 7.3 Hz, 3H). |
| XII-3 | [chemical structure] | 677.3 [M + H]⁺ | N/A |
| XII-4 | [chemical structure] | 681.35 [M + H]⁺ | N/A |
| XII-5 | [chemical structure] | 660.30 [M + H]⁺ | N/A |
| XII-6 | [chemical structure] | 661.50 [M + H]⁺ | N/A |
| XII-7 | [chemical structure] | 660.30 [M + H]⁺ | N/A |
| XII-8 | [chemical structure] | 650.35 [M + H]⁺ | N/A |
| XII-9 | [chemical structure] | 649.35 [M + H]⁺ | N/A |
| XII-10 | [chemical structure] | 661.30 [M + H]⁺ | N/A |
| XII-11 | [chemical structure] | 678.30 [M + H]⁺ | N/A |
| XII-12 | [chemical structure] | 677.35 [M + H]⁺ | N/A |
| XII-13 | [chemical structure] | 665.25 [M + H]⁺ | N/A |
| XII-14 | [chemical structure] | 693.40 [M + H]⁺ | N/A |
| XII-15 | [chemical structure] | 677.30 [M + H]⁺ | N/A |
| XII-16 | [chemical structure] | 681.40 [M + H]⁺ | N/A |

### Example 60—Synthesis of Additional 1-methyl-N-phenyl-3,4-dihydroisoquinoline-2(1H)-carboxamides and Related Compounds

The compounds in Table 13 below were prepared based on experimental procedures described in Example 9 and in the detailed description. All compounds in Table 13 were prepared as single enantiomers with the absolute stereochemistry as shown.

TABLE 13

| | | Mass Spec. | |
|---|---|---|---|
| No. | Chemical Structure | (ES, m/z) | ¹H NMR Spectrum |
| XIII-1 | [chemical structure] | 623.25 [M + H]⁺ | N/A |
| XIII-2 | [chemical structure] | 623.2 [M + H]⁺ | (400 MHz, DMSO-d6) δ 11.6 (s, 1H), 8.78 (s, 1H), 8.02 (m, 1H), 7.88 (m, 1H), 7.75 (m, 1H), 7.58 (m, 1H), 7.24 (m, 1H), 7.04 (m, 1H), 6.95 (m, 1H), 6.40 (m, 1H), 6.05 (s, 1H), 5.35 (m, 1H), 5.2 (m, 1H), 4.52 (m, 2H), 4.18 (m, 1H), 3.5 (s, 2H), 2.4-2.2 (m, 8H), 1.4 (m, 3H), 0.95 (m, 6H) |
| XIII-3 | [chemical structure] | 611.2 [M + H]⁺ | (400 MHz, DMSO-d6) δ 11.62 (s, 1H), 8.78 (s, 1H), 8.46 (m, 1H), 8.18 (m, 1H), 8.01 (m, 7.24 (m, 1H), 7.05 (m, 1H), 6.05 (s, 1H), 5.36 (m, 1H), 5.22 (m, 1H), 5.12 (m, 4.55 (m, 2H), 4.16 (m, 1H), 2.3 (m, 2H), 2.18 (m, 3H), 1.9 (m, 2H), 1.7 (m, 2H), 1.4 (m, 3H). |
| XIII-4 | [chemical structure] | 653.1 [M + H]⁺ | (400 MHz, DMSO-d6) δ 11.8 (s, 1H), 8.81 (s, 1H), 8.48 (m, 1H), 8.2 (m, 1H), 8.04 (m, 1H), 7.26 (m, 1H), 7.05 (m, 1H), 6.98 (m, 1H), 6.42 (m, 1H), 6.38 (m, 1 (s, 1H), 5.36 (m, 1H), 5.32 (m, 1H), 5.12 (m, 1H), 4.32 (m, 1H), 4.14 (m, 1H), 4.8 (m, 2H), 4.68 (m, 2H), 2.0 (m, 3H), 1.78 (m, 2H), 1.4 (m, 3H), 1.2 (m, 4H). |
| XIII-5 | [chemical structure] | 639.1 [M + H]⁺ | (400 MHz, DMSO-d6) δ 11.86 (s, 1H), 8.84 (s, 1H), 8.08 (m, 1H), 7.84 (m, 1H), 7.8 (m, 1H), 7.58 (m, 1H), 7.24 (m, 1H), 7.2 (m, 1H), 7.1 (m, 2H), 6.99 (m, 1H), 6.44 (m, 1H), 6.16 (s, 1H), 5.38 (m, 1H), 4.56 (m, 2H), 4.18 (m, 1H), 4.0-3.0 (m, 12H), 1.42 (m, 3H). |
| XIII-6 | [chemical structure] | 681.2 [M + H]⁺ | (400 MHz, DMSO-d6) δ 12.12 (s, 1H), 8.84 (s, 1H), 8.10 (m, 1H), 7.92 (m, 1H), 7.8 (m, 1H), 7.58 (m, 1H), 7.28 (m, 1H), 7.12 (m, 1H), 7.02 (m, 1H), 6.44 (m, 2H), 4.8 (m, 2H), 4.72 (m, 2H), 4.56 (m, 2H), 4.18 (m, 1H), 3.7-2.8 (m, 12H), 1.40 (m, 3H). |
| XIII-7 | [chemical structure] | 637.1 [M + H]⁺ | (400 MHz, DMSO-d6) δ 12.21 (s, 1H), 8.92 (s, 1H), 8.62 (br s, 2H), 8.15 (m, 1H), 7.92 (m, 1H), 7.80 (m, 1H), 7.59 (m, 1H), 7.28 (m, 1H), 7.15 (m, 1H), 7.04 (m, 1H), 6.48 (m, 2H), 5.38 (m, 2H), 4.8 (m, 2H), 4.72 (m, 2H), 4.18 (m, 1H), 3.66 (m, 2H), 3.36 (m, 1H), 3.12 (m, 4H), 2.84 (m, 1H), 2.62 (m, 4H), 1.44 (m, 3H). |
| XIII-8 | [chemical structure] | 595.1 [M + H]⁺ | (400 MHz, DMSO-d6) δ 12.15 (s, 1H), 8.88 (s, 1H), 8.6 (br s, 1H), 8.12 (m, 1H), 7.91 (m, 1H), 7.80 (m, 1H), 7.58 (m, 1H), 7.26 (m, 1H), 7.12 (m, 1H), 7.02 (m, 1H), 6.52 (m, 1H), 6.18 (s, 1H), 5.35 (m, 1H), 4.2 (m, 1H), 4.52 (m, 2H), 4.18 (m, 1H), 3.64 (m, 2H), 3.36 (m, 1H), 3.1 (m, 4H), 2.84 (m, 1H), 2.62 (m, 4H), 1.40 (m, 3H). |
| XIII-9 | [chemical structure] | 591.2 [M + H]⁺ | (400 MHz, DMSO-d6) δ 12.16 (s, 1H), 8.78 (s, 1H), 8.12 (m, 1H), 7.80 (m, 1H), 7.62 (m, 1H), 7.32-7.26 (m, 2H), 7.16 (m, 1H), 7.02 (m, 1H), 6.52 (m, 1H), 6.1 (s, 1H), 5.35 (m, 1H), 4.58 (m, 2H), 4.2 (m, 1H), 3.62 (m, 2H), 3.36 (m, 1H), 3.0-2.8 (m, 2H), 2.78 (s, 3H), 2.3 (m, 2H), 1.40 (m, 3H). |
| XIII-10 | [chemical structure] | 633.2 [M + H]⁺ | (400 MHz, DMSO-d6) δ 12.12 (s, 1H), 8.78 (s, 1H), 8.12 (m, 1H), 7.79 (m, 1H), 7.62 (m, 1H), 7.32-7.25 (m, 2H), 7.14 (m, 1H), 7.02 (m, 1H), 6.54 (m, 1H), 5.36 (m, 1H), 4.82 (m, 2H), 4.70 (m, 2H), 4.18 (m, 1H), 3.36 (m, 1H), 3.0-2.8 (m, 2H), 2.78 (s, 3H), 2.3 (m, 2H), 1.42 (m, 3H). |
| XIII-11 | [chemical structure] | 663.2 [M + H]⁺ | (400 MHz, DMSO-d6) δ 12.18 (s, 1H), 8.78 (s, 1H), 8.1 (m, 1H), 7.78 (m, 1H), 7.64 (m, 1H), 7.3-7.25 (m, 2H), 7.16 (m, 1H), 7.02 (m, 1H), 6.48 (m, 1H), 5.38 (m, 1H), 4.82 (m, 2H), 4.70 (m, 2H), 4.18 (m, 1H), 3.7-3.6 (m, 4H), 3.5- 3.3 (m, 4H), 3.18 (m, 2H), 3.1- 2.82 (m, 4H), 1.4 (m, 3H). |
| XIII-12 | [chemical structure] | 621.2 [M + H]⁺ | (400 MHz, DMSO-d6) δ 12.18 (s, 1H), 8.80 (s, 1H), 8.12 (m, 1H), 7.78 (m, 1H), 7.64 (m, 1H), 7.32-7.24 (m, 2H), 7.16 (m, 1H), 7.02 (m, 1H), 6.54 (m, 1H), 6.2 (s, 1H), 5.35 (m, 1H), 4.56 (m, 2H), 4.18 (m, 1H), 3.7-3.62 (m, 4H), 3.5-3.3 (m, 2H), 3.16 (m, 2H), 3.05- 2.82 (m, 4H), 1.41 (m, 3H). |
| XIII-13 | [chemical structure] | 645.1 [M + H]⁺ | 1H NMR (400 MHz, DMSO- d6) δ ppm 0.94 (t, J = 1.00 Hz, 3H) 1.42 (d, J = 1.00 Hz, 3H) 2.10-2.44 (m, 8H) 2.75-2.98 (m, 3H) 3.18-3.39 (m, 3H) 3.51-3.61 (m, 3H) 5.33 (q, J = 1.00 Hz, 1H) 6.89-7.08 (m, 2H) 7.08-7.21 (m, 2H) 7.21- 7.32 (m, 1H) 7.33-7.45 (m, 1H) 7.50-7.62 (m, 1H) 7.66- 7.81 (m, 1H) 7.83-7.94 (m, 1H) 8.20-8.34 (m, 1H) 8.72- 8.90 (m, 1H) 10.08-10.41 (m, 2H) |
| XIII-14 | [chemical structure] | 693.1 [M + H]⁺ | N/A |
| XIII-15 | [chemical structure] | 655.1 [M + H]+ | N/A |
| XIII-16 | [chemical structure] | 683.25 [M + H]⁺ | (400 MHz, Methanol-d4, ppm): δ 8.22 (s, 1H), 7.83 (s, 1H), 7.75-7.58 (m, 2H), 7.25 (d, J = 8.9 Hz, 1H), 6.98 (m, 2H), 5.94 (m, 1H), 5.33 (q, J = 6.7 Hz, 1H), 4.80 (s, 4H), 4.18 (m, 1H), 3.75 (s, 2H), 3.50 (M, 2H), 3.23 (q, J = 7.3 Hz, 3H), 3.17-2.80 (m, 5H), 2.49 (s, 2H), 1.52 (d, J = 6.7 Hz, 3H), 1.36 (t, J = 7.3 Hz, 3H) |
| XIII-17 | [chemical structure] | 695.2 [M + H]⁺ | N/A |

### Example 61—(8S)-3-[(2-Cyano-1H-pyrrolo[2,3-b]pyridin-4-yl)oxy]-8-ethyl-N-[4-[(4-methylpiperazin-1-yl)methyl]-3-(trifluoromethyl)phenyl]-7,8-dihydro-5H-1,6-naphthyridine-6-carboxamide (Compound No. 20)

[chemical structure]

### Part I—Synthesis of 4-[[(8S)-8-Ethyl-5,6,7,8-tetrahydro-1,6-naphthyridin-3-yl]oxy]-1H-pyrrolo[2,3-b]pyridine-2-carbonitrile

[chemical structure]

Into a 100 mL round-bottom flask was dissolved tert-butyl (8S)-3-[(2-cyano-1-[[2-(trimethylsilyl)ethoxy]methyl]pyrrolo[2,3-b]pyridin-4-yl)oxy]-8-ethyl-7,8-dihydro-5H-1,6-naphthyridine-6-carboxylate (12 g, 21.8 mmol) in dichloromethane (90 mL), added trifluoroacetic acid (30 mL) and stirred for 1.5 hours at room temperature. The mixture was concentrated under vacuum to yield crude title compound (3.6 g, 52%).

### Part II—Synthesis of (8S)-3-[(2-Cyano-1H-pyrrolo[2,3-b]pyridin-4-yl)oxy]-8-ethyl-N-[4-[(4-methylpiperazin-1-yl)methyl]-3-(trifluoromethyl)phenyl]-7,8-dihydro-5H-1,6-naphthyridine-6-carboxamide (Compound No. 20)

[chemical structure]

In a 100 mL round-bottom flask was dissolved 4-[[(8S)-8-ethyl-5,6,7,8-tetrahydro-1,6-naphthyridin-3-yl]oxy]-1H-pyrrolo[2,3-b]pyridine-2-carbonitrile (1.8 g, 5.6 mmol) in dichloromethane (120 mL), added triethylamine (1.6 mL, 11.3 mmol) and phenyl N-[4-[(4-methylpiperazin-1-yl)methyl]-3-(trifluoromethyl)phenyl]carbamate (2.7 g, 6.8 mmol). The reaction was stirred overnight at room temperature, then concentrated under vacuum. The crude product was purified by preparatory HPLC and pure fractions were combined and concentrated to yield the title compound (1.65 g, 40%) as the trifluoroacetate salt. H-NMR— (400 MHz, Methanol-d₄) δ 8.83 (d, J=2.5 Hz, 1H), 8.55 (d, J=6.1 Hz, 1H), 8.24 (d, J=2.6 Hz, 1H), 8.02 (s, 1H), 7.92-7.80 (m, 2H), 7.50 (d, J=1.2 Hz, 1H), 7.02 (d, J=6.1 Hz, 1H), 5.24 (d, J=17.7 Hz, 1H), 4.74 (d, J=17.6 Hz, 1H), 4.47 (d, J=16.5 Hz, 1H), 4.32 (s, 2H), 3.68 (s, 2H), 3.63 (dd, J=13.9, 3.8 Hz, 2H), 3.23 (q, J=7.2 Hz, 4H), 3.00 (s, 3H), 1.96-1.78 (m, 2H), 1.34 (t, J=7.3 Hz, 2H), 1.20 (t, J=7.3 Hz, 3H).

### Example 62—(8S)-3-[[2-(dimethylcarbamoyl)-1H-pyrrolo[2,3-b]pyridin-4-yl]oxy]-8-ethyl-N-[4-[(4-methylpiperazin-1-yl)methyl]-3-(trifluoromethyl)phenyl]-7,8-dihydro-5H-1,6-naphthyridine-6-carboxamide (Compound No. 21)

[chemical structure]

### Part I—Synthesis of 4-[[(8S)-8-ethyl-5,6,7,8-tetrahydro-1,6-naphthyridin-3-yl]oxy]-N,N-dimethyl-1H-pyrrolo[2,3-b]pyridine-2-carboxamide

[chemical structure]

In a 50 mL round-bottom flask was dissolved tert-butyl (8S)-3-[[2-(dimethylcarbamoyl)-1-[[2-(trimethylsilyl)ethoxy]methyl]-1H-pyrrolo[2,3-b]pyridin-4-yl]oxy]-8-ethyl-5,6,7,8-tetrahydro-1,6-naphthyridine-6-carboxylate (300 mg, 0.5 mmol) in dichloromethane (5 mL), added trifluoroacetic acid (1 mL) and stirred for 2 hours at room temperature. The resulting mixture was concentrated under vacuum, then added 7M ammonia in methanol (5 mL) and stirred for one hour at room temperature. The mixture was concentrated under vacuum to yield the crude title compound (170 mg, quant. crude yield) as a solid.

### Part II—Synthesis of (8S)-3-[[2-(dimethylcarbamoyl)-1H-pyrrolo[2,3-b]pyridin-4-yl]oxy]-8-ethyl-N-[4-[(4-methylpiperazin-1-yl)methyl]-3-(trifluoromethyl)phenyl]-7,8-dihydro-5H-1,6-naphthyridine-6-carboxamide (Compound No. 21)

[chemical structure]

In a 25 mL round-bottom flask was dissolved 4-[[(8S)-8-ethyl-5,6,7,8-tetrahydro-1,6-naphthyridin-3-yl]oxy]-N,N-dimethyl-1H-pyrrolo[2,3-b]pyridine-2-carboxamide (30 mg, 0.08 mmol) in dichloromethane (5 mL), added triethylamine (0.5 mL, 3.6 mmol) followed by phenyl N-[4-[(4-methylpiperazin-1-yl)methyl]-3-(trifluoromethyl)phenyl]carbamate (40 mg, 0.1 mmol). The solution was stirred overnight at 35° C., then concentrated. The crude product was purified by preparatory HPLC. Desired fractions were combined and concentrated to yield the title compound (27 mg, 49%) as a solid. 1H-NMR (400 MHz, Methanol-d₄) δ 8.40 (d, J=2.6 Hz, 1H), 8.25 (d, J=5.5 Hz, 1H), 7.82 (d, J=2.1 Hz, 1H), 7.73-7.62 (m, 2H), 7.57 (d, J=2.6 Hz, 1H), 6.79 (s, 1H), 6.59 (d, J=5.6 Hz, 1H), 4.97 (d, J=17.0 Hz, 1H), 4.65 (d, J=17.0 Hz, 1H), 4.25 (dd, J=13.6, 4.2 Hz, 1H), 3.65 (d, J=9.4 Hz, 3H), 3.35-3.33 (m, 3H), 3.16 (s, 3H), 3.04-2.94 (m, 1H), 2.53 (s, 8H), 2.31 (s, 3H), 2.01-1.87 (m, 1H), 1.78-1.62 (m, 1H), 1.13 (t, J=7.4 Hz, 3H).

### Example 63—Synthesis of Additional N-phenyl-5,6,7,8-tetrahydro-1,6-naphthyridine-6-carboxamides and Related Compounds

The compounds in Table 14 below were prepared based on experimental procedures described in Example 61 and 62 and in the detailed description.

TABLE 14

| | | Mass | |
|---|---|---|---|
| | | Spec. | |
| No. | Chemical Structure | (ES, m/z) | ¹H NMR Spectrum |
| XIV- 1 | [chemical structure] | 624.3 [M + H]⁺ | N/A |
| | Enantiomer B | | |
| XIV- 2 | [chemical structure] | 622.3 [M + H]⁺ | N/A |
| | Enantiomer B | | |
| XIV- 3 | [chemical structure] | 623 [M + H]⁺ | (300 MHz, Methanol-d4) δ 8.69 (d, J = 2.5 Hz, 1H), 8.26 (s, 1H), 8.05 (d, J = 2.5 Hz, 1H), 7.86 (d, J = 1.9 Hz, 1H), 7.77-7.65 (m, 2H), 6.36 (d, J = 1.1 Hz, 1H), 5.15 (d, J = 17.3 Hz, 1H), 4.70 (d, J = 17.3 Hz, 1H), 4.43 (dd, J = 13.8, 3.4 Hz, 1H), 3.82 (s, 2H), 3.59 (dd, J = 13.8, 3.8 Hz, 1H), 3.24 (q, J = 7.3 Hz, 2H), 3.13 (dd, J = 9.3, 4.4 Hz, 1H), 2.84 (s, 3H), 2.51 (d, J = 1.1 Hz, 3H), 1.97-1.86 (m, 1H), 1.77 (ddd, J = 13.9, 9.5, 7.1 Hz, 1H), 1.36 (t, J = 7.3 Hz, 3H), 1.17 (t, J = 7.4 Hz, 3H). |
| | Enantiomer B | | |
| XIV- 4 | [chemical structure] | 623 [M + H]⁺ | (300 MHz, Methanol-d4) δ 8.65 (s, 1H), 8.25 (s, 1H), 7.99 (s, 1H), 7.86 (s, 1H), 7.79-7.64 (m, 2H), 6.36 (d, J = 1.2 Hz, 1H), 5.13 (d, J = 17.2 Hz, 1H), 4.70 (d, J = 17.3 Hz, 1H), 4.41 (d, J = 13.6 Hz, 1H), 3.80 (s, 2H), 3.59 (dd, J = 13.7, 3.8 Hz, 1H), 3.24 (q, J = 7.3 Hz, 3H), 2.99 (d, J = 69.7 Hz, 5H), 2.51 (d, J = 1.1 Hz, 3H), 1.99-1.85 (m, 1H), 1.76 (dt, J = 14.9, 7.5 Hz, 1H), 1.36 (t, J = 7.3 Hz, 3H), 1.17 (t, J = 7.3 Hz, 3H). |
| | Enantiomer A | | |
| XIV- 5 | [chemical structure] | 607.29 [M + H]⁺ | (300 MHz, Methanol-d4) δ 8.51 (s, 1H), 8.32 (d, J = 6.7 Hz, 1H), 7.84 (s, 1H), 7.72 (d, J = 9.1 Hz, 3H), 7.58 (d, J = 3.5 Hz, 1H), 6.90 (d, J = 6.8 Hz, 1H), 6.70 (s, 1H), 5.02 (d, J = 17.2 Hz, 1H), 4.69 (d, J = 17.2 Hz, 1H), 4.30-4.16 (m, 1H), 3.77 (s, 2H), 3.75-3.66 (m, 1H), 3.23 (q, J = 7.3 Hz, 4H), 3.04 (s, 2H), 1.97 (s, 1H), 1.74 (d, J = 8.4 Hz, 1H), 1.35 (dt, J = 9.6, 7.3 Hz, 5H), 1.14 (t, J = 7.4 Hz, 3H). |
| XIV- 6 | [chemical structure] | 608 [M + H]⁺ | (400 MHz, Methanol-d4) δ 8.52 (d, J = 2.7 Hz, 1H), 8.33 (d, J = 6.7 Hz, 1H), 7.85 (d, J = 2.0 Hz, 1H), 7.80-7.67 (m, 3H), 7.60 (d, J = 3.6 Hz, 1H), 6.92 (d, J = 6.7 Hz, 1H), 6.71 (d, J = 3.6 Hz, 1H), 5.03 (d, J = 17.1 Hz, 1H), 4.70 (d, J = 17.2 Hz, 1H), 4.25 (dd, J = 13.7, 4.4 Hz, 1H), 3.79 (s, 2H), 3.71 (dd, J = 13.6, 4.1 Hz, 1H), 3.23 (q, J = 7.3 Hz, 3H), 3.05 (dd, J = 9.0, 4.6 Hz, 5H), 1.97 (dq, J = 14.3, 7.6 Hz, 1H), 1.74 (dq, J = 14.2, 7.4 Hz, 1H), 1.36 (t, J = 7.3 Hz, 3H), 1.15 (t, J = 7.4 Hz, 3H). |
| XIV- 7 | [chemical structure] | 623.2 [M + H]⁺ | NMR (400 MHz, DMSO-d6) δ ppm 1.15 (t, J = 1.00 Hz, 3 H) 1.32 (d, J = 1.00 Hz, 3 H) 2.23-2.44 (m, 2 H) 2.79-3.02 (m, 3 H) 3.04- 3.20 (m, 3 H) 3.30-3.58 (m, 3 H) 3.58-3.72 (m, 2 H) 3.81-4.03 (m, 1 H) 4.68 (s, 2 H) 6.85-7.09 (m, 1 H) 7.44-7.65 (m, 2 H) 7.69- 7.83 (m, 1 H) 7.83-7.97 (m, 1 H) 8.28-8.52 (m, 3 H) 8.86-9.06 (m, 1 H) 9.08-9.74 (m, 2H) 12.25- 12.95 (m, 2 H) |
| | Enantiomer B | | |
| XIV- 8 | [chemical structure] | 623.2 [M + H]⁺ | N/A |
| | Enantiomer B | | |
| XIV- 9 | [chemical structure] | 609.35 [M + H]⁺ | N/A |
| XIV- 10 | [chemical structure] | 609 [M + H]⁺ | (400 MHz, Methanol-d4) δ 8.95 (d, J = 2.4 Hz, 1H), 8.51 (d, J = 2.6 Hz, 1H), 8.38 (d, J = 2.4 Hz, 1H), 8.32 (d, J = 6.6 Hz, 1H), 7.73 (d, J = 2.6 Hz, 1H), 7.58 (d, J = 3.6 Hz, 1H), 6.89 (d, J = 6.7 Hz, 1H), 6.69 (d, J = 3.5 Hz, 1H), 5.03 (d, J = 17.1 Hz, 1H), 4.74 (d, J = 17.2 Hz, 1H), 4.25 (dd, J = 13.5, 4.6 Hz, 1H), 4.04 (s, 2H), 3.74 (dd, J = 13.5, 4.1 Hz, 1H), 3.23 (q, J = 7.3 Hz, 2H), 3.11-2.86 (m, 5H), 1.98 (ddd, J = 13.1, 7.5, 4.9 Hz, 1H), 1.74 (dp, J = 15.0, 7.5 Hz, 1H), 1.36 (t, J = 7.3 Hz, 3H), 1.15 (t, J = 7.4 Hz, 3H). |
| XIV- 11 | [chemical structure] | 684.38 [M + H]⁺ | N/A |
| | Enantiomer B | | |
| XIV- 12 | [chemical structure] | 611.4 [M + H]⁺ | N/A |
| | Enantiomer B | | |
| XIV- 13 | [chemical structure] | 624.2 [M + H]⁺ | N/A |
| | Enantiomer A | | |
| XIV- 14 | [chemical structure] | 624.25 [M + H]⁺ | (400 MHz, Methanol-d4) δ 8.48 (d, J = 2.6 Hz, 1H), 8.24 (d, J = 6.7 Hz, 1H), 7.84 (d, J = 2.1 Hz, 1H), 7.74-7.63 (m, 3H), 6.88 (d, J = 6.8 Hz, 1H), 6.56 (s, 1H), 4.93 (d, J = 17.2 Hz, 1H), 4.78 (d, J = 1.1 Hz, 3H), 3.92 (dd, J = 13.6, 4.6 Hz, 1H), 3.82 (dd, J = 13.7, 6.0 Hz, 1H), 3.75 (s, 2H), 3.22-3.02 (m, 3H), 2.56 (s, 2H), 1.44 (d, J = 7.0 Hz, 3H), 1.34 (t, J = 7.3 Hz, 3H). |
| XIV- 15 | [chemical structure] | 635.0 [M + H]⁺ | N/A |
| | Enantiomer A | | |
| XIV- 16 | [chemical structure] | 614.2 [M + H]⁺ | (400 MHz, DMSO-d6) δ ppm 0.95 (t, J = 1.00 Hz, 3H) 1.30 (d, J = 1.00 Hz, 3H) 2.24 (q, J = 1.00 Hz, 3H) 2.32-2.44 (m, 6H) 2.96-3.12 (m, 1H) 3.21-3.28 (m, 2H) 3.38-3.56 (m, 5H) 3.93 (dd, J = 1.00 Hz, 1H) 4.58-4.78 (m, 3H) 5.84-5.97 (m, 1H) 6.15 (dd, J = 1.00 Hz, 1H) 6.52 (t, J = 1.00 Hz, 1H) 7.36-7.47 (m, 1H) 7.49-7.61 (m, 1H) 7.66-7.78 (m, 1H) 7.78-7.92 (m, 2H) 8.22- 8.34 (m, 1H) 8.83-8.98 (m, 1H) |
| | Enantiomer B | | |
| XIV- 17 | [chemical structure] | 596.2 [M + H]⁺ | N /A |
| | Enantiomer A | | |
| XIV- 18 | [chemical structure] | 588.2 [M + H]⁺ | N/A |
| | Enantiomer A | | |
| XIV- 19 | [chemical structure] | 633.25 [M + H]⁺ | N/A |
| XIV- 20 | [chemical structure] | 633.25 [M + H]⁺ | N/A |
| XIV- 21 | [chemical structure] | 666.45 [M + H]⁺ | N/A |
| XIV- 22 | [chemical structure] | 667 [M + H]⁺ | (400 MHz, Methanol-d4) δ 8.53 (d, J = 2.5 Hz, 1H), 8.24 (d, J = 6.8 Hz, 1H), 7.86 (s, 1H), 7.77-7.73 (m, 1H), 7.71 (d, J = 3.6 Hz, 2H), 6.91 (d, J = 6.9 Hz, 1H), 6.49 (s, 1H), 5.04 (d, J = 17.2 Hz, 1H), 4.70 (d, J = 17.1 Hz, 1H), 4.26 (dd, J = 13.8, 4.3 Hz, 1H), 3.81 (s, 2H), 3.79-3.66 (m, 3H), 3.39 (d, J = 1.0 Hz, 5H), 3.24 (q, J = 7.3 Hz, 3H), 3.12 (t, J = 6.1 Hz, 3H), 3.05 (dd, J = 9.2, 4.6 Hz, 2H), 2.86 (s, 3H), 1.97 (dt, J = 12.9, 6.6 Hz, 1H), 1.74 (dq, J = 14.5, 7.7 Hz, 1H), 1.37 (t, J = 7.3 Hz, 3H), 1.15 (t, J = 7.4 Hz, 3H). |
| XIV- 23 | [chemical structure] | 647.75 [M + H]⁺ | (400 MHz, Methanol-d4) δ 8.46 (d, J = 2.6 Hz, 1H), 8.17 (d, J = 6.7 Hz, 1H), 7.84 (s, 1H), 7.69 (dd, J = 9.4, 3.0 Hz, 3H), 6.84 (d, J = 6.6 Hz, 1H), 6.27 (s, 1H), 5.01 (d, J = 17.2 Hz, 1H), 4.68 (d, J = 17.1 Hz, 1H), 4.24 (dd, J = 13.6, 4.4 Hz, 1H), 3.76 (s, 2H), 3.71 (dd, J = 13.5, 4.1 Hz, 1H), 3.55 (s, 2H), 3.23 (q, J = 7.3 Hz, 3H), 3.03 (dd, J = 9.0, 4.5 Hz, 4H), 2.49 (s, 2H), 2.13 (tt, J = 8.6, 5.0 Hz, 1H), 1.96 (dt, J = 13.1, 6.2 Hz, 1H), 1.72 (dt, J = 14.7, 7.7 Hz, 1H), 1.36 (t, J = 7.3 Hz, 3H), 1.14 (t, J = 7.4 Hz, 5H), 0.95-0.86 (m, 2H) |
| XIV- 24 | [chemical structure] | 648.74 [M + H]⁺ | (400 MHz, Methanol-d4) δ 8.46 (d, J = 2.6 Hz, 1H), 8.17 (d, J = 6.7 Hz, 1H), 7.84 (s, 1H), 7.69 (dd, J = 9.4, 3.0 Hz, 3H), 6.84 (d, J = 6.6 Hz, 1H), 6.27 (s, 1H), 5.01 (d, J = 17.2 Hz, 1H), 4.68 (d, J = 17.1 Hz, 1H), 4.24 (dd, J = 13.6, 4.4 Hz, 1H), 3.76 (s, 2H), 3.71 (dd, J = 13.5, 4.1 Hz, 1H), 3.55 (s, 2H), 3.23 (q, J = 7.3 Hz, 3H), 3.03 (dd, J = 9.0, 4.5 Hz, 4H), 2.49 (s, 2H), 2.13 (tt, J = 8.6, 5.0 Hz, 1H), 1.96 (dt, J = 13.1, 6.2 Hz, 1H), 1.72 (dt, J = 14.7, 7.7 Hz, 1H), 1.36 (t, J = 7.3 Hz, 3H), 1.14 (t, J = 7.4 Hz, 5H), 0.97-0.85 (m, 2H) |
| XIV- 25 | [chemical structure] | 633.68 [M + H]⁺ | (400 MHz, Methanol-d4) δ 8.95 (d, J = 2.4 Hz, 1H), 8.46 (d, J = 2.6 Hz, 1H), 8.41-8.31 (m, 2H), 7.66 (d, J = 2.6 Hz, 1H), 7.28 (s, 1H), 6.64 (d, J = 5.6 Hz, 1H), 5.02 (d, J = 17.0 Hz, 1H), 4.71 (d, J = 17.1 Hz, 1H), 4.28 (dd, J = 13.6, 4.3 Hz, 1H), 4.04 (s, 2H), 3.70 (dd, J = 13.6, 4.1 Hz, 1H), 3.23 (q, J = 7.3 Hz, 2H), 3.04 (dt, J = 8.8, 4.4 Hz, 5H), 2.04-1.88 (m, 1H), 1.80- 1.64 (m, 1H), 1.36 (t, J = 7.3 Hz, 3H), 1.14 (t, J = 7.4 Hz, 3H) |
| XIV- 26 | [chemical structure] | 667 [M + H]⁺ | (400 MHz, Methanol-d4, ppm): δ 8.96 (d, J = 2.4 Hz, 1H), 8.51 (t, J = 1.9 Hz, 1H), 8.38 (t, J = 1.9 Hz, 1H), 8.30-8.22 (m, 1H), 7.77- 7.69 (m, 1H), 6.93-6.86 (m, 1H), 6.47 (s, 1H), 5.03 (d, J = 17.1 Hz, 1H), 4.74 (d, J = 16.3 Hz, 1H), 4.25 (dd, J = 13.7, 4.5 Hz, 1H), 4.05 (s, 2H), 3.80-3.71 (m, 3H), 3.55-3.34 (m, 6H), 3.32 (s, 1H), 3.23 (q, J = 7.3 Hz, 2H), 3.18- 2.75 (m, 7H), 2.08-1.92 (m, 1H), 1.74 (m, 1H), 1.36 (t, J = 7.3 Hz, 3H), 1.15 (t, J = 7.4 Hz, 3H) |
| XIV- 27 | [chemical structure] | 609.2 [M + H]⁺ | N/A |
| | Enantiomer A | | |
| XIV- 28 | [chemical structure] | 638.30 [M + H]⁺ | (400 MHz, Methanol-d4) δ 8.51 (d, J = 2.5 Hz, 1H), 8.27 (d, J = 6.7 Hz, 1H), 7.85 (s, 1H), 7.77-7.64 (m, 3H), 6.90 (d, J = 6.8 Hz, 1H), 6.56 (s, 1H), 5.03 (d, J = 17.1 Hz, 1H), 4.80 (s, 2H), 4.69 (d, J = 17.1 Hz, 1H), 4.25 (dd, J = 13.7, 4.4 Hz, 1H), 3.78 (s, 2H), 3.71 (dd, J = 13.6, 4.0 Hz, 1H), 3.47 (s, 2H), 3.28-2.38 (m, 9H), 2.04-1.92 (m, 1H), 1.82-1.68 (m, 1H), 1.36 (t, J = 7.3 Hz, 3H), 1.14 (t, J = 7.4 Hz, 3H). |
| XIV- 29 | [chemical structure] | 638 [M + H]⁺ | (400 MHz, Methanol-d4) δ 8.49 (d, J = 2.6 Hz, 1H), 8.25 (d, J = 6.7 Hz, 1H), 7.87-7.81 (m, 1H), 7.78 7.64 (m, 3H), 6.89 (d, J = 13.0, 6.6 Hz, 1H), 6.54 (s, 1H), 5.01 (d, J = 17.1 Hz, 1H), 4.80 (d, J = 1.0 Hz, 2H), 4.69 (d, J = 17.2 Hz, 1H), 4.24 (dd, J = 13.6, 4.4 Hz, 1H), 3.80-3.69 (m, 3H), 3.61-3.46 (m, 2H), 3.28-2.94 (m, 8H), 2.49 (s, 1H), 1.96 (s, 1H), 1.72 (s, 1H), 1.37 (t, J = 7.3 Hz, 3H), 1.14 (t, J = 7.4 Hz, 3H) |
| XIV- 30 | [chemical structure] | 675.2 [M + H]⁺ | NMR (400 MHz, DMSO-d6) δ ppm 0.95 (t, J = 1.00 Hz, 3H) 1.31 (d, J = 1.00 Hz, 3H) 2.19-2.45 (m, 10H) 3.02-3.17 (m, 2H) 3.44- 3.59 (m, 3H) 3.85-4.02 (m, 3H) 4.65-4.80 (m, 2H) 6.59 (d, J = 1.00 Hz, 1H) 7.45-7.51 (m, 1H) 7.53- 7.63 (m, 1H) 7.67-7.78 (m, 1H) 7.82-7.91 (m, 1H) 8.01-8.20 (m, 2H) 8.29-8.52 (m, 2H) 8.77-9.08 (m, 2H) 13.11-13.53 (m, 2H). |
| | Enantiomer A | | |
| XIV- 31 | [chemical structure] | 647.75 [M + H]⁺ | (400 MHz, Methanol-d4) δ 8.48 (d, J = 2.6 Hz, 1H), 8.18 (d, J = 6.7 Hz, 1H), 7.85 (s, 1H), 7.70 (d, J = 3.5 Hz, 3H), 6.86 (d, J = 6.7 Hz, 1H), 6.28 (s, 1H), 5.02 (d, J = 17.2 Hz, 1H), 4.69 (d, J = 17.1 Hz, 1H), 4.25 (dd, J = 13.7, 4.3 Hz, 1H), 3.77 (s, 2H), 3.70 (dd, J = 13.6, 4.1 Hz, 1H), 3.23 (q, J = 7.3 Hz, 2H), 3.16-2.20 (m, 7H), 2.13 (td, J = 8.5, 4.4 Hz, 1H), 2.03-1.88 (m, 1H), 1.73 (dp, J = 15.0, 7.5 Hz, 1H), 1.36 (t, J = 7.3 Hz, 3H), 1.15 (dt, J = 10.5, 6.1 Hz, 5H), 0.96- 0.85 (m, 2H) |
| XIV- 32 | [chemical structure] | 648.74 [M + H]⁺ | (400 MHz, Methanol-d4) δ 9.00- 8.89 (m, 1H), 8.52-8.43 (m, 1H), 8.37 (d, J = 2.4 Hz, 1H), 8.17 (d, J = 6.7 Hz, 1H), 7.69 (d, J = 2.6 Hz, 1H), 6.85 (d, J = 6.7 Hz, 1H), 6.27 (s, 1H), 4.72 (d, J = 17.1 Hz, 1H), 4.24 (dd, J = 13.5, 4.5 Hz, 1H), 3.97 (s, 2H), 3.74 (dd, J = 13.6, 4.1 Hz, 1H), 3.58 (s, 2H), 3.22 (q, J = 7.3 Hz, 5H), 3.05 (dd, J = 9.1, 4.6 Hz, 1H), 2.93 (s, 3H), 2.13 (tt, J = 8.6, 5.0 Hz, 1H), 1.97 (dt, J = 13.3, 6.4 Hz, 1H), 1.73 (dp, J = 15.1, 7.6 Hz, 1H), 1.36 (t, J = 7.3 Hz, 3H), 1.14 (t, J = 7.3 Hz, 5H), 0.91 (t, J = 3.4 Hz, 2H) |
| XIV- 33 | [chemical structure] | 620.64 [M + H]⁺ | (400 MHz, Methanol-d4) δ 8.43 (t, J = 2.3 Hz, 2H), 8.35 (d, J = 5.6 Hz, 1H), 8.17 (dd, J = 16.7, 2.6 Hz, 1H), 7.63 (d, J = 2.6 Hz, 1H), 7.27 (s, 1H), 6.61 (d, J = 5.6 Hz, 1H), 4.99 (s, 1H), 4.68 (d, J = 17.0 Hz, 1H), 4.25 (dd, J = 13.5, 4.2 Hz, 1H), 3.73-3.59 (m, 2H), 3.53 (s, 1H), 3.21 (t, J = 13.8 Hz, 2H), 3.02 (dd, J = 9.1, 4.5 Hz, 1H), 2.95 (d, J = 4.1 Hz, 3H), 2.42 (dd, J = 53.9, 14.7 Hz, 2H), 2.16 (t, J = 14.4 Hz, 1H), 1.96 (dq, J = 12.5, 6.9 Hz, 2H), 1.72 (dq, J = 14.4, 7.4 Hz, 1H), 1.31 (s, 1H), 1.14 (t, J = 7.4 Hz, 3H) |
| XIV- 34 | [chemical structure] | 654.35 [M + H]⁺ | (400 MHz, Methanol-d4) δ 8.48 (d, J = 2.6 Hz, 1H), 8.43 (d, J = 2.6 Hz, 1H), 8.27-8.10 (m, 2H), 7.69 (d, J = 2.7 Hz, 1H), 6.86 (dd, J = 6.7, 1.2 Hz, 1H), 6.43 (d, J = 1.2 Hz, 1H), 5.58 (s, 1H), 4.99 (d, J = 17.1 Hz, 1H), 4.70 (d, J = 17.1 Hz, 1H), 4.22 (dd, J = 13.5, 4.4 Hz, 1H), 3.78-3.50 (m, 5H), 3.39 (s, 3H), 3.29-3.16 (m, 2H), 3.11 (t, J = 6.2 Hz, 2H), 3.04 (dq, J = 8.7, 4.2 Hz, 1H), 2.95 (d, J = 4.3 Hz, 3H), 2.49 (d, J = 14.1 Hz, 1H), 2.35 (d, J = 15.3 Hz, 1H), 2.22- 2.12 (m, 1H), 2.05-1.88 (m, 2H), 1.73 (ddd, J = 13.7, 9.1, 7.1 Hz, 1H), 1.15 (t, J = 7.4 Hz, 3H). |
| XIV- 35 | [chemical structure] | 654 [M + H]⁺ | (400 MHz, Methanol-d4) δ 8.52- 8.40 (m, 2H), 8.26-8.13 (m, 2H), 7.70 (s, 1H), 6.87 (d, J = 6.8 Hz, 1H), 6.44 (s, 1H), 5.58 (s, 1H), 4.99 (d, J = 17.2 Hz, 1H), 4.70 (d, J = 17.2 Hz, 1H), 4.22 (d, J = 14.0 Hz, 1H), 3.75 (t, J = 6.2 Hz, 2H), 3.71-3.60 (m, 1H), 3.52 (d, J = 12.8 Hz, 1H), 3.39 (s, 3H), 3.21 (t, J = 14.1 Hz, 2H), 3.11 (t, J = 6.1 Hz, 2H), 3.07-3.00 (m, 1H), 2.95 (d, J = 4.4 Hz, 3H), 2.49 (d, J = 13.7 Hz, 1H), 2.35 (d, J = 15.5 Hz, 1H), 2.17 (t, J = 14.3 Hz, 1H), 2.05- 1.88 (m, 2H), 1.73 (dt, J = 14.2, 7.9 Hz, 1H), 1.15 (t, J = 7.4 Hz, 3H) |
| XIV- 36 | [chemical structure] | 635.69 [M + H]⁺ | (400 MHz, Methanol-d4) δ 8.37 (d, J = 2.6 Hz, 1H), 8.31 (d, J = 2.6 Hz, 1H), 8.12 (d, J = 2.6 Hz, 1H), 8.01 (d, J = 5.6 Hz, 1H), 7.44 (d, J = 2.6 Hz, 1H), 6.57 (d, J = 5.6 Hz, 1H), 5.89 (s, 1H), 5.32 (s, 1H), 4.89 (s, 1H), 4.61 (d, J = 16.9 Hz, 1H), 4.24 (dd, J = 13.5, 4.2 Hz, 1H), 3.61 (dd, J = 13.5, 4.0 Hz, 1H), 2.97 (dq, J = 8.8, 4.3 Hz, 1H), 2.82 (s, 2H), 2.68 (s, 2H), 2.46 (s, 3H), 2.17-1.84 (m, 6H), 1.69 (ddt, J = 16.6, 14.4, 7.5 Hz, 1H), 1.61-1.46 (m, 3H), 1.12 (t, J = 7.4 Hz, 3H), 1.05-1.00 (m, 2H), 0.84-0.78 (m, 2H) |
| XIV- 37 | [chemical structure] | 649.35 [M + H]⁺ | N/A |
| XIV- 38 | [chemical structure] | 649.35 [M + H]⁺ | (400 MHz, Methanol-d4) δ 8.58 (d, J = 59.1 Hz, 1H), 8.25-8.21 (m, 1H), 8.00 (s, 1H), 7.86 (s, 1H), 7.75-7.67 (m, 2H), 6.26 (d, J = 7.2 Hz, 1H), 5.20-5.02 (m, 1H), 4.69 (d, J = 17.3 Hz, 1H), 4.41 (d, J = 14.7 Hz, 1H), 3.85-3.72 (m, 2H), 3.69-3.36 (m, 3H), 3.27- 2.33 (m, 9H), 2.11 (ddd, J = 13.5, 8.6, 5.1 Hz, 1H), 1.91 (s, 1H), 1.76 (s, 1H), 1.36 (t, J = 7.3 Hz, 3H), 1.16 (t, J = 7.4 Hz, 3H), 1.13- 1.05 (m, 2H), 0.93-0.85 (m, 2H). |
| XIV- 39 | [chemical structure] | 706.40 [M + H]⁺ | (400 MHz, Methanol-d4, ppm): δ 8.38 (d, J = 2.8 Hz, 1H), 8.19 (d, J = 3.7 Hz, 1H), 8.03 (s, 1H), 7.88 (s, 1H), 7.81 (d, J = 2.0 Hz, 1H), 7.72-7.64 (m, 2H), 7.44 (d, J = 2.7 Hz, 1H), 6.28 (s, 1H), 4.96 (s, 1H), 4.58 (d, J = 17.1 Hz, 1H), 4.28 (dd, J = 13.6, 4.0 Hz, 1H), 3.94 (s, 3H), 3.75 (s, 2H), 3.66- 3.49 (m, 3H), 3.23 (q, J = 7.3 Hz, 2H), 3.16-2.93 (m, 5H), 2.47 (m, 2H), 1.93-1.82 (m, 1H), 1.69 (m, 7.6 Hz, 1H), 1.36 (t, J = 7.3 Hz, 3H), 1.12 (t, J = 7.4 Hz, 3H). |
| XIV- 40 | [chemical structure] | 652.2 [M + H]⁺ | N/A |
| XIV- 41 | [chemical structure] | 666.2 [M + H]⁺ | N/A |
| XIV- 42 | [chemical structure] | 621.25 [M + H]⁺ | (400 MHz, Methanol-d₄) δ 8.43 (d, J = 2.7 Hz, 2H), 8.35 (d, J = 5.6 Hz, 1H), 8.16 (dd, J = 16.7, 2.6 Hz, 1H), 7.62 (s, 1H), 7.27 (s, 1H), 6.63-6.58 (m, 1H), 5.62-5.38 (m, 1H), 4.97 (d, J = 17.0 Hz, 1H), 4.68 (d, J = 17.0 Hz, 1H), 4.24 (dd, J = 13.5, 4.2 Hz, 1H), 3.70-3.49 (m, 3H), 3.21 (t, J = 13.4 Hz, 2H), 3.02 (dd, J = 9.1, 4.6 Hz, 1H), 2.95 (d, J = 4.1 Hz, 3H), 2.42 (dd, J = 54.0, 15.0 Hz, 2H), 2.15 (t, J = 14.4 Hz, 1H), 2.03-1.89 (m, 2H), 1.78-1.64 (m, 1H), 1.14 (t, J = 7.4 Hz, 3H). |
| XIV- 43 | [chemical structure] | 636.25 [M + H]⁺ | (400 MHz, Methanol-d₄) δ 8.43 (dd, J = 4.2, 2.7 Hz, 2H), 8.23- 8.09 (m, 2H), 7.64 (d, J = 2.6 Hz, 1H), 6.79 (d, J = 6.5 Hz, 1H), 6.20 (s, 1H), 5.62-5.38 (m, 1H), 4.97 (d, J = 17.1 Hz, 1H), 4.68 (d, J = 17.1 Hz, 1H), 4.22 (dd, J = 13.5, 4.4 Hz, 1H), 3.74-3.59 (m, 2H), 3.56-3.46 (m, 1H), 3.30-3.16 (m, 2H), 3.03 (dt, J = 9.1, 4.5 Hz, 1H), 2.95 (d, J = 4.3 Hz, 3H), 2.42 (dd, J = 54.7, 14.9 Hz, 2H), 2.21- 2.04 (m, 2H), 1.96 (ddd, J = 12.5, 7.5, 4.8 Hz, 2H), 1.73 (ddt, J = 16.4, 14.4, 7.3 Hz, 1H), 1.21- 1.03 (m, 5H), 0.89 (dt, J = 6.9, 4.6 Hz, 2H). |
| XIV- 44 | [chemical structure] | 619.30 [M + H]⁺ | (400 MHz, Methanol-d₄) δ 8.41 (d, J = 2.7 Hz, 1H), 8.34 (d, J = 5.6 Hz, 1H), 7.82 (d, J = 1.8 Hz, 1H), 7.71-7.66 (m, 2H), 7.61 (d, J = 2.6 Hz, 1H), 7.27 (s, 1H), 6.60 (d, J = 5.6 Hz, 1H), 5.05 (s, 1H), 4.66 (d, J = 16.1 Hz, 5H), 4.26 (dd, J = 13.7, 4.2 Hz, 1H), 3.65 (d, J = 13.4 Hz, 3H), 3.00 (dd, J = 9.3, 4.4 Hz, 1H), 2.66 (d, J = 47.0 Hz, 7H), 2.47 (s, 3H), 1.99-1.90 (m, 1H), 1.71 (dt, J = 14.7, 7.2 Hz, 1H), 1.50 (d, J = 13.8 Hz, 1H), 1.31 (s, 2H), 1.14 (t, J = 7.4 Hz, 4H), 0.91 (d, J = 9.3 Hz, 1H), 0.12 (d, J = 2.3 Hz, 1H). |
| XIV- 45 | [chemical structure] | 610.20 [M + H]⁺ | (400 MHz, Methanol-d₄) δ 8.49 (d, J = 2.6 Hz, 1H), 8.26 (d, J = 6.7 Hz, 1H), 7.85 (d, J = 1.8 Hz, 1H), 7.71 (d, J = 2.5 Hz, 3H), 6.88 (d, J = 6.7 Hz, 1H), 6.54 (s, 1H), 5.02 (d, J = 17.2 Hz, 1H), 4.80 (s, 2H), 4.69 (d, J = 17.1 Hz, 1H), 4.25 (dd, J = 13.6, 4.3 Hz, 1H), 3.77 (s, 2H), 3.71 (dd, J = 13.6, 4.1 Hz, 1H), 3.27 (t, J = 5.2 Hz, 4H), 3.04 (dd, J = 9.1, 4.6 Hz, 1H), 2.76 (t, J = 5.3 Hz, 4H), 2.01-1.92 (m, 1H), 1.78- 1.68 (m, 1H), 1.14 (t, J = 7.4 Hz, 3H). |
| XIV- 46 | [chemical structure] | 624.35 [M + H]⁺ | (400 MHz, Methanol-d4) δ 8.48 (s, 1H), 8.25 (d, J = 6.5 Hz, 1H), 7.84 (d, J = 2.2 Hz, 1H), 7.70 (d, J = 4.4 Hz, 3H), 6.87 (s, 1H), 6.53 (s, 1H), 5.01 (d, J = 16.9 Hz, 1H), 4.79 (s, 2H), 4.68 (d, J = 17.1 Hz, 1H), 4.25 (d, J = 12.9 Hz, 1H), 3.76 (s, 2H), 3.70 (d, J = 13.4 Hz, 1H), 3.60-3.33 (m, 3H), 3.30-3.15 (m, 2H), 3.03 (s, 2H), 2.92 (s, 3H), 2.49 (s, 2H), 1.96 (s, 1H), 1.73 (m, 1H), 1.14 (t, J = 7.3 Hz, 3H). |
| XIV- 47 | [chemical structure] | 606.25 [M + H]⁺ | (400 MHz, Methanol-d4) δ 8.48 (d, J = 2.7 Hz, 1H), 8.25 (d, J = 6.6 Hz, 1H), 7.75 (s, 1H), 7.70 (d, J = 2.7 Hz, 1H), 7.57 (d, J = 8.4 Hz, 1H), 7.40-7.33 (m, 1H), 7.24 (s, 1H), 6.87 (d, J = 6.7 Hz, 1H), 6.54 (s, 1H), 5.02 (d, J = 17.1 Hz, 1H), 4.80 (s, 2H), 4.68 (d, J = 17.1 Hz, 1H), 4.25 (dd, J = 14.0, 4.1 Hz, 1H), 3.76-3.65 (m, 3H), 3.50-3.40 (m, 2H), 3.30-3.15 (m, 2H), 3.0 (s, 2H), 2.91 (s, 3H), 2.48 (s, 2H), 1.96 (m, 1H), 1.73 (m, 1H), 1.14 (t, J = 7.4 Hz, 3H). |
| XIV- 48 | [chemical structure] | 652.30 [M + H]⁺ | (400 MHz, Methanol-d₄) δ 8.52 (d, J = 2.7 Hz, 1H), 8.29 (d, J = 6.7 Hz, 1H), 7.86 (d, J = 1.9 Hz, 1H), 7.78-7.68 (m, 3H), 6.90 (d, J = 6.7 Hz, 1H), 6.80 (s, 1H), 5.04 (d, J = 17.2 Hz, 1H), 4.70 (d, J = 17.2 Hz, 1H), 4.26 (dd, J = 13.7, 4.3 Hz, 1H), 3.80 (d, J = 1.7 Hz, 2H), 3.71 (dd, J = 13.6, 4.1 Hz, 1H), 3.28 (t, J = 5.2 Hz, 4H), 3.05 (dq, J = 8.9, 4.4 Hz, 1H), 2.79 (q, J = 5.0 Hz, 4H), 2.03-1.91 (m, 1H), 1.74 (ddt, J = 13.8, 9.1, 7.1 Hz, 1H), 1.15 (t, J = 7.4 Hz, 3H). |
| XIV- 49 | [chemical structure] | 652.25 [M + H]⁺ | (400 MHz, Methanol-d4) δ 8.53 (s, 1H), 8.28 (t, J = 8.6 Hz, 1H), 7.86 (d, J = 16.0 Hz, 1H), 7.74 (d, J = 17.2 Hz, 3H), 6.92 (d, J = 6.7 Hz, 1H), 6.81 (s, 1H), 5.04 (d, J = 17.2 Hz, 1H), 4.70 (d, J = 17.2 Hz, 1H), 4.26 (d, J = 13.8 Hz, 1H), 3.86 (d, J = 8.8 Hz, 2H), 3.71 (dd, J = 13.6, 4.1 Hz, 1H), 3.30 (s, 3H), 3.05 (s, 1H), 2.80 (d, J = 39.6 Hz, 4H), 1.97 (dt, J = 12.9, 6.5 Hz, 1H), 1.72 (dq, J = 14.4, 7.5, 6.9 Hz, 1H), 1.14 (t, J = 7.4 Hz, 3H). |
| XIV- 50 | [chemical structure] | 666.40 [M + H]⁺ | (400 MHz, Methanol-d4, ppm) δ 8.51 (d, J = 2.7 Hz, 1H), 8.28 (d, J = 6.6 Hz, 1H), 7.85 (d, J = 2.1 Hz, 1H), 7.75-7.66 (m, 3H), 6.89 (d, J = 6.6 Hz, 1H), 6.79 (s, 1H), 5.03 (d, J = 17.2 Hz, 1H), 4.98-4.92 (m, 4H), 4.69 (d, J = 17.2 Hz, 1H), 4.25 (m, 1H), 3.77 (s, 2H), 3.71 (dd, J = 13.6, 4.1 Hz, 1H), 3.52- 3.35 (m, 2H), 3.31-3.17 (m, 2H), 3.05 (m, 1H), 2.92 (m, 4H), 2.85- 2.40 (m, 3H), 2.04-1.88 (m, 1H), 1.79-1.68 (m, 1H), 1.14 (t, J = 7.4 Hz, 3H). |
| XIV- 51 | [chemical structure] | 666.25 [M + H]⁺ | (400 MHz, Methanol-d4) δ 8.50 (s, 1H), 8.27 (d, J = 6.9 Hz, 1H), 7.84 (s, 1H), 7.70 (s, 3H), 6.87 (d, J = 7.1 Hz, 1H), 6.76 (s, 1H), 5.02 (d, J = 16.8 Hz, 1H), 4.69 (d, J = 17.3 Hz, 1H), 4.25 (d, J = 12.4 Hz, 1H), 3.73 (d, J = 28.6 Hz, 4H), 3.04 (s, 2H), 2.92 (s, 4H), 2.55 (s, 3H), 1.96 (s, 1H), 1.77-1.68 (m, 1H), 1.14 (t, J = 7.4 Hz, 3H). |
| XIV- 52 | [chemical structure] | 648.40 [M + H]⁺ | (400 MHz, Methanol-d₄) δ 8.51 (d, J = 2.7 Hz, 1H), 8.28 (d, J = 6.7 Hz, 1H), 7.75 (dd, J = 4.6, 2.5 Hz, 2H), 7.57 (dd, J = 8.3, 2.3 Hz, 1H), 7.41-7.08 (m, 2H), 6.90 (d, J = 6.7 Hz, 1H), 6.80 (s, 1H), 5.03 (d, J = 17.2 Hz, 1H), 4.69 (d, J = 17.2 Hz, 1H), 4.26 (dd, J = 13.7, 4.3 Hz, 1H), 3.75 (s, 2H), 3.70 (dd, J = 13.6, 4.1 Hz, 1H), 3.45 (d, J = 6.3 Hz, 1H), 3.31-3.19 (m, 2H), 3.04 (dq, J = 8.9, 4.3 Hz, 1H), 2.91 (s, 3H), 2.86-2.32 (m, 3H), 2.03- 1.91 (m, 1H), 1.74 (ddt, J = 16.5, 14.4, 7.4 Hz, 1H), 1.15 (t, J = 7.4 Hz, 3H). |
| XIV- 53 | [chemical structure] | 648.30 [M + H]⁺ | (400 MHz, Methanol-d4) δ 8.51 (s, 1H), 8.28 (s, 1H), 7.75 (s, 2H), 7.57 (d, J = 8.3 Hz, 1H), 7.36 (d, J = 9.0 Hz, 1H), 7.17 (d, J = 55.0 Hz, 1H), 6.85 (dd, J = 43.5, 9.6 Hz, 2H), 5.05 (s, 1H), 4.69 (d, J = 16.9 Hz, 1H), 4.26 (d, J = 13.5 Hz, 1H), 3.71 (d, J = 21.3 Hz, 3H), 3.04 (s, 2H), 2.91 (s, 4H), 1.74 (s, 1H), 1.14 (t, J = 7.3 Hz, 3H). |
| XIV- 54 | [chemical structure] | 688.2 [M + H]⁺ | N/A |
| XIV- 55 | [chemical structure] | 610.25 [M + H]⁺ | (400 MHz, Methanol-d4) δ 8.45 (s, 1H), 8.22 (d, J = 6.4 Hz, 1H), 7.83 (s, 1H), 7.70 (s, 2H), 7.65 (s, 1H), 6.82 (d, J = 6.4 Hz, 1H), 6.48 (s, 1H), 5.00 (d, J = 17.2 Hz, 1H), 4.78 (s, 2H), 4.67 (d, J = 16.8 Hz, 1H), 4.24 (d, J = 15.5 Hz, 1H), 3.74 (s, 2H), 3.68 (s, 1H), 3.25 (s, 4H), 3.02 (s, 1H), 2.72 (s, 4H), 1.95 (s, 1H), 1.71 (s, 1H), 1.14 (t, J = 7.4 Hz, 3H). |
| XIV- 56 | [chemical structure] | 624.25 [M + H]⁺ | (400 MHz, Methanol-d₄) δ 8.46 (d, J = 2.6 Hz, 1H), 8.22 (d, J = 6.3 Hz, 1H), 7.84 (d, J = 2.1 Hz, 1H), 7.74-7.64 (m, 3H), 6.83 (d, J = 6.5 Hz, 1H), 6.48 (s, 1H), 5.00 (d, J = 17.1 Hz, 1H), 4.79 (s, 2H), 4.68 (d, J = 17.1 Hz, 1H), 4.25 (dd, J = 13.6, 4.3 Hz, 1H), 3.78-3.62 (m, 4H), 3.50 (s, 2H), 3.16 (s, 2H), 3.07-2.96 (m, 2H), 2.92 (s, 4H), 2.48 (s, 2H), 1.95 (s, 1H), 1.72 (dt, J = 14.4, 8.2 Hz, 1H), 1.31 (s, 1H), 1.14 (t, J = 7.4 Hz, 3H). |
| XIV- 57 | [chemical structure] | 606.30 [M + H]⁺ | (400 MHz, Methanol-d₄) δ 8.49 (d, J = 2.7 Hz, 1H), 8.26 (d, J = 6.6 Hz, 1H), 7.75 (d, J = 2.3 Hz, 1H), 7.71 (d, J = 2.6 Hz, 1H), 7.57 (d, J = 8.5 Hz, 1H), 7.40-7.32 (m, 1H), 7.17 (d, J = 55.3 Hz, 1H), 6.89 (d, J = 6.7 Hz, 1H), 6.55 (d, J = 1.1 Hz, 1H), 5.02 (d, J = 17.2 Hz, 1H), 4.80 (d, J = 0.9 Hz, 2H), 4.68 (d, J = 17.2 Hz, 1H), 4.26 (dd, J = 13.7, 4.3 Hz, 1H), 3.74 (s, 2H), 3.72-3.66 (m, 1H), 3.03 (dd, J = 9.1, 4.5 Hz, 4H), 2.91 (s, 4H), 2.74- 2.19 (m, 3H), 1.97 (ddd, J = 13.0, 7.5, 4.9 Hz, 1H), 1.80-1.67 (m, 1H), 1.14 (t, J = 7.4 Hz, 3H). |
| XIV- 58 | [chemical structure] | 620.30 [M + H]⁺ | (400 MHz, Methanol-d4) δ 8.47 (s, 1H), 8.17 (d, J = 5.8 Hz, 1H), 7.85 (s, 1H), 7.76-7.64 (m, 3H), 6.86 (s, 1H), 6.28 (s, 1H), 5.01 (d, J = 17.0 Hz, 1H), 4.68 (d, J = 17.2 Hz, 1H), 4.24 (d, J = 13.8 Hz, 1H), 3.84-3.66 (m, 3H), 3.27 (s, 4H), 3.04 (s, 1H), 2.13 (s, 1H), 1.96 (s, 1H), 1.73 (s, 1H), 1.23-1.07 (m, 5H), 0.91 (d, J = 5.3 Hz, 2H). |
| XIV- 59 | [chemical structure] | 616.45 [M + H]⁺ | (400 MHz, Methanol-d4) δ 8.47 (s, 1H), 8.17 (d, J = 6.2 Hz, 1H), 7.75 (s, 1H), 7.69 (s, 1H), 7.57 (d, J = 8.1 Hz, 1H), 7.36 (d, J = 9.5 Hz, 1H), 7.17 (d, J = 55.3 Hz, 1H), 6.86 (d, J = 6.5 Hz, 1H), 6.27 (s, 1H), 5.01 (d, J = 17.1 Hz, 1H), 4.67 (d, J = 17.2 Hz, 1H), 4.25 (d, J = 13.9 Hz, 1H), 3.71 (d, J = 20.6 Hz, 3H), 3.02 (s, 2H), 2.90 (s, 3H), 2.13 (s, 1H), 1.97 (d, J = 5.5 Hz, 1H), 1.79-1.67 (m, 1H), 1.14 (t, J = 7.6 Hz, 5H), 0.96-0.87 (m, 2H). |
| XIV- 60 | [chemical structure] | 664.30 [M + H]⁺ | (400 MHz, Methanol-d4) δ 8.47 (d, J = 2.7 Hz, 1H), 8.17 (d, J = 6.8 Hz, 1H), 7.84 (s, 1H), 7.80-7.64 (m, 3H), 6.85 (d, J = 6.8 Hz, 1H), 6.27 (s, 1H), 5.01 (d, J = 17.2 Hz, 2H), 4.68 (d, J = 17.2 Hz, 1H), 4.24 (dd, J = 13.5, 4.3 Hz, 1H), 3.89 (t, J = 5.2 Hz, 2H), 3.78 (s, 2H), 3.75-3.66 (m, 1H), 3.32- 3.08 (m, 5H), 3.03 (s, 1H), 2.82 (s, 3H), 2.13 (s, 1H), 1.96 (s, 1H), 1.74 (dd, J = 15.0, 7.8 Hz, 1H), 1.23-1.05 (m, 5H), 0.94-0.85 (m, 2H). |
| XIV- 61 | [chemical structure] | 690.2 [M + H]⁺ | N/A |
| XIV- 62 | [chemical structure] | 676.2 [M + H]⁺ | N/A |
| XIV- 63 | [chemical structure] | 678.2 [M + H]⁺ | NMR (400 MHz, DMSO-d6) δ ppm 0.90-1.03 (m, 4H) 1.05- 1.29 (m, 2H) 1.47-1.63 (m, 1H) 1.79-1.93 (m, 1H) 1.96-2.11 (m, 1H) 2.19-2.30 (m, 1H) 2.36 (m, 8H) 2.51-2.65 (m, 1H) 2.80-2.90 (m, 1H) 3.10-3.18 (m, 2H) 3.18- 3.41 (m, 1H) 3.50 (m, 2H) 3.58- 3.73 (m, 1H) 3.64-3.64 (m, 1H) 3.73-3.80 (m, 1H) 3.81-3.96 (m, 1H) 3.96-4.11 (m, 1H) 4.69 (dd, J = 1.00 Hz, 2H) 6.06 (s, 1H) 6.44 (br d, J = 5.28 Hz, 1H) 7.42 (br s, 1H) 7.49-7.67 (m, 1H) 7.73 (br d, J = 7.82 Hz, 1H) 7.87 (br s, 1H) 8.03 (br d, J = 5.28 Hz, 1H) 8.31- 8.44 (m, 1H) 8.92 (s, 1H) 11.77 (br s, 1H) |
| XIV- 64 | [chemical structure] | 620.45 [M + H]⁺ | (400 MHz, Methanol-d4) δ 8.46 (d, J = 2.7 Hz, 1H), 8.17 (d, J = 6.9 Hz, 1H), 7.84 (d, J = 2.2 Hz, 1H), 7.71 (d, J = 2.1 Hz, 2H), 7.68 (d, J = 3.2 Hz, 1H), 6.84 (dd, J = 6.7, 3.0 Hz, 1H), 6.29-6.25 (m, 1H), 5.01 (d, J = 17.1 Hz, 1H), 4.68 (d, J = 17.1 Hz, 1H), 4.25 (dd, J = 13.7, 4.4 Hz, 1H), 3.76 (s, 2H), 3.70 (dd, J = 13.6, 4.1 Hz, 1H), 3.26 (d, J = 5.9 Hz, 4H), 3.03 (dd, J = 9.1, 4.5 Hz, 1H), 2.75 (s, 4H), 2.13 (td, J = 8.7, 4.5 Hz, 1H), 2.01- 1.91 (m, 1H), 1.72 (dt, J = 16.2, 7.3 Hz, 1H), 1.14 (t, J = 7.4 Hz, 5H), 0.90 (dd, J = 6.5, 4.7 Hz, 2H). |
| XIV- 65 | [chemical structure] | 664.25 [M + H]⁺ | (400 MHz, Methanol-d4) δ 8.47 (d, J = 2.6 Hz, 1H), 8.17 (d, J = 6.7 Hz, 1H), 7.84 (d, J = 1.9 Hz, 1H), 7.76-7.66 (m, 3H), 6.85 (d, J = 6.7 Hz, 1H), 6.27 (s, 1H), 5.02 (d, J = 17.2 Hz, 1H), 4.68 (d, J = 17.2 Hz, 1H), 4.25 (dd, J = 13.6, 4.4 Hz, 1H), 3.95-3.87 (m, 2H), 3.78 (s, 2H), 3.71 (dd, J = 13.6, 4.1 Hz, 1H), 3.31-3.25 (m, 3H), 3.03 (dd, J = 8.9, 4.5 Hz, 1H), 2.78 (s, 4H), 2.14 (ddd, J = 13.4, 8.4, 5.0 Hz, 1H), 1.96 (ddd, J = 12.6, 7.5, 4.8 Hz, 1H), 1.73 (ddd, J = 13.9, 9.2, 7.2 Hz, 1H), 1.20-1.10 (m, 5H), 0.91 (dt, J = 6.8, 4.5 Hz, 2H). |
| XIV- 66 | [chemical structure] | 616.30 [M + H]⁺ | (400 MHz, Methanol-d₄) δ 8.52- 8.43 (m, 1H), 8.22-8.02 (m, 1H), 7.75 (d, J = 2.3 Hz, 1H), 7.69 (d, J = 2.5 Hz, 1H), 7.57 (dd, J = 8.3, 2.3 Hz, 1H), 7.42-7.06 (m, 2H), 6.85 (dd, J = 6.8, 2.0 Hz, 1H), 6.26 (d, J = 2.1 Hz, 1H), 5.01 (d, J = 17.1 Hz, 1H), 4.67 (d, J = 17.2 Hz, 1H), 4.25 (dd, J = 13.6, 4.4 Hz, 1H), 3.82-3.63 (m, 3H), 3.56- 3.39 (m, 1H), 3.29-3.12 (m, 2H), 3.07-3.00 (m, 1H), 2.90 (s, 4H), 2.48 (t, J = 28.0 Hz, 3H), 2.13 (ddd, J = 13.5, 8.6, 5.1 Hz, 1H), 2.03-1.88 (m, 1H), 1.73 (ddt, J = 16.6, 14.5, 7.4 Hz, 1H), 1.20- 1.10 (m, 5H), 0.91 (dt, J = 6.9, 4.6 Hz, 2H). |
| XIV- 67 | [chemical structure] | 596.25 [M + H]⁺ | (400 MHz, Methanol-d4) δ 8.43 (d, J = 2.7 Hz, 2H), 8.20-8.09 (m, 2H), 7.63 (s, 1H), 6.78 (m, 1H), 6.32-6.21 (m, 1H), 5.78 (s, 1H), 4.95 (d, J = 17.0 Hz, 1H), 4.66 (d, J = 17.3 Hz, 1H), 4.19 (m, 1H), 4.08 (s, 1H), 3.94 (d, J = 13.3 Hz, 1H), 3.83 (s, 1H), 3.71-3.63 (m, 1H), 3.56 (s, 1H), 3.04 (s, 1H), 2.99 (s, 3H), 2.75 (s, 1H), 2.52- 2.46 (m, 4H), 2.31 (s, 1H), 1.94 (m, 2H), 1.70 (m, 1H), 1.11 (t, J = 7.4 Hz, 3H). |
| XIV- 68 | [chemical structure] | 596.25 [M + H]⁺ | (400 MHz, Methanol-d₄) δ 8.42- 8.36 (m, 1H), 8.32 (d, J = 2.7 Hz, 1H), 8.12 (d, J = 2.6 Hz, 1H), 8.03 (d, J = 5.6 Hz, 1H), 7.45 (d, J = 2.7 Hz, 1H), 6.59 (d, J = 5.6 Hz, 1H), 5.98 (q, J = 1.0 Hz, 1H), 5.55 (tt, J = 6.5, 3.1 Hz, 1H), 4.94 (s, 1H), 4.62 (d, J = 16.9 Hz, 1H), 4.24 (dd, J = 13.5, 4.2 Hz, 1H), 3.62 (dd, J = 13.5, 4.1 Hz, 1H), 3.17-3.09 (m, 1H), 2.97 (dd, J = 9.0, 4.6 Hz, 1H), 2.85-2.72 (m, 2H), 2.69-2.61 (m, 1H), 2.49-2.29 (m, 7H), 2.10- 1.99 (m, 1H), 1.98-1.85 (m, 1H), 1.70 (ddt, J = 16.6, 14.4, 7.5 Hz, 2H), 1.13 (t, J = 7.4 Hz, 3H). |
| XIV- 69 | [chemical structure] | 610.30 [M + H]⁺ | (400 MHz, Methanol-d₄) δ 8.50- 8.33 (m, 2H), 8.25-8.10 (m, 2H), 7.73-7.62 (m, 1H), 6.84 (dd, J = 14.0, 6.7 Hz, 1H), 6.43-6.24 (m, 1H), 5.38 (td, J = 10.9, 5.4 Hz, 1H), 4.98 (d, J = 16.9 Hz, 1H), 4.69 (d, J = 17.4 Hz, 1H), 4.22 (dd, J = 13.4, 4.4 Hz, 1H), 3.70 (dd, J = 13.5, 4.1 Hz, 1H), 3.58-3.41 (m, 2H), 3.26-3.16 (m, 1H), 3.04 (p, J = 7.5, 6.8 Hz, 1H), 2.58-2.39 (m, 5H), 1.97 (dt, J = 13.0, 5.8 Hz, 1H), 1.90-1.57 (m, 3H), 1.41 (d, J = 6.5 Hz, 3H), 1.14 (t, J = 7.4 Hz, 3H). |
| | trans isomer at piperidinyl ring | | |
| XIV- 70 | [chemical structure] | 692.2 | N/A |
| XIV- 71 | [chemical structure] | N/A | (400 MHz, Methanol-d₄) δ 8.49 (t, J = 2.8 Hz, 1H), 8.43 (d, J = 2.6 Hz, 1H), 8.21 (dd, J = 6.6, 2.2 Hz, 1H), 8.14 (d, J = 2.6 Hz, 1H), 7.80-7.61 (m, 1H), 6.96-6.83 (m, 1H), 6.39 (dd, J = 2.8, 1.5 Hz, 1H), 5.48-5.26 (m, 2H), 4.99 (d, J = 17.3 Hz, 1H), 4.70 (d, J = 17.1 Hz, 1H), 4.21 (dd, J = 13.7, 4.4 Hz, 1H), 3.71 (dd, J = 13.3, 4.0 Hz, 1H), 3.59-3.42 (m, 2H), 3.25- 3.14 (m, 1H), 3.04 (dt, J = 9.3, 4.7 Hz, 1H), 2.57-2.41 (m, 5H), 1.97 (ddd, J = 13.8, 7.3, 4.8 Hz, 1H), 1.88-1.62 (m, 3H), 1.41 (d, J = 6.5 Hz, 3H), 1.15 (t, J = 7.4 Hz, 3H). |
| | cis isomer at piperidinyl ring | | |
| XIV- 72 | [chemical structure] | 634.25 [M + H]⁺ | (400 MHz, Methanol-d4) δ 8.47 (d, J = 1.9 Hz, 1H), 8.20-8.14 (m, 1H), 7.84 (s, 1H), 7.74-7.65 (m, 3H), 6.85 (dd, J = 6.7, 1.6 Hz, 1H), 6.27 (d, J = 1.9 Hz, 1H), 5.01 (d, J = 17.1 Hz, 1H), 4.68 (d, J = 17.2 Hz, 1H), 4.24 (dd, J = 13.7, 4.4 Hz, 1H), 3.76 (s, 2H), 3.71 (dd, J = 13.6, 4.0 Hz, 1H), 3.03 (dd, J = 9.0, 4.6 Hz, 2H), 2.92 (s, 4H), 2.51 (s, 1H), 2.13 (td, J = 8.6, 4.4 Hz, 1H), 2.01-1.90 (m, 1H), 1.72 (dq, J = 14.6, 7.4 Hz, 1H), 1.14 (td, J = 8.0, 7.5, 3.4 Hz, 5H), 0.91 (dt, J = 6.9, 4.5 Hz, 2H). |
| XIV- 73 | [chemical structure] | 634.25 [M + H]⁺ | (400 MHz, Methanol-d4) δ 8.47 (d, J = 6.7 Hz, 1H), 8.17 (t, J = 7.2 Hz, 1H), 7.85 (s, 1H), 7.76-7.65 (m, 3H), 6.90-6.79 (m, 1H), 6.27 (d, J = 10.9 Hz, 1H), 4.69 (d, J = 17.2 Hz, 1H), 4.25 (d, J = 13.9 Hz, 1H), 3.80-3.65 (m, 3H), 3.07- 2.95 (m, 2H), 2.92 (s, 4H), 2.74 (s, 2H), 2.15 (td, J = 8.5, 4.6 Hz, 1H), 2.02-1.90 (m, 1H), 1.72 (dq, J = 15.1, 7.7 Hz, 1H), 1.21-1.08 (m, 5H), 0.95-0.87 (m, 2H). |
| XIV- 74 | [chemical structure] | 624.25 [M + H]⁺ | (400 MHz, Methanol-d₄) δ 8.50 (d, J = 2.7 Hz, 1H), 8.26 (d, J = 6.7 Hz, 1H), 7.86 (d, J = 2.0 Hz, 1H), 7.75-7.67 (m, 3H), 6.90 (d, J = 6.7 Hz, 1H), 6.58 (s, 1H), 4.97 (s, 1H), 4.84-4.73 (m, 3H), 3.94 (dd, J = 13.6, 4.6 Hz, 1H), 3.85 (dd, J = 13.7, 6.0 Hz, 1H), 3.78 (s, 2H), 3.67-3.38 (m, 2H), 3.24 (p, J = 7.2 Hz, 4H), 3.10-2.90 (s, 3H), 2.52 (d, J = 29.3 Hz, 2H), 1.46 (d, J = 7.0 Hz, 3H), 1.36 (t, J = 7.3 Hz, 3H). |
| XIV- 75 | [chemical structure] | 610.30 [M + H]⁺ | (400 MHz, Methanol-d4) δ 8.32 (d, J = 2.6 Hz, 1H), 8.07 (d, J = 5.6 Hz, 1H), 7.81 (d, J = 2.0 Hz, 1H), 7.72-7.62 (m, 2H), 7.46 (d, J = 2.6 Hz, 1H), 6.58 (d, J = 5.5 Hz, 1H), 6.23 (s, 1H), 4.87 (d, J = 17.0 Hz, 1H), 4.73-4.64 (m, 3H), 3.89- 3.77 (m, 2H), 3.69 (s, 2H), 3.19 (q, J = 6.0 Hz, 1H), 3.09 (s, 4H), 2.74 (s, 3H), 2.67 (s, 4H), 1.40 (d, J = 7.0 Hz, 3H). |
| XIV- 76 | [chemical structure] | 664.30 [M + H]⁺ | (400 MHz, Methanol-d4) δ 8.45 (d, J = 2.6 Hz, 1H), 8.32 (d, J = 5.9 Hz, 1H), 7.81 (d, J = 2.1 Hz, 1H), 7.72-7.62 (m, 3H), 7.36 (s, 1H), 6.63 (d, J = 5.8 Hz, 1H), 4.99 (d, J = 17.2 Hz, 1H), 4.65 (d, J = 17.1 Hz, 1H), 4.24 (dd, J = 13.7, 4.2 Hz, 1H), 3.74 (s, 2H), 3.65 (dd, J = 13.6, 4.0 Hz, 1H), 3.48 (s, 2H), 3.20 (m, 3H), 3.02 (m, 4H), 2.48 (s, 2H), 1.92 (m, 1H), 1.70 (m, 1H), 1.33 (t, J = 7.3 Hz, 3H), 1.22 (t, J = 7.3 Hz, 3H), 1.11 (t, J = 7.4 Hz, 3H). |
| XIV- 77 | [chemical structure] | 652.25 [M + H]⁺ | (400 MHz, Methanol-d4) δ 8.42 (dd, J = 15.8, 2.6 Hz, 2H), 8.31 (d, J = 5.8 Hz, 1H), 8.14 (dd, J = 16.4, 2.6 Hz, 1H), 7.64 (s, 1H), 7.35 (s, 1H), 6.61 (d, J = 5.8 Hz, 1H), 5.55 (s, 1H), 4.96 (d, J = 17.1 Hz, 1H), 4.66 (d, J = 17.1 Hz, 1H), 4.22 (dd, J = 13.4, 4.2 Hz, 1H), 3.69-3.57 (m, 2H), 3.49 (d, J = 12.5 Hz, 1H), 3.24 (d, J = 14.2 Hz, 1H), 3.16 (d, J = 13.3 Hz, 1H), 3.02 (q, J = 7.5 Hz, 3H), 2.92 (d, J = 4.5 Hz, 3H), 2.46 (d, J = 14.0 Hz, 1H), 2.32 (d, J = 15.3 Hz, 1H), 2.14-1.93 (m, 3H), 1.70 (m, 1H), 1.22 (t, J = 7.4 Hz, 3H), 1.12 (t, J = 7.4 Hz, 3H). |
| XIV- 78 | [chemical structure] | 610.20 [M + H]⁺ | (400 MHz, Methanol-d₄) δ 8.34 (d, J = 2.6 Hz, 1H), 8.10 (d, J = 5.6 Hz, 1H), 7.83 (s, 1H), 7.72-7.63 (m, 2H), 7.48 (d, J = 2.7 Hz, 1H), 6.60 (d, J = 5.6 Hz, 1H), 6.25 (s, 1H), 4.86 (s, 2H), 4.75-4.68 (m, 3H), 4.62 (s, 3H), 3.89-3.81 (m, 2H), 3.64 (d, J = 1.7 Hz, 2H), 3.27- 3.20 (m, 1H), 2.54 (s, 5H), 2.32 (s, 3H), 1.43 (d, J = 7.0 Hz, 3H), 1.31 (s, 1H). |
| XIV- 79 | [chemical structure] | 664.25 [M + H]⁺ | (400 MHz, Methanol-d4) δ 8.49 (d, J = 2.8 Hz, 1H), 8.39-8.32 (m, 1H), 7.85 (d, J = 2.3 Hz, 1H), 7.75- 7.65 (m, 3H), 7.39 (p, J = 1.6 Hz, 1H), 6.67 (m, 1H), 5.02 (d, J = 17.0 Hz, 1H), 4.68 (d, J = 17.1 Hz, 1H), 4.27 (dd, J = 13.7, 4.2 Hz, 1H), 3.77 (s, 2H), 3.68 (dd, J = 13.6, 4.1 Hz, 1H), 3.23 (q, J = 7.3 Hz, 2H), 3.20-3.05 (m, 6H), 2.52 (s, 2H), 2.03-1.88 (m, 1H), 1.73 (m, 1H), 1.36 (t, J = 7.3 Hz, 3H), 1.25 (t, J = 7.4 Hz, 3H), 1.14 (t, J = 7.4 Hz, 3H). |
| XIV- 80 | [chemical structure] | 652.20 [M + H]⁺ | (400 MHz, Methanol-d4) δ 8.45 (m, 2H), 8.33 (d, J = 6.0 Hz, 1H), 8.17 (m, 1H), 7.65 (s, 1H), 7.36 (d, J = 1.2 Hz, 1H), 6.66-6.59 (m, 1H), 5.58 (s, 1H), 4.98 (d, J = 17.1 Hz, 1H), 4.68 (d, J = 17.0 Hz, 1H), 4.24 (m, 1H), 3.72-3.60 (m, 2H), 3.56-3.48 (m, 1H), 3.23 (m, 2H), 3.04 (q, J = 7.4 Hz, 3H), 2.95 (d, J = 4.3 Hz, 3H), 2.49 (d, J = 14.3 Hz, 1H), 2.35 (d, J = 15.3 Hz, 1H), 2.16 (t, J = 14.6 Hz, 1H), 1.96 (m, 2H), 1.73 (m, 1H), 1.25 (t, J = 7.4 Hz, 3H), 1.15 (t, J = 7.4 Hz, 3H). |
| XIV- 81 | [chemical structure] | 620.25 [M + H]⁺ | (400 MHz, Methanol-d4) δ 8.47 (s, 1H), 8.16 (t, J = 6.5 Hz, 1H), 7.85 (s, 1H), 7.76-7.63 (m, 3H), 6.84 (dd, J = 9.9, 6.5 Hz, 1H), 6.29 (d, J = 11.3 Hz, 1H), 4.77 (d, J = 17.1 Hz, 1H), 3.94 (dd, J = 13.7, 4.6 Hz, 1H), 3.84 (dd, J = 13.7, 6.2 Hz, 1H), 3.76 (s, 2H), 3.27 (s, 5H), 2.92 (s, 4H), 2.49 (s, 2H), 2.13 (d, J = 7.5 Hz, 1H), 1.45 (d, J = 7.0 Hz, 3H), 1.17 (dd, J = 8.9, 6.5 Hz, 2H), 0.98-0.85 (m, 2H). |
| XIV- 82 | [chemical structure] | 638.20 [M + H]⁺ | (400 MHz, Methanol-d4) δ 8.39 (d, J = 2.6 Hz, 1H), 8.21 (d, J = 5.6 Hz, 1H), 7.82 (d, J = 2.1 Hz, 1H), 7.73-7.63 (m, 2H), 7.51 (d, J = 2.7 Hz, 1H), 6.82 (s, 1H), 6.62 (d, J = 5.6 Hz, 1H), 4.96 (d, J = 17.0 Hz, 1H), 4.64 (d, J = 16.8 Hz, 1H), 4.28 (dd, J = 13.4, 4.0 Hz, 1H), 3.71 (s, 2H), 3.62 (dd, J = 13.5, 4.0 Hz, 1H), 3.14 (s, 4H), 2.97 (m, 1H), 2.78 (s, 3H), 2.70 (s, 4H), 1.93 (m, 1H), 1.67 (m, 1H), 1.13 (t, J = 7.4 Hz, 3H). |
| XIV- 82 | [chemical structure] | 620.25 [M + H]⁺ | (400 MHz, Methanol-d4) δ 8.46 (t, J = 2.0 Hz, 1H), 8.16 (dd, J = 6.7, 1.6 Hz, 1H), 7.85 (d, J = 2.0 Hz, 1H), 7.74-7.65 (m, 3H), 6.83 (dd, J = 6.8, 2.3 Hz, 1H), 6.28 (d, J = 2.6 Hz, 1H), 4.76 (d, J = 17.2 Hz, 1H), 3.94 (dd, J = 13.6, 4.6 Hz, 1H), 3.84 (dd, J = 13.6, 6.0 Hz, 1H), 3.75 (s, 2H), 3.45 (s, 2H), 3.27 (q, J = 6.2 Hz, 5H), 2.92 (s, 4H), 2.49 (s, 2H), 2.13 (td, J = 8.5, 4.3 Hz, 1H), 1.45 (d, J = 7.0 Hz, 3H), 1.19-1.10 (m, 2H), 0.94- 0.87 (m, 2H). |
| XIV- 84 | [chemical structure] | 637.20 [M + H]⁺ | (400 MHz, Methanol-d4) δ 8.40 (d, J = 2.8 Hz, 1H), 8.26 (d, J = 5.6 Hz, 1H), 7.81 (d, J = 2.1 Hz, 1H), 7.73-7.62 (m, 2H), 7.56 (d, J = 2.6 Hz, 1H), 7.16 (s, 1H), 6.59 (d, J = 5.5 Hz, 1H), 4.97 (d, J = 17.0 Hz, 1H), 4.65 (d, J = 17.0 Hz, 1H), 4.26 (dd, J = 13.6, 4.2 Hz, 1H), 3.70-3.58 (m, 3H), 2.99 (dd, J = 9.1, 4.5 Hz, 1H), 2.56-2.51 (m, 7H), 2.31 (s, 3H), 2.02-1.87 (m, 1H), 1.79-1.63 (m, 1H), 1.14 (t, J = 7.4 Hz, 3H). |
| XIV- 85 | [chemical structure] | 651.25 [M + H]⁺ | (400 MHz, Methanol-d4) δ 8.39 (d, J = 2.6 Hz, 1H), 8.25 (d, J = 5.5 Hz, 1H), 7.81 (d, J = 2.1 Hz, 1H), 7.72-7.62 (m, 2H), 7.55 (d, J = 2.6 Hz, 1H), 7.06 (s, 1H), 6.59 (d, J = 5.5 Hz, 1H), 4.97 (d, J = 17.0 Hz, 1H), 4.64 (d, J = 17.0 Hz, 1H), 4.25 (dd, J = 13.6, 4.3 Hz, 1H), 3.72-3.59 (m, 3H), 3.05-2.87 (m, 4H), 2.55-2.50 (m, 8H), 2.31 (s, 3H), 1.94 (s, 1H), 1.78-1.62 (m, 1H), 1.13 (t, J = 7.4 Hz, 3H). |
| XIV- 86 | [chemical structure] | 623.2 [M + H]⁺ | (400 MHz, Methanol-d4) δ 8.39 (d, J = 2.6 Hz, 1H), 8.26 (d, J = 5.5 Hz, 1H), 7.83 (d, J = 2.4 Hz, 1H), 7.73-7.63 (m, 2H), 7.56 (d, J = 2.6 Hz, 1H), 7.16 (s, 1H), 6.58 (d, J = 5.6 Hz, 1H), 4.93 (d, J = 17.1 Hz, 1H), 4.73 (d, J = 17.1 Hz, 1H), 3.94-3.80 (m, 2H), 3.64 (s, 2H), 3.28-3.18 (m, 1H), 2.55-2.50 (m, 8H), 2.31 (d, J = 1.4 Hz, 3H), 1.44 (d, J = 6.9 Hz, 3H |
| XIV- 87 | [chemical structure] | 660.1 [M + H]⁺ | N/A |
| XIV- 88 | [chemical structure] | 670.2 [M + H]⁺ | (400 MHz, Methanol-d4) δ 8.35 (d, J = 2.7 Hz, 1H), 8.25 (d, J = 3.6 Hz, 1H), 7.81 (d, J = 2.1 Hz, 1H), 7.72-7.59 (m, 2H), 7.46 (d, J = 2.7 Hz, 1H), 6.21 (s, 1H), 4.83 (q, J = 6.8 Hz, 5H), 4.64 (d, J = 17.1 Hz, 1H), 3.90-3.72 (m, 4H), 3.21 (q, J = 5.8 Hz, 1H), 2.90 (s, 3H), 1.39 (d, J = 6.9 Hz, 3H). |
| XIV- 89 | [chemical structure] | 610.25 [M + H]⁺ | (400 MHz, Methanol-d4) δ 8.31 (d, J = 2.7 Hz, 1H), 8.02 (d, J = 5.6 Hz, 1H), 7.61-7.55 (m, 1H), 7.48- 7.37 (m, 3H), 6.58 (d, J = 5.6 Hz, 1H), 5.99 (q, J = 1.0 Hz, 1H), 4.85 (d, 1H), 4.69 (d, J = 17.0 Hz, 1H), 3.90-3.79 (m, 2H), 3.57 (s, 2H), 3.25-3.16 (m, 1H), 2.55-2.50 (m, 9H), 2.43 (d, J = 1.0 Hz, 3H), 2.30 (s, 3H), 1.42 (d, J = 7.0 Hz, 3H). |
| XIV- 90 | [chemical structure] | 594.35 [M + H]⁺ | (400 MHz, Methanol-d4) δ 8.49 (d, J = 2.7 Hz, 1H), 8.22 (dd, J = 11.5, 6.8 Hz, 1H), 7.89-7.77 (m, 1H), 7.76-7.66 (m, 3H), 6.91 (dd, J = 19.2, 6.8 Hz, 1H), 6.42 (q, J = 1.0 Hz, 1H), 4.97 (d, 1H), 4.78 (d, J = 17.2 Hz, 1H), 3.99-3.90 (m, 1H), 3.89-3.81 (m, 1H), 3.78 (d, J = 1.5 Hz, 2H), 3.32-3.23 (m, 3H), 3.04-2.73 (m, 6H), 2.71- 2.32 (m, 6H), 1.46 (d, J = 7.0 Hz, 3H). |
| XIV- 91 | [chemical structure] | 594.2 [M + H]⁺ | (400 MHz, Methanol-d4) δ 8.31 (d, J = 2.7 Hz, 1H), 8.02 (d, J = 5.6 Hz, 1H), 7.82 (d, J = 2.0 Hz, 1H), 7.72-7.62 (m, 2H), 7.45 (d, J = 2.7 Hz, 1H), 6.58 (d, J = 5.6 Hz, 1H), 5.99 (q, J = 1.0 Hz, 1H), 4.88 (d, J = 17.1 Hz, 1H), 4.69 (d, J = 17.0 Hz, 1H), 3.88-3.80 (m, 2H), 3.64 (d, J = 1.6 Hz, 2H), 3.25- 3.16 (m, 1H), 2.55-2.51 (m, 9H), 2.43 (d, J = 1.0 Hz, 3H), 2.32 (s, 3H), 1.42 (d, J = 7.0 Hz, 3H). |
| XIV- 92 | [chemical structure] | 710.2 [M + H]⁺ | (400 MHz, DMSO-d6) δ ppm 0.99 (t, J = 1.00 Hz, 3H) 1.19 (t, J = 1.00 Hz, 3H) 1.46-1.64 (m, 1H) 1.80- 1.97 (m, 1H) 2.28-2.45 (m, 2H) 2.82-3.04 (m, 5H) 3.12 (q, J = 1.00 Hz, 2H) 3.37-3.53 (m, 2H) 3.58- 3.68 (m, 2H) 3.68-3.78 (m, 1H) 3.82 (s, 3H) 3.86-3.97 (m, 1H) 4.73 (dd, J = 1.00 Hz, 2H) 7.52- 7.64 (m, 2H) 7.74-7.81 (m, 1H) 7.84 (d, J = 1.00 Hz, 1H) 7.86-7.93 (m, 1H) 8.03 (s, 1H) 8.34 (s, 1H) 8.40-8.50 (m, 2H) 8.98 (s, 1H) 9.29 (br s, 2H) 10.66 (s, 1H) |
| XIV- 93 | [chemical structure] | 592.3 [M + H]⁺ | (400 MHz, Methanol-d4) δ 8.49 (d, J = 2.7 Hz, 1H), 8.21 (d, J = 6.8 Hz, 1H), 7.71 (d, J = 2.7 Hz, 1H), 7.50 (d, J = 2.0 Hz, 1H), 7.42 (d, J = 8.4 Hz, 1H), 7.35 (dd, J = 8.4, 2.1 Hz, 1H), 6.91-6.85 (m, 2H), 6.42 (d, J = 1.2 Hz, 1H), 4.96 (d, J = 17.3 Hz, 1H), 4.77 (d, J = 17.3 Hz, 1H), 3.98-3.90 (m, 3H), 3.84 (dd, J = 13.6, 6.1 Hz, 1H), 3.41- 3.37 (m, 3H), 3.32-3.23 (m, 2H), 3.08-3.04 (m, 4H), 2.92 (s, 3H), 2.54 (d, J = 1.1 Hz, 3H), 1.45 (d, J = 7.0 Hz, 3H). |
| XIV- 94 | [chemical structure] | 606.25 [M + H]⁺ | (400 MHz, Methanol-d4) δ 8.49 (d, J = 2.6 Hz, 1H), 8.21 (d, J = 6.8 Hz, 1H), 7.87 (d, J = 2.0 Hz, 1H), 7.77-7.67 (m, 3H), 6.88 (d, J = 6.8 Hz, 1H), 6.41 (d, J = 1.2 Hz, 1H), 4.95 (d, J = 17.2 Hz, 1H), 4.78 (d, J = 17.2 Hz, 1H), 3.94 (dd, J = 13.6, 4.6 Hz, 1H), 3.89-3.80 (m, 3H), 3.40 (t, J = 5.2 Hz, 2H), 3.32-3.18 (m, 1H), 2.93-2.86 (m, 2H), 2.73-2.69 (m, 2H), 2.54 (d, J = 1.1 Hz, 3H), 1.46 (d, J = 7.0 Hz, 3H), 1.37-1.22 (m, 1H), 1.13- 1.05 (m, 2H), 1.05-0.89 (m, 2H). |
| XIV- 95 | [chemical structure] | 642.35 [M + H]⁺ | (400 MHz, Methanol-d₄) δ 8.37 (d, J = 2.7 Hz, 1H), 8.24 (d, J = 3.7 Hz, 1H), 7.84 (d, J = 2.1 Hz, 1H), 7.74-7.60 (m, 2H), 7.48 (d, J = 2.7 Hz, 1H), 6.04 (s, 1H), 4.86 (s, 1H), 4.67 (d, J = 18.7 Hz, 3H), 3.94-3.80 (m, 2H), 3.77 (s, 2H), 3.57-3.44 (m, 1H), 3.23 (q, J = 7.3 Hz, 4H), 2.81 (d, J = 200.6 Hz, 5H), 1.42 (d, J = 6.9 Hz, 3H), 1.36 (t, J = 7.3 Hz, 3H). |
| XIV- 96 | [chemical structure] | 628.30 [M + H]⁺ | (400 MHz, Methanol-d4) δ 8.34 (d, J = 2.7 Hz, 1H), 8.21 (d, J = 3.8 Hz, 1H), 7.81 (d, J = 2.2 Hz, 1H), 7.72-7.61 (m, 2H), 7.44 (d, J = 2.7 Hz, 1H), 6.01 (s, 1H), 5.47 (s, 2H), 4.86 (d, J = 17.2 Hz, 1H), 4.67 (s, 2H), 3.90-3.76 (m, 2H), 3.73 (s, 2H), 3.21 (q, J = 5.7 Hz, 1H), 2.90 (s, 3H), 2.62 (s, 2H), 1.40 (d, J = 7.0 Hz, 3H) |
| XIV- 97 | [chemical structure] | 636.25 [M + H]⁺ | (400 MHz, Methanol-d4) δ 8.41 (d, J = 2.7 Hz, 1H), 8.30 (d, J = 5.6 Hz, 1H), 7.83 (d, J = 1.9 Hz, 1H), 7.73-7.63 (m, 2H), 7.59 (d, J = 2.6 Hz, 1H), 7.32 (s, 1H), 6.55 (d, J = 5.6 Hz, 1H), 4.92 (d, J = 17.0 Hz, 1H), 4.74 (d, J = 17.0 Hz, 1H), 3.95-3.80 (m, 2H), 3.64 (s, 2H), 3.27-3.20 (m, 1H), 3.08-2.98 (m, 2H), 2.56-2.51 (m, 8H), 2.32 (s, 3H), 1.45 (d, J = 7.0 Hz, 3H), 1.24 (t, J = 7.4 Hz, 3H). |
| XIV- 98 | [chemical structure] | 636.25 [M + H]⁺ | (400 MHz, Methanol-d4) δ 8.41 (d, J = 2.7 Hz, 1H), 8.30 (d, J = 5.5 Hz, 1H), 7.83 (d, J = 2.0 Hz, 1H), 7.73-7.63 (m, 2H), 7.59 (d, J = 2.6 Hz, 1H), 7.32 (s, 1H), 6.55 (d, J = 5.6 Hz, 1H), 4.92 (d, J = 17.0 Hz, 1H), 4.74 (d, J = 17.0 Hz, 1H), 3.95-3.80 (m, 2H), 3.64 (s, 2H), 3.24 (d, J = 6.1 Hz, 1H), 3.03 (q, J = 7.3 Hz, 2H), 2.56-2.51 (m, 8H), 2.32 (s, 3H), 1.45 (d, J = 7.0 Hz, 3H), 1.24 (t, J = 7.3 Hz, 3H). |
| XIV- 99 | [chemical structure] | 606.30 [M + H]⁺ | (400 MHz, Methanol-d4) δ 8.47 (d, J = 2.5 Hz, 1H), 8.20 (d, J = 6.7 Hz, 1H), 7.87 (s, 1H), 7.73 (s, 2H), 7.69 (s, 1H), 6.87 (d, J = 6.7 Hz, 1H), 6.39 (s, 1H), 4.97 (s, 0H), 4.78 (d, J = 17.2 Hz, 1H), 4.24- 4.20 (m, 1H), 4.10-4.02 (m, 1H), 4.00-3.90 (m, 2H), 3.89-3.80 (m, 1H), 3.80-3.76 (m, 1H), 3.75 (s, 2H), 3.30-3.23 (m, 1H), 3.21- 3.16 (m, 3H), 3.06-2.98 (m, 1H), 2.95 (s, 3H), 2.54 (s, 3H), 2.29-2.25 (m, 1H), 2.24-2.16 (m, 1H), 1.46 (d, J = 7.0 Hz, 3H). |
| XIV- 100 | [chemical structure] | 606.30 [M + H]⁺ | (400 MHz, Methanol-d4) δ 8.29 (d, J = 2.7 Hz, 1H), 8.00 (d, J = 5.6 Hz, 1H), 7.80 (d, J = 2.2 Hz, 1H), 7.71 (d, J = 8.6 Hz, 1H), 7.64 (d, J = 8.6 Hz, 1H), 7.42 (d, J = 2.6 Hz, 1H), 6.56 (d, J = 5.6 Hz, 1H), 5.97 (d, J = 1.2 Hz, 1H), 4.67 (d, J = 17.0 Hz, 1H), 3.89 (d, J = 14.9 Hz, 1H), 3.86-3.75 (m, 3H), 3.37 (s, 1H), 3.00 (s, 1H), 2.88 (d, J = 10.6 Hz, 1H), 2.70 (d, J = 10.0 Hz, 2H), 2.48 (s, 3H), 2.41 (d, J = 1.1 Hz, 3H), 1.85 (q, J = 10.5 Hz, 2H), 1.40 (d, J = 7.0 Hz, 3H), 1.29 (s, 2H), 0.88 (s, 1H), 0.10 (s, 1H). |
| XIV- 101 | [chemical structure] | 606.50 [M + H]⁺ | (400 MHz, Methanol-d4) δ 8.50 (d, J = 2.6 Hz, 1H), 8.22 (d, J = 6.8 Hz, 1H), 7.92 (d, J = 2.2 Hz, 1H), 7.85-7.71 (m, 3H), 6.89 (d, J = 6.8 Hz, 1H), 6.41 (s, 1H), 4.99 (d, 1H), 4.79 (d, J = 17.2 Hz, 1H), 3.99-3.91 (m, 3H), 3.95-3.81 (m, 1H), 3.71-3.66 (m, 2H), 3.46- 3.37 (m, 2H), 3.33-3.23 (m, 3H), 2.54 (s, 3H), 2.45-2.37 (m, 2H), 2.12-2.01 (m, 2H), 1.46 (d, J = 7.0 Hz, 3H). |
| XIV- 102 | [chemical structure] | 691.30 [M + H]⁺ | (400 MHz, Methanol-d₄) δ 8.50 (d, J = 2.7 Hz, 1H), 8.35 (s, 1H), 7.84 (d, J = 2.1 Hz, 1H), 7.75-7.65 (m, 3H), 7.02 (s, 1H), 6.78 (d, J = 6.1 Hz, 1H), 5.02 (d, J = 17.1 Hz, 1H), 4.68 (d, J = 17.2 Hz, 1H), 4.26 (dd, J = 13.6, 4.3 Hz, 1H), 3.85 (t, J = 6.7 Hz, 2H), 3.76 (s, 2H), 3.73- 3.65 (m, 3H), 3.56-3.35 (m, 2H), 3.33-3.18 (m, 2H), 3.27-3.12 (m, 2H), 3.07-2.99 (m, 4H), 2.53- 2.48 (m, 2H), 2.13-2.04 (m, 2H), 2.04-1.88 (m, 3H), 1.80- 1.64 (m, 1H), 1.15 (t, J = 7.3 Hz, 3H). |
| XIV- 103 | [chemical structure] | 693.30 [M + H]⁺ | (400 MHz, Methanol-d₄) δ 8.50 (d, J = 2.7 Hz, 1H), 8.35 (d, J = 6.0 Hz, 1H), 7.84 (d, J = 2.1 Hz, 1H), 7.76-7.65 (m, 3H), 6.86 (s, 1H), 6.75 (dd, J = 6.1, 1.1 Hz, 1H), 5.02 (d, J = 17.1 Hz, 1H), 4.76-4.64 (m, 2H), 4.46 (s, 1H), 4.34-4.22 3.76 (s, 2H), 3.68 (dd, J = 13.7, 4.1 Hz, 1H), 3.54-3.33 (m, 2H), 3.32- 3.14 (m, 2H), 3.07-2.99 (m, 1H), 2.92 (m, 4H), 2.63-2.58 (m, 3H), 2.02-1.89 (m, 1H), 1.80- 1.65 (m, 1H), 1.15 (t, J = 7.4 Hz, 3H). |
| XIV- 104 | [chemical structure] | 702.30 [M + H]⁺ | (400 MHz, Methanol-d₄) δ 8.47 (d, J = 2.7 Hz, 1H), 8.33 (d, J = 5.9 Hz, 1H), 7.84 (d, J = 2.2 Hz, 1H), 7.75-7.61 (m, 3H), 6.87 (s, 1H), 6.69 (d, J = 5.9 Hz, 1H), 5.02 (d, J = 17.1 Hz, 1H), 4.82-4.71 (m, 1H), 4.67 (d, J = 17.1 Hz, 1H), 4.62-4.47 (m, 1H), 4.40 (dd, J = 23.7, 9.7 Hz, 1H), 4.27 (dd, J = 13.7, 4.3 Hz, 1H), 3.88 (tt, J = 9.2, 6.1 Hz, 1H), 3.76 (s, 2H), 3.67 (d, J = 4.1 Hz, 1H), 3.58-3.36 (m, 2H), 3.29-3.13 (m, 2H), 3.06- 2.98 (m, 2H), 2.92 (s, 4H), 2.70- 2.37 (m, 2H), 2.04-1.88 (m, 1H), 1.81-1.65 (m, 1H), 1.15 (t, J = 7.4 Hz, 3H). |
| XIV- 105 | [chemical structure] | 695.30 [M + H]⁺ | (400 MHz, Methanol-d₄) δ 8.51 (d, J = 2.7 Hz, 1H), 8.36 (s, 1H), 7.84 (d, J = 2.1 Hz, 1H), 7.76-7.65 (m, 3H), 6.94 (s, 1H), 6.76 (d, J = 6.0 Hz, 1H), 5.65-5.34 (m, 1H), 5.03 (d, J = 17.2 Hz, 1H), 4.78-4.42 (m, 4H), 4.31-4.22 (m, 2H), 3.77 (s, 2H), 3.69 (dd, J = 13.6, 4.0 Hz, 1H), 3.60-3.40 (m, 2H), 3.30-3.22 (m, 2H), 3.08-2.99 (m, 1H), 2.92 (s, 4H), 2.74 (s, 2H), 2.02-1.91 (m, 1H), 1.80-1.65 (m, 1H), 1.15 (t, J = 7.4 Hz, 3H). |
| XIV- 106 | [chemical structure] | 695.25 [M + H]⁺ | (400 MHz, Methanol-d₄) δ 8.39 (d, J = 2.6 Hz, 1H), 8.24 (d, J = 5.6 Hz, 1H), 7.81 (d, J = 2.1 Hz, 1H), 7.75-7.62 (m, 2H), 7.56 (d, J = 2.6 Hz, 1H), 6.84 (s, 1H), 6.61 (d, J = 5.6 Hz, 1H), 4.97 (d, J = 17.0 Hz, 1H), 4.65 (d, J = 17.0 Hz, 1H), 4.25 (dd, J = 13.6, 4.3 Hz, 1H), 3.84 (t, J = 5.3 Hz, 2H), 3.74 (s, 2H), 3.65 (d, J = 9.3 Hz, 3H), 3.41 (d, J = 6.0 Hz, 1H), 3.15 (dd, J = 3.4, 1.7 Hz, 2H), 2.99 (dd, J = 9.1, 4.5 Hz, 1H), 2.83-2.36 (m, 8H), |
| | | | 2.31 (s, 3H), 1.99-1.87 (m, 1H), |
| | | | 1.71 (ddt, J = 16.5, 14.3, 7.4 Hz, |
| | | | 1H), 1.13 (t, J = 7.4 Hz, 3H). |
| XIV- 107 | [chemical structure] | 681.30 [M + H]⁺ | (400 MHz, Methanol-d₄) δ 8.39 (d, J = 2.7 Hz, 1H), 8.26 (d, J = 5.6 Hz, 1H), 7.81 (d, J = 2.1 Hz, 1H), 7.74-7.63 (m, 2H), 7.55 (d, J = 2.7 Hz, 1H), 7.12 (s, 1H), 6.59 (d, J = 5.5 Hz, 1H), 4.97 (d, J = 17.1 Hz, 1H), 4.64 (d, J = 17.0 Hz, 1H), 4.25 (dd, J = 13.5, 4.2 Hz, 1H), 3.74 (t, J = 5.8 Hz, 2H), 3.65 (dd, J = 10.8, 2.9 Hz, 3H), 3.54 (t, J = 5.8 Hz, 2H), 2.98 (dd, J = 9.1, 4.5 Hz, 1H), 2.78-2.34 (m, 8H), 2.32 (s, 3H), 1.94 (ddd, J = 13.8, 7.5, 4.6 |
| | | | Hz, 1H), 1.70 (ddt, J = 16.5, 14.4, |
| | | | 7.4 Hz, 1H), 1.13 (t, J = 7.4 Hz, |
| | | | 3H). |
| XIV- 108 | [chemical structure] | 695.30 [M + H]⁺ | (400 MHz, Methanol-d₄) δ 8.51- 8.41 (m, 1H), 8.36-8.25 (m, 1H), 7.85-7.77 (m, 1H), 7.74-7.58 (m, 3H), 7.24-7.13 (m, 1H), 6.76- 6.60 (m, 1H), 5.00 (d, J = 17.0 Hz, 1H), 4.67 (d, J = 17.2 Hz, 1H), 4.25 (dd, J = 13.6, 4.3 Hz, 1H), 3.78-3.72 (m, 2H), 3.71-3.58 (m, 3H), 3.55-3.50 (m, 2H), 3.27- 3.19 (m, 3H), 3.06-2.99 (m, 1H), 2.94-2.84 (m, 3H), 2.61- 2.36 (m, 2H), 2.01-1.92 (m, 1H), 1.90-1.79 (m, 2H), 1.76-1.64 |
| | | | (m, 1H), 1.33 (t, J = 7.3 Hz, 3H), |
| | | | 1.14 (t, J = 7.4 Hz, 3H). |
| XIV- 109 | [chemical structure] | 707.35 [M + H]⁺ | (400 MHz, Methanol-d₄) δ 8.52 (d, J = 2.5 Hz, 1H), 8.37 (s, 1H), 7.84 (d, J = 2.1 Hz, 1H), 7.71 (dt, J = 12.8, 5.5 Hz, 3H), 7.06 (d, J = 10.7 Hz, 1H), 6.80 (t, J = 5.3 Hz, 1H), 5.03 (d, J = 17.1 Hz, 1H), 4.69 (d, J = 17.0 Hz, 1H), 4.54 (d, J = 17.2 Hz, 1H), 4.30-4.21 (m, 1H), 4.03- 3.93 (m, 2H), 3.87-3.64 (m, 6H), 3.50 (s, 2H), 3.55-3.34 (m, 2H), 3.08-2.99 (m, 4H), 2.92 (s, 3H), 2.21-2.08 (m, 3H), 1.81- 1.65 (m, 1H), 1.15 (t, J = 7.4 Hz, 3H). |
| XIV- 110 | [chemical structure] | 707.55 [M + H]⁺ | (400 MHz, Methanol-d₄) δ 8.42 (d, J = 2.6 Hz, 1H), 8.27 (d, J = 5.6 Hz, 1H), 7.82 (d, J = 2.0 Hz, 1H), 7.72-7.61 (m, 2H), 7.59 (s, 1H), 6.87 (d, J = 13.3 Hz, 1H), 6.61 (dd, J = 5.6, 2.0 Hz, 1H), 4.98 (d, J = 17.0 Hz, 1H), 4.72-4.46 (m, 3H), 4.26 (d, J = 13.6 Hz, 1H), 4.00- 3.92 (m, 1H), 3.84-3.63 (m, 5H), 3.00 (dd, J = 9.1, 4.5 Hz, 1H), 2.73- 2.41 (m, 7H), 2.35 (s, 3H), 2.20- 1.92 (m, 3H), 1.80-1.66 (m, 1H), 1.14 (t, J = 7.4 Hz, 3H). |
| XIV- 111 | [chemical structure] | 709.30 [M + H]⁺ | (400 MHz, Methanol-d₄) δ 8.48 (s, 1H), 8.35-8.30 (m, 1H), 7.84 (d, J = 4.7 Hz, 1H), 7.75-7.65 (m, 3H), 6.98 (dd, J = 6.8, 4.4 Hz, 1H), 6.81-6.72 (m, 1H), 5.01 (d, J = 17.1 Hz, 1H), 4.68 (d, J = 17.1 Hz, 1H), 4.26 (dd, J = 13.6, 4.3 Hz, 1H), 3.80-3.61 (m, 7H), 3.55- 3.39 (m, 2H), 3.17 (dd, J = 34.8, 18.5 Hz, 3H), 3.06-3.01 (m, 1H), 2.92 (s, 4H), 2.70-2.38 (m, 2H), 1.94 (dp, J = 13.8, 6.9 Hz, 3H), 1.72 (dt, J = 14.4, 7.6 Hz, 1H), |
| | | | 1.14 (t, J = 7.4 Hz, 3H). |
| XIV- 112 | [chemical structure] | 676.25 [M + H]⁺ | (400 MHz, Methanol-d4) δ 8.47 (d, J = 2.6 Hz, 1H), 8.32 (d, J = 5.8 Hz, 1H), 7.83 (d, J = 2.1 Hz, 1H), 7.75-7.61 (m, 3H), 7.19 (s, 1H), 6.70 (d, J = 5.7 Hz, 1H),5.01 (d, J = 17.1 Hz, 1H), 4.67 (d, J = 17.1 Hz, 1H), 4.27 (dd, J = 13.6, 4.2 Hz, 1H), 3.76 (m, 2H), 3.68 (dd, J = 13.6, 4.1 Hz, 1H), 3.58-3.39 (m, 1H), 3.33-3.24 (m, 2H), 3.21 (m, 2H), 3.07-2.99 (m, 4H), 2.92 (s, 4H), 2.66 (s, 2H), 2.03-1.88 (m, 1H), 1.73 (s, 1H), 1.14 (t, J = 7.4 Hz, 3H). |
| XIV- 113 | [chemical structure] | 662.25 [M + H]⁺ | (400 MHz, Methanol-d4) δ 8.47 (d, J = 2.6 Hz, 1H), 8.32 (d, J = 5.8 Hz, 1H), 7.83 (d, J = 2.0 Hz, 1H), 7.74-7.69 (m, 2H), 7.67 (d, J = 2.6 Hz, 1H), 7.19 (s, 1H), 6.70 (d, J = 5.7 Hz, 1H), 5.01 (d, J = 17.1 Hz, 1H), 4.67 (d, J = 17.1 Hz, 1H), 4.27 (dd, J = 13.6, 4.2 Hz, 1H), 3.76 (s, 2H), 3.67 (dd, J = 13.6, 4.0 Hz, 1H), 3.26 (t, J = 5.2 Hz, 4H), 3.03 (dd, J = 9.1, 4.7 Hz, 1H), 2.74 (t, J = 5.3 Hz, 4H), 2.66 (s, 3H), 2.01-1.88 (m, 1H), 1.81-1.65 (m, 1H), 1.15 (t, J = 7.4 Hz, 3H). |
| XIV- 114 | [chemical structure] | 678.30 [M + H]⁺ | (400 MHz, Methanol-d4, ppm): δ 8.45 (d, J = 2.6 Hz, 1H), 8.29 (d, J = 5.8 Hz, 1H), 7.83 (d, J = 2.2 Hz, 1H), 7.75-7.60 (m, 3H), 7.00 (s, 1H), 6.68 (d, J = 5.8 Hz, 1H), 5.00 (d, J = 17.0 Hz, 1H), 4.67 (d, J = 17.1 Hz, 1H), 4.27 (dd, J = 13.5, 4.2 Hz, 1H), 3.75 (s, 2H), 3.67 (dd, J = 13.6, 4.1 Hz, 1H), 3.54-3.39 (m, 2H), 3.12-2.95 (m, 3H), 2.92 (s, 4H), 2.53-2.36 (m, 2H), 1.95 (dt, J = 13.3, 6.0 Hz, 1H), 1.72 (dt, J = 14.4, 7.6 Hz, 1H), 1.14 (t, J = 7.4 Hz, 3H). |
| XIV- 115 | [chemical structure] | 666.40 [M + H]⁺ | (400 MHz, Methanol-d4) δ 8.55- 8.48 (m, 2H), 8.38 (d, J = 5.4 Hz, 1H), 7.84 (d, J = 1.9 Hz, 1H), 7.76- 7.65 (m, 3H), 6.62 (d, J = 5.4 Hz, 1H), 5.01 (d, J = 17.0 Hz, 1H), 4.68 (d, J = 17.0 Hz, 1H), 4.25 (dd, J = 13.7, 4.3 Hz, 1H), 3.82-3.63 (m, 4H), 3.38 (s, 5H), 3.15 (s, 4H), 3.02 (dd, J = 8.9, 4.6 Hz, 2H), 2.92 (s, 6H), 1.95 (dt, J = 13.3, 6.2 Hz, 1H), 1.70 (dq, J = 15.1, 7.6 Hz, 1H), 1.14 (t, J = 7.4 Hz, 3H). |
| XIV- 116 | [chemical structure] | 652.50 [M + H]⁺ | (400 MHz, Methanol-d4) δ 8.52 (d, J = 8.1 Hz, 2H), 8.39 (d, J = 5.4 Hz, 1H), 7.85 (s, 1H), 7.77-7.67 (m, 3H), 6.66-6.60 (m, 1H), 5.02 (d, J = 17.1 Hz, 1H), 4.68 (d, J = 17.1 Hz, 1H), 4.26 (dd, J = 13.8, 4.2 Hz, 1H), 3.85-3.76 (m, 2H), 3.67 (dd, J = 13.6, 4.1 Hz, 1H), 3.37 (s, 3H), 3.31-3.23 (m, 4H), 3.15 (s, 3H), 3.03 (dd, J = 9.3, 4.5 Hz, 1H), 2.81 (s, 4H), 1.95 (ddd, J = 13.3, 7.3, 4.7 Hz, 1H), 1.71 (ddd, J = 13.8, 8.7, 7.0 Hz, 1H), 1.14 (t, J = 7.4 Hz, 3H). |
| XIV- 117 | [chemical structure] | 664.31 [M + H]⁺ | (400 MHz, Methanol-d4) δ 8.48 (d, J = 2.5 Hz, 1H), 8.11 (d, J = 1.8 Hz, 1H), 8.04 (d, J = 5.4 Hz, 1H), 7.84 (d, J = 2.1 Hz, 1H), 7.75- 7.65 (m, 3H), 6.96 (d, J = 1.8 Hz, 1H), 5.99 (d, J = 5.4 Hz, 1H), 5.02 (d, J = 17.0 Hz, 1H), 4.68 (d, J = 17.1 Hz, 1H), 4.26 (dd, J = 13.7, 4.3 Hz, 1H), 3.77 (s, 2H), 3.66 (dd, J = 13.8, 4.0 Hz, 1H), 3.15 (s, 3H), 3.02 (dd, J = 9.2, 4.6 Hz, 2H), 2.92 (s, 4H), 2.63 (d, J = 87.4 Hz, 3H), 2.02-1.87 (m, 1H), 1.71 (dp, J = 14.8, 7.5 Hz, 1H), 1.14 (t, J = 7.4 Hz, 3H) |
| XIV- 118 | [chemical structure] | 650.29 [M + H]⁺ | (400 MHz, Methanol-d4) δ 8.48 (d, J = 2.6 Hz, 1H), 8.23 (d, J = 1.8 Hz, 1H), 8.04 (d, J = 5.4 Hz, 1H), 7.84 (d, J = 2.1 Hz, 1H), 7.75- 7.62 (m, 3H), 7.11 (d, J = 1.9 Hz, 1H), 5.98 (d, J = 5.4 Hz, 1H), 5.02 (d, J = 17.1 Hz, 1H), 4.68 (d, J = 17.1 Hz, 1H), 4.26 (dd, J = 13.6, 4.2 Hz, 1H), 3.77 (s, 2H), 3.66 (dd, J = 13.6, 4.0 Hz, 1H), 3.02 (dd, J = 9.2, 4.5 Hz, 1H), 2.94 (d, J = 9.6 Hz, 7H), 2.76 (d, J = 12.2 Hz, 3H), 2.09-1.86 (m, 1H), 1.71 (dp, J = 14.9, 7.5 Hz, 1H), 1.14 (t, J = 7.4 Hz, 3H) |
| XIV- 119 | [chemical structure] | 666.35 [M + H]⁺ | (400 MHz, Methanol-d4) δ 8.51 (s, 2H), 8.38 (d, J = 5.4 Hz, 1H), 7.83 (s, 1H), 7.70 (d, J = 6.8 Hz, 3H), 6.62 (d, J = 5.4 Hz, 1H), 5.01 (d, J = 17.0 Hz, 1H), 4.68 (d, J = 17.1 Hz, 1H), 4.25 (dd, J = 13.2, 4.3 Hz, 1H), 3.76 (s, 2H), 3.72-3.63 (m, 1H), 3.45 (s, 2H), 3.38 (s, 3H), 3.16 (s, 4H), 3.02 (s, 2H), 2.92 (s, 4H), 2.51 (s, 2H), 1.95 (s, 1H), 1.77-1.64 (m, 1H), 1.14 (t, J = 7.3 Hz, 3H). |
| XIV- 120 | [chemical structure] | 652.3 [M + H]⁺ | (400 MHz, Methanol-d4) δ 8.54 (d, J = 26.8 Hz, 2H), 8.35 (d, J = 5.4 Hz, 1H), 7.83 (s, 1H), 7.70 (d, J = 5.7 Hz, 3H), 6.54 (d, J = 5.5 Hz, 1H), 5.01 (d, J = 17.1 Hz, 1H), 4.69 (d, J = 17.2 Hz, 1H), 4.24 (d, J = 13.0 Hz, 1H), 3.76 (s, 2H), 3.69 (d, J = 12.8 Hz, 1H), 3.50 (s, 2H), 3.20 (s, 2H), 2.99 (s, 5H), 2.92 (s, 4H), 1.96 (s, 1H), 1.76- 1.64 (m, 1H), 1.14 (t, J = 7.4 Hz, 3H). |
| XIV- 121 | [chemical structure] | 652.4 [M + H]⁺ | (400 MHz, Methanol-d4) δ 8.46 (d, J = 2.5 Hz, 1H), 8.39 (s, 1H), 7.84 (d, J = 2.1 Hz, 1H), 7.75- 7.64 (m, 3H), 7.25 (s, 1H), 5.03 (d, J = 17.0 Hz, 1H), 4.68 (d, J = 16.9 Hz, 1H), 4.30 (dd, J = 13.6, 4.0 Hz, 1H), 3.75 (s, 3H), 3.64 (dd, J = 13.5, 3.9 Hz, 1H), 3.22-3.13 (m, 2H), 2.98 (s, 6H), 2.92 (s, 4H), 2.61-2.36 (m, 2H), 2.02-1.87 (m, 1H), 1.80-1.64 (m, 1H), 1.15 (t, J = 7.4 Hz, 3H). |
| XIV- 122 | [chemical structure] | 650.29 [M + H]⁺ | (400 MHz, Methanol-d4) δ 8.49 (s, 1H), 8.34 (d, J = 6.2 Hz, 1H), 7.85 (s, 1H), 7.71 (d, J = 3.2 Hz, 3H), 6.97 (d, J = 2.0 Hz, 1H), 6.78 (d, J = 6.7 Hz, 1H), 5.02 (d, J = 17.0 Hz, 1H), 4.68 (d, J = 17.1 Hz, 1H), 4.26 (dd, J = 13.8, 4.3 Hz, 1H), 3.77 (s, 2H), 3.69 (dd, J = 13.7, 4.0 Hz, 1H), 3.27 (t, J = 5.2 Hz, 5H), 3.18 (s, 3H), 3.07-2.99 (m, 1H), 2.76 (s, 4H), 1.96 (dt, J = 13.0, 6.1 Hz, 1H), 1.72 (dt, J = 14.4, 7.4 Hz, 1H), 1.14 (t, J = 7.4 Hz, 3H). |
| XIV- 123 | [chemical structure] | 604.25 [M + H]⁺ | (400 MHz, Methanol-d4) δ 8.47 (d, J = 2.6 Hz, 1H), 8.37 (d, J = 5.6 Hz, 1H), 7.85 (d, J = 2.1 Hz, 1H), 7.71 (td, J = 9.0, 7.9, 5.5 Hz, 3H), 7.28 (s, 1H), 6.65 (d, J = 5.6 Hz, 1H), 5.02 (d, J = 17.1 Hz, 1H), 4.67 (d, J = 17.1 Hz, 1H), 4.28 (dd, J = 13.6, 4.2 Hz, 1H), 3.80 (s, 2H), 3.66 (dd, J = 13.5, 4.0 Hz, 1H), 3.28 (t, J = 5.2 Hz, 4H), 3.03 (dd, J = 9.2, 4.6 Hz, 1H), 2.79 (t, J = 5.1 Hz, 4H), 2.03-1.86 (m, 1H), 1.83- 1.65 (m, 1H), 1.14 (t, J = 7.4 Hz, 3H) |
| XIV- 124 | [chemical structure] | 651.30 [M + H]⁺ | (400 MHz, Methanol-d₄) δ 8.47 (d, J = 2.5 Hz, 1H), 8.11 (d, J = 1.7 Hz, 1H), 8.04 (d, J = 5.4 Hz, 1H), 7.84 (s, 1H), 7.71 (td, J = 8.5, 7.2, 5.4 Hz, 3H), 6.96 (d, J = 1.8 Hz, 1H), 5.99 (d, J = 5.4 Hz, 1H), 5.01 (d, J = 17.1 Hz, 1H), 4.68 (d, J = 17.1 Hz, 1H), 4.26 (dd, J = 13.8, 4.2 Hz, 1H), 3.77 (s, 2H), 3.67 (dd, J = 13.7, 4.0 Hz, 1H), 3.27 (t, J = 5.2 Hz, 5H), 3.16 (s, 3H), 3.01 (dd, J = 9.1, 4.6 Hz, 1H), 2.76 (s, 4H), 1.95 (dt, J = 12.8, 5.9 Hz, 1H), 1.71 (dt, J = 14.5, 7.5 Hz, 1H), 1.14 (t, J = 7.4 Hz, 3H). |
| XIV- 125 | [chemical structure] | 637.25 [M + H]⁺ | (400 MHz, Methanol-d₄) δ 8.47 (d, J = 2.6 Hz, 1H), 8.23 (d, J = 1.8 Hz, 1H), 8.04 (d, J = 5.4 Hz, 1H), 7.84 (s, 1H), 7.73-7.66 (m, 3H), 7.12 (d, J = 1.8 Hz, 1H), 5.97 (d, J = 5.4 Hz, 1H), 5.01 (d, J = 17.0 Hz, 1H), 4.68 (d, J = 17.1 Hz, 1H), 4.26 (dd, J = 13.7, 4.2 Hz, 1H), 3.79 (s, 2H), 3.67 (dd, J = 13.6, 4.1 Hz, 1H), 3.27 (d, J = 5.3 Hz, 4H), 3.02 (dd, J = 9.1, 4.6 Hz, 1H), 2.95 (s, 3H), 2.77 (s, 4H), 1.95 (dt, J = 12.9, 6.6 Hz, 1H), 1.77-1.66 (m, 1H), 1.14 (t, J = 7.4 Hz, 3H). |
| XIV- 126 | [chemical structure] | 652.45 [M + H]⁺ | (400 MHz, Methanol-d₄) δ 8.56- 8.50 (m, 1H), 8.40 (s, 1H), 7.87- 7.77 (m, 2H), 7.77-7.66 (m, 2H), 7.08 (s, 1H), 5.06 (d, J = 17.0 Hz, 1H), 4.69 (d, J = 17.0 Hz, 1H), 4.33 (dd, J = 13.6, 3.8 Hz, 1H), 3.77 (s, 2H), 3.63 (dd, J = 13.6, 3.9 Hz, 1H), 3.42-3.37 (m, 3H), 3.33- 3.23 (m, 5H), 3.22-3.16 (m, 4H), 3.04 (dd, J = 9.1, 4.4 Hz, 1H), 2.75 (s, 4H), 2.00-1.89 (m, 1H), 1.81-1.65 (m, 1H), 1.15 (t, J = 7.4 Hz, 3H). |
| XIV- 127 | [chemical structure] | 638.45 [M + H]⁺ | (400 MHz, Methanol-d₄) δ 8.50 (d, J = 2.5 Hz, 1H), 8.39 (s, 1H), 7.84 (d, J = 2.1 Hz, 1H), 7.78-7.67 (m, 3H), 7.25 (s, 1H), 5.05 (d, J = 17.0 Hz, 1H), 4.69 (d, J = 16.9 Hz, 1H), 4.32 (dd, J = 13.6, 4.0 Hz, 1H), 3.78-3.60 (m, 4H), 3.26 (t, J = 5.2 Hz, 4H), 3.08-2.94 (m, 4H), 2.75 (t, J = 5.1 Hz, 3H), 2.00- 1.87 (m, 1H), 1.81-1.65 (m, 1H), 1.15 (t, J = 7.4 Hz, 3H). |
| XIV- 128 | [chemical structure] | 652.30 [M + H]⁺ | (400 MHz, Methanol-d₄) δ 8.50 (d, J = 3.0 Hz, 2H), 8.38 (d, J = 5.4 Hz, 1H), 7.83 (d, J = 2.0 Hz, 1H), 7.75-7.64 (m, 3H), 6.61 (d, J = 5.4 Hz, 1H), 5.00 (d, J = 17.1 Hz, 2H), 4.68 (d, J = 17.1 Hz, 2H), 4.24 (dd, J = 13.6, 4.3 Hz, 2H), 3.75 (s, 2H), 3.68 (dd, J = 13.6, 4.1 Hz, 1H), 3.38 (s, 3H), 3.26 (t, J = 5.2 Hz, 4H), 3.15 (s, 3H), 3.01 (dd, J = 9.2, 4.6 Hz, 1H), 2.79-2.70 (m, 4H), 2.00-1.91 (m, 1H), 1.71 (dt, J = 13.9, 7.7 Hz, 1H), 1.14 (t, J = 7.4 Hz, 3H). |
| XIV- 129 | [chemical structure] | 638.25 [M + H]⁺ | (400 MHz, Methanol-d₄) δ 8.57 (s, 1H), 8.51 (dd, J = 4.5, 2.3 Hz, 1H), 8.37-8.34 (m, 1H), 7.84-7.82 (m, 1H), 7.70 (d, J = 4.2 Hz, 3H), 6.54 (d, J = 5.4 Hz, 1H), 5.04- 4.98 (m, 1H), 4.72-4.66 (m, 1H), 4.27-4.21 (m, 1H), 3.75-3.66 (m, 3H), 3.26 (dt, J = 4.9, 2.4 Hz, 4H), 3.06-2.98 (m, 4H), 2.80- 2.71 (m, 4H), 2.01-1.94 (m, 1H), 1.77-1.69 (m, 1H), 1.14 (t, J = 7.4 Hz, 3H). |
| XIV- 130 | [chemical structure] | 664.35 [M + H]⁺ | (400 MHz, Methanol-d4, ppm): δ 8.46 (d, J = 2.6 Hz, 1H), 8.29 (d, J = 5.8 Hz, 1H), 7.83 (d, J = 2.1 Hz, 1H), 7.75-7.63 (m, 3H), 7.01 (d, J = 1.8 Hz, 1H), 6.69 (dt, J = 5.7, 1.8 Hz, 1H), 5.01 (d, J = 17.1 Hz, 2H), 4.67 (d, J = 17.0 Hz, 1H), 4.27 (dd, J = 13.6, 4.2 Hz, 2H), 3.75 (s, 2H), 3.67 (dd, J = 13.5, 4.0 Hz, 1H), 3.26 (t, J = 5.2 Hz, 4H), 3.03 (dd, J = 9.0, 4.4 Hz, 1H), 2.74 (s, 4H), 2.01-1.90 (m, 1H), 1.80- 1.69 (m, 1H), 1.14 (t, J = 7.4 Hz, 3H). |
| XIV- 131 | [chemical structure] | 679.4 [M + H]⁺ | (400 MHz, Methanol-d4) δ 8.51 (d, J = 2.6 Hz, 1H), 8.35 (d, J = 6.2 Hz, 1H), 7.85 (d, J = 2.0 Hz, 1H), 7.75-7.65 (m, 3H), 6.99 (s, 1H), 6.80 (d, J = 6.2 Hz, 1H), 5.02 (d, J = 17.1 Hz, 1H), 4.69 (d, J = 17.2 Hz, 1H), 4.26 (dd, J = 13.7, 4.3 Hz, 1H), 3.82-3.62 (m, 3H), 3.43- 3.35 (m, 3H), 3.31-3.28 (m, 4H), 3.28-3.21 (m, 3H), 3.18 (s, 2H), 3.08-2.99 (m, 2H), 2.75-2.39 (m, 1H), 2.03-1.89 (m, 1H), 1.80- 1.65 (m, 1H), 1.36 (t, J = 7.3 Hz, 3H), 1.15 (t, J = 7.4 Hz, 3H). |
| XIV- 132 | [chemical structure] | 678.25 [M + H]⁺ | (400 MHz, Methanol-d4) δ 8.45 (s, 1H), 8.31 (d, J = 5.7 Hz, 1H), 7.83 (d, J = 2.0 Hz, 1H), 7.76-7.59 (m, 3H), 7.10 (s, 1H), 6.68-6.61 (m, 1H), 4.99 (d, J = 17.0 Hz, 1H), 4.66 (d, J = 17.1 Hz, 1H), 4.25 (dd, J = 13.8, 4.3 Hz, 1H), 3.76 (s, 2H), 3.72-3.66 (m, 1H), 3.01 (dt, J = 8.1, 4.1 Hz, 2H), 2.92 (s, 3H), 2.52 (s, 2H), 2.03-1.92 (m, 1H), 1.77- 1.66 (m, 1H), 1.14 (t, J = 7.4 Hz, 3H). |
| XIV- 133 | [chemical structure] | 676.30 [M + H]⁺ | (400 MHz, Methanol-d4) δ 8.49 (d, J = 2.5 Hz, 1H), 8.32 (d, J = 6.0 Hz, 1H), 7.84 (d, J = 2.1 Hz, 1H), 7.74-7.64 (m, 3H), 7.16 (d, J = 2.8 Hz, 1H), 6.78-6.71 (m, 1H), 5.02 (d, J = 17.1 Hz, 1H), 4.68 (d, J = 17.1 Hz, 1H), 4.28 (dd, J = 13.7, 4.2 Hz, 1H), 3.76 (s, 2H), 3.68 (dd, J = 13.6, 4.0 Hz, 1H), 3.04 (dd, J = 9.2, 4.7 Hz, 2H), 2.92 (s, 4H), 2.70 (s, 4H), 2.02-1.90 (m, 1H), 1.73 (dt, J = 14.7, 7.7 Hz, 1H), 1.15 (t, J = 7.4 Hz, 3H). |
| XIV- 134 | [chemical structure] | 666.40 [M + H]⁺ | (400 MHz, Methanol-d, ppm): δ 8.53 (d, J = 2.8 Hz, 1H), 8.40 (s, 1H), 7.88-7.60 (m, 4H), 7.07 (s, 1H), 5.06 (d, J = 16.9 Hz, 1H), 4.69 (d, J = 17.0 Hz, 1H), 4.32 (d, J = 13.7 Hz, 1H), 3.76 (s, 2H), 3.63 (dd, J = 13.3, 3.7 Hz, 1H), 3.53-3.34 (m, 5H), 3.31-3.12 (m, 5H), 3.09-2.98 (m, 2H), 2.96- 2.75 (m, 4H), 2.68-2.32 (m, 2H), 1.94 (tt, J = 12.2, 7.4 Hz, 1H), 1.82-1.65 (m, 1H), 1.15 (t, J = 7.4 Hz, 3H). |
| XIV- 135 | [chemical structure] | 664.25 [M + H]⁺ | (400 MHz, Methanol-d4) δ 8.46 (d, J = 2.6 Hz, 1H), 8.32 (d, J = 5.7 Hz, 1H), 7.84 (d, J = 2.0 Hz, 1H), 7.78-7.61 (m, 3H), 7.11 (s, 1H), 6.67 (d, J = 5.7 Hz, 1H), 5.00 (d, J = 17.0 Hz, 1H), 4.67 (d, J = 17.1 Hz, 1H), 4.25 (dd, J = 13.6, 4.3 Hz, 1H), 3.77 (s, 2H), 3.68 (dd, J = 13.6, 4.1 Hz, 1H), 3.27 (t, J = 5.2 Hz, 4H), 3.02 (dd, J = 9.2, 4.6 Hz, 1H), 2.75 (d, J = 5.7 Hz, 4H), 1.96 (dt, J = 12.5, 7.1 Hz, 1H), 1.71 (dq, J = 14.8, 7.6 Hz, 1H), 1.14 (t, J = 7.4 Hz, 3H). |
| XIV- 136 | [chemical structure] | 662.3 [M + H]⁺ | (400 MHz, Methanol-d4) δ 8.50 (d, J = 2.6 Hz, 1H), 8.32 (d, J = 5.9 Hz, 1H), 7.84 (d, J = 2.0 Hz, 1H), 7.75-7.65 (m, 3H), 7.17 (s, 1H), 6.76 (d, J = 6.0 Hz, 1H), 5.02 (d, J = 17.1 Hz, 1H), 4.68 (d, J = 17.1 Hz, 1H), 4.28 (dd, J = 13.6, 4.2 Hz, 1H), 3.77 (s, 2H), 3.68 (dd, J = 13.6, 4.1 Hz, 1H), 3.27 (t, J = 5.2 Hz, 4H), 3.04 (dd, J = 9.1, 4.6 Hz, 1H), 2.76 (s, 4H), 2.70 (s, 3H), 1.96 (dt, J = 12.3, 5.7 Hz, 1H), 1.73 (dp, J = 14.4, 7.5 Hz, 1H), 1.15 (t, J = 7.4 Hz, 3H). |
| XIV- 137 | [chemical structure] | 683.40 [M + H]⁺ | (400 MHz, Methanol-d4, ppm): δ 8.41 (d, J = 2.6 Hz, 1H), 8.25 (d, J = 5.5 Hz, 1H), 7.89 (d, J = 7.5 Hz, 1H), 7.66-7.53 (m, 2H), 6.79 (s, 1H), 6.60 (d, J = 5.6 Hz, 1H), 4.99 (d, J = 17.0 Hz, 1H), 4.65 (d, J = 17.0 Hz, 1H), 4.26 (dd, J = 13.6, 4.0 Hz, 1H), 3.79-3.54 (m, 4H), 3.31-3.05 (m, 6H), 2.99 (dd, J = 9.3, 4.6 Hz, 1H), 2.55 (s, 7H), 2.32 (s, 3H), 1.94 (dt, J = 12.7, 6.3 Hz, |
| | | | 1H), 1.73 (dq, J = 14.1, 7.3 Hz, |
| | | | 1H), 1.14 (t, J = 7.4 Hz, 3H). |
| XIV- 138 | [chemical structure] | 697.3 [M + H]⁺ | (400 MHz, Methanol-d4) δ 8.49 (d, J = 2.6 Hz, 1H), 8.33 (d, J = 6.1 Hz, 1H), 7.86 (d, J = 2.2 Hz, 1H), 7.77-7.66 (m, 3H), 6.95 (s, 1H), 6.75 (d, J = 6.1 Hz, 1H), 5.01 (d, J = 17.1 Hz, 1H), 4.81-4.74 (m, 1H), 4.68 (d, J = 17.1 Hz, 1H), 4.26 (dd, J = 13.6, 4.3 Hz, 1H), 3.84 (s, 2H), 3.69 (dd, J = 13.6, 4.1 Hz, 1H), 3.54 (t, J = 4.5 Hz, 1H), 3.47 (t, J = 4.5 Hz, 2H), 3.37 (s, 4H), 3.18 (s, 4H), 3.03 (dd, J = 9.0, 4.4 Hz, 2H), 2.87 (s, 4H), 1.96 (p, J = 6.9 Hz, 1H), 1.72 (dt, J = 14.7, 7.5 Hz, 1H), 1.14 (t, J = 7.4 Hz, 3H). |
| XIV- 139 | [chemical structure] | 715.30 [M + H]⁺ | (400 MHz, Methanol-d₄) δ 8.49 (s, 1H), 8.33 (d, J = 6.1 Hz, 1H), 8.02 (d, J = 2.3 Hz, 1H), 7.89 (d, J = 8.8 Hz, 1H), 7.77-7.65 (m, 2H), 6.96 (s, 1H), 6.76 (d, J = 6.2 Hz, 1H), 6.18-5.88 (m, 1H), 5.03 (d, J = 17.1 Hz, 1H), 4.70 (d, J = 17.3 Hz, 1H), 4.48-4.38 (m, 2H), 4.27 (dd, J = 13.5, 4.3 Hz, 1H), 3.70 (dd, J = 13.5, 4.1 Hz, 1H), 3.60-3.37 (m, 4H), 3.32-2.32 (m, 11H), 2.02- 1.89 (m, 1H), 1.71 (dt, J = 14.8, 7.7 Hz, 1H), 1.23 (t, J = 7.1 Hz, 1H), 1.14 (t, J = 7.4 Hz, 3H). |
| XIV- 140 | [chemical structure] | 733.3 [M + H]⁺ | (400 MHz, DMSO-d6) δ 12.26 (s, 1H), 8.95 (s, 1H), 8.44 (s, 1H), 8.31-8.14 (m, 1H), 7.98-7.89 (m, 1H), 7.82-7.66 (m, 1H), 7.67- 7.48 (m, 2H), 6.64 (s, 1H), 6.62- 6.41 (m, 1H), 4.85 (d, J = 8.3 Hz, 1H), 4.64 (d, J = 8.3 Hz, 1H), 4.01- 3.89 (m, 1H), 3.81-3.71 (m, 1H), 3.54 (s, 2H), 3.26-2.98 (m, 8H), 2.98-2.87 (m, 1H), 2.68- 2.60 (m, 4H), 2.46-2.35 (m, 4H), 2.01-1.81 (m, 1H), 1.67-1.49 (m, 1H), 1.02 (t, J = 5.2 Hz, 3H). |
| XIV- 141 | [chemical structure] | 674.31 [M + H]⁺ | (400 MHz, Methanol-d4) δ 8.53 (d, J = 2.6 Hz, 1H), 8.31 (d, J = 6.4 Hz, 1H), 7.85 (d, J = 2.1 Hz, 1H), 7.77-7.63 (m, 3H), 7.09 (s, 1H), 6.88 (d, J = 6.4 Hz, 1H), 5.04 (d, J = 17.1 Hz, 1H), 4.70 (d, J = 17.1 Hz, 1H), 4.27 (dd, J = 13.7, 4.2 Hz, 1H), 3.82-3.64 (m, 3H), 3.35 (s, 4H), 3.30 (s, 4H), 3.05 (dq, J = 8.9, 4.4 Hz, 2H), 2.92 (s, 4H), 2.82- 2.62 (s, 2H), 2.73 (d, J = 9.5 Hz, 2H), 2.55 (s, 3H), 1.97 (ddt, J = 14.8, 12.1, 7.3 Hz, 1H), 1.74 (ddt, J = 16.5, 14.4, 7.5 Hz, 1H), 1.15 (t, J = 7.4 Hz, 3H) |
| XIV- 143 | [chemical structure] | 683.45 [M + H]⁺ | (400 MHz, Methanol-d4) δ 8.40 (t, J = 3.2 Hz, 2H), 7.82 (d, J = 2.0 Hz, 1H), 7.73-7.64 (m, 2H), 7.48 (d, J = 2.7 Hz, 1H), 6.42 (s, 1H), 4.93 (d, J = 17.1 Hz, 1H), 4.59 (d, J = 17.1 Hz, 1H), 4.25 (dd, J = 13.5, 4.1 Hz, 1H), 3.75 (d, J = 1.5 Hz, 2H), 3.62 (dd, J = 13.6, 4.0 Hz, 1H), 3.56-3.35 (m, 3H), 3.26- 3.07 (m, 7H), 3.03-2.93 (m, 2H), 2.94-2.84 (m, 3H), 2.70-2.37 (m, 2H), 1.98-1.82 (m, 1H), 1.68 (ddt, J = 16.6, 14.5, 7.3 Hz, 1H), 1.11 (t, J = 7.4 Hz, 3H). |
| XIV- 144 | [chemical structure] | 666.30 [M + H]⁺ | N/A |
| XIV- 145 | [chemical structure] | 669.50 [M + H]⁺ | (400 MHz, Methanol-d, ppm): δ 8.48-8.30 (m, 2H), 7.81 (d, J = 1.8 Hz, 1H), 7.69 (d, J = 2.0 Hz, 2H), 7.46 (d, J = 2.7 Hz, 1H), 6.41 (s, 1H), 4.95 (s, 1H), 4.59 (d, J = 17.0 Hz, 1H), 4.25 (dd, J = 13.6, 4.1 Hz, 1H), 3.74 (d, J = 1.7 Hz, 2H), 3.62 (dd, J = 13.6, 4.0 Hz, 1H), 3.46-3.34 (m, 2H), 3.26- 3.07 (m, 8H), 3.01-2.93 (m, 1H), 2.85-2.63 (m, 4H), 1.90 (ddd, J = 12.9, 7.5, 4.7 Hz, 1H), 1.68 (ddt, J = 16.6, 14.4, 7.3 Hz, 1H), 1.11 (t, J = 7.4 Hz, 3H). |
| XIV- 146 | [chemical structure] | 652.25 [M + H]⁺ | (400 MHz, Methanol-d₄) δ 8.52 (s, 1H), 8.23 (d, J = 1.7 Hz, 1H), 8.09 (s, 1H), 7.84 (d, J = 2.8 Hz, 1H), 7.78-7.67 (m, 3H), 7.32 (d, J = 1.7 Hz, 1H), 5.04 (d, J = 17.1 Hz, 1H), 4.69 (d, J = 17.0 Hz, 1H), 4.28 (d, J = 13.4 Hz, 1H), 3.77 (s, 2H), 3.65 (dd, J = 13.7, 4.0 Hz, 1H), 3.27 (s, 5H), 3.17 (s, 4H), 3.02 (d, J = 3.9 Hz, 1H), 2.75 (s, 4H), 1.94 (dt, J = 12.6, 6.9 Hz, 1H), 1.72 (dp, J = 14.5, 7.6 Hz, 1H), 1.14 (t, J = 7.4 Hz, 3H). |
| XIV- 147 | [chemical structure] | 774.73 [M + H]⁺ | (400 MHz, Methanol-d4) δ 8.47 (d, J = 2.6 Hz, 1H), 8.37 (d, J = 5.6 Hz, 1H), 7.85 (d, J = 2.1 Hz, 1H), 7.73-7.65 (m, 3H), 7.28 (s, 1H), 6.65 (d, J = 5.6 Hz, 1H), 5.02 (d, J = 17.1 Hz, 1H), 4.67 (d, J = 17.1 Hz, 1H), 4.28 (dd, J = 13.6, 4.2 Hz, 1H), 3.80 (s, 2H), 3.66 (dd, J = 13.5, 4.0 Hz, 1H), 3.28 (t, J = 5.2 Hz, 4H), 3.03 (dd, J = 9.2, 4.6 Hz, 1H), 2.79 (t, J = 5.1 Hz, 4H), 2.03- 1.82 (m, 1H), 1.80-1.64 (m, 1H), 1.14 (t, J = 7.4 Hz, 3H) |
| XIV- 148 | [chemical structure] | 660.45 [M + H]⁺ | (400 MHz, Methanol-d4) δ 8.46 (d, J = 2.6 Hz, 1H), 8.20 (d, J = 6.2 Hz, 1H), 7.80 (dd, J = 20.7, 2.2 Hz, 2H), 7.75-7.60 (m, 3H), 6.90- 6.70 (m, 3H), 5.00 (d, J = 17.1 Hz, 1H), 4.67 (d, J = 17.0 Hz, 1H), 4.26 (dd, J = 13.7, 4.3 Hz, 1H), 3.75 (s, 2H), 3.69 (dd, J = 13.5, 4.1 Hz, 1H), 3.59-3.39 (m, 2H), 3.15 (dd, J = 3.4, 1.7 Hz, 2H), 3.02 (dd, J = 8.6, 4.0 Hz, 3H), 2.92 (s, 3H), 2.47 (s, 2H), 1.96 (dt, J = 12.6, 5.9 Hz, 1H), 1.71 (dq, J = 15.4, 7.9 Hz, 1H), 1.14 (t, J = 7.4 Hz, 3H). |
| XIV- 149 | [chemical structure] | 660.35 [M + H]⁺ | (400 MHz, Methanol-d4) δ 8.45 (d, J = 2.7 Hz, 1H), 8.27-8.02 (m, 3H), 7.83 (d, J = 2.1 Hz, 1H), 7.74- 7.54 (m, 3H), 6.77 (d, J = 6.3 Hz, 1H), 6.70 (s, 1H), 5.00 (d, J = 17.1 Hz, 2H), 4.67 (d, J = 17.1 Hz, 1H), 4.25 (dd, J = 13.6, 4.3 Hz, 1H), 3.75 (s, 2H), 3.69 (dd, J = 13.6, 4.0 Hz, 1H), 3.57-3.36 (m, 2H), 3.29- 2.96 (m, 3H), 2.92 (s, 3H), 2.47 (s, 2H), 1.96 (dt, J = 12.8, 6.4 Hz, 1H), 1.72 (dt, J = 14.1, 7.6 Hz, 1H), 1.32 (d, J = 8.5 Hz, 1H), 1.14 (t, J = 7.4 Hz, 3H). |
| XIV- 150 | [chemical structure] | 686.25 [M + H]⁺ | (400 MHz, Methanol-d4, ppm): δ 8.47 (d, J = 2.7 Hz, 1H), 8.27 (d, J = 6.2 Hz, 1H), 7.83 (d, J = 2.2 Hz, 1H), 7.76-7.63 (m, 3H), 6.81 (d, J = 6.2 Hz, 1H), 6.68 (s, 1H), 5.00 (d, J = 17.1 Hz, 1H), 4.73-4.62 (m, 3H), 4.25 (dd, J = 13.6, 4.3 Hz, 1H), 3.75 (s, 2H), 3.69 (dd, J = 13.6, 4.1 Hz, 1H), 3.58-3.39 (m, 3H), 3.13-2.97 (m, 5H), 2.92 (s, 3H), 2.57-2.38 (m, 2H), 2.03- 1.91 (m, 1H), 1.80-1.66 (m, 1H), 1.14 (t, J = 7.4 Hz, 3H). |
| XIV- 151 | [chemical structure] | 662.40 [M + H]⁺ | (400 MHz, Methanol-d4, ppm): δ 8.45 (d, J = 2.6 Hz, 1H), 8.22 (d, J = 6.2 Hz, 1H), 7.83 (d, J = 2.1 Hz, 1H), 7.78-7.57 (m, 3H), 6.76 (d, J = 6.1 Hz, 1H), 6.60 (t, J = 2.1 Hz, 1H), 6.46 (s, 1H), 5.09-4.93 (m, 3H), 4.92 (dd, J = 5.0, 2.1 Hz, 1H), 4.67 (d, J = 17.1 Hz, 1H), 4.25 (dd, J = 13.6, 4.3 Hz, 1H), 3.76 (s, 2H), 3.69 (dd, J = 13.6, 4.1 Hz, 1H), 3.55-3.35 (m, 3H), 3.23-2.97 (m, 4H), 2.92 (s, 3H), 2.48 (s, 2H), 1.96 (ddd, J = 12.9, 7.4, 4.8 Hz, 1H), 1.72 (ddd, J = 13.7, 9.1, 7.1 Hz, 1H), 1.14 (t, J = 7.4 Hz, 3H). |
| XIV- 152 | [chemical structure] | 634.30 [M + H]⁺ | (400 MHz, Methanol-d4) δ 8.36 (d, J = 2.6 Hz, 1H), 8.11 (d, J = 5.6 Hz, 1H), 7.81 (s, 1H), 7.74-7.59 (m, 2H), 7.51 (d, J = 2.7 Hz, 1H), 6.57 (d, J = 5.6 Hz, 1H), 6.34 (s, 1H), 5.66 (s, 1H), 5.21 (s, 1H), 4.96 (d, J = 16.9 Hz, 1H), 4.63 (d, J = 17.0 Hz, 1H), 4.26 (dd, J = 13.6, 4.1 Hz, 1H), 3.78-3.56 (m, 3H), 2.98 (dd, J = 9.2, 4.3 Hz, 1H), 2.53 (s,7H), 2.31 (s, 4H), 2.14 (s, 3H), 1.94 (td, J = 13.2, 12.5, 6.6 Hz, 1H), 1.80-1.59 (m, 1H), 1.13 (t, J = 7.4 Hz, 3H). |
| XIV- 153 | [chemical structure] | 636.40 [M + H]⁺ | (400 MHz, Methanol-d4) δ 8.33 (d, J = 2.7 Hz, 1H), 8.04 (d, J = 5.6 Hz, 1H), 7.81 (d, J = 2.1 Hz, 1H), 7.68 (t, J = 7.1 Hz, 2H), 7.47 (d, J = 2.6 Hz, 1H), 6.58 (d, J = 5.6 Hz, 1H), 5.98 (d, J = 0.9 Hz, 1H), 4.95 (d, J = 16.9 Hz, 1H), 4.62 (d, J = 17.0 Hz, 1H), 4.26 (dd, J = 13.5, 4.2 Hz, 1H), 3.63 (d, J = 8.6 Hz, 3H), 3.18-3.06 (m, 1H), 2.97 (dd, J = 9.1, 4.6 Hz, 1H), 2.53 (s, 7H), 2.32 (s, 3H), 1.92 (dt, J = 12.6, 6.2 Hz, 1H), 1.70 (dt, J = 14.8, 7.6 Hz, 1H), 1.35 (d, J = 6.9 Hz, 7H), 1.12 (t, J = 7.4 Hz, 3H). |
| XIV- 154 | [chemical structure] | 635.28 [M + H]⁺ | (400 MHz, Methanol-d4) δ 8.46 (s, 1H), 8.38-8.30 (m, 1H), 7.83 (d, J = 2.1 Hz, 1H), 7.75-7.62 (m, 3H), 7.41-7.35 (m, 1H), 6.66- 6.59 (m, 1H), 5.00 (d, J = 17.1 Hz, 1H), 4.68 (d, J = 17.1 Hz, 1H), 4.26 (dd, J = 13.6, 4.3 Hz, 1H), 3.76 (s, 2H), 3.68 (dd, J = 13.6, 4.1 Hz, 1H), 3.50 (s, 2H), 3.20 (s, 2H), 3.10 (s, 2H), 3.02 (dd, J = 9.1, 4.6 Hz, 2H), 2.92 (s, 3H), 2.61 (s, 3H), 2.4-2.6 (m, 1H), 2.04-1.89 (m, 1H), 1.72 (dp, J = 14.6, 7.6 Hz, 1H), 1.14 (t, J = 7.4 Hz, 3H) |
| XIV- 155 | [chemical structure] | 757.2 [M + H]⁺ | N/A |

### Example 64—Synthesis of Additional 4-alkyl-N-phenyl-3,4-dihydroisoquinoline-2(1H)-carboxamides and Related Compounds

The compounds in Table 15 below were prepared based on experimental procedures described in Examples 5 and 6 and in the detailed description above.

TABLE 15

| | | Mass | |
|---|---|---|---|
| | | Spec. | |
| No. | Chemical Structure | (ES, m/z) | ¹H NMR Spectrum |
| XV- 1 | [chemical structure] | 616.30 [M + H]⁺ | (400 MHz, Methanol-d₄) δ 8.22 (t, J = 3.1 Hz, 1H), 7.75 (s, 1H), 7.63- 7.54 (m, 1H), 7.41-7.03 (m, 5H), 5.97-5.85 (m, 1H), 4.94 (d, J = 9.5 Hz, 1H), 4.59 (d, J = 16.5 Hz, 1H), 4.22 (dd, J = 13.1, 3.8 Hz, 1H), 3.74 (s, 2H), 3.57-3.38 (m, 3H), 2.91 (s, 11H), 2.05 (ddd, J = 13.6, 8.6, 5.1 Hz, 1H), 1.73 (dh, J = 14.0, 6.7 Hz, 2H), 1.17- 0.96 (m, 5H), 0.87-0.77 (m, 2H). |
| XV- 2 | [chemical structure] | 646 [M + H]⁺ | (400 MHz, Methanol-d4) δ 8.23 (d, J = 7.5 Hz, 1H), 7.76 (s, 1H), 7.59 (d, J = 8.0 Hz, 1H), 7.36 (d, J = 8.8 Hz, 2H), 7.23 (s, 1H), 7.10 (s, 2H), 5.91 (d, J = 9.9 Hz, 1H), 4.59 (d, J = 16.5 Hz, 1H), 4.23 (dd, J = 13.1, 3.9 Hz, 1H), 3.88 (t, J = 5.2 Hz, 2H), 3.76 (d, J = 11.4 Hz, 2H), 3.47 (dd, J = 13.1, 3.7 Hz, 2H), 3.27 (t, J = 5.3 Hz, 3H), 2.86 (d, J = 35.9 Hz, 5H), 2.04 (td, J = 8.5, 4.3 Hz, 1H), 1.72 (dp, J = 13.9, 7.1 Hz, 2H), 1.11 (t, J = 7.4 Hz, 3H), 1.05 (dt, J = 8.4, 3.3 Hz, 2H), 0.87-0.78 (m, 2H). |
| XV- 3 | [chemical structure] | 616 [M + H]⁺ | (400 MHz, Methanol-d4) δ 8.25 (d, J = 2.4 Hz, 1H), 7.76 (d, J = 2.5 Hz, 1H), 7.58 (dd, J = 8.3, 2.1 Hz, 1H), 7.40-7.32 (m, 2H), 7.22 (s, 1H), 7.15-7.07 (m, 2H), 5.87 (s, 1H), 4.59 (d, J = 16.5 Hz, 1H), 4.22 (dd, J = 12.7, 3.7 Hz, 1H), 3.77 (d, J = 4.5 Hz, 2H), 3.47 (dd, J = 13.0, 3.8 Hz, 1H), 3.16 (d, J = 59.7 Hz, 3H), 2.91 (s, 4H), 2.73 (s, 3H), 2.04 (tt, J = 8.6, 5.1 Hz, 1H), 1.74 (dp, J = 14.8, 6.9 Hz, 2H), 1.15-1.00 (m, 5H), 0.85-0.78 (m, 2H). |
| XV- 4 | [chemical structure] | 609.40 [M + H]⁺ | (400 MHz, Methanol-d4) δ 8.18 (d, J = 6.8 Hz, 1H), 7.82 (d, J = 2.0 Hz, 1H), 7.72-7.62 (m, 2H), 7.43 (d, J = 8.9 Hz, 1H), 7.15 (d, J = 5.4 Hz, 2H), 6.79 (d, J = 6.8 Hz, 1H), 6.47 (s, 1H), 4.93 (d, J = 16.7 Hz, 1H), 4.75 (s, 2H), 4.58 (d, J = 16.7 Hz, 1H), 4.18 (d, J = 12.1 Hz, 1H), 3.72 (s, 2H), 3.48 (d, J = 12.6 Hz, 1H), 3.23 (t, J = 5.2 Hz, 4H), 2.91 (s, 1H), 2.71 (s, 4H), 1.72 (s, 2H), 1.71-1.64 (m, 0H), 1.08 (t, J = 7.4 Hz, 3H). |
| XV- 5 | [chemical structure] | 609.25 [M + H]⁺ | (400 MHz, Methanol-d4) δ 8.20 (d, J = 6.8 Hz, 1H), 7.75 (d, J = 2.3 Hz, 1H), 7.55 (dd, J = 8.3, 2.3 Hz, 1H), 7.47-7.40 (m, 1H), 7.37- 7.31 (m, 1H), 7.23-7.13 (m, 3H), 6.81 (d, J = 6.8 Hz, 1H), 6.49 (s, 1H), 4.92 (s, 2H), 4.76 (s, 2H), 4.59 (d, J = 16.8 Hz, 1H), 4.19 (dd, J = 13.1, 3.8 Hz, 1H), 3.74 (s, 2H), 3.48 (dd, J = 13.1, 3.8 Hz, 1H), 3.38 (s, 1H), 3.21 (s, 1H), 2.96-2.88 (m, 1H), 2.75 (s, 5H), 1.83-1.70 (m, 1H), 1.69 (dt, J = 13.7, 5.2 Hz, 1H), 1.09 (t, J = 7.4 Hz, 3H). |
| XV- 6 | [chemical structure] | 653.40 [M + H]⁺ | (400 MHz, Methanol-d4) δ 8.21 (d, J = 6.6 Hz, 1H), 7.85 (d, J = 2.2 Hz, 1H), 7.69 (t, J = 7.2 Hz, 2H), 7.46 (d, J = 8.9 Hz, 1H), 7.18 (d, J = 5.1 Hz, 2H), 6.81 (d, J = 6.8 Hz, 1H), 6.50 (s, 1H), 4.98 (d, J = 17.2 Hz, 1H), 4.78 (s, 2H), 4.61 (d, J = 16.7 Hz, 1H), 4.22 (d, J = 12.4 Hz, 1H), 3.89 (t, J = 5.1 Hz, 2H), 3.76 (s, 2H), 3.55-3.47 (m, 3H), 3.29- 3.85 (m, 4H), 2.94-2.68 (s, 2H), 1.75 (m, 2H), 1.11 (t, J = 7.4 Hz, 3H). |
| XV- 7 | [chemical structure] | 635.45 [M + H]⁺ | (400 MHz, Methanol-d4) δ 8.18 (d, J = 6.7 Hz, 1H), 7.72 (s, 1H), 7.55 (d, J = 8.4 Hz, 1H), 7.43 (d, J = 8.9 Hz, 1H), 7.33 (d, J = 8.8 Hz, 1H), 7.21-7.16 (m, 3H), 6.79 (d, J = 6.8 Hz, 1H), 6.47 (s, 1H), 4.94 (d, J = 17.2 Hz, 1H), 4.75 (s, 2H), 4.57 (d, J = 16.9 Hz, 1H), 4.18 (d, J = 12.9 Hz, 1H), 3.85 (s, 2H), 3.71 (s, 2H), 3.48 (m, 3H), 3.24 (m, 3H), 3.15-2.50 (m, 6H), 1.72 (t, J = 7.0 Hz, 2H), 1.08 (t, J = 7.4 Hz, 3H). |
| XV- 8 | [chemical structure] | 605.25 [M + H]⁺ | (400 MHz, Methanol-d4) δ 8.02 (d, J = 5.6 Hz, 1H), 7.73 (d, J = 2.2 Hz, 1H), 7.52 (d, J = 8.1 Hz, 1H), 7.37-7.24 (m, 3H), 7.04 (d, J = 9.5 Hz, 2H), 6.50 (d, J = 5.6 Hz, 1H), 6.24 (s, 1H), 4.91 (d, J = 17.2 Hz, 1H), 4.72 (s, 2H), 4.56 (d, J = 16.6 Hz, 1H), 4.20 (dd, J = 13.0, 3.9 Hz, 1H), 3.60 (s, 2H), 3.46 (dd, J = 12.9, 3.7 Hz, 1H), 2.87 (s, 1H), 2.48 (s, 8H), 2.29 (s, 3H), 1.72 (m, 2H), 1.10 (t, J = 7.4 Hz 3H). |
| XV- 9 | [chemical structure] | 605.25 [M + H]⁺ | (400 MHz, Methanol-d4) δ 8.20 (d, J = 6.8 Hz, 1H), 7.75 (d, J = 2.3 Hz, 1H), 7.55 (dd, J = 8.3, 2.3 Hz, 1H), 7.47-7.40 (m, 1H), 7.37- 7.31 (m, 1H), 7.23-7.13 (m, 3H), 6.81 (d, J = 6.8 Hz, 1H), 6.49 (s, 1H), 4.92 (s, 2H), 4.76 (s, 2H), 4.59 (d, J = 16.8 Hz, 1H), 4.19 (dd, J = 13.1, 3.8 Hz, 1H), 3.74 (s, 2H), 3.48 (dd, J = 13.1, 3.8 Hz, 1H), 3.38 (s, 1H), 3.21 (s, 1H), 2.96-2.88 (m, 1H), 2.88 (s, 3H), 2.75 (s, 5H), 1.83-1.70 (m, 1H), 1.69 (dt, J = 13.7, 5.2 Hz, 1H), 1.09 (t, J = 7.4 Hz, 3H). |
| XV- 10 | [chemical structure] | 651.30 [M + H]⁺ | (400 MHz, Methanol-d₄) δ 8.21 (s, 1H), 7.84 (s, 1H), 7.69 (t, J = 7.3 Hz, 2H), 7.45 (d, J = 8.6 Hz, 1H), 7.18 (s, 2H), 6.79 (s, 1H), 6.67 (s, 1H), 4.98 (s, 4H), 4.61 (d, J = 16.7 Hz, 1H), 4.21 (d, J = 12.0 Hz, 1H), 3.74 (s, 2H), 3.53 (s, 1H), 3.25 (s, 4H), 2.94 (s, 1H), 2.73 (s, 4H), 1.73 (s, 2H), 1.11 (t, J = 7.1 Hz, 3H). |
| XV- 11 | [chemical structure] | 651 [M + H]⁺ | (400 MHz, Methanol-d4) δ 8.04 (d, J = 5.7 Hz, 1H), 7.81 (d, J = 2.2 Hz, 1H), 7.73-7.62 (m, 2H), 7.34 (d, J = 8.2 Hz, 1H), 7.07 (d, J = 2.6 Hz, 1H), 7.04 (s, 2H), 6.49 (d, J = 5.6 Hz, 1H), 6.40 (s, 1H), 4.62- 4.51 (m, 2H), 4.19 (d, J = 13.7 Hz, 1H), 3.70 (s, 2H), 3.48-3.41 (m, 1H), 3.22 (t, J = 5.2 Hz, 5H), 2.87 (s, 1H), 2.69 (s, 4H), 1.69 (td, J = 14.1, 12.9, 6.9 Hz, 2H), 1.28 (s, 1H), 1.08 (t, J = 7.4 Hz, 3H). |
| XV- 12 | [chemical structure] | 665.25 [M + H]⁺ | (400 MHz, Methanol-d₄) δ 8.06 (d, J = 5.7 Hz, 1H), 7.83 (s, 1H), 7.67 (s, 2H), 7.36 (d, J = 8.1 Hz, 1H), 7.07 (d, J = 9.9 Hz, 2H), 6.52 (d, J = 5.6 Hz, 1H), 6.42 (s, 1H), 4.93 (s, 4H), 4.58 (d, J = 17.0 Hz, 3H), 4.20 (d, J = 14.0 Hz, 1H), 3.64 (s, 2H), 3.47 (d, J = 14.7 Hz, 1H), 2.89 (s, 1H), 2.54 (s, 6H), 2.32 (s, 3H), 1.73 (dt, J = 16.0, 7.5 Hz, 2H), 1.34-1.27 (m, 1H), 1.10 (t, J = 7.4 Hz, 3H), 0.90 (s, 1H), 0.12 (s, 2H). |
| XV- 13 | [chemical structure] | 665.25 [M + H]⁺ | (400 MHz, Methanol-d4) δ 8.22 (d, J = 6.8 Hz, 1H), 7.83 (d, J = 2.1 Hz, 1H), 7.73-7.62 (m, 2H), 7.48- 7.41 (m, 1H), 7.21-7.15 (m, 2H), 6.81 (d, J = 6.7 Hz, 1H), 6.68 (s, 1H), 4.94 (d, J = 16.7 Hz, 1H), 4.60 (d, J = 16.7 Hz, 1H), 4.19 (dd, J = 12.9, 3.7 Hz, 1H), 3.74 (s, 2H), 3.49 (dd, J = 13.2, 3.7 Hz, 1H), 2.90 (s, 4H), 1.73 (s, 1H), 1.71 (s, 1H), 1.09 (t, J = 7.4 Hz, 3H). |
| XV- 14 | [chemical structure] | 647.3 [M + H]⁺ | (400 MHz, Methanol-d₄) δ 8.23 (d, J = 6.7 Hz, 1H), 7.75 (d, J = 2.1 Hz, 1H), 7.60-7.54 (m, 1H), 7.49- 7.43 (m, 1H), 7.35 (d, J = 8.9 Hz, 1H), 7.27-7.08 (m, 3H), 6.83 (d, J = 6.7 Hz, 1H), 6.70 (s, 1H), 5.10 (s, 4H), 4.61 (d, J= 16.8 Hz, 1H), 4.25-4.16 (m, 1H), 3.73 (s, 2H), 3.55-3.45 (m, 1H), 3.20 (s, 2H), 2.90 (s, 5H), 2.50 (s, 2H), 1.74 (dp, J = 14.1, 7.1 Hz, 2H), 1.31 (s, 1H), 1.11 (t, J = 7.3 Hz, 3H). |
| XV- 15 | [chemical structure] | 647.30 [M + H]⁺ | (400 MHz, Methanol-d4) δ 8.24 (d, J = 6.8 Hz, 1H), 7.76 (d, J = 2.3 Hz, 1H), 7.56 (dd, J = 8.4, 2.4 Hz, 1H), 7.48-7.41 (m, 1H), 7.35 (d, J = 8.0 Hz, 1H), 7.19 (td, J = 4.6, 2.5 Hz, 2H), 6.83 (d, J = 6.9 Hz, 1H), 6.70 (s, 1H), 4.89 (s, 3H), 4.59 (d, J = 16.7 Hz, 1H), 4.20 (dd, J = 13.2, 3.8 Hz, 1H), 3.77 (s, 2H), 3.48 (dd, J = 13.0, 3.7 Hz, 1H), 3.34 (s, 1H), 3.25 (d, J = 13.3 Hz, 1H), 2.93 (t, J = 4.8 Hz, 1H), 2.89 (s, 3H), 2.79 (s, 3H), 1.71 (dp, J = 21.5, 7.2 Hz, 2H), 1.09 (t, J = 7.3 Hz, 3H). |
| XV- 16 | [chemical structure] | 604.25 [M + H]⁺ | (400 MHz, Methanol-d4) δ 8.30 (dd, J = 5.9, 1.7 Hz, 1H), 7.89- 7.79 (m, 1H), 7.78-7.63 (m, 2H), 7.46-7.37 (m, 1H), 7.22 (dd, J = 2.5, 1.5 Hz, 1H), 7.13 (d, J = 7.0 Hz, 2H), 6.57 (ddd, J = 5.9, 2.5, 1.6 Hz, 1H), 4.96 (s, 1H), 4.60 (d, J = 16.6 Hz, 1H), 4.22 (dd, J = 13.1, 3.8 Hz, 1H), 3.83-3.73 (m, 2H), 3.49 (dd, J = 13.0, 3.8 Hz, 1H), 3.26 (q, J = 4.5, 3.5 Hz, 4H), 2.92 (t, J = 7.0 Hz, 1H), 2.76 (s, 4H), 1.81-1.63 (m, 2H), 1.11 (t, J = 7.4 Hz, 3H). |
| XV- 17 | [chemical structure] | 604.35 [M + H]⁺ | (400 MHz, Methanol-d4) δ 8.29 (d, J = 5.6 Hz, 1H), 7.84 (d, J = 2.2 Hz, 1H), 7.76-7.64 (m, 2H), 7.41 (d, J = 8.3 Hz, 1H), 7.19 (s, 1H), 7.12 (d, J = 7.6 Hz, 2H), 6.55 (d, J = 5.6 Hz, 1H), 4.90 (d, J = 16.7 Hz, 1H), 4.60 (d, J = 16.7 Hz, 1H), 4.21 (dd, J = 13.0, 3.8 Hz, 1H), 3.74 (s, 2H), 3.52-3.45 (m, 1H), 3.25 (t, J = 5.2 Hz, 4H), 2.92 (s, 1H), 2.74 (d, J = 5.9 Hz, 4H), 1.73 (m, 2H), 1.10 (t, J = 7.4 Hz, 3H). |
| XV- 18 | [chemical structure] | 600.30 [M + H]⁺ | (400 MHz, Methanol-d₄) δ 8.31 (d, J = 5.8 Hz, 1H), 7.76 (d, J = 2.2 Hz, 1H), 7.60-7.55 (m, 1H), 7.45- 7.39 (m, 1H), 7.35 (d, J = 8.5 Hz, 1H), 7.26-7.08 (m, 4H), 6.59 (d, J = 5.8 Hz, 1H), 4.96 (s, 1H), 4.60 (d, J = 16.5 Hz, 1H), 4.22 (dd, J = 13.1, 3.9 Hz, 1H), 3.75 (s, 2H), 3.54-3.46 (m, 1H), 3.42- 3.34 (m, 2H), 3.30-3.13 (m, 3H), 2.91 (s, 7H, 1.73 (dddd, J = 15.3, 13.8, 11.0, 6.8 Hz, 2H), 1.11 (t, J = 7.4 Hz, 3H). |
| XV- 19 | [chemical structure] | 600.35 [M + H]⁺ | (400 MHz, Methanol-d4) δ 8.29 (d, J = 5.7 Hz, 1H), 7.78-7.72 (m, 1H), 7.61-7.54 (m, 1H), 7.44- 7.38 (m, 1H), 7.35-7.12 (m, 5H), 6.56 (d, J = 5.7 Hz, 1H), 4.93 (d, J = 16.5 Hz, 1H), 4.59 (d, J = 16.6 Hz, 1H), 4.21 (dd, J = 13.2, 3.8 Hz, 1H), 3.73 (s, 2H), 3.48 (dd, J = 12.9, 3.6 Hz, 1H), 3.22 (s, 4H), 2.90 (s, 4H), 2.51 (s, 2H), 1.72 (m, 2H), 1.10 (t, J = 7.4 Hz 3H). |
| XV- 20 | [chemical structure] | 630.35 [M + H]⁺ | (400 MHz, Methanol-d4) δ 8.30 (d, J = 5.7 Hz, 1H), 7.76 (d, J = 2.3 Hz, 1H), 7.58 (dd, J = 8.3, 2.3 Hz, 1H), 7.41 (d, J = 9.1 Hz, 1H), 7.38- 7.05 (m, 5H), 6.56 (d, J = 5.7 Hz, 1H), 4.95 (d, J = 6.7 Hz, 1H), 4.60 (d, J = 16.7 Hz, 1H), 4.22 (dd, J = 13.1, 3.8 Hz, 1H), 3.96- 3.83 (m, 2H), 3.75 (s, 2H), 3.60- 3.34 (m, 5H), 3.27 (dd, J = 6.2, 4.2 Hz, 3H), 2.98-2.61 (m, 4H), 1.73 (dq, J = 19.1, 7.0 Hz, 2H), 1.11 (t, J = 7.4 Hz, 3H). |
| XV- 21 | [chemical structure] | 630.35 [M + H]⁺ | (400 MHz, Methanol-d4) δ 8.33 (s, 1H), 7.76 (s, 1H), 7.59 (d, J = 8.3 Hz, 1H), 7.43 (d, J = 9.1 Hz, 1H), 7.37 (s, 1H), 7.29-7.19 (m, 2H), 7.12 (m, 3H), 6.62 (s, 1H), 4.98 (d, J = 16.7 Hz, 1H), 4.60 (d, J = 16.7 Hz, 1H), 4.22 (dd, J = 13.2, 3.8 Hz, 1H), 3.88 (t, J = 5.2 Hz, 2H), 3.77 (d, J = 13.5 Hz, 2H), 3.50 (m, 2H), 3.35 (m, 2H), 3.27 (m, 3H), 2.92 (m, 4H), 1.74 (m, 2H), 1.11 (t, J = 7.4 Hz, 3H). |
| XV- 22 | [chemical structure] | 653 [M + H]⁺ | (400 MHz, CD3OD-d4, ppm) δ 1.11 (t, J = 7.4 Hz, 3H), 1.73 (dt, J = 14.2, 8.1 Hz, 2H), 3.07-2.48 (m, 6H), 3.28 (d, J = 4.9 Hz, 3H), 3.63-3.38 (m, 3H), 3.77 (s, 2H), 3.89 (t, J = 5.2 Hz, 2H), 4.21 (dd, J = 13.1, 3.7 Hz, 1H), 4.61 (d, J = 16.8 Hz, 1H), 4.78 (s, 2H), 4.96 (d, J = 16.9 Hz, 1H), 6.50 (s, 1H), 6.82 (d, J = 6.7 Hz, 1H), 7.19 (s, 2H), 7.46 (d, J = 8.4 Hz, 1H), 7.70 (q, J = 8.7 Hz, 2H), 7.85 (d, J = 2.8 Hz, 1H), 8.21 (d, J = 6.7 Hz, 1H). |
| XV- 23 | [chemical structure] | 635 [M + H]⁺ | (400 MHz, CD3OD-d4, ppm) δ 1.11 (t, J = 7.3 Hz, 3H), 1.74 (dq, J = 17.9, 7.2 Hz, 2H), 3.05-2.48 (m, 6H), 3.27 (t, J = 5.3 Hz, 3H), 3.59-3.37 (m, 3H), 3.75 (s, 2H), 3.88 (t, J = 5.2 Hz, 2H), 4.21 (dd, J = 13.1, 3.8 Hz, 1H), 4.60 (d, J = 16.8 Hz, 1H), 4.78 (s, 2H), 4.96 (d, J = 16.8 Hz, 1H), 6.50 (s, 1H), 6.82 (d, J = 6.8 Hz, 1H), 7.25- 7.08 (m, 3H), 7.36 (d, J = 8.4 Hz, 1H), 7.46 (d, J = 9.1 Hz, 1H), 7.57 (d, J = 7.8 Hz, 1H), 7.76 (d, J = 2.3 Hz, 1H), 8.21 (d, J = 6.8 Hz, 1H). |
| XV- 24 | [chemical structure] | 645.40 [M + H]⁺ | (400 MHz, Methanol-d4) δ 8.13 (d, J = 6.8 Hz, 1H), 7.76 (d, J = 2.3 Hz, 1H), 7.58 (dd, J = 8.3, 2.3 Hz, 1H), 7.48-7.41 (m, 1H), 7.41- 7.31 (m, 1H), 7.24 (s, 1H), 7.19- 7.08 (m, 2H), 6.78 (d, J = 6.8 Hz, 1H), 6.20 (d, J = 0.7 Hz, 1H), 4.96 (d, J = 16.9 Hz, 1H), 4.60 (d, J = 16.8 Hz, 1H), 4.22 (dd, J = 13.1, 3.8 Hz, 1H), 3.93-3.84 (m, 2H), 3.74 (s, 2H), 3.62-3.35 (m, 4H), 3.29-2.48 (m, 8H), 2.12 (tt, J = 8.5, 5.0 Hz, 1H), 1.85-1.65 (m, 2H), 1.25-1.00 (m, 5H), 0.93- 0.82 (m, 2H). |
| XV- 25 | [chemical structure] | 645.40 [M + H]⁺ | (400 MHz, Methanol-d4) δ 8.13 (d, J = 6.8 Hz, 1H), 7.76 (d, J = 2.2 Hz, 1H), 7.61-7.54 (m, 1H), 7.45 (d, J = 9.1 Hz, 1H), 7.40-7.17 (m, 4H), 6.79 (d, J = 6.8 Hz, 1H), 6.21 (s, 1H), 4.96 (d, J = 16.7 Hz, 1H), 4.60 (d, J = 16.8 Hz, 1H), 4.22 (dd, J = 13.1, 3.9 Hz, 1H), 3.92- 3.85 (m, 2H), 3.75 (s, 2H), 3.50 (m, 4H), 3.27 (m, 3H), 2.94 (s, 1H), 2.77 (s, 4H), 2.12 (m, 1H), 1.74 (m, 2H), 1.19-1.07 (m, 5H), 0.89 (m, 2H). |
| XV- 26 | [chemical structure] | 663.45 [M + H]⁺ | (400 MHz, Methanol-(d₄) δ 8.13 (d, J = 6.8 Hz. 1H), 7.85 (d, J = 2.1 Hz, 1H), 7.80-7.62 (m, 2H), 7.45 (d, J = 9.1 Hz, 1H), 7.17 (dq, J = 5.4, 2.6 Hz, 2H), 6.79 (d, J = 6.8 Hz, 1H), 6.20 (d, J = 0.7 Hz, 1H), 4.98 (s, 1H), 4.61 (d, J = 16.7 Hz, 1H), 4.22 (dd, J = 13.1, 3.9 Hz, 1H), 3.93-3.83 (m, 2H), 3.77 (d, J = 1.6 Hz, 2H), 3.63-3.36 (m, 4H), 3.31-3.23 (m, 3H), 2.99- 2.68 (m, 4H), 2.12 (tt, J = 8.5, 5.1 Hz, 1H), 1.74 (dtd, J = 17.4, 14.0, 6.9 Hz, 2H), 1.18-1.07 (m, 5H), 0.91-0.85 (m, 2H). |
| XV- 27 | [chemical structure] | 663.40 [M + H]⁺ | (400 MHz, Methanol-d4) δ 8.13 (d, J = 7.0 Hz, 1H), 7.85 (d, J = 2.1 Hz, 1H), 7.76-7.65 (m, 2H), 7.45 (d, J = 9.1 Hz, 1H), 7.17 (m, 2H), 6.82-6.76 (m, 1H), 6.20 (d, J = 0.8 Hz, 1H), 4.96 (d, J = 16.9 Hz, 1H), 4.61 (d, J = 16.8 Hz, 1H), 4.22 (dd, J = 13.1, 3.9 Hz, 1H), 3.93-3.86 (m, 2H), 3.77 (s, 2H), 3.51 (m, 4H), 3.29 (dd, J = 6.3, 4.1 Hz, 3H), 2.80 (m, 5H), 2.12 (m, 1H), 1.83-1.65 (m, 2H), 1.19- 1.07 (m, 4H), 0.89 (dt, J = 6.8, 4.5 Hz, 2H). |
| XV- 28 | [chemical structure] | 618.35 [M + H]⁺ | (400 MHz, Methanol-d4) δ 8.31 (d, J = 5.8 Hz, 1H), 7.85 (d, J = 2.2 Hz, 1H), 7.76-7.70 (m, 1H), 7.68 (d, J = 8.6 Hz, 1H), 7.42 (d, J = 8.9 Hz, 1H), 7.23 (s, 1H), 7.13 (d, J = 6.9 Hz, 2H), 6.58 (d, J = 5.8 Hz, 1H), 4.94 (d, J = 16.6 Hz, 1H), 4.60 (d, J = 16.6 Hz, 1H), 4.22 (dd, J = 13.1, 3.8 Hz, 1H), 3.76 (s, 2H), 3.49 (dd, J = 13.0, 3.7 Hz, 1H), 3.45-3.15 (m, 4H), 2.92 (s, 3H), 2.58 (s, 4H), 1.73 (m, 2H), 1.11 (t, J = 7.4 Hz, 3H). |
| XV- 29 | [chemical structure] | 618.35 [M + H]⁺ | (400 MHz, Methanol-d4) δ 8.29 (d, J = 5.7 Hz, 1H), 7.84 (d, J = 2.2 Hz, 1H), 7.72 (dd, J = 8.6, 2.2 Hz, 1H), 7.67 (d, J = 8.6 Hz, 1H), 7.41 (d, J = 8.4 Hz, 1H), 7.19 (s, 1H), 7.12 (d, J = 7.2 Hz, 2H), 6.55 (d, J = 5.7 Hz, 1H), 4.94 (d, J = 16.6 Hz, 1H), 4.60 (d, J = 16.6 Hz, 1H), 4.22 (dd, J = 13.1, 3.8 Hz, 1H), 3.75 (s, 2H), 3.49 (dd, J = 13.0, 3.7 Hz, 1H), 3.40-3.04 (m, 6H), 2.92 (s, 4H), 2.48 (s, 2H), 1.72 (m, 2H), 1.11 (t, J = 7.4 Hz, 3H). |
| XV- 30 | [chemical structure] | 636.35 [M + H]⁺ | (400 MHz, Methanol-d4) δ 8.32 (d, J = 6.4 Hz, 1H), 7.85 (d, J = 2.1 Hz, 1H), 7.76-7.65 (m, 2H), 7.46 (d, J = 9.1 Hz, 1H), 7.40-7.35 (m, 1H), 7.22-7.16 (m, 2H), 6.75- 6.68 (m, 1H), 4.96 (d, J = 16.8 Hz, 1H), 4.62 (d, J = 16.8 Hz, 1H), 4.21 (dd, J = 13.1, 3.9 Hz, 1H), 3.76 (s, 2H), 3.51 (dd, J = 13.1, 3.8 Hz, 1H), 3.45-3.19 (m, 4H), 2.92 (s, 5H), 2.55 (s, 3H), 1.74 (m, 2H), 1.11 (t, J = 7.4 Hz, 3H). |
| XV- 31 | [chemical structure] | 622.25 [M + H]⁺ | (400 MHz, DMSO-d4, ppm) δ 1.29 (d, J = 6.9 Hz, 3H), 2.47- 2.35 (m, 2H), 2.81 (s, 3H), 3.14- 2.87 (m, 5H), 3.41 (s, 2H), 3.57 (dd, J = 13.1, 6.6 Hz, 1H), 3.68 (s, 2H), 3.75 (dd, J = 13.0, 4.5 Hz, 1H), 4.62 (d, J = 17.0 Hz, 1H), 4.79 (d, J = 17.0 Hz, 1H), 6.44 (d, J = 5.7 Hz, 1H), 7.14-7.07 (m, 2H), 7.24 (s, 1H), 7.42 (d, J = 8.3 Hz, 1H), 7.54 (s, 1H), 7.59 (d, J = 8.6 Hz, 1H), 7.83 (dd, J = 8.6, 2.2 Hz, 1H), 7.94 (d, J = 2.2 Hz, 1H), 8.04 (s, 1H), 8.23 (d, J = 5.7 Hz, 1H), 8.95 (s, 1H), 12.42 (s, 1H). |
| XV- 32 | [chemical structure] | 651.3 [M + H]⁺ | (400 MHz, Methanol-d4) δ 8.03 (d, J = 5.6 Hz, 1H), 7.81 (s, 1H), 7.65 (d, J = 1.5 Hz, 2H), 7.38 (d, J = 8.4 Hz, 1H), 7.10-7.00 (m, 2H), 6.48 (d, J = 5.6 Hz, 1H), 6.42 (s, 1H), 4.92 (d, J = 6.9 Hz, 2H), 4.83 (d, J = 13.2 Hz, 1H), 4.67- 4.57 (m, 1H), 3.74 (dd, J = 12.9, 4.4 Hz, 1H), 3.67 (dd, J = 13.0, 6.0 Hz, 1H), 3.62 (d, J = 1.5 Hz, 2H), 2.56 (s, 8H), 2.35 (s, 3H), 1.92 (s, 1H), 1.37 (d, J = 6.9 Hz, 3H), 1.29 (s, 0H). |
| XV- 33 | [chemical structure] | 633.35 [M + H]⁺ | (400 MHz, Methanol-d₄) δ 8.06 (d, J = 5.6 Hz, 1H), 7.74 (d, J = 2.3 Hz, 1H), 7.53 (dd, J = 8.3, 2.3 Hz, 1H), 7.44-7.37 (m, 1H), 7.34- 7.19 (m, 2H), 7.15-7.02 (m, 2H), 6.51 (d, J = 5.6 Hz, 1H), 6.45 (s, 1H), 4.97-4.91 (m, 2H), 4.85 (d, J = 14.5 Hz, 2H), 4.62 (s, 1H), 3.73 (qd, J = 13.0, 5.2 Hz, 2H), 3.61 (s, 2H), 3.24-3.08 (m, 2H), 2.82-2.36 (m, 7H), 2.33 (s, 3H), 1.39 (d, J = 6.9 Hz, 3H). |
| XV- 34 | [chemical structure] | 604.35 [M + H]⁺ | (400 MHz, Methanol-d4) δ 8.50 (s, 1H), 8.27 (d, J = 5.6 Hz, 1H), 7.84 (d, J = 2.1 Hz, 1H), 7.74-7.64 (m, 2H), 7.45 (d, J = 8.3 Hz, 1H), 7.18 (s, 1H), 7.16-7.07 (m, 2H), 6.52 (d, J = 5.6 Hz, 1H), 4.86 (s, 1H), 4.67 (d, J = 16.6 Hz, 1H), 3.84-3.64 (m, 4H), 3.22-3.01 (m, 5H), 2.82-2.53 (m, 7H), 1.40 (d, J = 6.9 Hz, 3H). |
| XV- 35 | [chemical structure] | 604.30 [M + H]⁺ | (400 MHz, Methanol-d4) δ 8.47 (s, 1H), 8.27 (d, J = 5.6 Hz, 1H), 7.84 (d, J = 2.2 Hz, 1H), 7.75-7.64 (m, 2H), 7.45 (d, J = 8.3 Hz, 1H), 7.19 (s, 1H), 7.16-7.07 (m, 2H), 6.52 (d, J = 5.6 Hz, 1H), 4.86 (d, 1H), 4.67 (d, J = 16.6 Hz, 1H), 3.83-3.64 (m, 4H), 3.24-3.02 (m, 5H), 2.87-2.57 (m, 7H), 1.40 (d, J = 6.9 Hz, 3H). |
| XV- 36 | [chemical structure] | 634.15 [M + H]⁺ | (400 MHz, Methanol-d4) δ 8.30 (d, J = 5.7 Hz, 1H), 7.85 (d, J = 2.2 Hz, 1H), 7.77-7.60 (m, 3H), 7.46 (d, J = 8.3 Hz, 1H), 7.22 (s, 1H), 7.17-7.08 (m, 2H), 6.56 (d, J = 5.7 Hz, 1H), 4.86 (d, 1H), 4.67 (d, J = 16.6 Hz, 1H), 3.93-3.82 (m, 2H), 3.77 (s, 3H), 3.82-3.66 (m, 1H), 3.38 (m, 4H), 3.32-3.25 (m, 2H), 3.22-3.15 (m, 1H), 2.81 (m, 4H), 1.40 (d, J = 6.9 Hz, 3H), 1.01- 0.91 (m, 2H). |
| XV- 37 | [chemical structure] | 634.2 [M + H]⁺ | (400 MHz, Methanol-d4) δ 8.30 (d, J = 5.7 Hz, 1H), 7.85 (d, J = 2.2 Hz, 1H), 7.77-7.60 (m, 3H), 7.46 (d, J = 8.3 Hz, 1H), 7.22 (s, 1H), 7.17-7.08 (m, 2H), 6.56 (d, J = 5.7 Hz, 1H), 4.86 (d, 1H), 4.67 (d, J = 16.6 Hz, 1H), 3.93-3.82 (m, 2H), 3.77 (s, 3H), 3.82-3.66 (m, 1H), 3.38 (m, 4H), 3.32-3.25 (m, 2H), 3.22-3.15 (m, 1H), 2.81 (m, 4H), 1.40 (d, J = 6.9 Hz, 3H), 1.01- 0.91 (m, 2H). |
| XV- 38 | [chemical structure] | 627.25 [M + H]⁺ | (400 MHz, Methanol-d4) δ 8.18 (d, J = 4.0 Hz, 1H), 7.84 (d, J = 2.1 Hz, 1H), 7.74-7.63 (m, 2H), 7.33 (d, J = 8.5 Hz, 1H), 7.02-6.96 (m, 1H), 6.93 (s, 1H), 5.84 (s, 1H), 4.80 (d, J = 16.5 Hz, 1H), 4.66- 4.57 (m, 3H), 3.78-3.72 (m, 3H), 3.67 (dd, J = 12.8, 6.1 Hz, 1H), 2.92 (s, 8H), 1.37 (d, J = 6.9 Hz, 3H). |
| XV- 39 | [chemical structure] | 668.2 [M + H]⁺ | (400 MHz, Methanol-d4) δ 8.34 (d, J = 3.9 Hz, 1H), 7.84 (d, J = 2.1 Hz, 1H), 7.74-7.64 (m, 2H), 7.37 (d, J = 8.5 Hz, 1H), 7.07 (dd, J = 8.5, 2.6 Hz, 1H), 7.01 (d, J = 2.6 Hz, 1H), 6.07 (s, 1H), 4.80 (s, 1H), 4.61 (d, J = 16.6 Hz, 1H), 3.79- 3.72 (m, 3H), 3.68 (dd, J = 12.9, 6.1 Hz, 1H), 3.11 (d, J = 22.9 Hz, 10H), 2.92 (s, 3H), 2.48 (s, 2H), 1.37 (d, J = 6.9 Hz, 3H). |
| XV- 40 | [chemical structure] | 758 [M + H]⁺ | N/A |
| XV- 41 | [chemical structure] | 648.30 [M + H]⁺ | N/A |
| XV- 42 | [chemical structure] | 646.30 [M + H]⁺ | N/A |

### Example 65—Synthesis of Additional 3-methyl-N-phenyl-3,4-dihydroisoquinoline-2(1H)-carboxamides and Related Compounds-2(1H)-carboxamides and Related Compounds

The compounds in Table 16 below were prepared based on experimental procedures described in Example 13 and in the detailed description above.

TABLE 16

| | | Mass | |
|---|---|---|---|
| | | Spec. | |
| No. | Chemical Structure | (ES, m/z) | ¹H NMR Spectrum |
| XVI- 1 | [chemical structure] | 650.30 [M + H]⁺ | (400 MHz, Methanol-d4, ppm): δ 8.19 (d, J = 5.6 Hz, 1H), 7.83 (d, J = 1.9 Hz, 1H), 7.72-7.62 (m, 2H), 7.35 (d, J = 9.0 Hz, 1H), 7.12 (dd, J = 4.5, 2.0 Hz, 2H), 6.72 (s, 1H), 6.54 (d, J = 5.6 Hz, 1H), 4.92 (d, J = 16.7 Hz, 1H), 4.80 (d, J = 5.4 Hz, 1H), 4.51 (d, J = 16.6 Hz, 1H), 3.64 (s, 2H), 3.22 (m, 8H), 2.82 (d, J = 14.0 Hz, 1H), 2.54 (s, 7H), 2.32 (s, 3H), 1.21 (d, J = 6.7 Hz, 3H). |
| XVI- 2 | [chemical structure] | 635.50 [M + H]⁺ | (400 MHz, Methanol-d4) δ 8.34 (s, 1H), 7.85 (d, J = 2.1 Hz, 1H), 7.77-7.65 (m, 2H), 7.42 (d, J = 8.2 Hz, 1H), 7.40-7.33 (m, 1H), 7.20 (d, J = 7.4 Hz, 2H), 6.76-6.65 (m, 1H), 4.96 (d, J = 16.8 Hz, 1H), 4.81 (td, J = 6.3, 2.4 Hz, 1H), 4.53 (d, J = 16.8 Hz, 1H), 3.76 (s, 2H), 3.48-3.39 (m, 1H), 3.25 (dd, J = 15.9, 5.7 Hz, 2H), 3.04 (q, J = 7.3 Hz, 3H), 2.92 (s, 3H), 2.90-2.31 (m, 4H), 1.41-1.13 (m, 6H). |
| XVI- 3 | [chemical structure] | 636.25 [M + H]⁺ | (400 MHz, Methanol-d4) δ 8.94 (d, J = 2.4 Hz, 1H), 8.37 (d, J = 2.5 Hz, 1H), 8.31 (d, J = 6.0 Hz, 1H), 7.42 (d, J = 8.9 Hz, 1H), 7.37- 7.29 (m, 1H), 7.19 (d, J = 5.3 Hz, 2H), 6.64 (dt, J = 6.1, 1.8 Hz, 1H), 4.97 (d, J = 16.8 Hz, 1H), 4.83- 4.73 (m, 1H), 4.56 (d, J = 16.7 Hz, 1H), 3.95 (d, J = 2.6 Hz, 2H), 3.29- 3.16 (m, 3H), 3.03 (q, J = 7.3 Hz, 3H), 2.98-2.59 (m, 7H), 1.30-0.99 (m, 6H). |
| XVI- 4 | [chemical structure] | 677.35 [M + H]⁺ | N/A |
| XVI- 5 | [chemical structure] | 678.30 [M + H]⁺ | N/A |
| XVI- 6 | [chemical structure] | 668.35 [M + H]⁺ | N/A |
| XVI- 7 | [chemical structure] | 652.25 [M + H]⁺ | N/A |
| XVI- 8 | [chemical structure] | 654.30 [M + H]⁺ | N/A |
| XVI- 9 | [chemical structure] | 653.30 [M + H]⁺ | N/A |
| XVI- 10 | [chemical structure] | 651.40 [M + H]⁺ | N/A |

### Example 66—Synthesis of 7-[[5-Fluoro-2-(1-methylpyrazol-4-yl)-1H-pyrrolo[2,3-b]pyridin-4-yl]amino]-N-[4-[(4-methylpiperazin-1-yl)methyl]-3-(trifluoromethyl)phenyl]-3,4-dihydro-1H-isoquinoline-2-carboxamide (Compound No. 22)

[chemical structure]

### Part I—Synthesis of tert-Butyl 7-[[1-(benzenesulfonyl)-5-fluoro-2-(1-methylpyrazol-4-yl)pyrrolo[2,3-b]pyridin-4-yl]amino]-3,4-dihydro-1H-isoquinoline-2-carboxylate

[chemical structure]

Into a 20-mL vial, purged and maintained with an inert atmosphere of nitrogen, was placed tert-butyl 7-amino-3,4-dihydro-1H-isoquinoline-2-carboxylate (200 mg, 0.8 mmol), toluene (10 mL), potassium carbonate (330 mg, 2.4 mmol), 4-[1-(benzenesulfonyl)-4-bromo-5-fluoropyrrolo[2,3-b]pyridin-2-yl]-1-methylpyrazole (386 mg, 0.9 mmol), 2-dicyclohexylphosphino-2′,4′,6′-triisopropylbiphenyl (77 mg, 0.16 mmol), and tris(dibenzylideneacetone)dipalladium(0) (74 mg, 0.08 mmol). The reaction was stirred overnight at 100° C. The cooled reaction was concentrated, and the residue was purified on a silica gel column with ethyl acetate/petroleum ether (1:1). Desired fractions were combined and concentrated to yield the title compound (300 mg, 62%) as a solid.

### Part II—Synthesis of tert-Butyl 7-[[5-fluoro-2-(1-methylpyrazol-4-yl)-1H-pyrrolo[2,3-b]pyridin-4-yl]amino]-3,4-dihydro-1H-isoquinoline-2-carboxylate

[chemical structure]

Into a 20-mL vial, purged and maintained with an inert atmosphere of nitrogen, was placed tert-butyl 7-[[1-(benzenesulfonyl)-5-fluoro-2-(1-methylpyrazol-4-yl)pyrrolo[2,3-b]pyridin-4-yl]amino]-3,4-dihydro-1H-isoquinoline-2-carboxylate (300 mg, 0.5 mmol), tetrahydrofuran (8 mL), 2,2,2-trifluoroethanol (4 mL), and cesium carbonate (405 mg, 1.2 mmol, 2.5). The reaction was heated in a microwave for 2 hours at 100° C. The cooled mixture was concentrated, and the residue was purified on a silica gel column with ethyl acetate/petroleum ether (2:1). Desired fractions were combined and concentrated to yield the title compound (200 mg, 87%) as a solid.

### Part III—Synthesis of N-[5-Fluoro-2-(1-methylpyrazol-4-yl)-1H-pyrrolo[2,3-b]pyridin-4-yl]-1,2,3,4-tetrahydroisoquinolin-7-amine

[chemical structure]

In a 25-mL round-bottom flask was dissolved tert-butyl 7-[[5-fluoro-2-(1-methylpyrazol-4-yl)-1H-pyrrolo[2,3-b]pyridin-4-yl]amino]-3,4-dihydro-1H-isoquinoline-2-carboxylate (100 mg, 0.22 mmol) in dichloromethane (5 mL), added trifluoroacetic acid (1 mL) and stirred for 1 hour at room temperature. The mixture was concentrated to yield the title compound (72 mg, 92%).

### Part IV—Synthesis of 7-[[5-Fluoro-2-(1-methylpyrazol-4-yl)-1H-pyrrolo[2,3-b]pyridin-4-yl]amino]-N-[4-[(4-methylpiperazin-1-yl)methyl]-3-(trifluoromethyl)phenyl]-3,4-dihydro-1H-isoquinoline-2-carboxamide (Compound No. 22)

[chemical structure]

In a 25-mL round-bottom flask was dissolved N-[5-fluoro-2-(1-methylpyrazol-4-yl)-1H-pyrrolo[2,3-b]pyridin-4-yl]-1,2,3,4-tetrahydroisoquinolin-7-amine (35 mg, 0.1 mmol) in dichloromethane (5 mL). Added triethylamine (0.1 mL, 0.8 mmol), phenyl N-[4-[(4-methylpiperazin-1-yl)methyl]-3-(trifluoromethyl)phenyl]carbamate (49 mg, 0.13 mmol) and stirred overnight at room temperature. The reaction was concentrated and purified by preparatory HPLC to yield the title compound as a trifluoroacetate salt (30.2 mg, 40%). (400 MHz, Methanol-d₄) δ 8.20 (d, J=6.4 Hz, 1H), 7.90 (s, 1H), 7.84 (s, 1H), 7.73 (s, 3H), 7.41 (s, 1H), 7.26 (d, J=8.1 Hz, 2H), 5.76 (s, 1H), 4.77 (s, 2H), 3.88 (d, J=2.8 Hz, 4H), 3.75 (s, 2H), 3.58-3.38 (m, 3H), 3.00 (s, 6H), 2.92 (s, 3H), 2.52 (s, 2H).

### Example 67—Synthesis of Additional N-phenyl-3,4-dihydroisoquinoline-2(1H)-carboxamides and Related Compounds

The compounds in Table 17 below were prepared based on experimental procedures described in Example 66 and in the detailed description.

TABLE 17

| | | Mass | |
|---|---|---|---|
| | | Spec. | |
| No. | Chemical Structure | (ES, m/z) | ¹H NMR Spectrum |
| XVII- 1 | [chemical structure] | 705.40 [M + H]⁺ | (400 MHz, Methanol-d4, ppm) δ 8.51 (s, 1H), 8.28 (d, J = 2.5 Hz, 1H), 8.09-7.96 (m, 2H), 7.87- 7.78 (m, 2H), 7.68 (d, J = 2.1 Hz, 2H), 7.31 (s, 1H), 6.15 (s, 1H), 4.93 (s, 1H), 4.58 (d, J = 16.7 Hz, 1H), 4.29 (dd, J = 13.6, 3.9 Hz, 1H), 3.92 (s, 3H), 3.71 (s, 2H), 3.61 (dd, J = 13.4, 3.9 Hz, 1H), 3.29-2.83 (m, 7H), 2.82-2.37 (m, 4H), 1.99-1.82 (m, 1H), 1.77- 1.60 (m, 1H), 1.29 (t, J = 7.3 Hz, 3H), 1.13 (t, J = 7.4 Hz, 3H). |
| XVII- 2 | [chemical structure] | 663.45 [M + H]⁺ | (400 MHz, Methanol-d4, ppm): δ 8.92 (d, J = 2.5 Hz, 1H), 8.35 (d, J = 2.5 Hz, 1H), 8.20 (d, J = 6.4 Hz, 1H), 7.90 (s, 1H), 7.70 (d, J = 0.8 Hz, 1H), 7.40 (d, J = 8.7 Hz, 1H), 7.31-7.22 (m, 2H), 5.76 (s, 1H), 4.79 (s, 2H), 3.92 (d, J = 16.2 Hz, 7H), 3.32-3.25 (m, 5H), 3.10 (t, J = 5.9 Hz, 2H), 2.90 (s, 6H). |
| XVII- 3 | [chemical structure] | 680.35 [M + H]⁺ | N/A |
| XVII- 4 | [chemical structure] | 681.40 [M + H]⁺ | N/A |

### Example 68—Synthesis of N-[4-[(4-Ethylpiperazin-1-yl)methyl]-3-(trifluoromethyl)phenyl]-8-methyl-3-[1H-pyrrolo[2,3-b]pyridin-4-yloxy]-5,6,7,8-tetrahydroquinoline-6-carboxamide (Compound No. 23)

[chemical structure]

### Part I—Synthesis of tert-Butyl 3-methyl-4-oxocyclohexane-1-carboxylate

[chemical structure]

Into a round-bottom flask, purged and maintained with an inert atmosphere of nitrogen, was dissolved tert-butyl 4-oxocyclohexane-1-carboxylate (10 g, 59 mmol) in tetrahydrofuran (100 mL) and cooled to −78° C. This was followed by the addition of 1M lithium bis(trimethylsilyl)amide in tetrahydrofuran (65 mL, 65 mmol) dropwise over 1 hour. To this was added iodomethane (8.3 g, 59 mmol) dropwise and stirred for 2 hours at room temperature. The reaction was then quenched by the addition of water, extracted with ethyl acetate (3×200 mL), washed with brine, dried over anhydrous sodium sulfate and concentrated to yield the title compound (7.8 g, 72%) as an oil.

### Part II—Synthesis of 8-Methyl-3-nitro-5,6,7,8-tetrahydroquinoline-6-carboxamide

[chemical structure]

Into a 50 mL sealed tube was placed tert-butyl 3-methyl-4-oxocyclohexane-1-carboxylate (2.7 g, 14.7 mmol) and 1-methyl-3,5-dinitro-1,2-dihydropyridin-2-one (2.9 g, 14.7 mmol). Added 7M ammonia in methanol (20 mL), sealed, and stirred overnight at 100° C. The mixture was cooled and concentrated. The crude product was purified by preparatory HPLC. Pure fractions were combined and concentrated to yield the title compound (1.7 g, 49%) as an oil.

### Part III—Synthesis of 3-Amino-8-methyl-5,6,7,8-tetrahydroquinoline-6-carboxamide

[chemical structure]

To a solution of 8-methyl-3-nitro-5,6,7,8-tetrahydroquinoline-6-carboxamide (1.5 g, 6.4 mmol) in tetrahydrofuran (40 mL) was added 10% palladium on carbon (0.07 g, 0.6 mmol) under nitrogen atmosphere. The atmosphere was purged and replaced with hydrogen gas and the mixture was stirred at room temperature for 6 hours under hydrogen atmosphere using a hydrogen balloon. The reaction was filtered through a Celite pad and concentrated under reduced pressure to yield the title compound (675 mg. 50%).

### Part IV—Synthesis of 3-Hydroxy-8-methyl-5,6,7,8-tetrahydroquinoline-6-carboxylic acid

[chemical structure]

In a 50 mL round-bottom flask was dissolved 3-amino-8-methyl-5,6,7,8-tetrahydroquinoline-6-carboxamide (675 mg, 3.3 mmol), in 30% sulfuric acid in water (15 mL). This was followed by the addition of a solution of sodium nitrite (272 mg, 3.9 mmol) in water (10 mL) at 0° C. over 30 minutes. The solution was stirred for 1 hour at 50° C., then concentrated. The residue was purified on a silica gel column eluting with chloroform/methanol (10:1). Pure fractions were combined and concentrated to yield the title compound (139 mg, 20%) as a solid.

### Part V—Synthesis of Methyl 3-hydroxy-8-methyl-5,6,7,8-tetrahydroquinoline-6-carboxylate

[chemical structure]

In a 25 mL round-bottom flask was dissolved 3-hydroxy-8-methyl-5,6,7,8-tetrahydroquinoline-6-carboxylic acid (140 mg, 0.68 mmol), in methanol (3 mL), added concentrated sulfuric acid (0.5 mL) and stirred for 2 hours at 67° C. The mixture was concentrated. The residue was purified on a silica gel column eluting with chloroform/methanol (10:1). Pure fractions were combined and concentrated to yield the title compound (142 mg, 95%) as a solid.

### Part VI—Synthesis of Methyl 3-[[1-(benzenesulfonyl)-1H-pyrrolo[2,3-b]pyridin-4-yl]oxy]-8-methyl-5,6,7,8-tetrahydroquinoline-6-carboxylate

[chemical structure]

In a round-bottom flask, purged and maintained with an inert atmosphere of nitrogen, was placed methyl 3-hydroxy-8-methyl-5,6,7,8-tetrahydroquinoline-6-carboxylate (224 mg, 1.0 mmol), 1-(benzenesulfonyl)-4-bromo-1H-pyrrolo[2,3-b]pyridine (512 mg, 1.5 mmol), potassium carbonate (350 mg, 2.5 mmol), 2-dicyclohexylphosphino-2′,4′,6′-triisopropylbiphenyl (97 mg, 0.2 mmol), and tris(dibenzylideneacetone)dipalladium(0) (93 mg, 0.1 mmol) in toluene (10 mL). The resulting solution was stirred for overnight at 110° C., concentrated, and the residue was purified on a silica gel column eluting with ethyl acetate/petroleum ether (2:1). Pure fractions were combined and concentrated to yield the title compound (293 mg, 61%) as a solid.

### Part VII—Synthesis of 3-[[1-(Benzenesulfonyl)-1H-pyrrolo[2,3-b]pyridin-4-yl]oxy]-8-methyl-5,6,7,8-tetrahydroquinoline-6-carboxylic

[chemical structure]

In a round-bottom flask was dissolved methyl 3-[[1-(benzenesulfonyl)-1H-pyrrolo[2,3-b]pyridin-4-yl]oxy]-8-methyl-5,6,7,8-tetrahydroquinoline-6-carboxylate (210 mg, 0.44 mmol) in tetrahydrofuran (5 mL) and water (1 mL), added lithium hydroxide (16 mg, 0.66 mmol). The solution was stirred for 8 hours at room temperature, the pH value of the solution was adjusted to 5 with 2M hydrogen chloride, extracted with ethyl acetate (3×50 mL) and concentrated to yield the title compound (161 mg, 79%) as a solid.

### Part VIII—Synthesis of 3-[[1-(Benzenesulfonyl)-1H-pyrrolo[2,3-b]pyridin-4-yl]oxy]-8-methyl-5,6,7,8-tetrahydroquinoline-6-carbonyl chloride

[chemical structure]

In a 25-mL round-bottom flask was dissolved 3-[[1-(benzenesulfonyl)-1H-pyrrolo[2,3-b]pyridin-4-yl]oxy]-8-methyl-5,6,7,8-tetrahydroquinoline-6-carboxylic acid (160 mg, 0.35 mmol) in dichloromethane (7 mL) at 0° C., added N,N-dimethylformamide (25 mg, 0.35 mmol), followed by the slow addition of oxalyl chloride (66 mg, 0.52 mmol) over 3 minutes. The resulting solution was stirred for 1 hour at room temperature and concentrated to yield the crude title compound (140 mg, 84%) as an oil.

### Part IX—Synthesis of 3-[[1-(Benzenesulfonyl)-1H-pyrrolo[2,3-b]pyridin-4-yl]oxy]-N-[4-[(4-ethylpiperazin-1-yl)methyl]-3-(trifluoromethyl)phenyl]-8-methyl-5,6,7,8-tetrahydroquinoline-6-carboxamide

[chemical structure]

In a 25 mL round-bottom flask was dissolved 3-[[1-(benzenesulfonyl)-1H-pyrrolo[2,3-b]pyridin-4-yl]oxy]-8-methyl-5,6,7,8-tetrahydroquinoline-6-carbonyl chloride (140 mg, 0.29 mmol) in dichloromethane (7 mL), added pyridine (34 mg, 0.44 mmol), 4-[(4-ethylpiperazin-1-yl)methyl]-3-(trifluoromethyl)aniline (117 mg, 0.41 mmol) and stirred for 1 hour at 50° C. The mixture was concentrated and the residue was purified on a silica gel column eluting with dichloromethane/methanol (95:5). Pure fractions were combined and concentrated to yield the title compound (160 mg, 75%) as a solid.

### Part X—Synthesis of N-[4-[(4-Ethylpiperazin-1-yl)methyl]-3-(trifluoromethyl)phenyl]-8-methyl-3-[1H-pyrrolo[2,3-b]pyridin-4-yloxy]-5,6,7,8-tetrahydroquinoline-6-carboxamide (Compound No. 23)

[chemical structure]

In a vial was added 3-[[1-(benzenesulfonyl)-1H-pyrrolo[2,3-b]pyridin-4-yl]oxy]-N-[4-[(4-ethylpiperazin-1-yl)methyl]-3-(trifluoromethyl)phenyl]-8-methyl-5,6,7,8-tetrahydroquinoline-6-carboxamide (10 mg, 0.014 mmol), ethanol (3 mL), water (1 mL), and lithium hydroxide (1.3 mg, 0.06 mmol). The solution was stirred overnight at room temperature then purified by preparatory-HPLC. Pure fractions were combined and concentrated to yield the title compound (2 mg, 25%). H-NMR— (400 MHz, DMSO-d₄, ppm) δ 0.99 (t, J=7.2 Hz, 3H), 1.36 (d, J=7.2 Hz, 3H), 1.95 (dd, J=14.1, 3.4 Hz, 1H), 2.19-2.09 (m, 1H), 2.46-2.21 (m, 9H), 2.98 (s, 3H), 3.10 (dq, J=10.6, 6.9 Hz, 1H), 3.54 (s, 2H), 6.26 (dd, J=3.5, 1.9 Hz, 1H), 6.45 (d, J=5.4 Hz, 1H), 7.42-7.39 (m, 1H), 7.46 (d, J=2.7 Hz, 1H), 7.67 (d, J=8.5 Hz, 1H), 7.82 (dd, J=8.5, 2.2 Hz, 1H), 8.09 (d, J=2.2 Hz, 1H), 8.11 (d, J=5.5 Hz, 1H), 8.32 (d, J=2.7 Hz, 1H), 10.36 (s, 1H), 11.81 (s, 1H).

### Example 69—Synthesis of 3-((1H-Pyrrolo[2,3-b]pyridin-4-yl)oxy)-N-(4-((4-ethylpiperazin-1-yl)methyl)-3-(trifluoromethyl)phenyl)-5,6,7,8-tetrahydroquinoline-6-carboxamide (Compound No. 24)

[chemical structure]

In a similar manner as Example 68, 3-((1H-pyrrolo[2,3-b]pyridin-4-yl)oxy)-N-(4-((4-ethylpiperazin-1-yl)methyl)-3-(trifluoromethyl)phenyl)-5,6,7,8-tetrahydroquinoline-6-carboxamide was synthesized by replacing tert-butyl 3-methyl-4-oxocyclohexane-1-carboxylate with ethyl 4-oxocyclohexane-1-carboxylate in Part II. H-NMR— (400 MHz, Methanol-d4) δ 8.48 (d, J=2.6 Hz, 1H), 8.31 (d, J=6.5 Hz, 1H), 8.06 (d, J=2.2 Hz, 1H), 7.85 (dt, J=5.5, 2.4 Hz, 2H), 7.75 (d, J=8.5 Hz, 1H), 7.55 (d, J=3.6 Hz, 1H), 6.91 (d, J=6.5 Hz, 1H), 6.66 (d, J=3.6 Hz, 1H), 3.77 (s, 2H), 3.54 (s, 2H), 3.23 (q, J=7.4 Hz, 4H), 3.17-2.93 (m, 7H), 2.51 (s, 2H), 2.37 (d, J=13.6 Hz, 1H), 2.18 (ddt, J=13.6, 9.8, 5.0 Hz, 1H), 1.36 (t, J=7.3 Hz, 3H).

### Example 70—Synthesis of Additional N-phenyl-3,4-dihydroisoquinoline-2(1H)-carboxamides and Related Compounds

The compounds in Table 18 below were prepared based on experimental procedures described above.

TABLE 18

| | | Mass | |
|---|---|---|---|
| | | Spec. | |
| No. | Chemical Structure | (ES, m/z) | ¹H NMR Spectrum |
| XVIII- 1 | [chemical structure] | 618.35 [M + H]⁺ | N/A |
| XVIII- 2 | [chemical structure] | 622.3 [M + H]⁺ | (400 MHz, Methanol-d4, ppm) δ 8.19 (d, J = 6.8 Hz, 1H), 7.86 (d, J = 2.1 Hz, 1H), 7.78-7.67 (m, 2H), 7.64 (s, 1H), 6.78 (d, J = 6.8 Hz, 1H), 6.38 (d, J = 1.3 Hz, 1H), 4.94 (d, J = 17.1 Hz, 1H), 4.68 (d, J = 17.0 Hz, 1H), 3.95 (dd, J = 13.6, 5.2 Hz, 1H), 3.84 (dd, J = 13.7, 4.3 Hz, 1H), 3.77 (s, 2H), 3.64-3.35 (m, 3H), 3.30-3.18 (m, 4H), 3.18- 2.75 (m, 4H), 2.54 (d, J = 1.1 Hz, 3H), 2.46 (s, 3H), 1.45 (d, J = 7.0 Hz, 3H), 1.36 (t, J = 7.3 Hz, 3H). |
| XVIII- 3 | [chemical structure] | 640.25 [M + H]⁺ | (400 MHz, Methanol-d4) δ 8.34 (d, J = 3.9 Hz, 1H), 7.84 (d, J = 2.1 Hz, 1H), 7.73-7.64 (m, 2H), 7.29 (d, J = 8.2 Hz, 1H), 7.07-7.00 (m, 2H), 6.06 (s, 1H), 4.69 (s, 2H), 3.86-3.71 (m, 4H), 3.26 (t, J = 5.2 Hz, 5H), 3.10 (d, J = 23.3 Hz, 6H), 2.99 (t, J = 6.0 Hz, 2H), 2.73 (t, J = 5.2 Hz, 4H), 1.32 (d, J = 9.6 Hz, 1H). |
| XVIII- 4 | [chemical structure] | 654.3 [M + H]⁺ | (400 MHz, Methanol-d4) δ 8.34 (d, J = 3.9 Hz, 1H), 7.84 (d, J = 2.1 Hz, 1H), 7.74-7.63 (m, 2H), 7.29 (d, J = 8.3 Hz, 1H), 7.11-6.98 (m, 2H), 6.07 (s, 1H), 4.69 (s, 2H), 3.82 (t, J = 5.9 Hz, 2H), 3.74 (s, 2H), 3.47 (s, 2H), 3.10 (d, J = 23.4 Hz, 8H), 2.99 (t, J = 6.0 Hz, 3H), 2.92 (s, 4H), 2.47 (s, 2H), 1.31 (s, 1H). |
| XVIII- 5 | [chemical structure] | 625.2 [M + H]⁺ | (400 MHz, DMSO-d6) δ 11.50 (s, 1H), 8.82 (s, 1H), 8.19 (d, J = 3.5 Hz, 1H), 7.89 (s, 1H), 7.75 (d, J = 8.5 Hz, 1H), 7.59 (d, J = 8.5 Hz, 1H), 7.21 (d, J = 8.3 Hz, 1H), 6.93- 6.84 (m, 2H), 6.15 (s, 1H), 5.85 (s, 1H), 4.61 (s, 2H), 3.72 (t, J = 6.0 Hz, 2H), 3.48 (s, 2H), 2.85 (s, 2H), 2.71 (d, J = 5.0 Hz, 4H), 2.30 (s, 4H), 1.07 (s, 3H). |
| XVIII- 6 | [chemical structure] | 639.25 [M + H]⁺ | (400 MHz, DMSO-d6) δ 11.50 (s, 1H), 8.82 (s, 1H), 8.19 (d, J = 3.4 Hz, 1H), 7.89 (d, J = 2.3 Hz, 1H), 7.75 (d, J = 7.9 Hz, 1H), 7.57 (d, J = 8.5 Hz, 1H), 7.21 (d, J = 8.3 Hz, 1H), 6.94-6.83 (m, 2H), 6.15 (s, 1H), 5.85 (s, 1H), 4.61 (s, 2H), 3.72 (t, J = 5.8 Hz, 2H), 3.51 (s, 2H), 2.85 (t, J = 5.8 Hz, 2H), 2.47- 2.22 (m, 8H), 2.15 (s, 3H), 1.07 (s, 4H). |
| XVIII- 7 | [chemical structure] | 621.27 [M + H]⁺ | (400 MHz, Methanol-d4) δ 8.31 (t, J = 5.4 Hz, 1H), 7.86 (d, J = 2.2 Hz, 1H), 7.74-7.65 (m, 2H), 7.42 (d, J = 8.0 Hz, 1H), 7.18 (d, J = 8.4 Hz, 2H), 6.91 (d, J = 4.2 Hz, 1H), 6.77 (d, J = 7.4 Hz, 1H), 4.77 (s, 2H), 3.85 (t, J = 5.9 Hz, 2H), 3.76 (s, 2H), 3.26 (t, J = 5.1 Hz, 7H), 3.17 (s, 3H), 3.05 (t, J = 6.0 Hz, 2H), 2.75 (s, 4H). |
| XVIII- 8 | [chemical structure] | 635.28 [M + H]⁺ | (400 MHz, Methanol-d4) δ 8.31 (d, J = 6.3 Hz, 1H), 7.86 (d, J = 2.3 Hz, 1H), 7.75-7.59 (m, 2H), 7.42 (d, J = 8.1 Hz, 1H), 7.18 (d, J = 8.4 Hz, 2H), 6.91 (t, J = 3.1 Hz, 1H), 6.78 (dd, J = 9.5, 6.4 Hz, 1H), 4.77 (s, 2H), 3.85 (t, J = 5.9 Hz, 2H), 3.76 (s, 2H), 3.35 (s, 8H), 3.17 (s, 4H), 3.05 (t, J = 6.0 Hz, 2H), 2.92 (s, 3H), 2.68 (s, 2H) |
| XVIII- 9 | [chemical structure] | 607.35 [M + H]⁺ | (400 MHz, Methanol-d4, ppm): δ 8.38-8.27 (m, 1H), 7.85 (d, J = 3.8 Hz, 1H), 7.76-7.57 (m, 2H), 7.47-7.31 (m, 2H), 7.23-7.09 (m, 2H), 6.74-6.56 (m, 1H), 4.77 (s, 2H), 3.85 (dt, J = 5.9, 3.0 Hz, 2H), 3.80-3.73 (m, 2H), 3.28- 3.20 (m, 4H), 3.10-2.96 (m, 4H), 2.75 (s, 4H), 1.24 (t, J = 7.3 Hz, 3H). |
| XVIII- 10 | [chemical structure] | 621.35 [M + H]⁺ | (400 MHz, Methanol-d4, ppm): δ 8.38-8.21 (m, 1H), 7.85 (d, J = 2.5 Hz, 1H), 7.77-7.59 (m, 2H), 7.42 (d, J = 7.0 Hz, 2H), 7.17 (d, J = 6.7 Hz, 2H), 6.69 (d, J = 6.3 Hz, 1H), 4.77 (s, 2H), 3.86 (t, J = 5.9 Hz, 2H), 3.76 (s, 2H), 3.52-3.42 (m, 1H), 3.31-3.23 (m, 1H), 3.13- 2.96 (m, 5H), 2.92 (s, 4H), 2.76- 2.12 (m, 2H), 1.24 (t, J = 7.3 Hz, 3H). |
| XVIII- 11 | [chemical structure] | 580.2 [M + H]⁺ | N/A |
| XVIII- 12 | [chemical structure] | 689.45 [M + H]⁺ | N/A |
| XVIII- 13 | [chemical structure] | 704.25 [M + H]⁺ | N/A |
| XVIII- 14 | [chemical structure] | 633.20 [M + H]⁺ | N/A |
| XVIII- 15 | [chemical structure] | 647.35 [M + H]⁺ | N/A |
| XVIII- 16 | [chemical structure] | 608.25 [M + H]⁺ | N/A |
| XVIII- 17 | [chemical structure] | 608.25 [M + H]⁺ | N/A |
| XVIII- 18 | [chemical structure] | 594.25 [M + H]⁺ | N/A |
| XVIII- 19 | [chemical structure] | 594.25 [M + H]⁺ | N/A |

### Example 71—Biological Assays for Inhibition of HPK1 Activity

Exemplary compounds from the above Examples were tested for ability to inhibit HPK1 using a human HPK1 protein TR-FRET Assay. Assay procedures and results are described below.

### Part I—Procedures for Human HPK1 Protein TR-FRET Assay

N-terminal GST-tagged recombinant human HPK1 protein (a.a. 1-346) was expressed in Sf9 insect cells using a baculovirus expression system (Signalchem Catalog #M23-11G). The enzyme was diluted in assay buffer (50 mM Tris pH 7.5, 1 mM MgCl₂, 0.01% NP-40 and 2 mM DTT) to obtain a 4× working solution at a concentration of 40 nM HPK1 enzyme.

A 5 μM stock of ULight™ CREBtide peptide RRPSYRK (Perkin Elmer Product No. TRF0107-M) derived from human cAMP Response Element Binding (CREB) Protein was prepared in storage buffer containing 50 mM Tris-HCl (pH 7.4), 0.9% NaCl, 0.1% BSA, and 0.05% sodium azide. The CREBtide substrate was diluted in assay buffer (50 mM Tris pH 7.5, 1 mM MgCl₂, 0.01% NP-40, 2 mM DTT) with 2 mM ATP to a final concentration of 200 nM CREBtide peptide to obtain a 2× working solution of substrate. HPK1 enzyme and CREBtide substrate solutions were mixed with compounds diluted in DMSO to obtain a final concentration of enzyme, substrate, and ATP of 10 nM, 100 nM, and 1 mM, respectively, in 1% DMSO. The final assay mixture was incubated at room temperature for 60 minutes.

A solution of Europium-labeled anti-phospho-CREB (Ser133) antibody (Perkin Elmer Product No. TRF0200-M), EDTA and LANCE™ Detection Buffer (Perkin Elmer Product No. CR97-100) were added to each well to obtain a final concentration of 2 nM antibody, 10 mM EDTA and 1× LANCE buffer. The resulting mixture was incubated at room temperature for 60 minutes.

The fluorescence resonance energy transfer (FRET) signal was measured on an Envision plate reader: Excitation filter=340 nm, APC emission=665 nm, Europium emission=615 nm, dichroic mirror=D400/D630, delay time=100 μs, and integration time=200 μs. The half maximal inhibitory concentration (IC₅₀) values for compounds were calculated from the quotient of the fluorescence signal at 665 nm divided by the fluorescence signal at 615 nm. The quotient of the fluorescence signals in the presence of DMSO without compound is set as 100 percent enzymatic activity. A 4-parameter logistic model in PRISM (GraphPad) was used to calculate the IC₅₀ for a given compound.

### Part II—Results

Experimental results are provided in Tables 19 and 20 below. The symbol “++++” indicates an IC₅₀ less than 0.5 μM. The symbol “+++” indicates an IC₅₀ in the range of 0.5 μM to 5 μM. The symbol “++” indicates an IC₅₀ in the range of greater than 5 μM to 10 μM. The symbol “+” indicates an IC₅₀ greater than 10 μM. The symbol “N/A” indicates that no data was available.

TABLE 19

| Compound No. | IC₅₀ |
|---|---|
| 1 | ++++ |
| 2 | ++++ |
| 3 | ++++ |
| 4 | ++++ |
| 5 | ++++ |
| Enantiomer A | |
| 5 | ++++ |
| Enantiomer B | |
| 6 | ++++ |
| 7 | ++++ |
| 8 | ++++ |
| 9 | ++++ |
| 10 | ++++ |
| 11 | +++ |
| 12 | +++ |
| 13 | ++++ |
| 14 | +++ |
| 15 | ++++ |
| 16 | ++++ |
| 17 | ++++ |
| 18 | ++++ |
| II-1 | ++++ |
| II-2 | ++++ |
| II-3 | ++++ |
| II-4 | ++++ |
| II-5 | ++++ |
| II-6 | ++++ |
| II-7 | ++++ |
| II-8 | ++++ |
| II-9 | ++++ |
| II-10 | ++++ |
| II-11 | ++++ |
| II-12 | ++++ |
| II-13 | ++++ |
| II-14 | ++++ |
| II-15 | ++++ |
| II-16 | +++ |
| II-17 | ++++ |
| II-18 | ++++ |
| II-19 | ++++ |
| II-20 | ++++ |
| II-21 | +++ |
| II-22 | ++++ |
| II-23 | ++++ |
| II-24 | ++++ |
| II-25 | ++++ |
| II-26 | ++++ |
| III-1 | ++++ |
| III-2 | ++++ |
| III-3 | ++++ |
| III-4 | ++++ |
| III-5 | ++++ |
| III-6 | +++ |
| III-7 | +++ |
| III-8 | ++++ |
| III-9 | ++++ |
| III-11 | ++++ |
| III-12 | +++ |
| III-13 | ++++ |
| IV-1 | +++ |
| IV-2 | ++++ |
| IV-3 | ++++ |
| IV-4 | ++++ |
| IV-5 | +++ |
| IV-6 | ++++ |
| IV-7 | +++ |
| IV-8 | ++ |
| IV-9 | ++++ |
| IV-10 | +++ |
| IV-11 | +++ |
| IV-12 | +++ |
| IV-13 | ++++ |
| IV-14 | ++++ |
| IV-15 | ++++ |
| IV-16 | +++ |
| IV-17 | ++++ |
| IV-18 | ++++ |
| IV-19 | ++++ |
| IV-20 | ++++ |
| IV-21 | ++++ |
| IV-22 | ++++ |
| IV-23 | ++++ |
| IV-24 | +++ |
| IV-25 | ++++ |
| IV-26 | +++ |
| IV-27 | ++++ |
| IV-28 | ++++ |
| IV-29 | ++++ |
| IV-30 | +++ |
| IV-31 | ++++ |
| IV-32 | + |
| IV-33 | ++++ |
| IV-34 | + |
| IV-35 | ++++ |
| IV-36 | ++++ |
| IV-37 | ++++ |
| IV-38 | ++++ |
| IV-39 | ++++ |
| IV-40 | ++++ |
| IV-41 | ++++ |
| V-1 | ++++ |
| V-2 | ++++ |
| V-3 | ++++ |
| V-4 | ++++ |
| V-5 | ++++ |
| V-6 | ++++ |
| V-7 | ++++ |
| V-8 | ++++ |
| V-9 | ++++ |
| V-10 | ++++ |
| V-11 | +++ |
| V-12 | +++ |
| V-13 | ++++ |
| V-14 | +++ |
| V-15 | +++ |
| V-16 | +++ |
| V-17 | ++++ |
| V-18 | ++++ |
| V-19 | ++++ |
| V-20 | +++ |
| V-21 | +++ |
| V-22 | ++++ |
| V-23 | ++++ |
| V-24 | ++++ |
| V-25 | ++++ |
| V-26 | ++++ |
| V-27 | ++++ |
| V-28 | ++++ |
| V-29 | ++++ |
| VI-1 | ++++ |
| VI-2 | ++++ |
| VI-3 | ++++ |
| VI-4 | +++ |
| VI-5 | ++++ |
| VI-6 | +++ |
| VI-7 | ++++ |
| VI-8 | ++++ |
| VI-9 | +++ |
| VI-10 | +++ |
| VI-11 | +++ |
| VI-12 | +++ |
| VI-13 | +++ |
| VI-14 | ++ |
| VI-15 | +++ |
| VI-16 | +++ |
| VI-17 | +++ |
| VI-18 | ++++ |
| VII-1 | ++++ |
| VII-2 | +++ |
| VII-3 | ++++ |
| VII-4 | ++++ |
| VII-5 | ++++ |
| VII-6 | ++++ |
| VII-7 | ++++ |
| VII-8 | ++++ |
| VII-9 | +++ |
| VII-10 | ++++ |
| VII-11 | +++ |
| VII-12 | ++++ |
| VII-13 | ++++ |
| VII-14 | ++++ |
| VII-15 | ++++ |
| VII-16 | ++++ |
| VII-17 | ++++ |
| VII-18 | ++++ |
| VII-19 | ++++ |
| VII-20 | ++++ |
| VII-21 | ++++ |
| VII-22 | ++++ |
| VII-23 | ++++ |
| VIII-1 | +++ |
| VIII-2 | ++++ |
| VIII-3 | +++ |
| VIII-4 | +++ |
| VIII-5 | +++ |
| VIII-6 | +++ |
| IX-1 | ++++ |
| IX-2 | +++ |
| IX-3 | ++ |
| X-1 | +++ |
| X-2 | +++ |
| X-3 | +++ |
| X-4 | + |
| X-5 | ++++ |
| X-6 | ++++ |
| X-7 | ++++ |
| X-8 | ++ |

TABLE 20

| Compound No. | IC₅₀ |
|---|---|
| XI-1 | +++ |
| XI-2 | +++ |
| XI-3 | ++++ |
| XI-4 | +++ |
| XI-5 | ++++ |
| XI-6 | ++++ |
| XI-7 | +++ |
| XI-8 | ++ |
| XI-9 | ++++ |
| XI-10 | ++++ |
| XI-11 | ++++ |
| XI-12 | + |
| 19 | ++++ |
| XII-1 | ++++ |
| XII-2 | ++++ |
| XII-3 | ++++ |
| XII-4 | ++++ |
| XII-5 | ++++ |
| XII-6 | ++++ |
| XII-7 | ++++ |
| XII-8 | ++++ |
| XII-9 | ++++ |
| XII-10 | ++++ |
| XII-11 | ++++ |
| XII-12 | ++++ |
| XII-13 | ++++ |
| XII-14 | ++++ |
| XII-15 | ++++ |
| XII-16 | ++++ |
| XIII-1 | ++++ |
| XIII-2 | ++++ |
| XIII-3 | ++++ |
| XIII-4 | ++++ |
| XIII-5 | ++++ |
| XIII-6 | ++++ |
| XIII-7 | ++++ |
| XIII-8 | ++++ |
| XIII-9 | ++++ |
| XIII-10 | ++++ |
| XIII-11 | ++++ |
| XIII-12 | ++++ |
| XIII-13 | ++++ |
| XIII-14 | ++++ |
| XIII-15 | ++++ |
| XIII-16 | ++++ |
| XIII-17 | ++++ |
| 20 | ++++ |
| 21 | ++++ |
| XIV-1 | ++++ |
| XIV-2 | ++++ |
| XIV-3 | ++++ |
| XIV-4 | ++++ |
| XIV-5 | ++++ |
| XIV-6 | ++++ |
| XIV-7 | +++ |
| XIV-8 | +++ |
| XIV-9 | ++++ |
| XIV-10 | ++++ |
| XIV-11 | ++++ |
| XIV-12 | ++++ |
| XIV-13 | ++++ |
| XIV-14 | ++++ |
| XIV-15 | ++++ |
| XIV-16 | +++ |
| XIV-17 | + |
| XIV-18 | ++ |
| XIV-19 | ++++ |
| XIV-20 | ++++ |
| XIV-21 | ++++ |
| XIV-22 | ++++ |
| XIV-23 | ++++ |
| XIV-24 | ++++ |
| XIV-25 | ++++ |
| XIV-26 | ++++ |
| XIV-27 | ++ |
| XIV-28 | ++++ |
| XIV-29 | ++++ |
| XIV-30 | ++++ |
| XIV-31 | ++++ |
| XIV-32 | ++++ |
| XIV-33 | ++++ |
| XIV-34 | ++++ |
| XIV-35 | ++++ |
| XIV-36 | ++++ |
| XIV-37 | ++++ |
| XIV-38 | ++++ |
| XIV-39 | ++++ |
| XIV-40 | ++++ |
| XIV-41 | ++ |
| XIV-42 | ++++ |
| XIV-43 | ++++ |
| XIV-44 | ++++ |
| XIV-45 | ++++ |
| XIV-46 | ++++ |
| XIV-47 | ++++ |
| XIV-48 | ++++ |
| XIV-49 | ++++ |
| XIV-50 | ++++ |
| XIV-51 | ++++ |
| XIV-52 | ++++ |
| XIV-53 | ++++ |
| XIV-54 | ++++ |
| XIV-55 | ++++ |
| XIV-56 | ++++ |
| XIV-57 | ++++ |
| XIV-58 | ++++ |
| XIV-59 | ++++ |
| XIV-60 | ++++ |
| XIV-61 | ++++ |
| XIV-62 | ++++ |
| XIV-63 | ++++ |
| XIV-64 | ++++ |
| XIV-65 | ++++ |
| XIV-66 | ++++ |
| XIV-67 | + |
| XIV-68 | +++ |
| XIV-69 | ++++ |
| XIV-70 | ++++ |
| XIV-71 | +++ |
| XIV-72 | ++++ |
| XIV-73 | ++++ |
| XIV-74 | ++++ |
| XIV-75 | ++++ |
| XIV-76 | ++++ |
| XIV-77 | ++++ |
| XIV-78 | ++++ |
| XIV-79 | ++++ |
| XIV-80 | ++++ |
| XIV-81 | ++++ |
| XIV-82 | ++++ |
| XIV-82 | ++++ |
| XIV-84 | ++++ |
| XIV-85 | ++++ |
| XIV-86 | ++++ |
| XIV-87 | ++++ |
| XIV-88 | ++++ |
| XIV-89 | ++++ |
| XIV-90 | ++++ |
| XIV-91 | ++++ |
| XIV-92 | ++++ |
| XIV-93 | ++++ |
| XIV-94 | ++++ |
| XIV-95 | ++++ |
| XIV-96 | ++++ |
| XIV-97 | ++++ |
| XIV-98 | ++++ |
| XIV-99 | +++ |
| XIV-100 | ++ |
| XIV-101 | +++ |
| XIV-102 | ++++ |
| XIV-103 | ++++ |
| XIV-104 | ++++ |
| XIV-105 | ++++ |
| XIV-106 | ++++ |
| XIV-107 | ++++ |
| XIV-108 | ++++ |
| XIV-109 | ++++ |
| XIV-110 | ++++ |
| XIV-111 | ++++ |
| XIV-112 | ++++ |
| XIV-113 | ++++ |
| XIV-114 | ++++ |
| XIV-115 | ++ |
| XIV-116 | ++ |
| XIV-117 | ++++ |
| XIV-118 | ++++ |
| XIV-119 | +++ |
| XIV-120 | +++ |
| XIV-121 | ++++ |
| XIV-122 | ++++ |
| XIV-123 | ++++ |
| XIV-124 | ++++ |
| XIV-125 | ++++ |
| XIV-126 | ++++ |
| XIV-127 | ++++ |
| XIV-128 | ++ |
| XIV-129 | +++ |
| XIV-130 | ++++ |
| XIV-131 | ++++ |
| XIV-132 | ++++ |
| XIV-133 | ++++ |
| XIV-134 | ++++ |
| XIV-135 | ++++ |
| XIV-136 | ++++ |
| XIV-137 | ++++ |
| XIV-138 | ++++ |
| XIV-139 | +++ |
| XIV-140 | + |
| XIV-141 | ++++ |
| XIV-143 | ++++ |
| XIV-144 | ++++ |
| XIV-145 | ++++ |
| XIV-146 | ++++ |
| XIV-147 | ++++ |
| XIV-148 | ++++ |
| XIV-149 | ++++ |
| XIV-150 | ++++ |
| XIV-151 | ++++ |
| XIV-152 | ++++ |
| XIV-153 | ++++ |
| XIV-154 | ++++ |
| XIV-155 | ++++ |
| XV-1 | ++++ |
| XV-2 | ++++ |
| XV-3 | ++++ |
| XV-4 | ++++ |
| XV-5 | ++++ |
| XV-6 | ++++ |
| XV-7 | ++++ |
| XV-8 | ++++ |
| XV-9 | ++++ |
| XV-10 | ++++ |
| XV-11 | ++++ |
| XV-12 | ++++ |
| XV-13 | ++++ |
| XV-14 | ++++ |
| XV-15 | ++++ |
| XV-16 | ++++ |
| XV-17 | ++++ |
| XV-18 | ++++ |
| XV-19 | ++++ |
| XV-20 | ++++ |
| XV-21 | ++++ |
| XV-22 | ++++ |
| XV-23 | ++++ |
| XV-24 | ++++ |
| XV-25 | ++++ |
| XV-26 | ++++ |
| XV-27 | ++++ |
| XV-28 | ++++ |
| XV-29 | ++++ |
| XV-30 | ++++ |
| XV-31 | ++++ |
| XV-32 | ++++ |
| XV-33 | ++++ |
| XV-34 | ++++ |
| XV-35 | ++++ |
| XV-36 | ++++ |
| XV-37 | ++++ |
| XV-38 | ++++ |
| XV-39 | ++++ |
| XV-40 | ++++ |
| XV-41 | ++++ |
| XV-42 | ++++ |
| XVI-1 | ++++ |
| XVI-2 | ++++ |
| XVI-3 | ++++ |
| XVI-4 | ++++ |
| XVI-5 | ++++ |
| XVI-6 | ++++ |
| XVI-7 | ++++ |
| XVI-8 | ++++ |
| XVI-9 | ++++ |
| XVI-10 | ++++ |
| 22 | ++++ |
| XVII-1 | ++++ |
| XVII-2 | ++++ |
| XVII-3 | ++++ |
| XVII-4 | ++++ |
| 23 | ++++ |
| trans isomer | |
| 23 | ++++ |
| cis isomer | |
| 24 | ++++ |
| XVIII-1 | ++++ |
| XVIII-2 | ++++ |
| XVIII-3 | ++++ |
| XVIII-4 | ++++ |
| XVIII-5 | ++++ |
| XVIII-6 | ++++ |
| XVIII-7 | ++++ |
| XVIII-8 | ++++ |
| XVIII-9 | ++++ |
| XVIII-10 | ++++ |
| XVIII-11 | +++ |
| XVIII-12 | ++++ |
| XVIII-13 | ++++ |
| XVIII-14 | ++++ |
| XVIII-15 | ++++ |
| XVIII-16 | ++++ |
| XVIII-17 | ++++ |
| XVIII-18 | ++++ |
| XVIII-19 | ++++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: [chemical structure]
or a pharmaceutically acceptable salt thereof; wherein:
R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);
R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;
R³ represents independently for each occurrence hydrogen, C₁₋₄ alkyl, or C₃₋₅ cycloalkyl;
R⁴ represents independently for each occurrence halogen, C₁₋₄ alkyl, C₁₋₄ haloalkyl, cyano, C₁₋₄ alkoxyl, C₁₋₄ haloalkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN;
R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring optionally substituted with 1 or 2 substituents independently selected from the group consisting of halogen, hydroxyl, C₁₋₄ alkyl, C₁₋₄ haloalkyl, cyano, and C₁₋₄ alkoxyl;
A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, cyano, C₁₋₄ alkoxyl, —(C₁₋₄ alkylene)-(C₁₋₄ alkoxyl), —(C₁₋₄ alkylene)-S(O)ₜ—(C₁₋₄ alkyl), —C(O)N(R³)(R⁵), —C(O)OR³, —C(O)R³, —N(R³)(R⁵), —N(R³)C(O)R⁵, —N(R³)CO₂(C₁₋₄ alkyl), —N(R³)C(O)—(5-6 membered heteroaryl), —(C₁₋₄ alkylene)-N(R³)(R⁵), —(C₁₋₄ alkylene)-N(R³)C(O)R⁵, —N(R³)—(5-6 membered heteroaryl), 3-7 membered heterocyclyl, 5-6 membered unsaturated oxo-heterocyclyl, and aryl; wherein said C₃₋₇ cycloalkyl, C₅₋₇ cycloalkenyl, 3-7 membered heterocyclyl, 5-6 membered heteroaryl, 5-6 membered unsaturated oxo-heterocyclyl, and aryl are each optionally substituted with 1 or 2 substituents independently selected from the group consisting of halogen, hydroxyl, C₁₋₄ alkyl, C₁₋₄ haloalkyl, C₁₋₄ hydroxyalkyl, —(C₁₋₄ alkylene)-N(R³)(R⁵), cyano, —N(R³)(R⁵), —C(O)R³ and —C(O)N(R³)R⁵;
A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;
X¹ is —O—, —N(R³)—, —S(O)ₜ—, or a bond;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, oxo, cyano, C₁₋₄ alkoxyl, —(C₁₋₄ alkylene)-(C₁₋₄ alkoxyl), C₃₋₇ cycloalkyl, 3-7 membered heterocycloalkyl, and 5-6 membered heteroaryl;
m and n are each independently 0, 1, 2, or 3; and
t represents independently for each occurrence 0, 1 or 2.

2. The compound of claim 1, wherein A¹ is a 5-6 membered heteroaromatic ring containing at least one ring nitrogen atom.

3. The compound of claim 1, wherein A¹ is

[chemical structure]

4. The compound of claim 1, wherein A¹ is a 6-membered unsaturated oxo-heterocyclic ring containing at least one ring nitrogen atom.

5. The compound of claim 1, wherein A¹ is a 6-membered carbocyclic aromatic ring.

6. The compound of any one of claims 1-5, wherein A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

7. The compound of any one of claims 1-5, wherein A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

8. The compound of any one of claims 1-5, wherein A² is one of the following [chemical structure]
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

9. The compound of any one of claims 1-5, wherein A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

10. The compound of any one of claims 1-5, wherein A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

11. The compound of any one of claims 1-5, wherein, A² is a 8-10 membered bicyclic heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

12. The compound of any one of claims 1-5, wherein A² is [chemical structure]
substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

13. The compound of any one of claims 1-12, wherein A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.

14. The compound of any one of claims 1-12, wherein A³ is phenyl substituted by (i) —(C₁₋₄ alkylene)-X² and (ii) 1 or 2 occurrences of R⁴.

15. The compound of any one of claims 1-12, wherein A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.

16. The compound of any one of claims 1-12, wherein A³ is pyridinyl, pyrazinyl, or pyrimidinyl; each of which is substituted by (i) —(C₁₋₄ alkylene)-X² or —O—X², and (ii) 1 occurrence of R⁴.

17. The compound of any one of claims 1-16, wherein X¹ is —O—.

18. The compound of any one of claims 1-16, wherein X¹ is —N(R³)— or —S—.

19. The compound of any one of claims 1-18, wherein R² is C₁₋₄ alkyl, and m is 1 or 2.

20. The compound of claim 1, wherein the compound is represented by Formula I-A: [chemical structure]
or a pharmaceutically acceptable salt thereof; wherein:
R¹ represents independently for each occurrence halogen or C₁₋₄ alkyl;
R^{2A}, R^{2B}, and R^{2C} each represent independently hydrogen, C₁₋₄ alkyl, C₁₋₄ haloalkyl, C₁₋₄ hydroxyalkyl, or —(C₁₋₄ alkylene)-(C₁₋₄ alkoxyl);
R³ represents independently for each occurrence hydrogen or C₁₋₄ alkyl;
R⁴ represents independently for each occurrence C₁₋₄ alkyl, C₁₋₄ haloalkyl, C₃₋₅ cycloalkyl, C₃₋₅ halocycloalkyl, -(cyclopropylene)-(C₁₋₄ haloalkyl), or -(cyclopropylene)-CN;
R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;
A¹ is a 6-membered heteroaromatic ring containing at least one ring nitrogen atom, or a 6-membered carbocyclic aromatic ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);
A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl; and
n is 0, 1, or 2.

21. The compound of claim 20, wherein A¹ is a 6-membered heteroaromatic ring containing at least one ring nitrogen atom.

22. The compound of claim 20, wherein A¹ is

[chemical structure]

23. The compound of claim 20, wherein A¹ is a 6-membered carbocyclic aromatic ring.

24. The compound of claim 1, wherein the compound is represented by Formula I-A1: [chemical structure]
or a pharmaceutically acceptable salt thereof; wherein:
R¹ represents independently for each occurrence halogen or C₁₋₄ alkyl;
R^{2A}, R^{2B}, and R^{2C} each represent independently hydrogen, C₁₋₄ alkyl or C₁₋₄ haloalkyl;
R³ represents independently for each occurrence hydrogen or C₁₋₄ alkyl;
R⁴ represents independently for each occurrence C₁₋₄ alkyl or C₁₋₄ haloalkyl;
R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);
A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;
Y¹ is N, C(H), or C(R¹); and
n is 0 or 1.

25. The compound of claim 24, wherein Y¹ is N.

26. The compound of claim 24, wherein Y¹ is C(H).

27. The compound of any one of claims 20-26, wherein A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

28. The compound of any one of claims 20-26, wherein A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

29. The compound of any one of claims 20-26, wherein A² is one of the following [chemical structure]
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

30. The compound of any one of claims 20-26, wherein A² is one of the following [chemical structure]
each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl).

31. The compound of any one of claims 20-26, wherein A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

32. The compound of claim 1, wherein the compound is represented by Formula I-A2: [chemical structure]
or a pharmaceutically acceptable salt thereof; wherein:
R¹ represents independently for each occurrence halogen or C₁₋₄ alkyl;
R^{2A}, R^{2B}, and R^{2C} each represent independently hydrogen, C₁₋₄ alkyl or C₁₋₄ haloalkyl;
R³ represents independently for each occurrence hydrogen or C₁₋₄ alkyl;
R⁴ represents independently for each occurrence halogen, C₁₋₄ alkyl, or C₁₋₄ haloalkyl;
R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring optionally substituted with 1 or 2 substituents independently selected from the group consisting of halogen, hydroxyl, C₁₋₄ alkyl, C₁₋₄ haloalkyl, cyano, and C₁₋₄ alkoxyl;
A² is one of the following:
5-10 membered heteroaryl, or
8-10 membered partially unsaturated bicyclic heterocyclyl;
each of which is substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵);
A³ is one of the following:
phenyl substituted by (i) —(C₁₋₄ alkylene)-X² and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, alkylene)-(C₁₋₄ alkoxyl), and C₃₋₇ cycloalkyl;
Y¹ is N, C(H), or C(R¹); and
n is 0 or 1.

33. The compound of claim 32, wherein Y¹ is N.

34. The compound of claim 32, wherein Y¹ is C(H).

35. The compound of any one of claims 32-34, wherein A² is a 5-10 membered heteroaryl substituted with 1, 2, or 3 substituents independently selected from the group consisting of halogen, C₁₋₄ alkyl, C₂₋₄ alkenyl, C₁₋₄ haloalkyl, C₁₋₄ hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

36. The compound of any one of claims 32-34, wherein A² is a 8-10 membered bicyclic heteroaryl substituted with 1, 2, or 3 substituents independently selected from the group consisting of halogen, C₁₋₄ alkyl, C₂₋₄ alkenyl, haloalkyl, hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

37. The compound of any one of claims 32-34, wherein A² is [chemical structure]
substituted with 1, 2, or 3 substituents independently selected from the group consisting of halogen, C₁₋₄ alkyl, C₂₋₄ alkenyl, haloalkyl, hydroxyalkyl, C₃₋₄ hydroxyalkenyl, C₃₋₇ cycloalkyl, C₅₋₇ cycloalkenyl, and 3-7 membered heterocyclyl; wherein said C₃₋₇ cycloalkyl, C₅₋₇ cycloalkenyl, and 3-7 membered heterocyclyl are each optionally substituted with 1 or 2 substituents independently selected from the group consisting of halogen, hydroxyl, C₁₋₄ alkyl, C₁₋₄ hydroxyalkyl, —(C₁₋₄ alkylene)-N(R³)(R⁵), and —N(R³)(R⁵).

38. The compound of any one of claims 20-37, wherein A³ is phenyl substituted by (i) —(C₁₋₄ alkylene)-X², and (ii) 1 or 2 occurrences of R⁴.

39. The compound of any one of claims 20-31, wherein A³ is phenyl substituted by (i) —O—X², and (ii) 1 occurrence of R⁴.

40. The compound of any one of claims 20-37, wherein A³ is 5-6 membered heteroaryl substituted by (i) —(C₁₋₄ alkylene)-X² or —O—X², and (ii) 1 occurrence of R⁴.

41. The compound of any one of claims 20-37, wherein A³ is 6-membered heteroaryl substituted by (i) —(C₁₋₄ alkylene)-X² or —O—X², and (ii) 1 occurrence of R⁴.

42. The compound of any one of claims 20-37, wherein A³ is pyridinyl, pyrazinyl, or pyrimidinyl; each of which is substituted by (i) —(C₁₋₄ alkylene)-X² or —O—X², and (ii) 1 occurrence of R⁴.

43. The compound of any one of claims 20-37, wherein A³ is pyridinyl substituted by (i) —(C₁₋₄ alkylene)-X² or —O—X², and (ii) 1 occurrence of R⁴.

44. The compound of any one of claims 20-43, wherein R^{2A} is C₁₋₄ alkyl; and R^{5C} is hydrogen.

45. The compound of any one of claims 20-43, wherein R^{2A} is ethyl; and R^{5C} is hydrogen.

46. The compound of any one of claims 20-43, wherein R^{2A} is methyl; and R^{5C} is hydrogen.

47. The compound of any one of claims 20-46, wherein R^{2B} is hydrogen.

48. The compound of any one of claims 20-46, wherein R^{2B} is C₁₋₄ alkyl.

49. The compound of any one of claims 1-48, wherein X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl.

50. The compound of any one of claims 1-48, wherein X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl.

51. The compound of any one of claims 1-48, wherein X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, C₁₋₄ hydroxyalkyl, and C₃₋₇ cycloalkyl.

52. The compound of any one of claims 1-48, wherein X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl.

53. The compound of any one of claims 1-48, wherein X² is piperidinyl or piperazinyl; each of which is optionally substituted with 1 or 2 substituents independently selected from the group consisting of C₁₋₄ alkyl and C₃₋₇ cycloalkyl.

54. The compound of any one of claims 1-53, wherein R³ is hydrogen.

55. The compound of any one of claims 1-54, wherein R⁴ is C₁₋₄ haloalkyl.

56. The compound of any one of claims 1-54, wherein R⁴ is trifluoromethyl.

57. The compound of any one of claims 1-56, wherein R⁵ is hydrogen, C₁₋₄ alkyl, C₁₋₄ haloalkyl, C₁₋₄ hydroxyalkyl, or —(C₁₋₄ alkylene)-(C₁₋₄ alkoxyl).

58. The compound of any one of claims 1-57, wherein n is O.

59. The compound of any one of claims 1-57, wherein n is 1.

60. A compound represented by Formula II: [chemical structure]
or a pharmaceutically acceptable salt thereof; wherein:
R¹ represents independently for each occurrence halogen, C₁₋₄ alkyl, C₁₋₄ haloalkyl, cyano, C₁₋₄ alkoxyl, C₃₋₇ cycloalkyl, 3-7 membered heterocycloalkyl, or —N(R³)(R⁵);
R² represents independently for each occurrence hydrogen, halogen, hydroxyl, C₁₋₄ alkyl, C₁₋₄ haloalkyl, C₁₋₄ hydroxyalkyl, cyano, C₁₋₄ alkoxyl, —(C₁₋₄ alkylene)-(C₁₋₄ alkoxyl), C₃₋₇ cycloalkyl, or 3-7 membered heterocycloalkyl; or two occurrences of R² attached to the same carbon atom are taken together to represent an oxo group; or two occurrences of R² are taken together with the carbon atom or carbon atoms to which they are attached to form a 3-6 membered saturated ring;
R³ represents independently for each occurrence hydrogen or C₁₋₄ alkyl;
R⁴ represents independently for each occurrence halogen, C₁₋₄ alkyl, C₁₋₄ haloalkyl, cyano, C₁₋₄ alkoxyl, C₃₋₅ cycloalkyl, C₃₋₅ halocycloalkyl, —(C₃₋₅ cycloalkylene)-(C₁₋₄ haloalkyl), or —(C₃₋₅ cycloalkylene)-CN
R⁵ is hydrogen, C₁₋₄ alkyl, C₁₋₄ haloalkyl, C₁₋₄ hydroxyalkyl, alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;
A¹ is a 5-6 membered heteroaromatic ring containing at least one ring nitrogen atom, a 6-membered unsaturated oxo-heterocyclic ring containing at least one ring nitrogen atom, or a 6-membered carbocyclic aromatic ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵;
A³ is one of the following:
phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴;
5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴; or
naphthalenyl, quinolinyl, or a 9-10 membered partially unsaturated bicyclic aza-heterocyclyl; each of which is substituted by (i) X², —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 0, 1, 2, or 3 occurrences of R⁴;
X¹ is —O—, —N(R³)—, or —S(O)ₜ—;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, oxo, cyano, C₁₋₄ alkoxyl, —(C₁₋₄ alkylene)-(C₁₋₄ alkoxyl), C₃₋₇ cycloalkyl, 3-7 membered heterocycloalkyl, and 5-6 membered heteroaryl;
Y¹ is —C(R³)₂— or —N(R³)—; and
m and n are each independently 0, 1, 2, or 3; and
t is 0, 1 or 2;
provided that if m is 0 and Y¹ is —C(R³)₂—, then A² is not a 6-membered heteroaryl substituted with —C(O)N(R³)(R⁵), —N(R³)(R⁵), or —N(R³)C(O)R⁵.

61. The compound of claim 60, wherein A¹ is a 5-6 membered heteroaromatic ring containing at least one ring nitrogen atom.

62. The compound of claim 60, wherein A¹ is

[chemical structure]

63. The compound of claim 60, wherein A¹ is a 6-membered unsaturated oxo-heterocyclic ring containing at least one ring nitrogen atom.

64. The compound of claim 60, wherein A¹ is a 6-membered carbocyclic aromatic ring.

65. The compound of any one of claims 60-64, wherein A² is a 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

66. The compound of any one of claims 60-64, wherein A² is a 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

67. The compound of any one of claims 60-64, wherein A² is one of the following [chemical structure]
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, and —(C₁₋₄ alkylene)-(C₁₋₄ alkoxyl).

68. The compound of any one of claims 60-64, wherein A² is a 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), —N(R³)(R⁵), —N(R³)C(O)R⁵, —(C₁₋₄ alkylene)-N(R³)(R⁵), —(C₁₋₄ alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

69. The compound of any one of claims 60-64, wherein A² is 8-10 membered bicyclic oxo-heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, alkylene)-(C₁₋₄ alkoxyl), —C(O)N(R³)(R⁵), —N(R³)(R⁵), —N(R³)C(O)R⁵, alkylene)-N(R³)(R⁵), alkylene)-N(R³)C(O)R⁵, —N(R³)—(5-6 membered heteroaryl), 3-7 membered heterocycloalkyl, 5-6 membered heteroaryl, and aryl; wherein said C₃₋₇ cycloalkyl, 3-7 membered heterocycloalkyl, 5-6 membered heteroaryl, and aryl are each optionally substituted with 1 or 2 substituents independently selected from the group consisting of halogen, hydroxyl, C₁₋₄ alkyl, C₁₋₄ haloalkyl, cyano, —N(R³)(R⁵), and —C(O)N(R³)R⁵.

70. The compound of any one of claims 60-69, wherein A³ is phenyl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.

71. The compound of any one of claims 60-69, wherein A³ is 5-6 membered heteroaryl substituted by (i) —(C₁₋₄ alkylene)-X², —N(R³)—X², —O—X², —O-(5-6 membered heteroaryl), —O—(C₁₋₄ alkylene)-N(R³)₂, or —O—(C₁₋₄ alkylene)-(5-6 membered heteroaryl), and (ii) 1, 2, or 3 occurrences of R⁴.

72. The compound of any one of claims 60-71, wherein X¹ is —O—.

73. The compound of any one of claims 60-71, wherein X¹ is —N(R³)— or —S—.

74. The compound of any one of claims 60-73, wherein X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, and hydroxyl.

75. The compound of any one of claims 60-74, wherein Y¹ is —C(R³)₂—.

76. The compound of any one of claims 60-74, wherein Y¹ is —N(R³)—.

77. The compound of any one of claims 60-76, wherein R² is C₁₋₄ alkyl, and m is 1 or 2

78. The compound of claim 60, wherein the compound is represented by Formula II-A: [chemical structure]
or a pharmaceutically acceptable salt thereof; wherein:
R¹ represents independently for each occurrence halogen or C₁₋₄ alkyl;
R^{2A} and R^{2B} each represent independently hydrogen, C₁₋₄ alkyl, C₁₋₄ haloalkyl, C₁₋₄ hydroxyalkyl, or —(C₁₋₄ alkylene)-(C₁₋₄ alkoxyl);
R³ represents independently for each occurrence hydrogen or C₁₋₄ alkyl;
R⁴ represents independently for each occurrence C₁₋₄ alkyl, C₁₋₄ haloalkyl, C₃₋₅ cycloalkyl, C₃₋₅ halocycloalkyl, -(cyclopropylene)-(C₁₋₄ haloalkyl), or -(cyclopropylene)-CN;
R⁵ is hydrogen, C₁₋₄ alkyl, C₁₋₄ haloalkyl, C₁₋₄ hydroxyalkyl, alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;
A¹ is a 6-membered heteroaromatic ring containing at least one ring nitrogen atom, or a 6-membered carbocyclic aromatic ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);
A³ is one of the following:
phenyl substituted by (i) —(C₁₋₄ alkylene)-X², —N(R³)—X², or —O—X², and (ii) 1 or 2 occurrences of R⁴; or
5-6 membered heteroaryl substituted by (i) —(C₁₋₄ alkylene)-X², —N(R³)—X², or —O—X², and (ii) 1 or 2 occurrences of R⁴;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;
Y¹ is —C(R³)₂— or —N(R³)—; and
provided that if R^{2A} and R^{2B} are both hydrogen and Y¹ is —C(R³)₂—, then A² is not a 6-membered heteroaryl substituted with —C(O)N(R³)(R⁵) or —N(R³)(R⁵).

79. The compound of claim 78, wherein A¹ is a 6-membered heteroaromatic ring containing at least one ring nitrogen atom.

80. The compound of claim 78, wherein A¹ is

[chemical structure]

81. The compound of claim 78, wherein A¹ is a 6-membered carbocyclic aromatic ring.

82. The compound of claim 60, wherein the compound is represented by Formula II-A1: [chemical structure]
or a pharmaceutically acceptable salt thereof; wherein:
R¹ represents independently for each occurrence halogen or C₁₋₄ alkyl;
R^{2A} and R^{2B} each represent independently hydrogen, C₁₋₄ alkyl or C₁₋₄ haloalkyl;
R³ represents independently for each occurrence hydrogen or C₁₋₄ alkyl;
R⁴ represents independently for each occurrence C₁₋₄ alkyl or C₁₋₄ haloalkyl;
R⁵ is hydrogen, C₁₋₄ alkyl, C₁₋₄ haloalkyl, C₁₋₄ hydroxyalkyl, —(C₁₋₄ alkylene)-(C₁₋₄ alkoxyl), or C₃₋₇ cycloalkyl; or an occurrence of R³ and R⁵ attached to the same nitrogen atom are taken together with the nitrogen atom to which they are attached to form a 3-7 membered ring;
A² is one of the following:
5-10 membered heteroaryl,
8-10 membered partially unsaturated bicyclic heterocyclyl, or
8-10 membered bicyclic oxo-heterocyclyl;
each of which is optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, cyano, C₁₋₄ alkoxyl, —(C₁₋₄ alkylene)-(C₁₋₄ alkoxyl), —C(O)N(R³)(R⁵), and —N(R³)(R⁵);
A³ is one of the following:
phenyl substituted by (i) —(C₁₋₄ alkylene)-X², —N(R³)—X², or —O—X², and (ii) 1 or 2 occurrences of R⁴;
5-6 membered heteroaryl substituted by (i) —(C₁₋₄ alkylene)-X², —N(R³)—X², or —O—X², and (ii) 1 or 2 occurrences of R⁴;
X² is a 3-10 membered aza-heterocycloalkyl optionally substituted with 1, 2, or 3 substituents independently selected from the group consisting of halogen, C₁₋₄ alkyl, C₁₋₄ haloalkyl, C₁₋₄ hydroxyalkyl, hydroxyl, cyano, C₁₋₄ alkoxyl, —(C₁₋₄ alkylene)-(C₁₋₄ alkoxyl), and C₃₋₇ cycloalkyl;
Y¹ is —C(R³)₂— or —N(R³)—;
Y² is N, C(H), or C(R¹); and
n is 0, 1, or 2;
provided that if R^{2A} and R^{2B} are both hydrogen and Y¹ is —C(R³)₂—, then A² is not a 6-membered heteroaryl substituted with —C(O)N(R³)(R⁵) or —N(R³)(R⁵).

83. The compound of any one of claims 78-82, wherein A² is 5-10 membered heteroaryl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

84. The compound of any one of claims 78-82, wherein A² is 8-10 membered bicyclic heteroaryl optionally substituted with 1 or 2 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

85. The compound of any one of claims 78-82, wherein A² is one of the following [chemical structure]
each of which is optionally substituted with 1, 2, or 3 substituents independently selected from the group consisting of halogen, hydroxyl, C₁₋₄ alkyl, C₁₋₄ haloalkyl, hydroxyalkyl, C₃₋₇ cycloalkyl, and cyano.

86. The compound of any one of claims 78-82, wherein A² is one of the following [chemical structure]
each of which is optionally substituted with 1 or 2 substituents independently selected from the group consisting of C₁₋₄ alkyl, C₁₋₄ haloalkyl, C₃₋₇ cycloalkyl, cyano, C₁₋₄ hydroxyalkyl, and —(C₁₋₄ alkylene)-(C₁₋₄ alkoxyl).

87. The compound of any one of claims 78-82, wherein A² is 8-10 membered partially unsaturated bicyclic heterocyclyl optionally substituted with 1, 2, or 3 substituents independently selected from the group consisting of halogen, hydroxyl, C₁₋₄ alkyl, C₁₋₄ haloalkyl, C₁₋₄ hydroxyalkyl, C₃₋₇ cycloalkyl, and cyano.

88. The compound of any one of claims 78-87, wherein A³ is phenyl substituted by (i) —(C₁₋₄ alkylene)-X² or —O—X², and (ii) 1 occurrence of R⁴.

89. The compound of any one of claims 78-87, wherein A³ is 5-6 membered heteroaryl substituted by (i) —(C₁₋₄ alkylene)-X² or —O—X², and (ii) 1 occurrence of R⁴.

90. The compound of any one of claims 78-87, wherein A³ is 6-membered heteroaryl substituted by (i) —(C₁₋₄ alkylene)-X² or —O—X², and (ii) 1 occurrence of R⁴.

91. The compound of any one of claims 78-87, wherein A³ is pyridinyl, pyrazinyl, or pyrimidinyl; each of which is substituted by (i) —(C₁₋₄ alkylene)-X² or —O—X², and (ii) 1 occurrence of R⁴.

92. The compound of any one of claims 78-87, wherein A³ is pyridinyl substituted by (i) —(C₁₋₄ alkylene)-X² or —O—X², and (ii) 1 occurrence of R⁴.

93. The compound of any one of claims 78-92, wherein X² is a 3-10 membered aza-heterocycloalkyl optionally substituted with 1, 2, or 3 substituents independently selected from the group consisting of halogen, C₁₋₄ alkyl, C₁₋₄ haloalkyl, and C₃₋₇ cycloalkyl.

94. The compound of any one of claims 78-92, wherein X² is a 3-7 membered aza-heterocycloalkyl substituted with 1 or 2 substituents independently selected from the group consisting of C₁₋₄ alkyl, C₁₋₄ haloalkyl, and C₃₋₇ cycloalkyl.

95. The compound of any one of claims 78-92, wherein X² is piperidinyl, piperazinyl, morpholinyl, or pyrrolidinyl; each of which is optionally substituted with 1, 2, or 3 substituents independently selected from the group consisting of halogen, C₁₋₄ alkyl, C₁₋₄ haloalkyl, and C₃₋₇ cycloalkyl.

96. The compound of any one of claims 78-92, wherein X² is piperidinyl or piperazinyl; each of which is optionally substituted with 1 or 2 substituents independently selected from the group consisting of C₁₋₄ alkyl and C₃₋₇ cycloalkyl.

97. The compound of any one of claims 78-96, wherein R^{2A} is C₁₋₄ alkyl.

98. The compound of any one of claims 78-96, wherein R^{2A} is ethyl.

99. The compound of any one of claims 78-96, wherein R^{2A} is methyl.

100. The compound of any one of claims 78-99, wherein R^{2B} is hydrogen.

101. The compound of any one of claims 78-99, wherein R^{2B} is C₁₋₄ alkyl.

102. The compound of any one of claims 60-101, wherein R¹ is C₁₋₄ alkyl.

103. The compound of any one of claims 60-102, wherein R⁴ is trifluoromethyl.

104. The compound of any one of claims 60-103, wherein R³ is hydrogen, and R⁵ is hydrogen, C₁₋₄ alkyl, C₁₋₄ haloalkyl, or C₁₋₄ hydroxyalkyl.

105. The compound of any one of claims 60-104, wherein n is 0.

106. The compound of any one of claims 60-104, wherein n is 1.

107. The compound of any one of claims 60-106, wherein Y¹ is —C(R³)₂—.

108. The compound of any one of claims 60-106, wherein Y¹ is —N(R³)—.

109. A compound in any one of Tables 1-10 and 19 herein, or a pharmaceutically acceptable salt thereof.

110. A compound in any one of Tables 1A, 11-18, and 20 herein, or a pharmaceutically acceptable salt thereof.

111. A pharmaceutical composition comprising a compound of any one of claims 1-110 and a pharmaceutically acceptable carrier.

112. A method of treating cancer in a subject, comprising administering a therapeutically effective amount of a compound of any one of claims 1-110 to a subject in need thereof to treat the cancer.

113. The method of claim 112, wherein the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma.

114. The method of claim 112, wherein the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma.

115. The method of any one of claims 112-114, wherein the subject is a human.

116. A method of inhibiting the activity of HPK1, comprising exposing a HPK1 to an effective amount of a compound of any one of claims 1-110 to inhibit the activity of said HPK1.
